(19) United States
(12) Patent Application Publication
(10) Pub. No.: US 2025/0388620 A1
(43) Pub. Date: Dec. 25, 2025

(54) EFFICIENT METHOD FOR MAKING HIGHLY PURIFIED 5’- CAPPED OLIGONUCLEOTIDES

(71) Applicant: TriLink BioTechnologies, LLC, San Diego, CA (US)

(72) Inventors: Chanfeng Zhao, Rancho Santa Fe, CA (US); Tao Tom Jiang, Rancho Santa Fe, CA (US); Hengyuan Lang, San Diego, CA (US); Paul Theodore Ludford, III, San Diego, CA (US); Chunping Xu, San Diego, CA (US)

(73) Assignee: TriLink BioTechnologies, LLC, San Diego, CA (US)

(21) Appl. No.: 19/062,789

(22) Filed: Feb. 25, 2025

Related U.S. Application Data
(63) Continuation of application No. PCT/US23/72901, filed on Aug. 25, 2023.
(60) Provisional application No. 63/401,544, filed on Aug. 26, 2022.
(60) Provisional application No. 63/414,361, filed on Oct. 7, 2022.
(60) Provisional application No. 63/430,987, filed on Dec. 7, 2022.

(51) Int. Cl. C07H 21/04; C12N 15/11

(52) U.S. Cl. CPC C07H 21/04; C12N 15/11; C12N 2310/317; C12N 2310/344

(57) ABSTRACT

Described herein are highly pure, chemically synthesized, stabilized, 5′-capped oligonucleotides. Additionally, described herein are methods for making and using said oligonucleotides.

### CROSS-REFERENCE TO RELATED APPLICATIONS

This application is a continuation of International Application No. PCT/US2023/072901, filed Aug. 25, 2023, which claims priority to U.S. provisional application No. 63/401,544, filed on Aug. 26, 2022, to U.S. provisional application No. 63/414,361, filed on Oct. 7, 2022, and to U.S. provisional application No. 63/430,987, filed on Dec. 7, 2022, the disclosures of all of which are incorporated herein by reference in their entireties.

### SEQUENCE LISTING

This application contains a Sequence Listing which has been submitted electronically in XML format and is hereby incorporated by reference in its entirety. Said XML copy, created on Aug. 23, 2023, is named “2023 Aug. 23-01355-0001-00PCT-ST26” and is 20,115 bytes in size.

### TECHNICAL FIELD

The field of this invention relates to highly purified, chemically synthesized, stabilized 5′-capped oligonucleotides and the methods of making and using said 5′-capped oligonucleotides.

### BACKGROUND

In vitro transcribed messenger RNAs (mRNAs) have numerous in vivo applications, such as vaccination, where mRNA encoding specific antigen(s) is administered to elicit protective immunity in a patient; cell therapy, where mRNA is transfected into cells ex vivo to alter cell phenotype or function prior to delivery of these altered cells to a patient; or replacement therapy, where mRNA encoding a therapeutic protein is administered to the patient.

A primary structural element of an mRNA molecule that is utilized in in vivo applications includes a Cap structure on the 5′-end of the mRNA. Naturally occurring Cap structures include a 7-methylguanosine (^{7m}G or ^{m7}G) linked through a 5′- to 5′-triphosphate chain at the 5′-end of the mRNA molecule. The Cap must be present for the mRNA to retain template activity for protein synthesis. The chemical structure of the Cap can drive translation efficiency in a cell. Therefore, effective Cap structures are necessary.

Traditionally, 5′-capped mRNAs have been prepared using enzymatic methods. Non-enzymatic methods of making mRNAs can potentially be more cost effective and more amenable to scale-up. However, there are technical challenges in making high purity, stable, synthetic mRNA, especially making 5′-caped-mRNA 40 or longer bases

Accordingly, there exists a need to develop robust non-enzymatic methods of preparing stable 5′-capped mRNAs with high purity levels that are amenable to scale-up.

### SUMMARY

Described herein are methods for making highly pure, chemically synthesized, stabilized oligonucleotides comprising cap analogs on their 5′ end. Furthermore, described herein are highly pure, chemically synthesized, stabilized oligonucleotides comprising cap analogs on their 5′ end, which can be produced by the disclosed methods.

In one aspect, the disclosure provides an efficient method of making a 5′-capped oligonucleotide comprising reacting a 5′-phosphate-oligonucleotide with a modified Im-m⁷GDP that includes a cleavable hydrophobic moiety that can be removed following the reaction. For instance, the hydrophobic group can be removed following purification of the product generated from the reaction of the 5′-phosphate-oligonucleotide with a modified Im-m⁷GDP. Alternatively, the disclosure provides an efficient method of making a 5′-capped oligonucleotide comprising reacting a 5′-phosphate-oligonucleotide with a modified Im-m⁷GDP that includes a non-cleavable hydrophobic moiety, yet the 5′-capped oligonucleotide allows for an efficient translation.

The disclosure further provides modified Im-m⁷GDP compounds that can react with 5′-phosphate-oligonucleotide molecules to produce 5′-capped oligonucleotide molecules. In embodiments, the Im-m⁷GDP is modified with either removable or non-removable hydrophobic group(s) at the 2′ and/or 3′ and/or N2, and/or N7 position (in any combination). In embodiments, the 2′-position and/or 3′-position of the Im-m⁷GDP is modified with a removable hydrophobic group. In embodiments, the 2′-position and/or 3′-position of the Im-m⁷GDP is modified with a non-removable hydrophobic group. In embodiments, the N7-methylated GDP moiety of the Im-m⁷GDP is modified with a removable hydrophobic group. In embodiments, the N7-methylated GDP moiety of the Im-m⁷GDP is modified with a non-removable hydrophobic group. In embodiments, both the 2′-position and the N7-methylated GDP moiety of the Im-m⁷GDP are modified with removable hydrophobic groups. In embodiments, both the 2′-position and the N7-methylated GDP moiety of the Im-m⁷GDP are modified with non-removable hydrophobic groups. In embodiments, both the 3′-position and the N7-methylated GDP moiety of the Im-m⁷GDP are modified with removable hydrophobic groups. In embodiments, both the 3′-position and the N7-methylated GDP moiety of the Im-m⁷GDP are modified with non-removable hydrophobic groups.

As disclosed herein, following reaction of the modified Im-m⁷GDP compounds with 5′-phosphate-oligonucleotide molecules, 5′-capped oligonucleotide with one or more removable or non-removable hydrophobic groups are generated. The 5′-capped oligonucleotide molecule bearing the one or more hydrophobic groups can then be readily separated from impurities in the reaction mixture, including uncapped oligonucleotide molecules. In embodiments, the hydrophobic group or groups may be chemically removed from the purified 5′-capped oligonucleotide generating highly pure and readily translatable 5′-capped oligonucleotide. In other embodiments, the hydrophobic group or groups may remain on the 5′-capped oligonucleotide generating highly pure and readily translatable 5′-capped oligonucleotide.

In an aspect, provided herein is an oligonucleotide comprising 50-12000 nucleotides, whose 5′ end comprises a structure of formula (I)

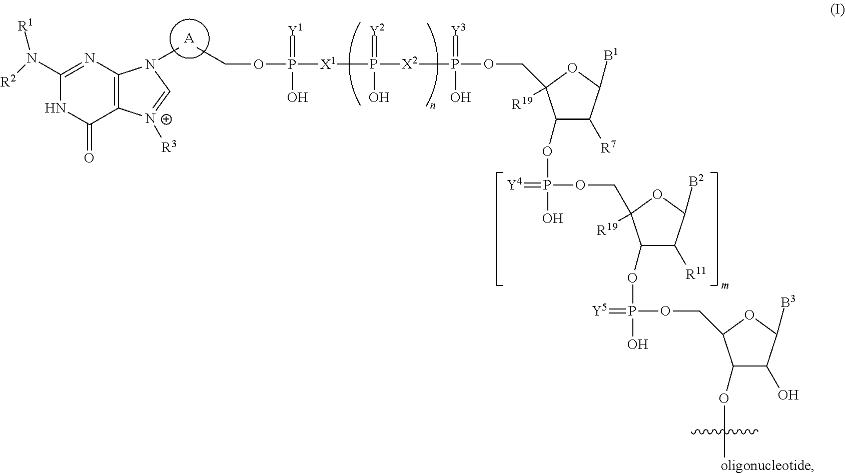

or an enantiomer, a mixture of enantiomers, a mixture of two or more diastereomers, a tautomer, a mixture of two or more tautomers, or an isotopic variant thereof, or a pharmaceutically acceptable salt, solvate, or hydrate thereof;
wherein:
   B¹ and B³ are each independently a natural, modified, or unnatural nucleoside base;
   each B² is independently a natural, modified, or unnatural nucleoside base;
   Ring A is a substituted or unsubstituted cycloalkylene, substituted or unsubstituted heterocycloalkylene, substituted or unsubstituted arylene, or substituted or unsubstituted heteroarylene;
   X¹ and X² are each independently —O—, —CH₂—, —CX₂—, —N(R¹⁰¹)—, —BH—, or —S—;
   Y¹, Y², Y³, Y⁴, and Y⁵ are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R⁷ is independently hydrogen,
      halogen, —CCl₃, —CBr₃, —CF₃, —CI₃, —CHCl₂, —CHBr₂,
      —CHF₂, —CHI₂, —CH₂Cl, —CH₂Br, —CH₂F, —CH₂I, —CN, —OR^{7A}, —NR^{7A}R^{7B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁹ is independently hydrogen,
   halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{19A}, —NR^{19A}R^{19B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁷ and R¹⁹ together with the carbon atoms to which they are connected form a substituted or unsubstituted cycloalkylene or substituted or unsubstituted heterocycloalkylene;
each R¹¹ is independently hydrogen,
   halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{11A}, —NR^{11A}R^{11B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R¹¹ and R¹⁹ together with the carbon atoms to which they are connected form a substituted or unsubstituted cycloalkylene or substituted or unsubstituted heterocycloalkylene;
each R^{1A}, R^{2A}, R^{3A}, R^{7A}, R^{7B}, R^{11A}, R^{11B}, R^{19A}, and R^{19B} is independently hydrogen, —CX₃,
   —CHX₂, —CH₂X, —C(O)OH, —C(O)NH₂, —CN, —OH, —NH₂, —COOH, —CONH₂, —NO₂, —SH, —SO₃H,
   —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R^{7A} and R^{7B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
m is an integer from 0 to 8;
n is an integer from 0 to 3; and
each X is independently —Cl, —Br, —I or —F.
   Here and throughout the disclosure,

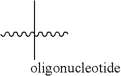

indicates the point of attachment of a structure to the remainder (e.g., body and 3′ end) of the oligonucleotide.

In an aspect, provided herein is an oligonucleotide comprising 50-12000 nucleotides, whose 5′ end comprises a structure of formula (II):

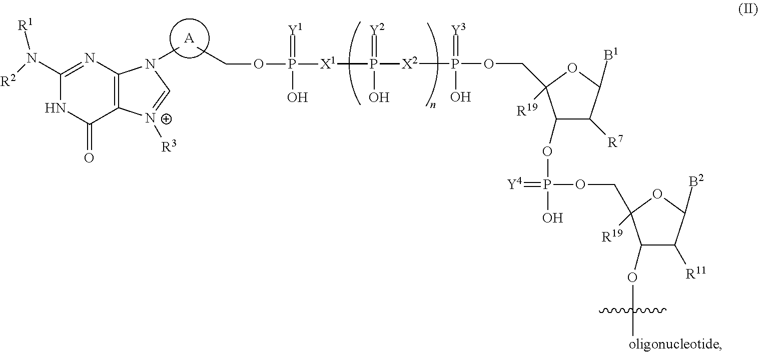

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   B¹ and B² are each independently a natural, modified, or unnatural nucleoside base;
   Ring A is a substituted or unsubstituted cycloalkylene, substituted or unsubstituted heterocycloalkylene, substituted or unsubstituted arylene, or substituted or unsubstituted heteroarylene;
   X¹ and X² are each independently —O—, —CH₂—, —CX₂—, —N(R¹⁰¹)—, —BH—, or —S—;
   Y¹, Y², Y³, and Y⁴ are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁷ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR⁷⁴, —NR^{7A}R^{7B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R¹⁹ is independently hydrogen,
      halogen, —CCl₃, —CBr₃, —CF₃, —CI₃, —CHCl₂, —CHBr₂,
      —CHF₂, —CHI₂, —CH₂Cl, —CH₂Br, —CH₂F, —CH₂I, —CN, —OR^{19A}, —NR^{19A}R^{19B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁷ and R¹⁹ together with the carbon atoms to which they are connected form a substituted or unsubstituted cycloalkylene or substituted or unsubstituted heterocycloalkylene;
each R¹¹ is independently hydrogen,
   halogen, —CCl₃, —CBr₃, —CF₃, —CI₃, —CHCl₂, —CHBr₂,
   —CHF₂, —CHI₂, —
   CH₂Cl, —CH₂Br, —CH₂F, —CH₂I, —CN, —OR^{11A}, —NR^{11A}R^{11B}, —COOH, —CONH₂,
   —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R¹¹ and R¹⁹ together with the carbon atoms to which they are connected form a substituted or unsubstituted cycloalkylene or substituted or unsubstituted heterocycloalkylene;
each R^{1A}, R^{2A}, R^{3A}, R^{7A}, R^{7B}, R^{11A}, R^{11B}, R^{19A}, and R^{19B} is independently
   hydrogen, —CX₃,
   —CHX₂, —CH₂X, —C(O)OH, —C(O)NH₂, —CN, —OH, —NH₂, —COOH, —CONH₂, —NO₂, —SH, —SO₃H,
   —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R^{7A} and R^{7B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer from 0 to 3; and
each X is independently —Cl, —Br, —I or —F;
with the proviso that R¹¹ is not OH.

The disclosed methods can also be used to prepare translatable 5′-capped mRNA molecules with site-specific modifications in the 5′-capped mRNA molecules. These site-specific modifications can potentially increase translation activity and reduce immunogenicity. In embodiments, provided herein is an oligonucleotide as described herein, wherein the oligonucleotide is prepared via chemical synthesis. In embodiments, provided herein is an oligonucleotide as described herein, wherein the oligonucleotide comprises 3 or more modified nucleosides and 2 or more nucleotides that are linked together by a modified internucleotide linkage, at its 3′ end. In embodiments, provided herein is an oligonucleotide as described herein, wherein the oligonucleotide comprises 3 or more modified nucleosides at its 3′ end. In embodiments, provided herein is an oligonucleotide as described herein, wherein the oligonucleotide comprises 2 or more nucleotides that are linked together by a modified internucleotide linkage at its 3′ end. In embodiments, provided herein is an oligonucleotide as described herein, wherein the structure of formula (I) or formula (II) comprises one or more removable hydrophobic group(s). In embodiments, provided herein is an oligonucleotide as described herein, wherein the structure of formula (I) or formula (II) comprises one or more non-removable hydrophobic group(s). In embodiments, provided herein is an oligonucleotide as described herein, wherein the pharmaceutically acceptable salt is a sodium salt, a lithium salt, or a potassium salt. In embodiments, provided herein is an oligonucleotide as described herein, wherein the pharmaceutically acceptable salt is a sodium salt.

In embodiments, R¹, R², and R³ are each independently a removable hydrophobic group. In embodiments, R¹ is independently a removable hydrophobic group. In embodiments, R² is independently a removable hydrophobic group. In embodiments, R³ is independently a removable hydrophobic group.

In an aspect, provided herein is an oligonucleotide whose 5′ end comprises a compound of the following structure:

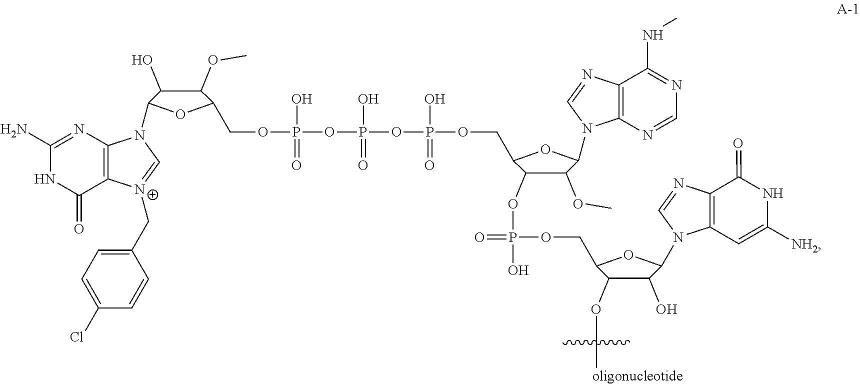

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof.

In an aspect, provided herein is an oligonucleotide whose 5′ end comprises a compound of the following structure:

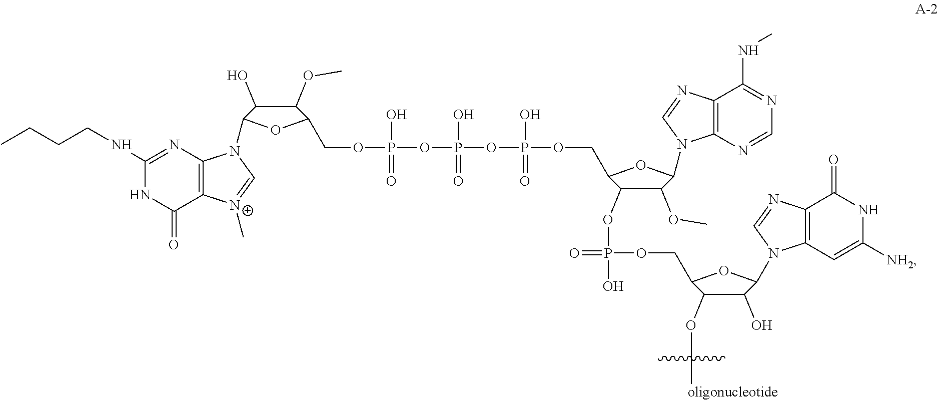

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof.

In an aspect, provided herein is an oligonucleotide whose 5′ end comprises a compound of the following structure:

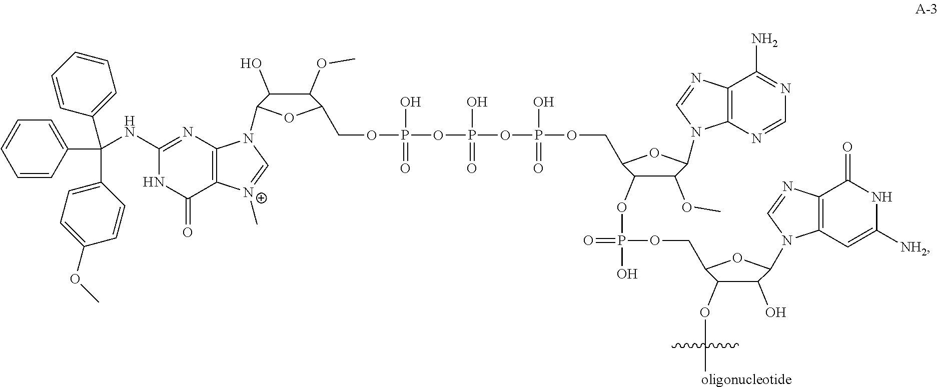

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof.

In an aspect, provided herein is a protected 5′-capped oligonucleotide as described herein, which is prepared by reaction of a structure of formula (III):

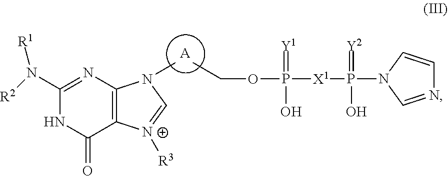

or an enantiomer, a mixture of enantiomers, a mixture of two or more diastereomers, a tautomer, a mixture of two or more tautomers, or an isotopic variant thereof, or a pharmaceutically acceptable salt, solvate, or hydrate thereof,
wherein:
   Ring A is a substituted or unsubstituted cycloalkylene, substituted or unsubstituted heterocycloalkylene, substituted or unsubstituted arylene, or substituted or unsubstituted heteroarylene;
   X¹ is independently —O—, —CH₂—, —CX₂—, —N(R¹⁰¹)—, —BH—, or —S—;
   Y¹ and Y² are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR³⁴, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R^{1A}, R^{2A}, and R^{3A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
      —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each X is independently —Cl, —Br, —I or —F;
   with a 5′-phosphate-oligonucleotide

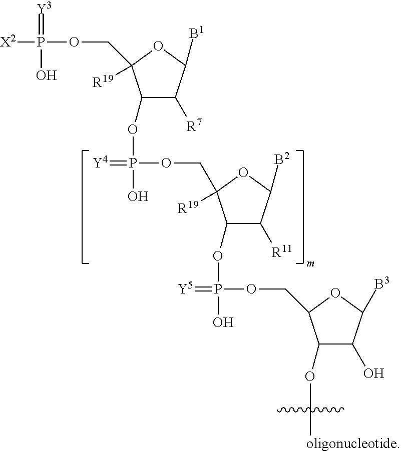

In embodiments, provided herein is a protected 5′-capped oligonucleotide described herein is prepared via chemical synthesis. In embodiments, provided herein is a 5′-capped oligonucleotide prepared by removing the protecting group(s) of the protected 5′-capped oligonucleotide described herein. In embodiments, the 5′-capped oligonucleotide described herein is substantially free of enzymatic byproducts.

In an aspect, provided herein is a composition comprising the chemically synthesized oligonucleotide as described herein, wherein the composition comprises less than 1%, less than 0.5%, less than 0.25%, less than 0.1%, or less than 0.05% by weight of oligonucleotide whose 5′ end does not comprise a structure of formula (I) or formula (II). In embodiments, provided herein is a composition comprising the chemically synthesized oligonucleotide as described herein, wherein the composition is substantially free of oligonucleotide whose 5′ end does not comprise a structure of formula (I) or formula (II). In embodiments, provided herein is a composition comprising the chemically synthesized oligonucleotide as described herein, wherein the composition is substantially free of enzymatic byproducts.

In an aspect, provided herein is a composition comprising (a) more than 90% of 5′-capped oligonucleotide without protecting group(s); (b) less than 10% of 5′-capped oligonucleotide with protecting group(s); and optionally (c) less than 1% of oligonucleotide whose 5′ end does not comprise a structure of formula (I) or formula (II). In an aspect, provided herein is a composition comprising (a) more than 95% of 5′-capped oligonucleotide without protecting group(s); (b) less than 5% of 5′-capped oligonucleotide with protecting group(s); and optionally (c) less than 1% of oligonucleotide whose 5′ end does not comprise a structure of formula (I) or formula (II).

In an aspect, provided herein is a process for preparing an oligonucleotide comprising 50-12000 nucleotides, whose 5′ end comprises a structure of formula (I):

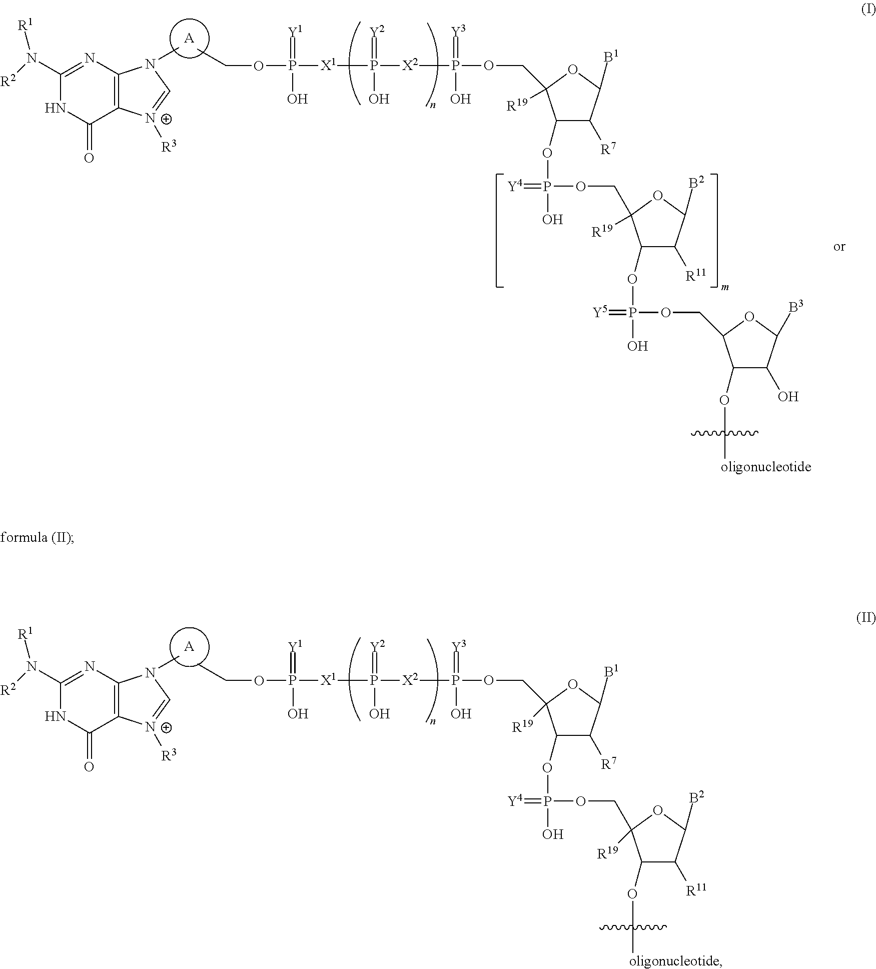

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
comprising (a) reacting an imidazolide of formula (III)

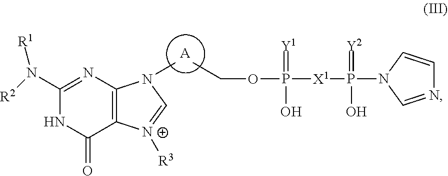

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   Ring A is a substituted or unsubstituted cycloalkylene, substituted or unsubstituted heterocycloalkylene, substituted or unsubstituted arylene, or substituted or unsubstituted heteroarylene;
   X¹ is independently —O—, —CH₂—, —CX₂—, —N(R¹⁰¹)—, —BH—, or —S—;
   Y¹ and Y² are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R^{1A}, R^{2A}, and R^{3A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
      —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each X is independently —Cl, —Br, —I or —F;
with a 5′-phosphate-oligonucleotide

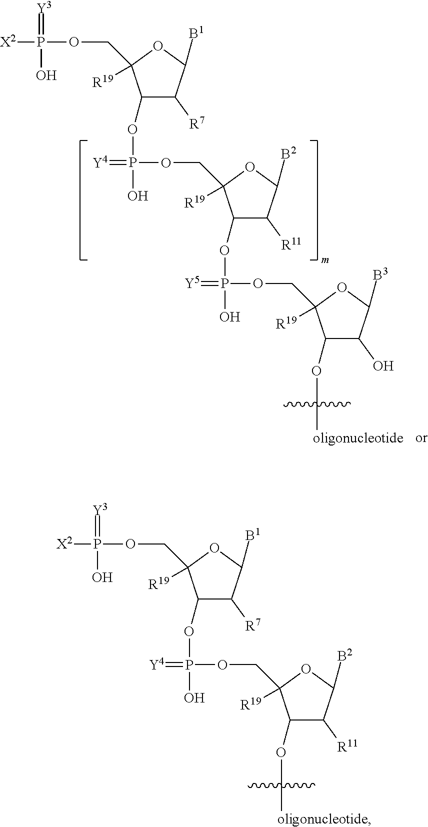

and (b) optionally removing the removable hydrophobic group(s).

In another aspect, the disclosure provides hybrid mRNAs produced using enzymatic or chemical ligation methods as disclosed herein. In some embodiments, a 5′-capped mRNA produced in accordance with the methods of the disclosure, can be ligated to another RNA molecule to increase the size (i.e., the number of nucleotide bases) of the mRNA. In some embodiments, a 5′-capped mRNA produced in accordance with the methods of the disclosure can be ligated to another RNA molecule comprising at least about 500 nucleotide bases to no more than 12,000 nucleotide bases. In some embodiments, a 5′-capped mRNA produced in accordance with the methods of the disclosure can be ligated to another RNA molecule comprising at least about 500 nucleotide bases and no more than 8,000 nucleotide bases.

In some embodiments, the 5′-capped mRNA transcript and the second (uncapped) RNA transcript are ligated enzymatically. The RNA ligase catalyzes the formation of a 3′→5′ phosphodiester bond between the 3′-OH group on capped mRNA and the 5′-phosphate group on the second (uncapped) transcript. In some embodiments, the ligation reaction is a template-independent ligation reaction. In some embodiments, the RNA ligase is a T4 RNA ligase 1. In some embodiments, the 5′-capped mRNA transcript and the second (uncapped) transcript are ligated chemically. For instance, the 5′-capped mRNA transcript and the second (uncapped) can be ligated though a Click reaction.

In some embodiments, the ligation methods disclosed herein (e.g., RNA ligase mediated ligation or click ligation) can produce hybrid RNA transcripts that have modified (i.e., unnatural) nucleosides or modified internucleoside linkages at the 5′-end of the transcript and unmodified (i.e., naturally occurring) nucleosides that include phosphodiester linkages at the 3′-end of the transcript.

In some embodiments, the first 50 to 150 nucleotide bases (along with the 5′-cap) of a transcript are generated using synthetic methods disclosed herein and are then ligated to a longer transcript produced by in vitro transcription. The longer transcript will include naturally occurring nucleotide bases and a phosphodiester backbone. However, the 5′-capped RNA transcript produced in accordance with the disclosure can include at least one modified nucleoside and/or at least one modified internucleoside linkage.

### BRIEF DESCRIPTION OF THE DRAWINGS

FIG. 1A shows an IP-RP HPLC chromatogram of Wasabi mRNA capped with ^{m7}G_{3′OMe}ppp^{m6}A_{2′OMe}pG (A-control)—top trace. An IP-RP HPLC chromatogram of Wasabi mRNA capped with ^{N7-(4-chlorobenzyl)}G_{3′OMe}ppp^{m6}A_{2′OMe}pG (A-1)—middle trace. An IP-RP HPLC chromatogram of co-injection of Wasabi mRNA capped with ^{m7}G_{3′OMe}ppp^{m6}A_{2′OMe}pG (A-control) and Wasabi mRNA capped with ^{N7-(4-chlorobenzyl)}G_{3′OMe}ppp^{m6}A_{2′OMe}pG (A-1)—bottom trace.

FIG. 1B shows an IP-RP HPLC chromatogram of Wasabi mRNA capped with ^{m7}G_{3′OMe}ppp^{m6}A_{2′OMe}pG (A-control)—top trace. An IP-RP HPLC chromatogram of Wasabi mRNA capped with ^{m7, N2-butyl}G_{3′OMe}ppp^{m6}A_{2′OMe}pG (A-2)—middle trace. An IP-RP HPLC chromatogram of co-injection of Wasabi mRNA capped with ^{m7}G_{3′OMe}ppp^{m6}A_{2′OMe}pG (A-control) and Wasabi mRNA capped with ^{m7, N2-butyl}G_{3′OMe}ppp^{m6} A_{2′OMe}pG (A-2)—bottom trace.

FIG. 1C shows an IP-RP HPLC chromatogram of FLuc mRNA capped with ^{m7}G_{3′OMe}ppp^{m6}A_{2′OMe}pG (A-control)—top trace. An IP-RP HPLC chromatogram of FLuc mRNA capped with ^{m7, N2-butyl}G_{3′OMe}ppp^{m6}A_{2′OMe}pG (A-2)—middle trace. An IP-RP HPLC chromatogram of co-injection of FLuc mRNA capped with ^{m7}G_{3′OMe}ppp^{m6}A_{2′OMe}pG (A-control) and FLuc mRNA capped with ^{m7, N2-butyl}G_{3′OMe}ppp^{m6}A_{2′OMe}pG (A-2)—bottom trace.

FIGS. 2A-B are bar graphs showing translation of m Wasabi encoding mRNA with different cap analogs 24 hours post transfection. FIG. 2A in HEK293T cells. FIG. 2B in HeLa cells.

FIG. 3A shows an IP-RP HPLC chromatogram of 10-mer oligo (SEQ ID NO: 2) capped with compound 9 (Oligonucleotide A-5). FIG. 3B shows a LCMS chromatogram of the HPLC fractions containing the capped 10-mer oligo (where the cap comprises a removable hydrophobic group—MMT).

FIG. 4A shows an IP-RP HPLC chromatogram of 10-mer oligo (SEQ ID NO: 2) capped with compound 9 and following removal of MMT (Oligonucleotide A-6). FIG. 4B shows an LCMS chromatogram of the HPLC fractions containing the capped 10-mer oligo (where the MMT has been removed from the cap).

FIG. 5A shows an IP-RP HPLC chromatogram of 100-mer oligo (SEQ ID NO: 1) capped with compound 9 (Oligonucleotide A-3). FIG. 5B shows an LCMS chromatogram of the HPLC fractions containing the capped 100-mer oligo (where the cap comprises a removable hydrophobic group—MMT).

FIG. 6A shows an IP-RP HPLC chromatogram of modified 80-mer oligo (SEQ ID NO: 7) capped with compound 9 (Oligonucleotide A-11). FIG. 6B shows an LCMS chromatogram of the HPLC fractions containing the capped modified 80-mer oligo (where the cap comprises a removable hydrophobic group—MMT).

FIG. 7A shows an IP-RP HPLC chromatogram of modified 90-mer oligo (SEQ ID NO: 5) capped with compound 9 (Oligonucleotide A-8). FIG. 7B shows an LCMS chromatogram of the HPLC fractions containing the capped modified 90-mer oligo (where the cap comprises a removable hydrophobic group—MMT).

FIGS. 8A-C show LCMS chromatograms of crude unmodified 90-mer oligo (SEQ ID NO: 4) following digestion with RNAse at t=0 (FIG. 8A); t=2 hrs. (FIG. 8B); and t=4 hrs. (FIG. 8C).

FIGS. 9A-C show LCMS chromatograms of crude modified 90-mer oligo (SEQ ID NO: 5) following digestion with RNAse at t=0 (FIG. 9A); t=2 hrs. (FIG. 9B); and t=4 hrs. (FIG. 9C).

### DETAILED DESCRIPTION

The following description recites various examples of the present methods. No particular example is intended to define the scope of the methods. Rather, these are non-limiting, exemplary methods. The description is to be read from the perspective of one of ordinary skill in the art; therefore, information well known to the skilled artisan is not necessarily included.

As used in the specification and the appended claims, the singular forms “a,” “an,” and “the” include plural referents unless the context clearly dictates otherwise. For example, reference to “a transcript” or “the transcript” may include a plurality of transcripts.

“or” is used in the inclusive sense, i.e., equivalent to “and/or”, unless the context clearly indicates otherwise.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The use of any and all examples or exemplary language (e.g., “such as”) provided herein, is intended merely to better illustrate the invention and does not pose a limitation on the scope of the invention unless otherwise indicated.

### I. Definitions

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, P, and S where the ring is saturated or unsaturated, but not aromatic. The 3 or 4 membered ring contains 1 heteroatom selected from the group consisting of O, N, P, and S. The 5 membered ring can contain zero or one double bond and one, two or three heteroatoms selected from the group consisting of O, N, P, and S. The 6 or 7 membered ring contains zero, one or two double bonds and one, two or three heteroatoms selected from the group consisting of O, N, P, and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

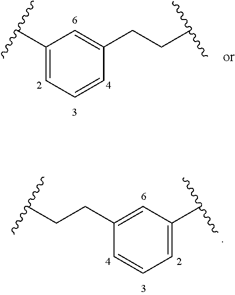 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R′″)═NR″, —NR—C(NR′R″)═NR″, —S(O)R′, —S(O)₂R′, —S(O)₂NR′R″, —NRSO₂R′, —NR′NR″R″, —ONR′R″, —NR′C(O)NR″NR″R″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R″)═NR″, —NR—C(NR′R″)═NR″, —S(O)R′, —S(O)₂R′, —S(O)₂NR′R″, —NRSO₂R′, —NR′NR″R″, —ONR′R″, —NR′C(O)NR″NR″R″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), and silicon (Si).

A “substituent group,” as used herein, means a group selected from the following moieties:
(A) oxo,
   halogen, —CCl₃, —CBr₃, —CF₃, —CI₃, —CH₂Cl, —CH₂Br, —CH₂F, —CH₂I, —CHCl₂,
   —CHBr₂, —CHF₂, —CHI₂, —CN, —OH, —NH₂, —COOH, —CONH₂, —NO₂, —SH, —SO₃H,
   —SO₄H, —SO₂NH₂, —NHNH₂, —ONH₂, —NHC(O)NHNH₂, —NHC(O)NH₂, —NHSO₂H,
   —NHC(O)H, —NHC(O)OH, —NHOH, —OCCl₃, —OCF₃, —OCBr₃, —OCI₃, —OCHCl₂,
   —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo,
   halogen, —CCl₃, —CBr₃, —CF₃, —CI₃, —CH₂Cl, —CH₂Br, —CH₂F, —CH₂I, —CHCl₂,
   —CHBr₂, —CHF₂, —CHI₂, —CN, —OH, —NH₂, —COOH, —CONH₂, —NO₂, —SH, —SO₃H,
   —SO₄H, —SO₂NH₂, —NHNH₂, —ONH₂, —NHC(O)NHNH₂, —NHC(O)NH₂, —NHSO₂H,
   —NHC(O)H, —NHC(O)OH, —NHOH, —OCCl₃, —OCF₃, —OCBr₃, —OCI₃, —OCHCl₂,
   —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₈alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo,
   halogen, —CCl₃, —CBr₃, —CF₃, —CI₃, —CH₂Cl, —CH₂Br,
   —CH₂F, —CH₂I, —CHCl₂, —CHBr₂, —CHF₂, —CHI₂, —CN, —OH, —NH₂, —COOH, —CONH₂, —NO₂, —S H,
   —SO₃H, —SO₄H, —SO₂NH₂, —NHNH₂, —ONH₂, —NHC(O)NHNH₂,
   —NHC(O)NH₂, —NHSO₂H, —NHC(O)H, —NHC(O)OH, —NHOH, —OCCl₃, —OCF₃,
   —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each nucleoside base position that contains more than one possible nucleoside base. It is specifically contemplated that each member of the Markush group should be considered separately, thereby comprising another embodiment, and the Markush group is not to be read as a single unit.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

As used herein, the term “cap analog” means a structural derivative of an RNA cap. The term “cap” refers to trinucleotide, tetranucleotide or longer structures that facilitate/improve translation and/or prevents degradation of the mRNA transcript (the oligonucleotide) when incorporated at the 5′ end of the mRNA transcript (oligonucleotide).

As used herein, the term “complement,” “complementary,” or “complementarity” refers to specific base pairing between nucleotides or nucleic acids. Complementary nucleotides are, generally, A and T (or A and U), and G and C. Complementarity, for example, between a capped oligonucleotide primer and a single stranded DNA template or one strand of dsDNA template, may be “complete” or “total” where all of the nucleotide bases of two nucleic acid strands are matched according to recognized base pairing rules, it may be “partial” in which only some of the nucleotide bases of an initiating capped oligonucleotide primer and a DNA template are matched according to recognized base pairing rules, or it may be “absent” where none of the nucleotide bases of two nucleic acid strands are matched according to recognized base pairing rules. Complementarity can also be “substantial complementarity” where the nucleotide bases of two nucleic acids are matched according to recognized base pairing rules, but include one or more mismatches (e.g., 1, 2, 3, 4) from total complementarity.

As used herein, a “deoxyribonuclease (DNase)” is an enzyme that catalyzes the hydrolytic cleavage of phosphodiester linkages in the DNA backbone, thus degrading DNA.

As used herein, the term “hybridize” or “specifically hybridize” refers to a process where initiating trinucleotide primer anneals to a DNA template under appropriately stringent conditions during a transcription reaction. Hybridizations to DNA are conducted with an initiating capped oligonucleotide primer which, in certain embodiments, is 3-10 nucleotides in length including the 5′-5′ inverted cap structure. Nucleic acid hybridization techniques are well known in the art (e.g., Sambrook, et al., Molecular Cloning: A Laboratory Manual, Second Edition, Cold Spring Harbor Press, Plainview, N.Y. (1989); Ausubel, F. M., et al., Current Protocols in Molecular Biology, John Wiley & Sons, Secaucus, N.J. (1994)).

As used herein, “locked nucleic acid” (LNA) means a ribonucleotide having a bridge between the 2′O and 4′C methylene bicyclonucleotide monomers. An LNA moiety can have the following structure:

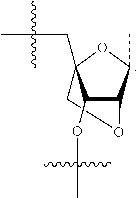

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

Ranges can be expressed herein as from one particular value, and/or to another particular value. When such a range is expressed, also specifically contemplated and considered disclosed is the range from the one particular value and/or to the other particular value unless the context specifically indicates otherwise. It should be understood that all of the individual values and sub-ranges of values contained within an explicitly disclosed range are also specifically contemplated and should be considered disclosed unless the context specifically indicates otherwise. Further, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers.

As used herein, “messenger RNA transcript,” or “mRNA transcript,” is a transcript transcribed from a DNA template encoding a desired polypeptide. The mRNA transcript may contain coding and non-coding regions. For example, the DNA template can comprise an RNA polymerase promoter sequence, a 5′ UTR sequence, an open reading frame, and a 3′ UTR sequence. In some examples, the DNA template also comprises a nucleic acid sequence encoding a poly(A) tail.

As used herein, the terms “5′-capped oligonucleotide” and “5′-capped mRNA” are used interchangeably and refer to a capped oligonucleotide that is chemically synthesized or a hybrid capped oligonucleotide composed of two or more chemically synthesized oligonucleotides which are ligated together. Alternatively, a hybrid capped oligonucleotide may be composed of two or more oligonucleotides where at least one oligonucleotide is chemically synthesized and another oligonucleotide may be produced through an in vitro transcription process, and the two oligonucleotides are then ligated. In embodiments, chemically synthesized oligonucleotides may be modified to improve their stability. For example, chemically synthesized oligonucleotides may have modified internucleotide linkages, modified sugars, or modified nucleobases as described in the specification. Various alternatives for ligation are described in the specification.

As used herein, the term “nucleoside” refers to a nitrogenous base linked to a 5-pentose sugar (e.g., ribose or deoxyribose). The term includes all nucleosides, including all forms of nucleoside bases and furanosides. Base rings include purine and pyrimidine rings. Purine rings include, for example, adenine, guanine, and N6-methyladenine. Pyrimidine rings include, for example, cytosine, thymine, 5-methylcytosine, and pseudouracil.

As used herein, the term “nucleoside triphosphate,” “nucleoside 5′ triphosphate” or “NTP” refers to a nucleoside linked to three phosphate groups. The term encompasses natural NTPs (for example, adenosine triphosphate (ATP), uridine triphosphate (UTP), guanine triphosphate (GTP), and cytosine triphosphate (CTP)) as well as modified NTPs (for example, pseudouridine and N¹-methyl-pseudouridine triphosphates).

“Inorganic pyrophosphatase” refers to an enzyme that catalyzes the conversion of one ion of pyrophosphate to two phosphate ions, thus inhibiting aggregation and in some instances preventing interaction of pyrophosphate with magnesium ions during T7 transcription reactions.

As used herein, the term “internucleotide linkage” refers to the bond or bonds that connect two nucleosides of an oligonucleotide or nucleic acid and may be a natural phosphodiester linkage or modified linkage.

As used herein, the term “substantially free” refers to a state in which little or no impurity is present in a sample (e.g., prematurely aborted RNA sequences, uncapped RNA, DNA, and/or double-stranded RNA). “Substantially free of impurities” means impurities are present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample. For example, “substantially free of double-stranded RNA” means double-stranded RNA is present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample. In embodiments, “Substantially free of impurities” means impurities are present at a level less than approximately 5%. impurities are present at a level less than approximately 4%. impurities are present at a level less than approximately 3%. impurities are present at a level less than approximately 2%. impurities are present at a level less than approximately 1%. impurities are present at a level less than approximately 0.9%. impurities are present at a level less than approximately 0.8%. impurities are present at a level less than approximately 0.7%. impurities are present at a level less than approximately 0.6%. impurities are present at a level less than approximately 0.5%. impurities are present at a level less than approximately 0.4%. impurities are present at a level less than approximately 0.3%. impurities are present at a level less than approximately 0.2%. impurities are present at a level less than approximately 0.1%.

As used herein, the term “impurities” refers to substances which differ from the chemical composition of the target material (e.g., mRNA transcripts). Impurities are also referred to as contaminants.

As used herein, the term “enzymatic byproduct” refers to undesired products from IVT reaction such as for example, uncapped RNAs, truncated RNAs (not fully synthesized RNAs or RNAs missing the tail end), dsRNAs. Enzymatic byproducts may be considered impurities.

As used herein, “tangential flow filtration (TFF)” is a type of filtration wherein the material to be filtered is passed tangentially across a filter rather than through it. In TFF, undesired permeate passes through the filter, while the desired retentate passes along the filter and is collected downstream. In TFF, the desired material is typically contained in the retentate, which is the opposite of what is encountered when performing traditional membrane or dead-end filtration.

As used herein, the term “in vitro” refers to a process that takes place outside a living organism (e.g., a multi-cellular organism, such as a human or a non-human animal), for example, in a test tube, culture dish, or elsewhere outside a living organism.

As used herein, the term “in vivo” refers to events that occur within a living organism.

As used herein, the term “transcription” refers to enzymatically making or synthesizing RNA that is complementary to a DNA template, thereby producing a number of RNA complements of a DNA sequence. The RNA molecule synthesized in a transcription reaction is an “RNA transcript,” “primary transcript,” or “transcript.” Transcription reactions involving the compositions and methods provided herein employ initiating capped oligonucleotide primers described herein. Transcription of a DNA template may be exponential, nonlinear or linear. A DNA template may be a double-stranded linear DNA, a partially double-stranded linear DNA, circular double-stranded DNA, DNA plasmid, PCR amplified product, or a modified nucleic acid template that is compatible with RNA polymerase.

As used herein, the term “modified oligonucleotide” includes, for example, an oligonucleotide containing a modified nucleoside, a modified internucleotide linkage, or having any combination of modified nucleosides and internucleotide linkages. Examples of internucleotide linkage modifications include phosphorothioate, phosphotriester and methylphosphonate derivatives (Stec, W. J., et al., Chem. Int. Ed. Engl., 33:709-722 (1994); Lebedev, A. V., et al., E., Perspect. Drug Discov. Des., 4:17-40 (1996); and Zon, et al., U.S. patent application No. 20070281308). Other examples of internucleotide linkage modifications may be found in Waldner, et al., Bioorg. Med. Chem. Letters 6:2363-2366 (1996).

As used herein, “oligo dT purification” is an affinity chromatography method for purification of mRNA comprising or including a poly-A tail.

As used herein, “phosphorothioate linkage” refers to a linkage between nucleosides in which the phosphorodiester linkage is modified by replacing one of the oxygen atoms, connected to a phosphorus atom, with a sulfur atom.

As used herein, the term “prematurely aborted RNA transcript” refers to incomplete products of an in vitro transcription reaction. Prematurely aborted RNA sequences may be any length that is less than the intended length of the desired transcriptional product.

The term “promoter” as used herein refers to a nucleotide sequence in a DNA template that directs and controls the initiation of transcription of a particular DNA sequence. Promoters are typically immediately adjacent to (or partially overlap with) the DNA sequence to be transcribed. Promoter sequences are typically located directly upstream or at the S′ end of the transcription initiation site. Nucleotide positions in the promoter are designated relative to the transcriptional start site, where transcription of DNA begins (position+1).

As used herein, the term “nucleoside” refers to a nitrogenous base linked to a 5-carbon sugar (e.g., ribose or deoxyribose). The term includes all nucleosides, including all forms of nucleoside bases and furanosides. Base rings include purine and pyrimidine rings. Purine rings include, for example, adenine, guanine, and N6-methyladenine. Pyrimidine rings include, for example, cytosine, thymine, 5-methylcytosine, and pseudouracil. Other nucleosides include, but are not limited to, ribo, 2′-O-methyl or 2′-deoxyribo derivatives of adenosine, guanosine, cytidine, thymidine, uridine, inosine, 7-methylguanosine or pseudouridine. The term “natural nucleoside” refers to adenosine, guanosine, cytidine, uridine and thymidine. According to Aduri et al (Aduri, R. et al., AMBER force field parameters for the naturally occurring modified nucleotides in RNA. Journal of Chemical Theory and Computation. 2006. 3 (4): 1464-75) there are 107 naturally occurring nucleosides, including 1-methyladenosine, 2-methylthio-N6-hydroxynorvalyl carbamoyladenosine, 2-methyladenosine, 2-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N₆,N₆-dimethyladenosine, N6-(cis-hydroxyisopentenyl)adenosine, N6-hydroxynorvalylcarbamoyladenosine, 1,2-O-dimethyladenosine, N6,2-O-dimethyladenosine, 2-O-methyladenosine, N6,N6,O-2-trimethyladenosine, 2-methylthio-N6-(cis-hydroxyisopentenyl) adenosine, 2-methylthio-No-methyladenosine, 2-methylthio-N6-isopentenyladenosine, 2-methylthio-No-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-formyl-2-O-methylcytidine, 5,2-O-dimethylcytidine, 2-O-methylcytidine, N_{4,2}-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-aminomethyl-7-deazaguanosine, 7-cyano-7-deazaguanosine, N2,N₂-dimethylguanosine, 4-demethylwyosine, epoxyqueuosine, hydroxywybutosine, isowyosine, N2,7,2-O-trimethylguanosine, N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldibydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl) pseudouridine, 1-methylpseudouridine, 3-methylpseudouridine, 2-O-methylpseudouridine, inosine, 1-methylinosine, 1,2-O-dimethylinosine and 2-O-methylinosine. Each of these or the modified nucleobase thereof may be components of nucleic acids of the present invention.

All other nucleosides (not including the ones described above as natural nucleosides or modified natural nucleosides) are unnatural nucleosides.

As used herein, the terms “nucleoside analogs,” “modified nucleosides,” or “nucleoside derivatives” include synthetic nucleosides as described herein. Nucleoside derivatives also include nucleosides having modified base or/and sugar moieties, with or without protecting groups and include, for example, 2′-deoxy-2′-fluorouridine, 5-fluorouridine and the like. The compounds and methods provided herein include such base rings and synthetic analogs thereof, as well as unnatural heterocycle-substituted base sugars, and acyclic substituted base sugars. Other nucleoside derivatives that may be utilized with the present disclosure include, for example, LNA nucleosides, halogen-substituted purines (e.g., 6-fluoropurine), halogen-substituted pyrimidines, N⁶-ethyladenine, N⁴-(alkyl)-cytosines, 5-ethylcytosine, and the like (e.g., U.S. Pat. No. 6,762,298).

As used herein, the terms “nucleoside base” or “nucleobase” refer to the base portion of a nucleotide or nucleoside (a nitrogenous base); nucleobases include natural, modified, and unnatural nucleobases. A “natural nucleoside base” includes purine and pyrimidine rings. Purine rings include, for example, adenine and guanine. Pyrimidine rings include, for example, cytosine, thymine, and uracil.

As used herein, the terms “modified nucleoside base” or “modified nucleobase” describe natural modified nucleoside bases, including but is not limited to, for example, pseudouracil, 5-methylcytosine, N6-methyladenine, hypoxanthine, 5-hydroxymethylcytosine, 5-carboxylcytosine, N4-acetylcytosine, N4-methylcytosine, N1-methyladenine, N2,N2-dimethylguanine and the like. Also, see naturally occurring modified nucleosides above.

As used herein, the terms “unnatural nucleoside base” or “unnatural nucleobase” refer to all nucleoside bases that are not natural (whether modified or not; see naturally occurring modified nucleosides above) including but is not limited to, for example, N1-methylpseudouracil, 7-deazaadenine, 2-aminoadenine, 5-methylisocytosine, 5-fluorouracil, 5-bromouracil, 5-iodouracil, 2-methylthioadenine, 2-thio-5-methyluracil, 2-amino-6-methylthiopurine and the like. Natural nucleosides are described above.

As used herein, the term “RNA polymerase” refers to an enzyme that synthesizes RNA using a DNA template. For in vitro transcription methods, single subunit phage RNA polymerases derived from T7, T3, SP6, K1-5, K1E, K1F or K11 bacteriophages, or variants thereof, are typically used. This family of polymerases has simple, minimal promoter sequences of about 17 nucleotides which require no accessory proteins and have minimal constraints of the initiating nucleotide sequence.

As used herein, the term “purified” or “purify” refers to separating a substance from at least some of the components (e.g., impurities or contaminants) with which it was associated when initially produced. For example, RNA transcripts are purified by removal of contaminating proteins or other undesired nucleic acid species (e.g., double-stranded RNA, DNA, and/or incomplete or aborted RNA transcripts). Purified substances (e.g., capped mRNA transcripts) can be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated.

Salts of one or more compounds as described herein can be used in the disclosed methods. The term “salt(s),” as used herein, refers to derivatives of the compounds described herein prepared by the reaction of an acidic or basic moiety of the compound with a mineral or organic acid or base. Optionally, the salts can be pharmaceutically acceptable salts. As used herein, the term “pharmaceutically acceptable salt(s)” refers to those salts of the compounds described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. Salts may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1; and Remington: The Science and Practice of Pharmacy, 23d Edition, Adejare et al. eds., Academic Press (2020); which are incorporated herein by reference in their entireties.)

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms” or “optionally” means that the subsequently described event or circumstance can or cannot occur, and that the description includes instances where said event or circumstance occurs and instances where it does not.

As used herein, the term “subject” or “patient” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

The term “immunization” or “vaccination” describes the process of treating a subject for therapeutic or prophylactic reasons.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, the term “pharmaceutically acceptable carrier” and “pharmaceutically acceptable excipient” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the terms “hydrophobic moiety” or “hydrophobic group” may be used interchangeably and refer to hydrophobic substituent or a combination of hydrophobic substituents that are carbon rich. The hydrophobicity of a substituent can be determined, measured or calculated through the value of its partition coefficient (log P). The partition coefficient (log P) of a substance defines the ratio of its solubility in two immiscible solvents, normally octanol:water. When this value is calculated rather than measured, it is called cLog P. In embodiments, a hydrophobic group has a cLog P of at least 2 or a combination of two, three or four “partial hydrophobic groups” has a collective value of cLog P of at least 2. Nucleoside bases cytosine, thymine, uracil, adenine, and guanine, whose cLog P is less than 2, are not considered “hydrophobic groups” as defined herein. Non-limiting examples of hydrophobic groups include C₆-C₂₄ alkyl, C₄-C₂₄ alkenyl, C₄-C₂₄ alkynyl, C₃-C₈cycloalkyls, C₆-C₁₀aryls, trityls, silyls, lipids, and steroids. Fluoro substituents or fluorosubstituted groups can be used to increase the hydrophobicity of a hydrophobic group.

As used herein, the terms “cleavable hydrophobic moiety”, “cleavable hydrophobic group”, “removable hydrophobic moiety”, or “removable hydrophobic group” may be used interchangeably and refer to hydrophobic groups such as for example saturated alkyl groups C₃-C₂₀ or longer, cycloalkyl rings, aryl rings, silyls, and the like, which can be chemically or thermally removed from the 5′-capped oligonucleotide using mild conditions. In embodiments, such group(s) can be removed under mild acidic conditions, with pH no lower than about 5 (at room temperature for 1 hour). In embodiments, such group(s) can be removed under mild basic conditions, with a pH no higher than about 9 (at room temperature for 1 hour). In embodiments, such group(s) can be removed by mild heating, at no higher than about 65° C. for 1 hour. In embodiments, such group(s) can be removed by reductive amination. In embodiments, such group(s) can be removed by desilylation. In embodiments, such group(s) can be removed by oxidation. In embodiments, such group(s) can be removed by photolysis. In embodiments, these terms exclude photocleavable (photolabile) groups. In this application, a hydrophobic group is considered “cleavable” or “removable” if it can be removed under conditions wherein 5′-capped RNA is not denatured. In embodiments, once these group(s) are removed, highly pure and readily translatable 5′-capped oligonucleotide is generated.

As used herein, the terms “non-cleavable hydrophobic moiety” or “non-cleavable hydrophobic group” may be used interchangeably and refer to hydrophobic groups such as for example saturated alkyl groups C₃-C₂₀ or longer, C₃-C₈cycloalkyls, C₆-C₁₀aryls, and the like, which cannot be easily removed from the 5′-capped oligonucleotide. In embodiments, said hydrophobic groups cannot be removed using mild conditions described above for the cleavable hydrophobic groups. In embodiments, the non-cleavable hydrophobic groups cannot be removed at pH between about 5 to about 9 (at room temperature for 1 hour). In embodiments, the non-cleavable hydrophobic groups cannot be removed by heating below about 65° C. for 1 hour. In embodiments, non-cleavable hydrophobic group(s) do not interfere with the translation of the 5′-capped oligonucleotide. In embodiments, at most 5% of the non-cleavable hydrophobic group(s) are cleaved from the protected 5′-capped oligonucleotide. In embodiments, at most 10% of the non-cleavable hydrophobic group(s) are cleaved from the protected 5′-capped oligonucleotide. In embodiments, at most 15% of the non-cleavable hydrophobic group(s) are cleaved from the protected 5′-capped oligonucleotide. In embodiments, at most 20% of the non-cleavable hydrophobic group(s) are cleaved from the protected 5′-capped oligonucleotide.

As used herein, the term “translation permissible hydrophobic group” refers to any hydrophobic group that does not inhibit (or interfere with) translation of the 5′-capped RNA. Typically, most non-removable hydrophobic groups are translation permissible. In embodiments, removable hydrophobic group may be translation permissible. In such case, the groups may not allowed to stay even though they may be “removable”.

As used herein, the terms “photocleavable group” or “photolabile group” may be used interchangeably and refer to groups such as for example nitrobenzyls, nitrobenzyl derivatives, phenacyls, benzyls, and the like, which can be removed by irradiation with light of certain frequency.

As used herein, the term “purification handle” refers to a hydrophobic group which is covalently linked to the 5′-capped oligonucleotide, such group may be removable or non-removable. The purification handle allows for HPLC separation of 5′-capped oligonucleotides, which comprise covalently linked purification handle, from the uncapped oligonucleotides and other truncated oligonucleotides. In embodiments, the purification handle may be a protecting group. In embodiments, the purification handle includes, for example, but is not limited to C₆-C₂₄ alkyl, C₄-C₂₄ alkenyl, C₄-C₂₄ alkynyl, C₃-C₈cycloalkyls, C₆-C₁₀aryls, silyl compounds, trityl compounds, vinyl ether compounds, modified and unmodified Fmoc compounds, and the like. Typically, hydrophobic groups are purification handles.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically, a protecting group is bound to a heteroatom (e.g., O or N) during a part of a multipart synthesis wherein it is not desired to have the heteroatom react (e.g., a chemical reduction) with the reagent. Following protection, the protecting group may be removed (e.g., by modulating the pH or temperature). In embodiments the protecting group is an alcohol protecting group. Non-limiting examples of alcohol protecting groups include acyls, acetyl, benzoyl, benzyl, methoxymethyl ether (MOM), tetrahydropyranyl (THP), tert-butyldimethyl silyl (TBDMS), and silyl ether (e.g., trimethylsilyl (TMS)). In embodiments the protecting group is an amine protecting group. Non-limiting examples of amine protecting groups include trityl, monomethoxytrityl (MMT), dimethoxytrityl (DMT), or other modified trityls, dimethylcarbobenzyloxy (Cbz), tert-butyloxycarbonyl (Boc), 9-Fluorenylmethyloxycarbonyl (Fmoc), acyls, acetyl, benzoyl, benzyl, carbamate, p-methoxybenzyl ether (PMB), tert-butyldiphenyl silyl (TBDPS), and tosyl (Ts).

### II. 5′-Capped Oligonucleotides

In an aspect, provided herein is an oligonucleotide comprising 50-12000 nucleotides, whose 5′ end comprises a structure of formula (I)

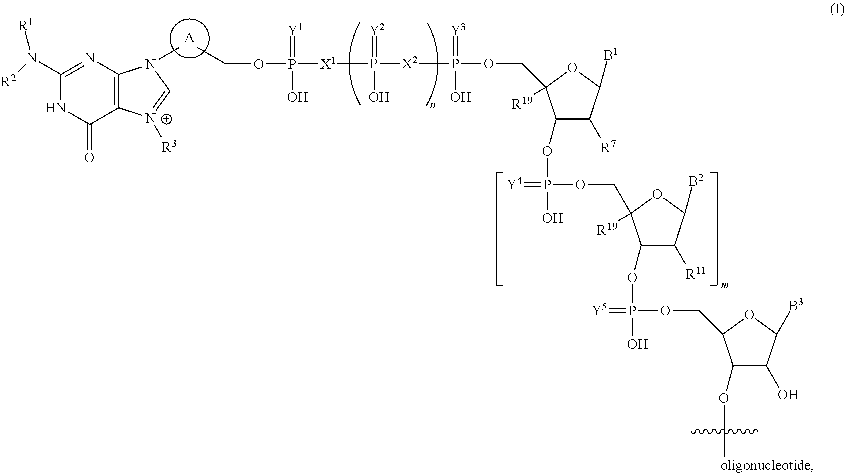

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   B¹ and B³ are each independently a natural, modified, or unnatural nucleoside base;
   each B² is independently a natural, modified, or unnatural nucleoside base;
   Ring A is a substituted or unsubstituted cycloalkylene, substituted or unsubstituted heterocycloalkylene, substituted or unsubstituted arylene, or substituted or unsubstituted heteroarylene;
   X¹ and X² are each independently —O—, —CH₂—, —CX₂—, —N(R¹⁰¹)—, —BH—, or —S—;
   Y¹, Y², Y³, Y⁴, and Y⁵ are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R⁷ is independently hydrogen,
      halogen, —CCl₃, —CBr₃, —CF₃, —CI₃, —CHCl₂, —CHBr₂,
      —CHF₂, —CHI₂, —CH₂Cl, —CH₂Br, —CH₂F, —CH₂I, —CN, —OR⁷⁴, —NR^{7A}R^{7B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁹ is independently hydrogen,
   halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{19A}, —NR^{19A}R^{19B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁷ and R¹⁹ together with the carbon atoms to which they are connected form a substituted or unsubstituted cycloalkylene or substituted or unsubstituted heterocycloalkylene;
each R¹¹ is independently hydrogen,
   halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{11A}, —NR^{11A}R^{11B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R¹¹ and R¹⁹ together with the carbon atoms to which they are connected form a substituted or unsubstituted cycloalkylene or substituted or unsubstituted heterocycloalkylene;
each R^{1A}, R^{2A}, R^{3A}, R^{7A}, R^{7B}, R^{11A}, R^{11B}, R^{19A}, and R^{19B} is independently
   hydrogen, —CX₃,
   —CHX₂, —CH₂X, —C(O)OH, —C(O)NH₂, —CN, —OH, —NH₂, —COOH, —CONH₂, —NO₂, —SH, —SO₃H,
   —SO₄H, —SO₂NH₂, —NHNH₂, —ONH₂, —NHC═(O)NHNH₂, —NHC═(O)NH₂, —NHSO₂H,
   —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R^{7A} and R^{7B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
m is an integer from 0 to 8;
n is an integer from 0 to 3; and
each X is independently —Cl, —Br, —I or —F.
   Here and throughout the disclosure,

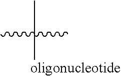

indicates the point of attachment of a structure to the remainder (e.g., body and 3′ end) of the oligonucleotide.

In embodiments, R¹ is independently hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹ (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R¹ is independently substituted with a plurality of groups selected from substituent groups, size-limited substituent groups, and lower substituent groups; each substituent group, size-limited substituent group, and/or lower substituent group may optionally be different. In embodiments, when R¹ is independently substituted, it is substituted with at least one substituent group. In embodiments, when R¹ is independently substituted, it is substituted with at least one size-limited substituent group. In embodiments, when R¹ is independently substituted, it is substituted with at least one lower substituent group.

In embodiments, R¹ is independently hydrogen, substituted or unsubstituted alkyl, or substituted or unsubstituted aryl. In embodiments, R¹ is independently hydrogen, substituted (e.g., with a substituent group, a size-limited substituent group or a lower substituent group) or unsubstituted alkyl (e.g., C₁-C₈ alkyl, C₁-C₆ alkyl, or C₁-C₄ alkyl), or substituted (e.g. with a substituent group, a size-limited substituent group or a lower substituent group) or unsubstituted aryl (e.g., C₆-C₁₀ aryl, C₁₀ aryl, or phenyl). In embodiments, R¹ is independently hydrogen. In embodiments, R¹ is independently unsubstituted alkyl (e.g., C₁-C₈alkyl, C₁-C₆ alkyl, or C₁-C₄ alkyl). In embodiments, R¹ is independently substituted (e.g., with a substituent group, a size-limited substituent group or a lower substituent group) alkyl (e.g., C₁-C₈ alkyl, C₁-C₆ alkyl, or C₁-C₄ alkyl). In embodiments, R¹ is independently unsubstituted aryl (e.g., C₆-C₁₀ aryl, C₁₀ aryl, or phenyl). In embodiments, R¹ is independently substituted (e.g., with a substituent group, a size-limited substituent group or a lower substituent group) aryl (e.g., C₆-C₁₀ aryl, C₁₀ aryl, or phenyl).

In embodiments, R¹ is independently hydrogen, methyl, ethyl, propyl, isopropyl, butyl, isobutyl, t-butyl, pentyl, isopentyl, hexyl, phenyl, benzyl,

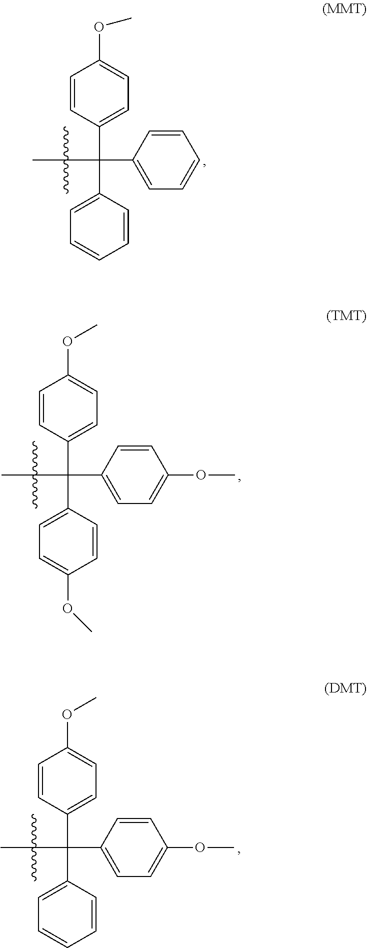 [SMILES: *C(c1ccccc1)(c1ccccc1)c1ccc(OC)cc1 ; *C(c1ccc(OC)cc1)(c1ccc(OC)cc1)c1ccc(OC)cc1 ; *C(c1ccccc1)(c1ccc(OC)cc1)c1ccc(OC)cc1]

or modified trityl.

R¹ is independently hydrogen, methyl, ethyl, propyl, isopropyl, isobutyl, t-butyl, pentyl, isopentyl, hexyl, phenyl, benzyl,

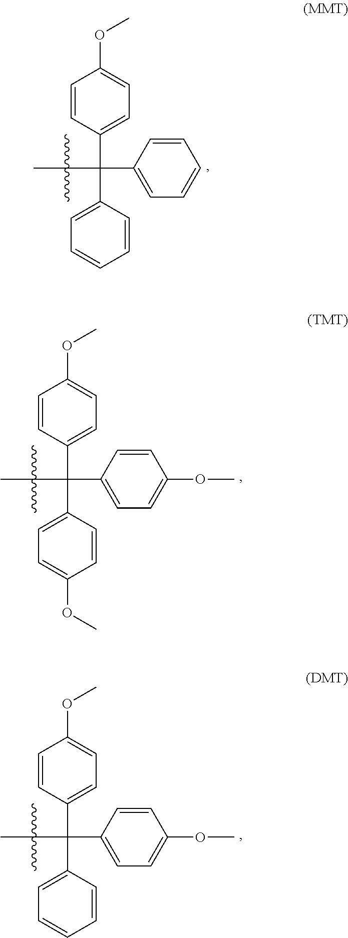 [SMILES: *C(c1ccccc1)(c1ccccc1)c1ccc(OC)cc1 ; *C(c1ccc(OC)cc1)(c1ccc(OC)cc1)c1ccc(OC)cc1 ; *C(c1ccccc1)(c1ccc(OC)cc1)c1ccc(OC)cc1]

or modified trityl.

In embodiments, R¹ is independently hydrogen or methyl. In embodiments, R¹ is independently hydrogen or

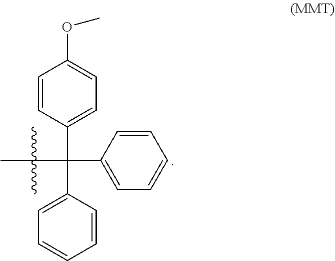 [SMILES: *C(c1ccccc1)(c1ccccc1)c1ccc(OC)cc1]

In embodiments, R¹ is independently hydrogen. In embodiments, R¹ is independently methyl. In embodiments, R¹ is independently ethyl. In embodiments, R¹ is independently propyl. In embodiments, R¹ is independently isopropyl. In embodiments, R¹ is independently butyl. In embodiments, R¹ is independently isobutyl. In embodiments, R¹ is independently t-butyl. In embodiments, R¹ is independently pentyl. In embodiments, R¹ is independently isopentyl. In embodiments, R¹ is independently hexyl. In embodiments, R¹ is independently phenyl. In embodiments, R¹ is independently benzyl. In embodiments, R¹ is independently

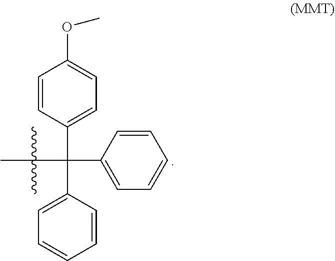 [SMILES: *C(c1ccccc1)(c1ccccc1)c1ccc(OC)cc1]

In embodiments, R¹ is independently

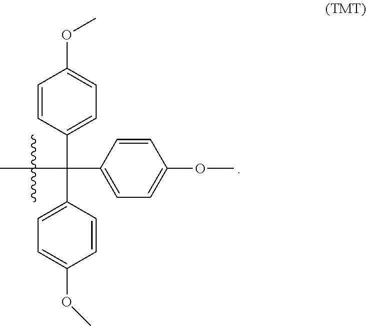 [SMILES: *C(c1ccc(OC)cc1)(c1ccc(OC)cc1)c1ccc(OC)cc1]

In embodiments, R¹ is independently

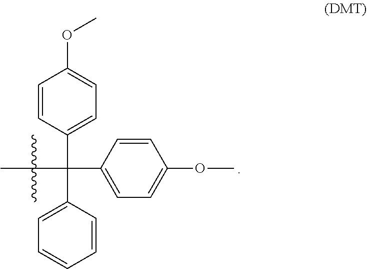 [SMILES: *C(c1ccccc1)(c1ccc(OC)cc1)c1ccc(OC)cc1]

In embodiments, R¹ is independently modified trityl.

In embodiments, R² is independently hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R² (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R² is independently substituted with a plurality of groups selected from substituent groups, size-limited substituent groups, and lower substituent groups; each substituent group, size-limited substituent group, and/or lower substituent group may optionally be different. In embodiments, when R² is independently substituted, it is substituted with at least one substituent group. In embodiments, when R² is independently substituted, it is substituted with at least one size-limited substituent group. In embodiments, when R² is independently substituted, it is substituted with at least one lower substituent group.

In embodiments, R² is independently hydrogen, substituted or unsubstituted alkyl, or substituted or unsubstituted aryl. In embodiments, R² is independently hydrogen, substituted (e.g., with a substituent group, a size-limited substituent group or a lower substituent group) or unsubstituted alkyl (e.g., C₁-C₈ alkyl, C₁-C₆ alkyl, or C₁-C₄ alkyl), or substituted (e.g. with a substituent group, a size-limited substituent group or a lower substituent group) or unsubstituted aryl (e.g., C₆-C₁₀ aryl, C₁₀ aryl, or phenyl). In embodiments, R² is independently hydrogen. In embodiments, R² is independently unsubstituted alkyl (e.g., C₁-C₈alkyl, C₁-C₆ alkyl, or C₁-C₄ alkyl). In embodiments, R² is independently substituted (e.g., with a substituent group, a size-limited substituent group, or a lower substituent group) alkyl (e.g., C₁-C₈ alkyl, C₁-C₆ alkyl, or C₁-C₄ alkyl). In embodiments, R² is independently unsubstituted aryl (e.g., C₆-C₁₀ aryl, C₁₀ aryl, or phenyl). In embodiments, R² is independently substituted (e.g., with a substituent group, a size-limited substituent group, or a lower substituent group) aryl (e.g., C₆-C₁₀ aryl, C₁₀ aryl, or phenyl).

In embodiments, R² is independently hydrogen, methyl, ethyl, propyl, isopropyl, butyl, isobutyl, t-butyl, pentyl, isopentyl, hexyl, phenyl, benzyl,

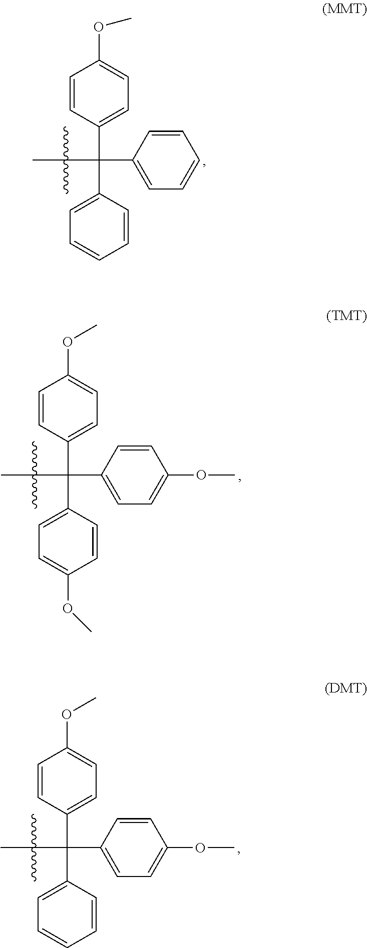 [SMILES: *C(c1ccccc1)(c1ccccc1)c1ccc(OC)cc1 ; *C(c1ccc(OC)cc1)(c1ccc(OC)cc1)c1ccc(OC)cc1 ; *C(c1ccccc1)(c1ccc(OC)cc1)c1ccc(OC)cc1]

or modified trityl.

R¹ is independently hydrogen, methyl, ethyl, propyl, isopropyl, isobutyl, t-butyl, pentyl, isopentyl, hexyl, phenyl, benzyl,

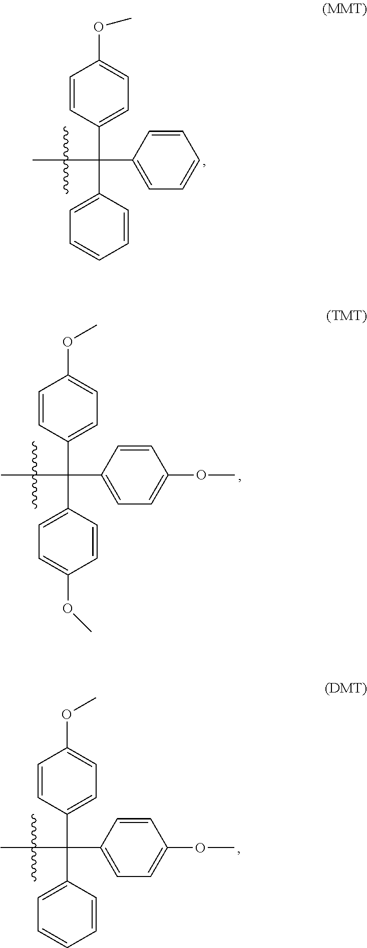 [SMILES: *C(c1ccccc1)(c1ccccc1)c1ccc(OC)cc1 ; *C(c1ccc(OC)cc1)(c1ccc(OC)cc1)c1ccc(OC)cc1 ; *C(c1ccccc1)(c1ccc(OC)cc1)c1ccc(OC)cc1]

or modified trityl.

In embodiments, R² is independently hydrogen or methyl. In embodiments, R² is independently hydrogen or

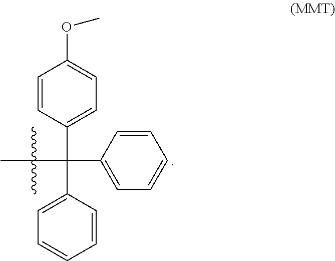 [SMILES: *C(c1ccccc1)(c1ccccc1)c1ccc(OC)cc1]

In embodiments, R² is independently hydrogen. In embodiments, R² is independently methyl. In embodiments, R² is independently ethyl. In embodiments, R² is independently propyl. In embodiments, R² is independently isopropyl. In embodiments, R² is independently butyl. In embodiments, R² is independently isobutyl. In embodiments, R² is independently t-butyl. In embodiments, R² is independently pentyl. In embodiments, R² is independently isopentyl. In embodiments, R² is independently hexyl. In embodiments, R² is independently phenyl. In embodiments, R² is independently benzyl. In embodiments, R² is independently

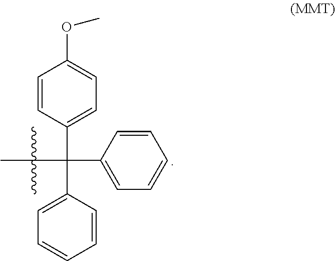 [SMILES: *C(c1ccccc1)(c1ccccc1)c1ccc(OC)cc1]

In embodiments, R² is independently

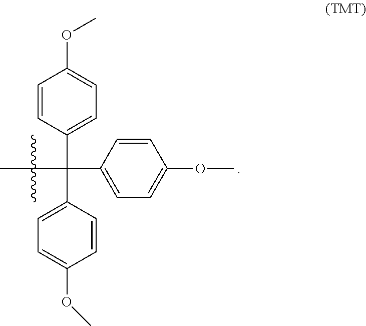 [SMILES: *C(c1ccc(OC)cc1)(c1ccc(OC)cc1)c1ccc(OC)cc1]

In embodiments, R² is independently

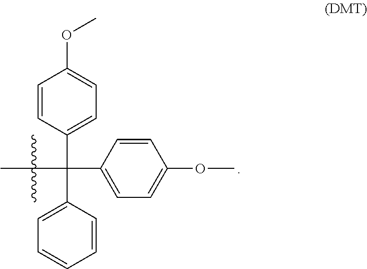 [SMILES: *C(c1ccccc1)(c1ccc(OC)cc1)c1ccc(OC)cc1]

In embodiments, R² is independently modified trityl.

In embodiments, R³ is hydrogen, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen, substituted or unsubstituted alkyl, or substituted or unsubstituted aryl. In embodiments, R³ is hydrogen, substituted (e.g., with a substituent group, a size-limited substituent group or a lower substituent group) or unsubstituted alkyl (e.g., C₁-C₈alkyl, C₁-C₆ alkyl, or C₁-C₄ alkyl), or substituted (e.g., with a substituent group, a size-limited substituent group or a lower substituent group) or unsubstituted aryl (e.g., C₆-C₁₀ aryl, C₁₀ aryl, or phenyl). In embodiments, R³ is hydrogen. In embodiments, R³ is unsubstituted alkyl (e.g., C₁-C₈ alkyl, C₁-C₆ alkyl, or C₁-C₄ alkyl). In embodiments, R³ is substituted (e.g., with a substituent group, a size-limited substituent group, or a lower substituent group) alkyl (e.g., C₁-C₈alkyl, C₁-C₆ alkyl, or C₁-C₄ alkyl). In embodiments, R³ is unsubstituted aryl (e.g., C₆-C₁₀ aryl, C₁₀ aryl, or phenyl). In embodiments, R³ is substituted (e.g., with a substituent group, a size-limited substituent group, or a lower substituent group) aryl (e.g., C₆-C₁₀ aryl, C₁₀ aryl, or phenyl).

In embodiments, R³ is hydrogen, methyl, ethyl, propyl, isopropyl, butyl, isobutyl, t-butyl, pentyl, isopentyl, hexyl, phenyl, benzyl, or 4-chlorobenzyl. In embodiments, R³ is methyl or 4-chlorobenzyl.

In embodiments, R³ is hydrogen. In embodiments, R³ is methyl. In embodiments, R³ is ethyl. In embodiments, R³ is propyl. In embodiments, R³ is isopropyl. In embodiments, R³ is butyl. In embodiments, R³ is isobutyl. In embodiments, R³ is t-butyl. In embodiments, R³ is pentyl. In embodiments, R³ is isopentyl. In embodiments, R³ is hexyl. In embodiments, R³ is phenyl. In embodiments, R³ is benzyl. In embodiments, R³ is 4-chlorobenzyl.

In embodiments, R⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{7A}, —NR^{7A}R^{7B}, —NO₂,

—SH, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is independently hydrogen, halogen, or —OR^{7A}. In embodiments, R⁷ is independently hydrogen. In embodiments, R⁷ is independently halogen. In embodiments, R⁷ is independently —OR^{7A}. In embodiments, R⁷ is independently chloro. In embodiments, R⁷ is independently bromo. In embodiments, R⁷ is independently iodo. In embodiments, R⁷ is independently fluoro.

In embodiments, R⁷ is independently hydroxy, methoxy, ethoxy, propoxy, butoxy, or t-butoxy. In embodiments, R⁷ is independently hydroxy. In embodiments, R⁷ is independently methoxy. In embodiments, R⁷ is independently ethoxy. In embodiments, R⁷ is independently propoxy. In embodiments, R⁷ is independently butoxy. In embodiments, R⁷ is independently t-butoxy.

In embodiments, R^{7A} is hydrogen or substituted or unsubstituted alkyl. In embodiments, R^{7A} is hydrogen. In embodiments, R^{7A} is substituted alkyl. In embodiments, R^{7A} is an unsubstituted alkyl.

In embodiments, R^{7A} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, R^{7A} is hydrogen or substituted or unsubstituted alkyl. In embodiments, R^{7A} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{7A} is hydrogen. In embodiments, R^{7A} is unsubstituted alkyl (e.g., C₁-C₈ alkyl, C₁-C₆ alkyl, or C₁-C₄ alkyl). In embodiments, R^{7A} is substituted (e.g. with a substituent group, a size-limited substituent group or a lower substituent group) alkyl (e.g., C₁-C₈ alkyl, C₁-C₆ alkyl, or C₁-C₄ alkyl).

In embodiments, R^{7A} is hydrogen, methyl, ethyl, propyl, isopropyl, butyl, isobutyl, t-butyl, pentyl, isopentyl, hexyl. In embodiments, R^{7A} is hydrogen. In embodiments, R^{7A} is methyl. In embodiments, R^{7A} is ethyl. In embodiments, R^{7A} is propyl. In embodiments, R^{7A} is isopropyl. In embodiments, R^{7A} is butyl. In embodiments, R^{7A} is isobutyl. In embodiments, R^{7A} is t-butyl. In embodiments, R^{7A} is pentyl. In embodiments, R^{7A} is hexyl.

In embodiments, R¹¹ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{7A}, —NR^{7A}R^{7B}, —NO₂, —SH, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, R¹¹ is independently hydrogen, halogen, or —OR^{11A}. In embodiments, R¹¹ is independently hydrogen. In embodiments, R¹¹ is independently halogen. In embodiments, R¹¹ is independently —OR^{11A}. In embodiments, R¹¹ is independently chloro. In embodiments, R¹¹ is independently bromo. In embodiments, R¹¹ is independently iodo. In embodiments, R¹¹ is independently fluoro.

In embodiments, R¹¹ is independently hydroxy, methoxy, ethoxy, propoxy, butoxy, or t-butoxy. In embodiments, R¹¹ is independently hydroxy. In embodiments, R¹¹ is independently methoxy. In embodiments, R¹¹ is independently ethoxy. In embodiments, R¹¹ is independently propoxy. In embodiments, R¹¹ is independently butoxy. In embodiments, R¹¹ is independently t-butoxy.

In embodiments, R^{11A} is hydrogen or substituted or unsubstituted alkyl. In embodiments, R^{11A} is hydrogen. In embodiments, R^{11A} is substituted alkyl. In embodiments, R^{1A} is an unsubstituted alkyl.

In embodiments, R^{11A} is hydrogen, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl).

In embodiments, a substituted R^{11A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, R^{11A} is hydrogen or substituted or unsubstituted alkyl. [0005] In embodiments, R^{11A} is hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{11A} is hydrogen. In embodiments, R^{11A} is unsubstituted alkyl (e.g., C₁-C₈ alkyl, C₁-C₆ alkyl, or C₁-C₄ alkyl). In embodiments, R^{11A} is substituted (e.g., with a substituent group, a size-limited substituent group or a lower substituent group) alkyl (e.g., C₁-C₈ alkyl, C₁-C₆ alkyl, or C₁-C₄ alkyl).

In embodiments, R^{11A} is hydrogen, methyl, ethyl, propyl, isopropyl, butyl, isobutyl, t-butyl, pentyl, isopentyl, hexyl. In embodiments, R^{11A} is hydrogen. In embodiments, R^{11A} is methyl. In embodiments, R^{11A} is ethyl. In embodiments, R^{11A} is propyl. In embodiments, R^{11A} is isopropyl. In embodiments, R^{11A} is butyl. In embodiments, R^{11A} is isobutyl. In embodiments, R^{11A} is t-butyl. In embodiments, R^{11A} is pentyl. In embodiments, R^{11A} is hexyl.

In embodiments, R¹⁹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{19A}, —NR^{19A}R^{19B}, —CONH₂, —COOH, —NHC(O)OH, —NO₂, —SH, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁹ is substituted with a plurality of groups selected from substituent groups, size-limited substituent groups, and lower substituent groups; each substituent group, size-limited substituent group, and/or lower substituent group may optionally be different. In embodiments, when R¹⁹ is substituted, it is substituted with at least one substituent group. In embodiments, when R¹⁹ is substituted, it is substituted with at least one size-limited substituent group. In embodiments, when R¹⁹ is substituted, it is substituted with at least one lower substituent group.

In embodiments, R¹⁹ is hydrogen.

In embodiments, R⁷ and R¹⁹ together with the carbon atoms to which they are connected form a substituted or unsubstituted cycloalkylene or substituted or unsubstituted heterocycloalkylene. In embodiments, R⁷ and R¹⁹ together with the carbon atoms to which they are connected form a substituted or unsubstituted cycloalkylene. In embodiments, R⁷ and R¹⁹ together with the carbon atoms to which they are connected form a substituted or unsubstituted heterocycloalkylene.

In embodiments, R⁷ and R¹⁹ together with the carbon atoms to which they are connected form a substituted (e.g., substituted with at least one substituent group, size-limited substituent group, or lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, R⁷ and R¹⁹ together with the carbon atoms to which they are connected form a substituted (e.g., substituted with at least one substituent group, size-limited substituent group, or lower substituent group) or unsubstituted cycloalkylene (e.g., C₃-C₈ cycloalkylene, C₃-C₆ cycloalkylene, or C₅-C₆ cycloalkylene).

In embodiments, a substituted heterocycloalkylene formed by the joining of R⁷ and R¹⁹ together with the carbon atoms to which they are connected is substituted with at least one substituent group, size-limited substituent group, or lower substituent group; wherein if the substituted heterocycloalkylene is substituted with a plurality of groups selected from substituent groups, size-limited substituent groups, and lower substituent groups; each substituent group, size-limited substituent group, and/or lower substituent group may optionally be different. In embodiments, when heterocycloalkylene formed by the joining of R⁷ and R¹⁹ together with the carbon atoms to which they are connected is substituted, it is substituted with at least one substituent group. In embodiments, when heterocycloalkylene formed by the joining of R⁷ and R¹⁹ together with the carbon atoms to which they are connected is substituted, it is substituted with at least one size-limited substituent group. In embodiments, when heterocycloalkylene formed by the joining of R⁷ and R¹⁹ together with the carbon atoms to which they are connected is substituted, it is substituted with at least one lower substituent group.

In embodiments, a substituted cycloalkylene formed by the joining of R⁷ and R¹⁹ together with the carbon atoms to which they are connected is substituted with at least one substituent group, size-limited substituent group, or lower substituent group; wherein if the substituted cycloalkylene is substituted with a plurality of groups selected from substituent groups, size-limited substituent groups, and lower substituent groups; each substituent group, size-limited substituent group, and/or lower substituent group may optionally be different. In embodiments, when cycloalkylene formed by the joining of R⁷ and R¹⁹ together with the carbon atoms to which they are connected is substituted, it is substituted with at least one substituent group. In embodiments, when cycloalkylene formed by the joining of R⁷ and R¹⁹ together with the carbon atoms to which they are connected is substituted, it is substituted with at least one size-limited substituent group. In embodiments, when cycloalkylene formed by the joining of R⁷ and R¹⁹ together with the carbon atoms to which they are connected is substituted, it is substituted with at least one lower substituent group.

In embodiments, R⁷ and R¹⁹ together with the carbon atoms to which they are connected form a substituted or unsubstituted 3 to 6 membered heterocycloalkylene. In embodiments, R⁷ and R¹⁹ together with the carbon atoms to which they are connected form a substituted or unsubstituted 4 membered heterocycloalkylene.

In embodiments, R⁷ and R¹⁹ together with the carbon atoms to which they are connected form a substituted 4 membered heterocycloalkylene. In embodiments, R⁷ and R¹⁹ together with the carbon atoms to which they are connected form an unsubstituted 4 membered heterocycloalkylene.

In embodiments, R⁷ and R¹⁹ together with the carbon atoms to which they are connected form a locked nucleic acid (LNA).

In embodiments, R¹¹ and R¹⁹ together with the carbon atoms to which they are connected form a substituted or unsubstituted cycloalkylene or substituted or unsubstituted heterocycloalkylene. In embodiments, R¹¹ and R¹⁹ together with the carbon atoms to which they are connected form a substituted or unsubstituted cycloalkylene. In embodiments, R¹¹ and R¹⁹ together with the carbon atoms to which they are connected form a substituted or unsubstituted heterocycloalkylene.

In embodiments, R¹¹ and R¹⁹ together with the carbon atoms to which they are connected form a substituted (e.g., substituted with at least one substituent group, size-limited substituent group, or lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, R¹¹ and R¹⁹ together with the carbon atoms to which they are connected form a substituted (e.g., substituted with at least one substituent group, size-limited substituent group, or lower substituent group) or unsubstituted cycloalkylene (e.g., C₃-C₈ cycloalkylene, C₃-C₆ cycloalkylene, or C₅-C₆ cycloalkylene).

In embodiments, a substituted heterocycloalkylene formed by the joining of R¹¹ and R¹⁹ together with the carbon atoms to which they are connected is substituted with at least one substituent group, size-limited substituent group, or lower substituent group; wherein if the substituted heterocycloalkylene is substituted with a plurality of groups selected from substituent groups, size-limited substituent groups, and lower substituent groups; each substituent group, size-limited substituent group, and/or lower substituent group may optionally be different. In embodiments, when heterocycloalkylene formed by the joining of R¹¹ and R¹⁹ together with the carbon atoms to which they are connected is substituted, it is substituted with at least one substituent group. In embodiments, when heterocycloalkylene formed by the joining of R¹¹ and R¹⁹ together with the carbon atoms to which they are connected is substituted, it is substituted with at least one size-limited substituent group. In embodiments, when heterocycloalkylene formed by the joining of R¹¹ and R¹⁹ together with the carbon atoms to which they are connected is substituted, it is substituted with at least one lower substituent group.

In embodiments, a substituted cycloalkylene formed by the joining of R¹¹ and R¹⁹ together with the carbon atoms to which they are connected is substituted with at least one substituent group, size-limited substituent group, or lower substituent group; wherein if the substituted cycloalkylene is substituted with a plurality of groups selected from substituent groups, size-limited substituent groups, and lower substituent groups; each substituent group, size-limited substituent group, and/or lower substituent group may optionally be different. In embodiments, when cycloalkylene formed by the joining of R¹¹ and R¹⁹ together with the carbon atoms to which they are connected is substituted, it is substituted with at least one substituent group. In embodiments, when cycloalkylene formed by the joining of R¹¹ and R¹⁹ together with the carbon atoms to which they are connected is substituted, it is substituted with at least one size-limited substituent group. In embodiments, when cycloalkylene formed by the joining of R¹¹ and R¹⁹ together with the carbon atoms to which they are connected is substituted, it is substituted with at least one lower substituent group.

In embodiments, R¹¹ and R¹⁹ together with the carbon atoms to which they are connected form a substituted or unsubstituted 3 to 6 membered heterocycloalkylene. In embodiments, R¹¹ and R¹⁹ together with the carbon atoms to which they are connected form a substituted or unsubstituted 4 membered heterocycloalkylene.

In embodiments, R¹¹ and R¹⁹ together with the carbon atoms to which they are connected form a substituted 4 membered heterocycloalkylene. In embodiments, R¹¹ and R¹⁹ together with the carbon atoms to which they are connected form an unsubstituted 4 membered heterocycloalkylene.

In embodiments, R¹¹ and R¹⁹ together with the carbon atoms to which they are connected form a locked nucleic acid (LNA).

In embodiments, each R^{1A}, R^{2A}, R^{3A}, R^{7A}, R^{11A}, R^{11B}, R^{19A}, and R^{19B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{1A}, R^{2A}, R^{3A}, R^{7A}, R^{11A}, R^{11B}, R^{19A}, and R^{19B} is independently substituted with one or more substituent groups. In embodiments, each R^{1A}, R^{2A}, R^{3A}, R^{7A}, R^{11A}, R^{11B}, R^{19A}, and R^{19B} is independently substituted with one or more size-limited substituent groups. In embodiments, each R^{1A}, R^{2A}, R^{3A}, R^{7A}, R^{11A}, R^{11B}, R^{19A}, and R^{19B} is independently substituted with one or more lower substituent groups.

In embodiments, R^{7A} and R^{7B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{7A} and R^{7B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{7A} and R^{7B} substituents bonded to the same nitrogen atom is substituted, it is substituted with at least one substituent group. In embodiments, when a heterocycloalkyl formed by the joining of R^{7A} and R^{7B} substituents bonded to the same nitrogen atom is substituted, it is substituted with at least one size-limited substituent group. In embodiments, when a heterocycloalkyl formed by the joining of R^{7A} and R^{7B} substituents bonded to the same nitrogen atom is substituted, it is substituted with at least one lower substituent group. In embodiments, when a heteroaryl formed by the joining of R^{7A} and R^{7B} substituents bonded to the same nitrogen atom is substituted, it is substituted with at least one substituent group. In embodiments, when a heteroaryl formed by the joining of R^{7A} and R^{7B} substituents bonded to the same nitrogen atom is substituted, it is substituted with at least one size-limited substituent group. In embodiments, when a heteroaryl formed by the joining of R^{7A} and R^{7B} substituents bonded to the same nitrogen atom is substituted, it is substituted with at least one lower substituent group.

In embodiments, R^{11A} and R^{11B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{11A} and R^{11B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{11A} and R^{11B} substituents bonded to the same nitrogen atom is substituted, it is substituted with at least one substituent group. In embodiments, when a heterocycloalkyl formed by the joining of R^{11A} and R^{11B} substituents bonded to the same nitrogen atom is substituted, it is substituted with at least one size-limited substituent group. In embodiments, when a heterocycloalkyl formed by the joining of R^{11A} and R^{11B} substituents bonded to the same nitrogen atom is substituted, it is substituted with at least one lower substituent group. In embodiments, when a heteroaryl formed by the joining of R^{11A} and R^{11B} substituents bonded to the same nitrogen atom is substituted, it is substituted with at least one substituent group. In embodiments, when a heteroaryl formed by the joining of R^{11A} and R^{11B} substituents bonded to the same nitrogen atom is substituted, it is substituted with at least one size-limited substituent group. In embodiments, when a heteroaryl formed by the joining of R^{11A} and R^{11B} substituents bonded to the same nitrogen atom is substituted, it is substituted with at least one lower substituent group.

In embodiments, R^{19A} and R^{19B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{19A} and R^{19B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{19A} and R^{19B} substituents bonded to the same nitrogen atom is substituted, it is substituted with at least one substituent group. In embodiments, when a heterocycloalkyl formed by the joining of R19^{17A} and R^{19B} substituents bonded to the same nitrogen atom is substituted, it is substituted with at least one size-limited substituent group. In embodiments, when a heterocycloalkyl formed by the joining of R^{19A} and R^{19B} substituents bonded to the same nitrogen atom is substituted, it is substituted with at least one lower substituent group. In embodiments, when a heteroaryl formed by the joining of R^{19A} and R^{19B} substituents bonded to the same nitrogen atom is substituted, it is substituted with at least one substituent group. In embodiments, when a heteroaryl formed by the joining of R^{19A} and R^{19B} substituents bonded to the same nitrogen atom is substituted, it is substituted with at least one size-limited substituent group. In embodiments, when a heteroaryl formed by the joining of R^{19A} and R^{19B} substituents bonded to the same nitrogen atom is substituted, it is substituted with at least one lower substituent group.

In embodiments, B¹ and B³ are each independently a natural, modified, or unnatural nucleoside base. In embodiments, B¹ and B³ are each independently a natural nucleoside base. In embodiments, B¹ and B³ are each independently a modified nucleoside base. In embodiments, B¹ and B³ are each independently an unnatural nucleoside base.

In embodiments, B¹ and B³ are each independently adenine, guanine, cytosine, uracil, or thymine. In embodiments, B¹ is adenine. In embodiments, B¹ is guanine. In embodiments, B¹ is cytosine. In embodiments, B¹ is uracil. In embodiments, B¹ is thymine. In embodiments, B³ is adenine. In embodiments, B³ is guanine. In embodiments, B³ is cytosine. In embodiments, B³ is uracil. In embodiments, B³ is thymine.

In embodiments, B¹ is independently 5-methylcytosine, pseudouracil, hypoxanthine, N1-methylpseudouracil, N6-methyladenine, N6-ethyladenine, 7-deazaadenine, N-(alkyl)-cytosines, or 5-ethylcytosine, and the like (U.S. Pat. No. 6,762,298).

In embodiments, B¹ includes, but is not limited to, pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine.

In embodiments, B³ includes, but is not limited to, adenine, guanine, cytosine, uracil, pseudouracil, 5-methylcytosine, N6-methyladenine, hypoxanthine, 5-hydroxymethylcytosine, 5-carboxylcytosine, N4-acetylcytosine, N4-methylcytosine, N1-methyladenine, N2,N2-dimethylguanine and the like.

In embodiments, each B² is independently a natural, modified, or unnatural nucleoside base. In embodiments, each B² is independently a natural nucleoside base. In embodiments, each B² is independently a modified nucleoside base. In embodiments, each B² is independently an unnatural nucleoside base.

In embodiments, each B² is independently adenine, guanine, cytosine, uracil, or thymine. In embodiments, B² is adenine. In embodiments, B² is guanine. In embodiments, B² is cytosine. In embodiments, B² is uracil. In embodiments, B² is thymine.

In embodiments, each B² is independently 5-methylcytosine, pseudouracil, hypoxanthine, N1-methylpseudouracil, N6-methyladenine, N6-ethyladenine, 7-deazaadenine, N-(alkyl)-cytosines, or 5-ethylcytosine, and the like (U.S. Pat. No. 6,762,298).

In embodiments, each B² includes, but is not limited to, pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine.

Ring A is a substituted or unsubstituted cycloalkylene, substituted or unsubstituted heterocycloalkylene, substituted or unsubstituted arylene, or substituted or unsubstituted heteroarylene.

In embodiments, Ring A is a substituted or unsubstituted heterocycloalkylene. In embodiments, Ring A is a substituted heterocycloalkylene. In embodiments, Ring A is an unsubstituted heterocycloalkylene.

In embodiments, Ring A is a substituted or unsubstituted heteroarylene. In embodiments, Ring A is a substituted heteroarylene. In embodiments, Ring A is an unsubstituted heteroarylene.

In embodiments, Ring A is a 3 to 10 membered substituted heterocycloalkylene. In embodiments, Ring A is a 3 membered substituted heterocycloalkylene. In embodiments, Ring A is a 4 membered substituted heterocycloalkylene. In embodiments, Ring A is a 5 membered substituted heterocycloalkylene. In embodiments, Ring A is a 6 membered substituted heterocycloalkylene. In embodiments, Ring A is a 7 membered substituted heterocycloalkylene. In embodiments, Ring A is a 8 membered substituted heterocycloalkylene. In embodiments, Ring A is a 9 membered substituted heterocycloalkylene. In embodiments, Ring A is a 10 membered substituted heterocycloalkylene.

In embodiments, Ring A is a 3 to 10 membered unsubstituted heterocycloalkylene. In embodiments, Ring A is a 3 membered unsubstituted heterocycloalkylene. In embodiments, Ring A is a 4 membered unsubstituted heterocycloalkylene. In embodiments, Ring A is a 5 membered unsubstituted heterocycloalkylene. In embodiments, Ring A is a 6 membered unsubstituted heterocycloalkylene. In embodiments, Ring A is a 7 membered unsubstituted heterocycloalkylene. In embodiments, Ring A is a 8 membered unsubstituted heterocycloalkylene. In embodiments, Ring A is a 9 membered unsubstituted heterocycloalkylene. In embodiments, Ring A is a 10 membered unsubstituted heterocycloalkylene.

In embodiments, Ring A is a substituted or unsubstituted tetrahydrofuranylene. In embodiments, Ring A is a substituted tetrahydrofuranylene. In embodiments, Ring A is an unsubstituted tetrahydrofuranylene. In embodiments, Ring A is a substituted or unsubstituted morpholinylene. In embodiments, Ring A is a substituted morpholinylene. In embodiments, Ring A is an unsubstituted morpholinylene.

In embodiments, Ring A is

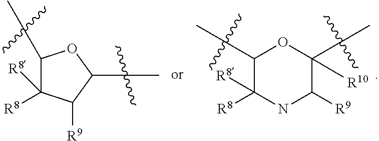

In embodiments,
Ring A is

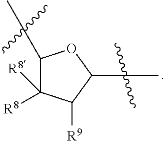

In embodiments, Ring A is

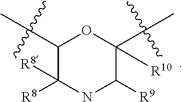

In embodiments, X¹ and X² are each independently —O—, —CH₂—, —CX₂—, —N(R¹⁰¹)—, —BH—, or —S—.

In embodiments, X¹ is —O—, —CH₂—, or —CX₂—. In embodiments, X¹ is —O—, —CH₂—, or —CF₂—. In embodiments, X¹ is —O—. In embodiments, X¹ is —CH₂—. In embodiments, X¹ is —CX₂—. In embodiments, X¹ is —CF₂—. In embodiments, X¹ is —N(R¹⁰¹)—. In embodiments, X¹ is —NH—. In embodiments, X¹ is —BH—. In embodiments, X¹ is —S—.

In embodiments, X² is —O—, —CH₂—, —CX₂—, —N(R¹⁰¹)—, —BH—, or —S—. In embodiments, X² is —O—, —CH₂—, —CF₂—, —NH—, —BH—, or —S—. In embodiments, X² is —O—, —CH₂—, or

—NH—. In embodiments, X² is —CH₂— or —NH—.

In embodiments, X² is —O—. In embodiments, X² is —CH₂—. In embodiments, X² is —CX₂—. In embodiments, X² is —CF₂—. In embodiments, X² is —N(R¹⁰¹)—. In embodiments, X² is —NH—. In embodiments, X² is —BH—. In embodiments, X² is —S—.

In embodiments, Y¹, Y², Y³, Y⁴, and Y⁵, are each independently O, S, or Se. In embodiments, Y¹, Y², Y³, and Y⁴, are each independently O, S, or Se. In embodiments, Y¹, Y², Y³, Y⁴, and Y⁵, are each independently O or S. In embodiments, Y¹, Y², Y³, and Y⁴, are each independently O or S. In embodiments, Y¹, Y², Y³, Y⁴, and Y⁵, are each independently O. In embodiments, Y¹, Y², Y³, and Y⁴, are each independently O. In embodiments, Y¹ is O. In embodiments, Y¹ is S. In embodiments, Y¹ is Se. In embodiments, Y² is O. In embodiments, Y² is S. In embodiments, Y² is Se. In embodiments, Y³ is O. In embodiments, Y³ is S. In embodiments, Y³ is Se. In embodiments, Y⁴ is O. In embodiments, Y⁴ is S. In embodiments, Y⁴ is Se. In embodiments, Y⁵ is O or S. In embodiments, Y⁵ is O. In embodiments, Y⁵ is S. In embodiments, Y⁵ is Se.

In embodiments, each R¹⁰¹ is independently hydrogen, oxo,

halogen, —CCl₃, —CBr₃,
—CF₃, —CI₃, —CHCl₂, —CHBr₂, —CHF₂, —CHI₂, —
CH₂Cl, —CH₂Br, —CH₂F, —CH₂I, —CN, —OH, —NH₂,
—COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl). In embodiments, R¹⁰¹ is substituted with one or more substituent groups. In embodiments, R¹⁰¹ is substituted with one or more size-limited substituent groups. In embodiments, R¹⁰¹ is substituted with one or more lower substituent groups.

In embodiments, each X is independently —Cl, —Br, —I or —F. In embodiments, X is independently —Cl. In embodiments, X is independently —Br. In embodiments, X is independently —I. In embodiments, X is independently —F.

In embodiments, nis an integer from 0 to 3. In embodiments, n is 1 or 2.

In embodiments, n is 0. In embodiments, n is 1. In embodiments, n is 2. In embodiments, n is 3.

In embodiments, m is an integer from 0 to 8. In embodiments, m is 0 or 1. In embodiments, mis 1. In embodiments, mis 2. In embodiments, mis 3. In embodiments, m is 4. In embodiments, mis 5. In embodiments, mis 6. In embodiments, mis 7. In embodiments, mis 8.

In embodiments, provided herein is a structure of formula (IA)

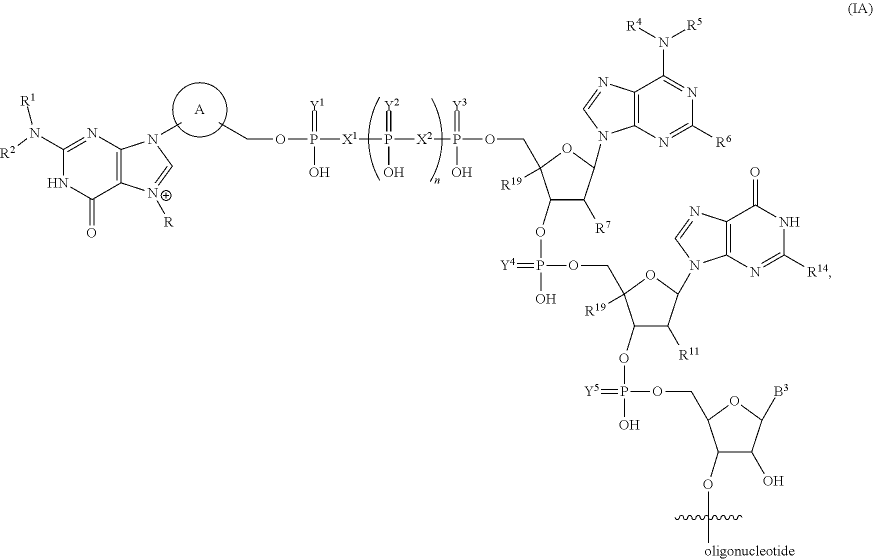

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein:
B³ is a natural nucleoside base;
R⁴ is independently hydrogen, —C(O)R^{4A}, —C(O)OR^{4A}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently hydrogen, —C(O)R^{5A}, —C(O)OR^{5A}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R⁴ and R⁵ together with the nitrogen atom to which they are connected form a substituted or unsubstituted heteroaryl or a substituted or unsubstituted heterocyclyl;
R⁶ is hydrogen, halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{6A}, —NR^{6A}R^{6B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ is hydrogen, halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{14A}, —NR^{14A}R^{14B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{4A}, R^{5A}, R^{6A}, R^{6B}, R^{14A}, and R^{14B} is independently hydrogen, —CX₃, —CHX₂, —CH₂X,
   —C(O)OH, —C(O)NH₂, —CN, —OH, —NH₂, —COOH, —CONH₂, —NO₂, —SH, —SO₃H, —SO₄H, —SO₂NH₂,
   —NHNH₂, —ONH₂, —NHC═(O)NHNH₂, —NHC═(O)NH₂, —NHSO₂H, —NHC═(O)H, —NHC(O)OH,
   —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{6A} and R^{6B} substituents bonded to the same nitrogen atom may optionally be joined to form a substituted or unsubstituted heterocycloalkyl or substituted or unsubstituted heteroaryl; and R^{14A} and R^{14B} substituents bonded to the same nitrogen atom may optionally be joined to form a substituted or unsubstituted heterocycloalkyl or substituted or unsubstituted heteroaryl.
   R¹, R², R³, R⁷, R¹¹, R¹⁹, X¹, X², Y¹, Y², Y³, Y⁴, Y⁵, n, m, and Ring A are each as defined herein, including in embodiments.

In embodiments, R⁴ is independently hydrogen, —C(O)R^{4A}, —C(O)OR^{4A}, —OR^{4A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R⁴ (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R⁴ is independently substituted with a plurality of groups selected from substituent groups, size-limited substituent groups, and lower substituent groups; each substituent group, size-limited substituent group, and/or lower substituent group may optionally be different. In embodiments, when R⁴ is independently substituted, it is substituted with at least one substituent group. In embodiments, when R⁴ is independently substituted, it is substituted with at least one size-limited substituent group. In embodiments, when R⁴ is independently substituted, it is substituted with at least one lower substituent group.

In embodiments, R⁴ is independently hydrogen, substituted or unsubstituted alkyl, or substituted or unsubstituted aryl. In embodiments, R⁴ is independently hydrogen, substituted (e.g., with a substituent group, a size-limited substituent group, or a lower substituent group) or unsubstituted alkyl (e.g., C₁-C₈alkyl, C₁-C₆ alkyl, or C₁-C₄ alkyl), or substituted (e.g., with a substituent group, a size-limited substituent group, or a lower substituent group) or unsubstituted aryl (e.g., C₆-C₁₀ aryl, C₁₀ aryl, or phenyl). In embodiments, R⁴ is independently hydrogen. In embodiments, R⁴ is independently unsubstituted alkyl (e.g., C₁-C₈alkyl, C₁-C₆ alkyl, or C₁-C₄ alkyl). In embodiments, R⁴ is independently substituted (e.g., with a substituent group, a size-limited substituent group or a lower substituent group) alkyl (e.g., C₁-C₈alkyl, C₁-C₆ alkyl, or C₁-C₄ alkyl). In embodiments, R⁴ is independently unsubstituted aryl (e.g., C₆-C₁₀ aryl, C₁₀ aryl, or phenyl). In embodiments, R⁴ is independently substituted (e.g., with a substituent group, a size-limited substituent group or a lower substituent group) aryl (e.g., C₆-C₁₀ aryl, C₁₀ aryl, or phenyl).

In embodiments, R⁴ is independently hydrogen, methyl, ethyl, propyl, isopropyl, butyl, isobutyl, t-butyl, pentyl, isopentyl, hexyl, phenyl, benzyl, or 4-chlorobenzyl.

In embodiments, R⁴ is independently hydrogen. In embodiments, R⁴ is independently methyl. In embodiments, R⁴ is independently ethyl. In embodiments, R⁴ is independently propyl. In embodiments, R⁴ is independently isopropyl. In embodiments, R⁴ is independently butyl. In embodiments, R⁴ is independently isobutyl. In embodiments, R⁴ is independently t-butyl. In embodiments, R⁴ is independently pentyl. In embodiments, R⁴ is independently isopentyl. In embodiments, R⁴ is independently hexyl. In embodiments, R⁴ is independently phenyl. In embodiments, R⁴ is independently benzyl. In embodiments, R⁴ is independently 4-chlorobenzyl.

In embodiments, R⁵ is independently hydrogen, —C(O)R^{5A}, —C(O)OR^{5A}, —OR^{5A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R⁵ (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R⁵ is independently substituted with a plurality of groups selected from substituent groups, size-limited substituent groups, and lower substituent groups; each substituent group, size-limited substituent group, and/or lower substituent group may optionally be different. In embodiments, when R⁵ is independently substituted, it is substituted with at least one substituent group. In embodiments, when R⁵ is independently substituted, it is substituted with at least one size-limited substituent group. In embodiments, when R⁵ is independently substituted, it is substituted with at least one lower substituent group.

In embodiments, R⁵ is independently hydrogen, substituted or unsubstituted alkyl, or substituted or unsubstituted aryl. In embodiments, R⁵ is independently hydrogen, substituted (e.g., with a substituent group, a size-limited substituent group, or a lower substituent group) or unsubstituted alkyl (e.g., C₁-C₈ alkyl, C₁-C₆ alkyl, or C₁-C₄ alkyl), or substituted (e.g., with a substituent group, a size-limited substituent group, or a lower substituent group) or unsubstituted aryl (e.g., C₆-C₁₀ aryl, C₁₀ aryl, or phenyl). In embodiments, R⁵ is independently hydrogen. In embodiments, R⁵ is independently unsubstituted alkyl (e.g., C₁-C₈ alkyl, C₁-C₆ alkyl, or C₁-C₄ alkyl). In embodiments, R⁵ is independently substituted (e.g., with a substituent group, a size-limited substituent group or a lower substituent group) alkyl (e.g., C₁-C₈ alkyl, C₁-C₆ alkyl, or C₁-C₄ alkyl). In embodiments, R⁵ is independently unsubstituted aryl (e.g., C₆-C₁₀ aryl, C₁₀ aryl, or phenyl). In embodiments, R⁵ is independently substituted (e.g., with a substituent group, a size-limited substituent group or a lower substituent group) aryl (e.g., C₆-C₁₀ aryl, C₁₀ aryl, or phenyl).

In embodiments, R⁵ is independently hydrogen, methyl, ethyl, propyl, isopropyl, butyl, isobutyl, t-butyl, pentyl, isopentyl, hexyl, phenyl, benzyl, or 4-chlorobenzyl.

In embodiments, R⁵ is independently hydrogen. In embodiments, R⁵ is independently methyl. In embodiments, R⁵ is independently ethyl. In embodiments, R⁵ is independently propyl. In embodiments, R⁵ is independently isopropyl. In embodiments, R⁵ is independently butyl. In embodiments, R⁵ is independently isobutyl. In embodiments, R⁵ is independently t-butyl. In embodiments, R⁵ is independently pentyl. In embodiments, R⁵ is independently isopentyl. In embodiments, R⁵ is independently hexyl. In embodiments, R⁵ is independently phenyl. In embodiments, R⁵ is independently benzyl. In embodiments, R⁵ is independently 4-chlorobenzyl.

In embodiments, R⁶ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{6A}, —NR^{6A}R^{6B}, —NO₂, —SH, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is hydrogen, halogen, or —NR^{6A}R^{6B} In embodiments, R⁶ is hydrogen. In embodiments, R⁶ is halogen. In embodiments, R⁶ is —NR^{6A}R^{6B} In embodiments, R⁶ is —Cl. In embodiments, R⁶ is —Br. In embodiments, R⁶ is —I. In embodiments, R⁶ is —F. In embodiments, R⁶ is —NHMe. In embodiments, R⁶ is —NH₂. In embodiments, R⁶ is —NMe₂.

In embodiments, R⁶ is hydrogen, —F, —NHMe, —NH₂, or —NMe₂. In embodiments, R⁶ is hydrogen or —NHMe. In embodiments, R⁶ is hydrogen.

In embodiments, R^{6A} is hydrogen or substituted or unsubstituted alkyl. In embodiments, R^{6A} is hydrogen. In embodiments, R^{6A} is substituted alkyl. In embodiments, R^{6A} is an unsubstituted alkyl. In embodiments, R^{6B} is hydrogen or substituted or unsubstituted alkyl. In embodiments, R^{6B} is hydrogen. In embodiments, R^{6B} is substituted alkyl. In embodiments, R^{6B} is an unsubstituted alkyl.

In embodiments, R^{6A} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, R^{6B} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group. In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, R^{6A} is hydrogen or substituted or unsubstituted alkyl. In embodiments, R^{6A} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{6A} is hydrogen. In embodiments, R^{6A} is unsubstituted alkyl (e.g., C₁-C₈ alkyl, C₁-C₆ alkyl, or C₁-C₄ alkyl). In embodiments, R^{6A} is substituted (e.g. with a substituent group, a size-limited substituent group or a lower substituent group) alkyl (e.g., C₁-C₈ alkyl, C₁-C₆ alkyl, or C₁-C₄ alkyl). In embodiments, R^{6B} is hydrogen or substituted or unsubstituted alkyl. In embodiments, R^{6B} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{6B} is hydrogen. In embodiments, R^{6B} is unsubstituted alkyl (e.g., C₁-C₈ alkyl, C₁-C₆ alkyl, or C₁-C₄ alkyl). In embodiments, R^{6B} is substituted (e.g. with a substituent group, a size-limited substituent group or a lower substituent group) alkyl (e.g., C₁-C₈ alkyl, C₁-C₆ alkyl, or C₁-C₄ alkyl).

In embodiments, R^{6A} is hydrogen, methyl, ethyl, propyl, isopropyl, butyl, isobutyl, t-butyl, pentyl, isopentyl, hexyl. In embodiments, R^{6A} is hydrogen. In embodiments, R^{6A} is methyl. In embodiments, R^{6A} is ethyl. In embodiments, R^{6A} is propyl. In embodiments, R^{6A} is isopropyl. In embodiments, R^{6A} is butyl. In embodiments, R^{6A} is isobutyl. In embodiments, R^{6A} is t-butyl. In embodiments, R^{6A} is pentyl. In embodiments, R^{6A} is hexyl. In embodiments, R^{6B} is hydrogen, methyl, ethyl, propyl, isopropyl, butyl, isobutyl, t-butyl, pentyl, isopentyl, hexyl. In embodiments, R^{6B} is hydrogen. In embodiments, R^{6B} is methyl. In embodiments, R^{6B} is ethyl. In embodiments, R^{6B} is propyl. In embodiments, R^{6B} is isopropyl. In embodiments, R^{6B} is butyl. In embodiments, R^{6B} is isobutyl. In embodiments, R^{6B} is t-butyl. In embodiments, R^{6B} is pentyl. In embodiments, R^{6B} is hexyl.

In embodiments, R¹⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{14A}, —NR^{14A}R^{14B}, —NO₂, —S H, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁴ is substituted with a plurality of groups selected from substituent groups, size-limited substituent groups, and lower substituent groups; each substituent group, size-limited substituent group, and/or lower substituent group may optionally be different. In embodiments, when R¹⁴ is substituted, it is substituted with at least one substituent group. In embodiments, when R¹⁴ is substituted, it is substituted with at least one size-limited substituent group. In embodiments, when R¹⁴ is substituted, it is substituted with at least one lower substituent group.

In embodiments, R¹⁴ is hydrogen, halogen, or —NR^{14A}R^{14B}. In embodiments, R¹⁴ is hydrogen. In embodiments, R¹⁴ is halogen. In embodiments, R¹⁴ is —NR^{14A}R^{14B} In embodiments, R¹⁴ is —Cl. In embodiments, R¹⁴ is —Br. In embodiments, R¹⁴ is —I. In embodiments, R¹⁴ is —F. In embodiments, R¹⁴ is —NHMe. In embodiments, R¹⁴ is —NH₂. In embodiments, R¹⁴ is —NMe₂.

In embodiments, R¹⁴ is hydrogen, —F, —NHMe, —NH₂, or —NMe₂. In embodiments, R¹⁴ is hydrogen or —NH₂. In embodiments, R¹⁴ is hydrogen.

In embodiments, R^{14A} is hydrogen or substituted or unsubstituted alkyl. In embodiments, R^{14A} is hydrogen. In embodiments, R^{14A} is substituted alkyl. In embodiments, R^{14A} is an unsubstituted alkyl. In embodiments, R^{14B} is hydrogen or substituted or unsubstituted alkyl. In embodiments, R^{14B} is hydrogen. In embodiments, R^{14B} is substituted alkyl. In embodiments, R^{6B} is an unsubstituted alkyl.

In embodiments, R^{14A} is hydrogen, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl). In embodiments, R^{14B} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{14A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{14A} is substituted with a plurality of groups selected from substituent groups, size-limited substituent groups, and lower substituent groups; each substituent group, size-limited substituent group, and/or lower substituent group may optionally be different. In embodiments, when R^{14A} is substituted, it is substituted with at least one substituent group. In embodiments, when R^{14A} is substituted, it is substituted with at least one size-limited substituent group. In embodiments, when R^{14A} is substituted, it is substituted with at least one lower substituent group. In embodiments, a substituted R^{14B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{14B} is substituted with a plurality of groups selected from substituent groups, size-limited substituent groups, and lower substituent groups; each substituent group, size-limited substituent group, and/or lower substituent group may optionally be different. In embodiments, when R^{14B} is substituted, it is substituted with at least one substituent group. In embodiments, when R^{14B} is substituted, it is substituted with at least one size-limited substituent group. In embodiments, when R^{14B} is substituted, it is substituted with at least one lower substituent group.

In embodiments, R^{14A} is hydrogen or substituted or unsubstituted alkyl. [0011] In embodiments, R^{14A} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{14A} is hydrogen. In embodiments, R^{14A} is unsubstituted alkyl (e.g., C₁-C₈ alkyl, C₁-C₆ alkyl, or C₁-C₄ alkyl). In embodiments, R^{14A} is substituted (e.g. with a substituent group, a size-limited substituent group or a lower substituent group) alkyl (e.g., C₁-C₈ alkyl, C₁-C₆ alkyl, or C₁-C₄ alkyl). In embodiments, R^{14B} is hydrogen or substituted or unsubstituted alkyl. In embodiments, R^{14B} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{14B} is hydrogen. In embodiments, R^{14B} is unsubstituted alkyl (e.g., C₁-C₈alkyl, C₁-C₆ alkyl, or C₁-C₄ alkyl). In embodiments, R^{14B} is substituted (e.g. with a substituent group, a size-limited substituent group or a lower substituent group) alkyl (e.g., C₁-C₈alkyl, C₁-C₆ alkyl, or C₁-C₄ alkyl).

In embodiments, R^{14A} is hydrogen, methyl, ethyl, propyl, isopropyl, butyl, isobutyl, t-butyl, pentyl, isopentyl, hexyl. In embodiments, R^{14A} is hydrogen. In embodiments, R^{14A} is methyl. In embodiments, R^{14A} is ethyl. In embodiments, R^{14A} is propyl. In embodiments, R^{14A} is isopropyl. In embodiments, R^{14A} is butyl. In embodiments, R^{14A} is isobutyl. In embodiments, R^{14A} is t-butyl. In embodiments, R^{14A} is pentyl. In embodiments, R14^{6A} is hexyl. In embodiments, R^{14B} is hydrogen, methyl, ethyl, propyl, isopropyl, butyl, isobutyl, t-butyl, pentyl, isopentyl, hexyl. In embodiments, R^{14B} is hydrogen. In embodiments, R^{14B} is methyl. In embodiments, R^{14B} is ethyl. In embodiments, R^{14B} is propyl. In embodiments, R^{14B} is isopropyl. In embodiments, R^{14B} is butyl. In embodiments, R^{14B} is isobutyl. In embodiments, R^{14B} is t-butyl. In embodiments, R^{14B} is pentyl. In embodiments, R^{14B} is hexyl.

In embodiments, each R^{4A}, R^{5A}, R^{6A}, R^{6B}, R^{14A}, and R^{14B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g., with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{4A}, R^{5A}, R^{6A}, R^{6B}, R^{14A}, and R^{14B} is independently substituted with one or more substituent groups. In embodiments, each R^{4A}, R^{5A}, R^{6A}, R^{6B}, R^{14A}, and R^{14B} is independently substituted with one or more size-limited substituent groups. In embodiments, each R^{4A}, R^{5A}, R^{6A}, R^{6B}, R^{14A}, and R^{14B} is independently substituted with one or more lower substituent groups.

In embodiments, R^{6A} and R^{6B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{6A} and R^{6B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{6A} and R^{9B} substituents bonded to the same nitrogen atom is substituted, it is substituted with at least one substituent group. In embodiments, when a heterocycloalkyl formed by the joining of R^{6A} and R^{6B} substituents bonded to the same nitrogen atom is substituted, it is substituted with at least one size-limited substituent group. In embodiments, when a heterocycloalkyl formed by the joining of R^{6A} and R^{6B} substituents bonded to the same nitrogen atom is substituted, it is substituted with at least one lower substituent group. In embodiments, when a heteroaryl formed by the joining of R^{6A} and R^{6B} substituents bonded to the same nitrogen atom is substituted, it is substituted with at least one substituent group. In embodiments, when a heteroaryl formed by the joining of R^{6A} and R^{6B} substituents bonded to the same nitrogen atom is substituted, it is substituted with at least one size-limited substituent group. In embodiments, when a heteroaryl formed by the joining of R^{6A} and R^{6B} substituents bonded to the same nitrogen atom is substituted, it is substituted with at least one lower substituent group.

In embodiments, R^{14A} and R^{14B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{14A} and R^{14B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{14A} and R^{14B} substituents bonded to the same nitrogen atom is substituted, it is substituted with at least one substituent group. In embodiments, when a heterocycloalkyl formed by the joining of R^{14A} and R^{14B} substituents bonded to the same nitrogen atom is substituted, it is substituted with at least one size-limited substituent group. In embodiments, when a heterocycloalkyl formed by the joining of R^{14A} and R^{14B} substituents bonded to the same nitrogen atom is substituted, it is substituted with at least one lower substituent group. In embodiments, when a heteroaryl formed by the joining of R^{14A} and R^{14B} substituents bonded to the same nitrogen atom is substituted, it is substituted with at least one substituent group. In embodiments, when a heteroaryl formed by the joining of R^{14A} and R^{14B} substituents bonded to the same nitrogen atom is substituted, it is substituted with at least one size-limited substituent group. In embodiments, when a heteroaryl formed by the joining of R^{14A} and R^{14B} substituents bonded to the same nitrogen atom is substituted, it is substituted with at least one lower substituent group.

In embodiments, each X is independently —Cl, —Br, —I or —F. In embodiments, X is independently —Cl. In embodiments, X is independently —Br. In embodiments, X is independently —I. In embodiments, X is independently —F.

In embodiments, provided herein is a structure of formula (IB)

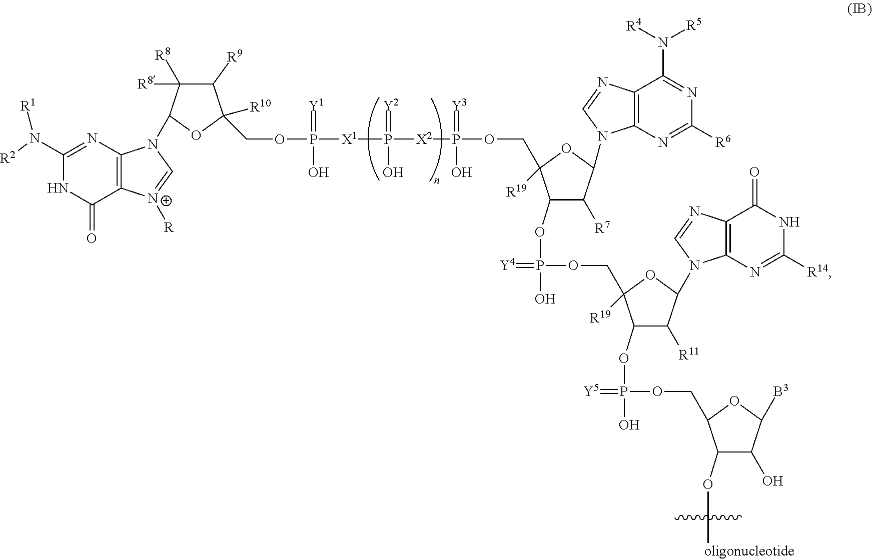

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein:
R⁸ is independently hydrogen, halogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OR^{8A}, —NR^{8A}R^{8B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8′} is independently hydrogen, halogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OR^{8′A}, —NR^{8′A}R^{8′B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8′A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R⁹ is independently hydrogen,
halogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OR^{9A}, —NR^{9A}R^{9B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{9A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   or R⁸ and R⁹ or R^{8′} and R⁹ together with the carbon atoms to which they are connected form a substituted or unsubstituted heterocycloalkylene;
   R¹⁰ is hydrogen, halogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OR^{10A}, —NR^{10A}R^{10B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   or R⁸ and R¹⁰ or R^{8′} and R¹⁰ together with the carbon atoms to which they are connected form a substituted or unsubstituted cycloalkylene or substituted or unsubstituted heterocycloalkylene;
   each R^{8A}, R^{8B}, R^{8′A}, R^{8′B}, R^{9A}, R^{9B}, R^{10A}, and R^{10B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H,
—SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8A} and R^{8B} substituents bonded to the same nitrogen atom may optionally be joined to form a substituted or unsubstituted heterocycloalkyl or substituted or unsubstituted heteroaryl; R^{8′A} and R^{8′B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; and R^{10A} and R^{10B} substituents bonded to the same nitrogen atom may optionally be joined to form a substituted or unsubstituted heterocycloalkyl or substituted or unsubstituted heteroaryl.
R¹, R², R³, R⁴, R⁵, R⁶, R⁷, R¹¹, R¹⁴, R¹⁹, X¹, X², Y¹, Y², Y³, Y⁴, Y⁵, B³, and n, are each as defined herein, including in embodiments.

In embodiments, R⁸ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{8A}, —NR^{8A}R^{8B}, —CONH₂, —COOH, —NHC(O)R^{8A}, —NO₂, —SH, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is independently hydrogen, halogen, —OR^{8A}, or —NHC(O)R^{8A}. In embodiments, R⁸ is independently hydrogen. In embodiments, R⁸ is independently halogen. In embodiments, R⁸ is independently —OR^{8A}. In embodiments, R⁸ is independently —NHC(O)R^{8A}. In embodiments, R⁸ is independently chloro. In embodiments, R⁸ is independently bromo. In embodiments, R⁸ is independently iodo. In embodiments, R⁸ is independently fluoro.

In embodiments, R⁸ is independently hydrogen, hydroxy, methoxy, ethoxy, propoxy, butoxy, or t-butoxy. In embodiments, R⁸ is independently hydrogen or hydroxy.

In embodiments, R⁸ is independently hydroxy. In embodiments, R⁸ is independently methoxy. In embodiments, R⁸ is independently ethoxy. In embodiments, R⁸ is independently propoxy. In embodiments, R⁸ is independently butoxy. In embodiments, R⁸ is independently t-butoxy.

In embodiments, R^{8A} is independently hydrogen or substituted or unsubstituted alkyl. In embodiments, R^{8A} is hydrogen. In embodiments, R^{8A} is a substituted alkyl. In embodiments, R^{8A} is an unsubstituted alkyl.

In embodiments, R^{8A} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{8A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, R^{8A} is hydrogen or substituted or unsubstituted alkyl. In embodiments, R^{8A} is hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{8A} is hydrogen. In embodiments, R^{8A} is unsubstituted alkyl (e.g., C₁-C₈ alkyl, C₁-C₆ alkyl, or C₁-C₄ alkyl). In embodiments, R^{8A} is substituted (e.g., with a substituent group, a size-limited substituent group or a lower substituent group) alkyl (e.g., C₁-C₈alkyl, C₁-C₆ alkyl, or C₁-C₄ alkyl).

In embodiments, R^{8A} is hydrogen, methyl, ethyl, propyl, isopropyl, butyl, isobutyl, t-butyl, pentyl, isopentyl, hexyl. In embodiments, R^{8A} is hydrogen. In embodiments, R^{8A} is methyl. In embodiments, R^{8A} is ethyl. In embodiments, R^{8A} is propyl. In embodiments, R^{8A} is isopropyl. In embodiments, R^{8A} is butyl. In embodiments, R^{8A} is isobutyl. In embodiments, R^{8A} is t-butyl. In embodiments, R^{8A} is pentyl. In embodiments, R^{8A} is hexyl.

In embodiments, R^{8′} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{8′A}, —NR^{8′A}R^{8′B}, —CONH₂, —COOH, —NHC(O)R^{8′A}, —NO₂, —SH, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R^{8′} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8′} is substituted with a plurality of groups selected from substituent groups, size-limited substituent groups, and lower substituent groups; each substituent group, size-limited substituent group, and/or lower substituent group may optionally be different. In embodiments, when R^{8′} is substituted, it is substituted with at least one substituent group. In embodiments, when R^{8′} is substituted, it is substituted with at least one size-limited substituent group. In embodiments, when R^{8′} is substituted, it is substituted with at least one lower substituent group.

In embodiments, R^{8′} is independently hydrogen, halogen, —OR^{8′A}, or —NHC(O)R^{8′A}. In embodiments, R^{8′} is independently hydrogen. In embodiments, R^{8′} is independently halogen. In embodiments, R^{8′} is independently —OR^{8′A}. In embodiments, R^{8′} is independently —NHC(O)R^{8′A}. In embodiments, R^{8′} is independently chloro. In embodiments, R^{8′} is independently bromo. In embodiments, R^{8′} is independently iodo. In embodiments, R^{8′} is independently fluoro.

In embodiments, R^{8′} is independently hydrogen, hydroxy, methoxy, ethoxy, propoxy, butoxy, or t-butoxy. In embodiments, R^{8′} is independently hydrogen or hydroxy.

In embodiments, R^{8′} is independently hydroxy. In embodiments, R^{8′} is independently methoxy. In embodiments, R^{8′} is independently ethoxy. In embodiments, R^{8′} is independently propoxy. In embodiments, R^{8′} is independently butoxy. In embodiments, R^{8′} is independently t-butoxy.

In embodiments, R^{8′A} is independently hydrogen or substituted or unsubstituted alkyl. In embodiments, R^{8′A} is hydrogen. In embodiments, R^{8′A} is a substituted alkyl. In embodiments, R^{8′A} is an unsubstituted alkyl.

In embodiments, R^{8′A} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{8′A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8′A} is substituted with a plurality of groups selected from substituent groups, size-limited substituent groups, and lower substituent groups; each substituent group, size-limited substituent group, and/or lower substituent group may optionally be different. In embodiments, when R^{8′A} is substituted, it is substituted with at least one substituent group. In embodiments, when R^{8′A} is substituted, it is substituted with at least one size-limited substituent group. In embodiments, when R^{8′A} is substituted, it is substituted with at least one lower substituent group.

In embodiments, R^{8′A} is hydrogen or substituted or unsubstituted alkyl. In embodiments, R^{8′A} is hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{8′A} is hydrogen. In embodiments, R^{8′A} is unsubstituted alkyl (e.g., C₁-C₈alkyl, C₁-C₆ alkyl, or C₁-C₄ alkyl). In embodiments, R^{8′A} is substituted (e.g., with a substituent group, a size-limited substituent group or a lower substituent group) alkyl (e.g., C₁-C₈ alkyl, C₁-C₆ alkyl, or C₁-C₄ alkyl).

In embodiments, R^{8′A} is hydrogen, methyl, ethyl, propyl, isopropyl, butyl, isobutyl, t-butyl, pentyl, isopentyl, hexyl. In embodiments, R^{8′A} is hydrogen. In embodiments, R^{8′A} is methyl. In embodiments, R^{8′A} is ethyl. In embodiments, R^{8′A} is propyl. In embodiments, R^{8′A} is isopropyl. In embodiments, R^{8′A} is butyl. In embodiments, R^{8′A} is isobutyl. In embodiments, R^{8′A} is t-butyl. In embodiments, R^{8′A} is pentyl. In embodiments, R^{8′A} is hexyl.

In embodiments, R⁹ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{9A}, —NR^{9A}R^{9B}, —CONH₂, —COOH, —NHC(O)R^{9A}, —NO₂, —SH, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is independently hydrogen, halogen, —OR^{9A}, or —NHC(O)R^{9A} In embodiments, R⁹ is independently hydrogen. In embodiments, R⁹ is independently halogen. In embodiments, R⁹ is independently —OR^{9A}. In embodiments, R⁹ is independently —NHC(O)R^{9A}. In embodiments, R⁹ is independently chloro. In embodiments, R⁹ is independently bromo. In embodiments, R⁹ is independently iodo. In embodiments, R⁹ is independently fluoro.

In embodiments, R⁹ is independently hydrogen, hydroxy, methoxy, ethoxy, propoxy, butoxy, or t-butoxy.

In embodiments, R⁹ is independently hydrogen. In embodiments, R⁹ is independently hydroxy. In embodiments, R⁹ is independently methoxy. In embodiments, R⁹ is independently ethoxy. In embodiments, R⁹ is independently propoxy. In embodiments, R⁹ is independently butoxy. In embodiments, R⁹ is independently t-butoxy.

In embodiments, R^{9A} is independently hydrogen or substituted or unsubstituted alkyl. In embodiments, R^{9A} is hydrogen. In embodiments, R^{9A} is a substituted alkyl. In embodiments, R^{9A} is an unsubstituted alkyl.

In embodiments, R^{9A} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{9A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9A} is substituted with a plurality of groups selected from substituent groups, size-limited substituent groups, and lower substituent groups; each substituent group, size-limited substituent group, and/or lower substituent group may optionally be different. In embodiments, when R^{9A} is substituted, it is substituted with at least one substituent group. In embodiments, when R^{9A} is substituted, it is substituted with at least one size-limited substituent group. In embodiments, when R^{9A} is substituted, it is substituted with at least one lower substituent group.

In embodiments, R^{9A} is hydrogen or substituted or unsubstituted alkyl. In embodiments, R^{9A} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{9A} is hydrogen. In embodiments, R^{9A} is unsubstituted alkyl (e.g., C₁-C₈ alkyl, C₁-C₆ alkyl, or C₁-C₄ alkyl). In embodiments, R^{9A} is substituted (e.g. with a substituent group, a size-limited substituent group or a lower substituent group) alkyl (e.g., C₁-C₈ alkyl, C₁-C₆ alkyl, or C₁-C₄ alkyl).

In embodiments, R^{9A} is hydrogen, methyl, ethyl, propyl, isopropyl, butyl, isobutyl, t-butyl, pentyl, isopentyl, or hexyl. In embodiments, R^{9A} is hydrogen. In embodiments, R^{9A} is methyl. In embodiments, R^{9A} is ethyl. In embodiments, R^{9A} is propyl. In embodiments, R^{9A} is isopropyl. In embodiments, R^{9A} is butyl. In embodiments, R^{9A} is isobutyl. In embodiments, R^{9A} is t-butyl. In embodiments, R^{9A} is pentyl. In embodiments, R^{9A} is isopentyl. In embodiments, R^{9A} is hexyl.

In embodiments, (R⁸ and R⁹) or (R^{8′} and R⁹) together with the carbon atoms to which they are connected form a substituted or unsubstituted heterocycloalkylene. In embodiments, (R⁸ and R⁹) or (R^{8′} and R⁹) together with the carbon atoms to which they are connected form a substituted (e.g., substituted with at least one substituent group, size-limited substituent group, or lower substituent group) or unsubstituted heterocycloalkylene (e.g., 3 to 8 membered heterocycloalkylene, 3 to 6 membered heterocycloalkylene, or 5 to 6 membered heterocycloalkylene).

In embodiments, a substituted heterocycloalkylene formed by the joining of (R⁸ and R⁹) or (R^{8′} and R⁹) together with the carbon atoms to which they are connected is substituted with at least one substituent group, size-limited substituent group, or lower substituent group; wherein if the substituted heterocycloalkylene is substituted with a plurality of groups selected from substituent groups, size-limited substituent groups, and lower substituent groups; each substituent group, size-limited substituent group, and/or lower substituent group may optionally be different. In embodiments, when heterocycloalkylene formed by the joining of (R⁸ and R⁹) or (R^{8′} and R⁹) together with the carbon atoms to which they are connected is substituted, it is substituted with at least one substituent group. In embodiments, when heterocycloalkylene formed by the joining of (R⁸ and R⁹) or (R^{8′} and R⁹) together with the carbon atoms to which they are connected is substituted, it is substituted with at least one size-limited substituent group. In embodiments, when heterocycloalkylene formed by the joining of (R⁸ and R⁹) or (R^{8′} and R⁹) together with the carbon atoms to which they are connected is substituted, it is substituted with at least one lower substituent group.

In embodiments, (R⁸ and R⁹) or (R^{8′} and R⁹) together with the carbon atoms to which they are connected form a substituted or unsubstituted 5 to 6 membered heterocycloalkylene. In embodiments, (R⁸ and R⁹) or (R^{8′} and R⁹) together with the carbon atoms to which they are connected form a substituted or unsubstituted 5 membered heterocycloalkylene. In embodiments, (R⁸ and R⁹) or (R^{8′} and R⁹) together with the carbon atoms to which they are connected form a substituted or unsubstituted 6 membered heterocycloalkylene.

In embodiments, (R⁸ and R⁹) or (R^{8′} and R⁹) together with the carbon atoms to which they are connected form a substituted 5 membered heterocycloalkylene. In embodiments, (R⁸ and R⁹) or (R^{8′} and R⁹) together with the carbon atoms to which they are connected form an unsubstituted 5 membered heterocycloalkylene. In embodiments, (R⁸ and R⁹) or (R^{8′} and R⁹) together with the carbon atoms to which they are connected form a substituted 6 membered heterocycloalkylene. In embodiments, (R⁸ and R⁹) or (R^{8′} and R⁹) together with the carbon atoms to which they are connected form an unsubstituted 6 membered heterocycloalkylene.

In embodiments, R¹⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{10A}, —NR^{10A}R^{10B}, —CONH₂, —COOH, —NHC(O)OH, —NO₂, —SH, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen.

In embodiments, (R⁸ and R¹⁰) or (R^{8′} and R¹⁰) together with the carbon atoms to which they are connected form a substituted or unsubstituted cycloalkylene or substituted or unsubstituted heterocycloalkylene. In embodiments, (R⁸ and R¹⁰) or (R^{8′} and R¹⁰) together with the carbon atoms to which they are connected form a substituted or unsubstituted cycloalkylene. In embodiments, (R⁸ and R¹⁰) or (R^{8′} and R¹⁰) together with the carbon atoms to which they are connected form a substituted or unsubstituted heterocycloalkylene.

In embodiments, (R⁸ and R¹⁰) or (R^{8′} and R¹⁰) together with the carbon atoms to which they are connected form a substituted (e.g., substituted with at least one substituent group, size-limited substituent group, or lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, (R⁸ and R¹⁰) or (R^{8′} and R¹⁰) together with the carbon atoms to which they are connected form a substituted (e.g., substituted with at least one substituent group, size-limited substituent group, or lower substituent group) or unsubstituted cycloalkylene (e.g., C₃-C₈ cycloalkylene, C₃-C₆ cycloalkylene, or C₅-C₆ cycloalkylene).

In embodiments, a substituted heterocycloalkylene formed by the joining of (R⁸ and R¹⁰) or (R^{8′} and R¹⁰) together with the carbon atoms to which they are connected is substituted with at least one substituent group, size-limited substituent group, or lower substituent group; wherein if the substituted heterocycloalkylene is substituted with a plurality of groups selected from substituent groups, size-limited substituent groups, and lower substituent groups; each substituent group, size-limited substituent group, and/or lower substituent group may optionally be different. In embodiments, when heterocycloalkylene formed by the joining of (R⁸ and R¹⁰) or (R^{8′} and R¹⁰) together with the carbon atoms to which they are connected is substituted, it is substituted with at least one substituent group. In embodiments, when heterocycloalkylene formed by the joining of (R⁸ and R¹⁰) or (R^{8′} and R¹⁰) together with the carbon atoms to which they are connected is substituted, it is substituted with at least one size-limited substituent group. In embodiments, when heterocycloalkylene formed by the joining of (R⁸ and R¹⁰) or (R^{8′} and R¹⁰) together with the carbon atoms to which they are connected is substituted, it is substituted with at least one lower substituent group.

In embodiments, a substituted cycloalkylene formed by the joining of (R⁸ and R¹⁰) or (R^{8′} and R¹⁰) together with the carbon atoms to which they are connected is substituted with at least one substituent group, size-limited substituent group, or lower substituent group; wherein if the substituted cycloalkylene is substituted with a plurality of groups selected from substituent groups, size-limited substituent groups, and lower substituent groups; each substituent group, size-limited substituent group, and/or lower substituent group may optionally be different. In embodiments, when cycloalkylene formed by the joining of (R⁸ and R¹⁰) or (R^{8′} and R¹⁰) together with the carbon atoms to which they are connected is substituted, it is substituted with at least one substituent group. In embodiments, when cycloalkylene formed by the joining of (R⁸ and R¹⁰) or (R^{8′} and R¹⁰) together with the carbon atoms to which they are connected is substituted, it is substituted with at least one size-limited substituent group. In embodiments, when cycloalkylene formed by the joining of (R⁸ and R¹⁰) or (R^{8′} and R¹⁰) together with the carbon atoms to which they are connected is substituted, it is substituted with at least one lower substituent group.

In embodiments, (R⁸ and R¹⁰) or (R^{8′} and R¹⁰) together with the carbon atoms to which they are connected form a substituted or unsubstituted 3 to 6 membered heterocycloalkylene. In embodiments, (R⁸ and R¹⁰) or (R^{8′} and R¹⁰) together with the carbon atoms to which they are connected form a substituted or unsubstituted 4 membered heterocycloalkylene.

In embodiments, (R⁸ and R¹⁰) or (R^{8′} and R¹⁰) together with the carbon atoms to which they are connected form a substituted 4 membered heterocycloalkylene. In embodiments, (R⁸ and R¹⁰) or (R^{8′} and R¹⁰) together with the carbon atoms to which they are connected form an unsubstituted 4 membered heterocycloalkylene.

In embodiments, (R⁸ and R¹⁰) or (R^{8′} and R¹⁰) together with the carbon atoms to which they are connected form a locked nucleic acid (LNA).

In embodiments, each R^{8A}, R^{8B}, R^{8′A}, R^{8′B}, R^{9A}, R^{9B}, R^{10A}, and R^{10B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{8A}, R^{8B}, R^{8′A}, R^{8′B}, R^{9A}, R^{9B}, R^{10A}, and R^{10B} is independently substituted with one or more substituent groups. In embodiments, each R^{8A}, R^{8B}, R^{8′A}, R^{8′B}, R^{9A}, R^{9B}, R^{10A}, and R^{10B} is independently substituted with one or more size-limited substituent groups. In embodiments, each R^{8A}, R^{8B}, R^{8′A}, R^{8′B}, R^{9A}, R^{9B}, R^{10A}, and R^{10B} is independently substituted with one or more lower substituent groups.

In embodiments, R^{8A} and R^{8B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{8′A} and R^{8′B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, a substituted heterocycloalkyl or substituted heteroaryl formed by the joining of R^{8A} and R^{8B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different.

In embodiments, a substituted heterocycloalkyl or substituted heteroaryl formed by the joining of R^{8′A} and R^{8′B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different.

In embodiments, when a heterocycloalkyl formed by the joining of R^{8A} and R^{8B} substituents bonded to the same nitrogen atom is substituted, it is substituted with at least one substituent group. In embodiments, when a heterocycloalkyl formed by the joining of R^{8A} and R^{8B} substituents bonded to the same nitrogen atom is substituted, it is substituted with at least one size-limited substituent group. In embodiments, when a heterocycloalkyl formed by the joining of R^{8A} and R^{8B} substituents bonded to the same nitrogen atom is substituted, it is substituted with at least one lower substituent group.

In embodiments, when a heterocycloalkyl formed by the joining of R^{8′A} and R^{8′B} substituents bonded to the same nitrogen atom is substituted, it is substituted with at least one substituent group. In embodiments, when a heterocycloalkyl formed by the joining of R^{8′A} and R^{8′B} substituents bonded to the same nitrogen atom is substituted, it is substituted with at least one size-limited substituent group. In embodiments, when a heterocycloalkyl formed by the joining of R^{8′A} and R^{8′B} substituents bonded to the same nitrogen atom is substituted, it is substituted with at least one lower substituent group.

In embodiments, when a heteroaryl formed by the joining of R^{8A} and R^{8B} substituents bonded to the same nitrogen atom is substituted, it is substituted with at least one substituent group. In embodiments, when a heteroaryl formed by the joining of R^{8A} and R^{8B} substituents bonded to the same nitrogen atom is substituted, it is substituted with at least one size-limited substituent group. In embodiments, when a heteroaryl formed by the joining of R^{8A} and R^{8B} substituents bonded to the same nitrogen atom is substituted, it is substituted with at least one lower substituent group.

In embodiments, when a heteroaryl formed by the joining of R^{8′A} and R^{8′B} substituents bonded to the same nitrogen atom is substituted, it is substituted with at least one substituent group. In embodiments, when a heteroaryl formed by the joining of R^{8′A} and R^{8′B} substituents bonded to the same nitrogen atom is substituted, it is substituted with at least one size-limited substituent group. In embodiments, when a heteroaryl formed by the joining of R^{8′A} and R^{8′B} substituents bonded to the same nitrogen atom is substituted, it is substituted with at least one lower substituent group.

In embodiments, R^{9A} and R^{9B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, a substituted heterocycloalkyl or substituted heteroaryl formed by the joining of R^{9A} and R^{9B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different.

In embodiments, when a heterocycloalkyl formed by the joining of R^{9A} and R^{9B} substituents bonded to the same nitrogen atom is substituted, it is substituted with at least one substituent group. In embodiments, when a heterocycloalkyl formed by the joining of R^{9A} and R^{9B} substituents bonded to the same nitrogen atom is substituted, it is substituted with at least one size-limited substituent group. In embodiments, when a heterocycloalkyl formed by the joining of R^{9A} and R^{9B} substituents bonded to the same nitrogen atom is substituted, it is substituted with at least one lower substituent group.

In embodiments, when a heteroaryl formed by the joining of R^{9A} and R^{9B} substituents bonded to the same nitrogen atom is substituted, it is substituted with at least one substituent group. In embodiments, when a heteroaryl formed by the joining of R^{9A} and R^{9B} substituents bonded to the same nitrogen atom is substituted, it is substituted with at least one size-limited substituent group. In embodiments, when a heteroaryl formed by the joining of R^{9A} and R^{9B} substituents bonded to the same nitrogen atom is substituted, it is substituted with at least one lower substituent group.

In embodiments, R^{10A} and R^{10B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, a substituted heterocycloalkyl or substituted heteroaryl formed by the joining of R^{10A} and R^{10B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different.

In embodiments, when a heterocycloalkyl formed by the joining of R^{10A} and R^{10B} substituents bonded to the same nitrogen atom is substituted, it is substituted with at least one substituent group. In embodiments, when a heterocycloalkyl formed by the joining of R^{10A} and R^{10B} substituents bonded to the same nitrogen atom is substituted, it is substituted with at least one size-limited substituent group. In embodiments, when a heterocycloalkyl formed by the joining of R^{10A} and R^{10B} substituents bonded to the same nitrogen atom is substituted, it is substituted with at least one lower substituent group.

In embodiments, when a heteroaryl formed by the joining of R^{10A} and R^{10B} substituents bonded to the same nitrogen atom is substituted, it is substituted with at least one substituent group. In embodiments, when a heteroaryl formed by the joining of R^{10A} and R^{10B} substituents bonded to the same nitrogen atom is substituted, it is substituted with at least one size-limited substituent group. In embodiments, when a heteroaryl formed by the joining of R^{10A} and R^{10B} substituents bonded to the same nitrogen atom is substituted, it is substituted with at least one lower substituent group.

In embodiments, each X is independently —Cl, —Br, —I or —F. In embodiments, X is independently —Cl. In embodiments, X is independently —Br. In embodiments, X is independently —I. In embodiments, X is independently —F.

In embodiments, provided herein is a structure of formula (IC):

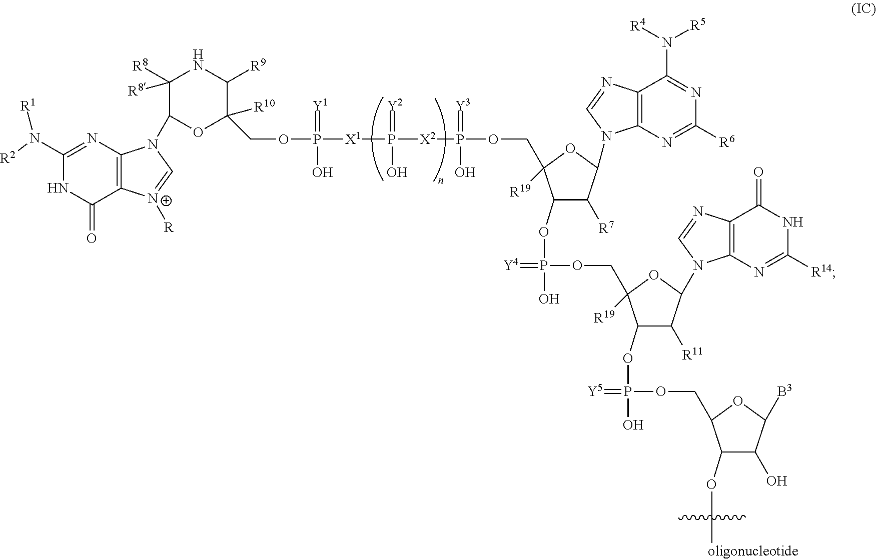

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁶, R⁷, R⁸, R^{8′}, R⁹, R¹⁰, R¹¹, R¹⁴, R¹⁹, X¹, X², Y¹, Y², Y³, Y⁴, Y⁵, B³, and n, are each as defined herein, including in embodiments.

In embodiments, provided herein is a structure of formula (ID):

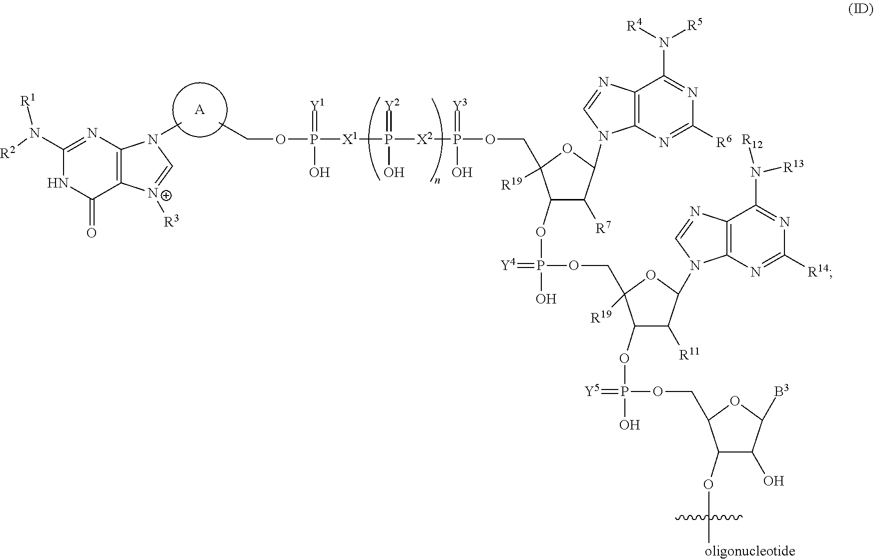

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof,
wherein:
R¹² is independently hydrogen, —C(O)R^{12A}, —C(O)OR^{12A}, —OR^{12A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹³ is independently hydrogen, —C(O)R^{13A}, —C(O)OR^{13A}, —OR^{13A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R⁴ and R⁵ together with the nitrogen atom to which they are connected form a substituted or unsubstituted heteroaryl or a substituted or unsubstituted heterocyclyl;
each R^{12A} and R^{13A} is independently hydrogen, —CX₃, —CHX₂, CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
—OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹, R², R³, R⁴, R⁵, R⁶, R⁷, R¹¹, R¹⁴, R¹⁹, X¹, X², Y¹, Y², Y³, Y⁴, Y⁵, B³, Ring A, and n, are each as defined herein, including in embodiments.

In embodiments, R¹² is independently hydrogen, —C(O)R^{12A}, —C(O)OR^{12A}, —OR^{12A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹² (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R¹² is independently substituted with a plurality of groups selected from substituent groups, size-limited substituent groups, and lower substituent groups; each substituent group, size-limited substituent group, and/or lower substituent group may optionally be different. In embodiments, when R¹² is independently substituted, it is substituted with at least one substituent group. In embodiments, when R¹² is independently substituted, it is substituted with at least one size-limited substituent group. In embodiments, when R¹² is independently substituted, it is substituted with at least one lower substituent group.

In embodiments, R¹² is independently hydrogen or substituted or unsubstituted alkyl. In embodiments, R¹² is independently hydrogen or substituted (e.g., with a substituent group, a size-limited substituent group or a lower substituent group) or unsubstituted alkyl (e.g., C₁-C₈alkyl, C₁-C₆ alkyl, or C₁-C₄ alkyl). In embodiments, R¹² is independently hydrogen. In embodiments, R¹² is independently unsubstituted alkyl (e.g., C₁-C₈ alkyl, C₁-C₆ alkyl, or C₁-C₄ alkyl). In embodiments, R¹² is independently substituted (e.g., with a substituent group, a size-limited substituent group or a lower substituent group) alkyl (e.g., C₁-C₈alkyl, C₁-C₆ alkyl, or C₁-C₄ alkyl).

In embodiments, R¹² is independently hydrogen, methyl, ethyl, propyl, isopropyl, butyl, isobutyl, t-butyl, pentyl, isopentyl, or hexyl.

In embodiments, R¹² is independently hydrogen or methyl.

In embodiments, R¹² is independently hydrogen. In embodiments, R¹² is independently methyl. In embodiments, R¹² is independently ethyl. In embodiments, R¹² is independently propyl. In embodiments, R¹² is independently isopropyl. In embodiments, R¹² is independently butyl. In embodiments, R¹² is independently isobutyl. In embodiments, R¹² is independently t-butyl. In embodiments, R¹² is independently pentyl. In embodiments, R¹² is independently isopentyl. In embodiments, R¹² is independently hexyl.

In embodiments, R¹³ is independently hydrogen, —C(O)R^{13A}, —C(O)OR^{13A}, —OR^{13A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹³ (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R¹³ is independently substituted with a plurality of groups selected from substituent groups, size-limited substituent groups, and lower substituent groups; each substituent group, size-limited substituent group, and/or lower substituent group may optionally be different. In embodiments, when R¹³ is independently substituted, it is substituted with at least one substituent group. In embodiments, when R¹³ is independently substituted, it is substituted with at least one size-limited substituent group. In embodiments, when R¹³ is independently substituted, it is substituted with at least one lower substituent group.

In embodiments, R¹³ is independently hydrogen or substituted or unsubstituted alkyl. In embodiments, R¹³ is independently hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R¹³ is independently hydrogen. In embodiments, R¹³ is independently unsubstituted alkyl (e.g., C₁-C₈ alkyl, C₁-C₆ alkyl, or C₁-C₄ alkyl). In embodiments, R¹³ is independently substituted (e.g., with a substituent group, a size-limited substituent group or a lower substituent group) alkyl (e.g., C₁-C₈alkyl, C₁-C₆ alkyl, or C₁-C₄ alkyl).

In embodiments, R¹³ is independently hydrogen, methyl, ethyl, propyl, isopropyl, butyl, isobutyl, t-butyl, pentyl, isopentyl, or hexyl.

In embodiments, R¹³ is independently hydrogen or methyl.

In embodiments, R¹³ is independently hydrogen. In embodiments, R¹³ is independently methyl. In embodiments, R¹³ is independently ethyl. In embodiments, R¹³ is independently propyl. In embodiments, R¹³ is independently isopropyl. In embodiments, R¹³ is independently butyl. In embodiments, R¹³ is independently isobutyl. In embodiments, R¹³ is independently t-butyl. In embodiments, R¹³ is independently pentyl. In embodiments, R¹³ is independently isopentyl. In embodiments, R¹³ is independently hexyl.

In embodiments, each R^{12A} and R^{13A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H,

—SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g., with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{12A} and R^{13A} is independently substituted with one or more substituent groups. In embodiments, each R^{12A} and R^{13A} is independently substituted with one or more size-limited substituent groups. In embodiments, each R^{12A} and R^{13A} is independently substituted with one or more lower substituent groups.

In embodiments, each X is independently —Cl, —Br, —I or —F. In embodiments, X is independently —Cl. In embodiments, X is independently —Br. In embodiments, X is independently —I. In embodiments, X is independently —F.

In embodiments, provided herein is a structure of formula (IE):

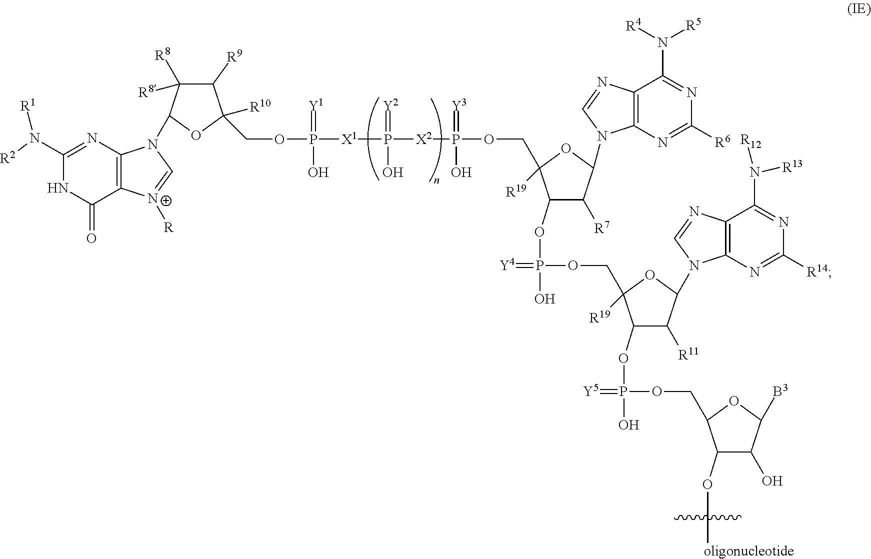

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁴, R⁵, R⁶, R⁷, R⁸, R^{8′}, R⁹, R¹⁰, R¹¹, R¹², R¹³, R¹⁴, R¹⁹, X¹, X², Y¹, Y², Y³, Y⁴, Y⁵, B³, and n, are each as defined herein, including in embodiments.

In embodiments, provided herein is a structure of formula (IF):

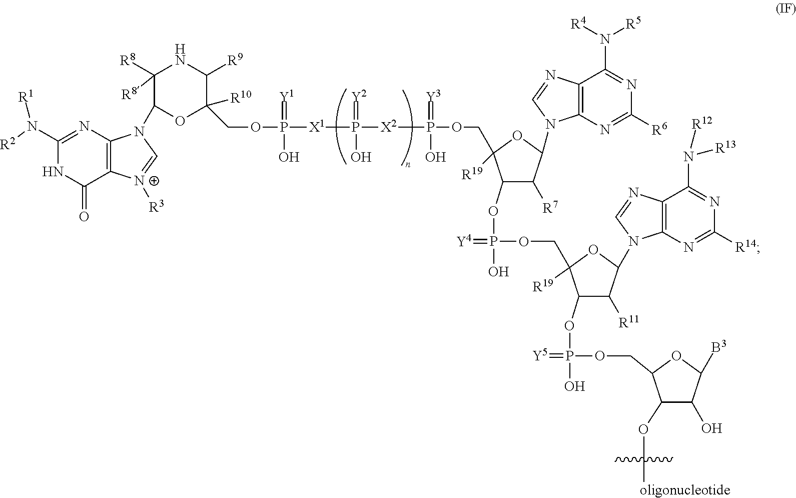

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein: R¹, R², R³, R⁴, R⁵, R⁶, R⁷, R⁸, R^{8′}, R⁹, R¹⁰, R¹¹, R¹², R¹³, R¹⁴, R¹⁹, X¹, X², Y¹, Y², Y³, Y⁴, Y⁵, B³, and n, are each as defined herein, including in embodiments.

In embodiments, provided herein is a structure of formula (IG):

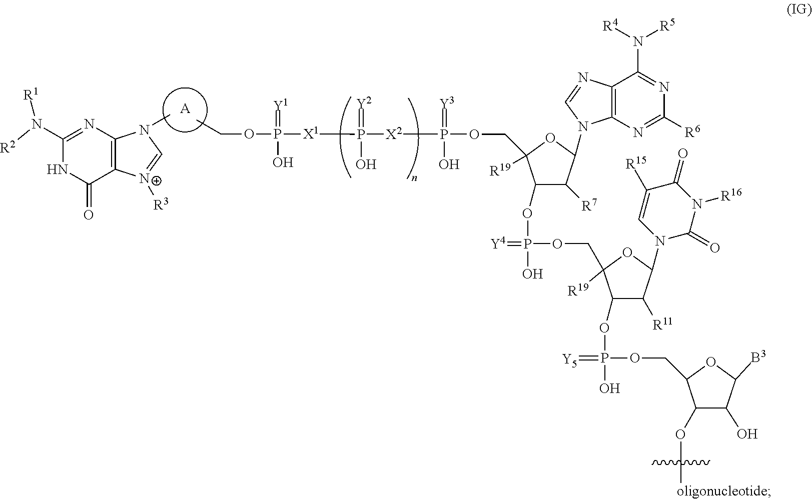

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein
R¹⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{15A}, —NR^{15A}R^{15B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁶ is independently hydrogen, —C(O)R^{16A}, —C(O)OR^{16A}, —OR^{16A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{15A}, R^{15B}, and R^{16A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
—OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{15A} and R^{15B} substituents bonded to the same nitrogen atom may optionally be joined to form a substituted or unsubstituted heterocycloalkyl or substituted or unsubstituted heteroaryl.
   R¹, R², R³, R⁴, R⁵, R⁶, R⁷, R¹¹, R¹⁹, X¹, X², Y¹, Y², Y³, Y⁴, Y³, B³, Ring A, and n, are each as defined herein, including in embodiments.

In embodiments, R¹⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{15A}, —NR^{15A}R^{15B}, —NO₂, —S H, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, R¹⁵ is hydrogen, halogen, or —NR^{15A}R^{15B}. In embodiments, R¹⁵ is hydrogen. In embodiments, R¹⁵ is halogen. In embodiments, R¹⁵ is —NR^{15A}R^{15B}. In embodiments, R¹⁵ is —Cl. In embodiments, R¹⁵ is —Br. In embodiments, R¹⁵ is —I. In embodiments, R¹⁵ is —F. In embodiments, R¹⁵ is —NHMe. In embodiments, R¹⁵ is —NH₂. In embodiments, R¹⁵ is —NMe₂.

In embodiments, R¹⁵ is hydrogen, —F, —NHMe, —NH₂, or —NMe₂. In embodiments, R¹⁵ is hydrogen, —NHMe, or —NH₂.

In embodiments, R^{15A} is hydrogen or substituted or unsubstituted alkyl. In embodiments, R^{15A} is hydrogen. In embodiments, R^{15A} is substituted alkyl. In embodiments, R^{15A} is an unsubstituted alkyl. In embodiments, R^{15B} is hydrogen or substituted or unsubstituted alkyl. In embodiments, R^{15B} is hydrogen. In embodiments, R^{15B} is substituted alkyl. In embodiments, R^{15B} is an unsubstituted alkyl.

In embodiments, R^{15A} is hydrogen, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl). In embodiments, R^{15B} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{15A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15A} is substituted with a plurality of groups selected from substituent groups, size-limited substituent groups, and lower substituent groups; each substituent group, size-limited substituent group, and/or lower substituent group may optionally be different. In embodiments, when R^{15A} is substituted, it is substituted with at least one substituent group. In embodiments, when R^{15A} is substituted, it is substituted with at least one size-limited substituent group. In embodiments, when R^{15A} is substituted, it is substituted with at least one lower substituent group. In embodiments, a substituted R^{15B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15B} is substituted with a plurality of groups selected from substituent groups, size-limited substituent groups, and lower substituent groups; each substituent group, size-limited substituent group, and/or lower substituent group may optionally be different. In embodiments, when R^{15B} is substituted, it is substituted with at least one substituent group. In embodiments, when R^{15B} is substituted, it is substituted with at least one size-limited substituent group. In embodiments, when R^{15B} is substituted, it is substituted with at least one lower substituent group.

In embodiments, R^{15A} is hydrogen or substituted or unsubstituted alkyl. [0018] In embodiments, R^{15A} is hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{15A} is hydrogen. In embodiments, R^{15A} is unsubstituted alkyl (e.g., C₁-C₈ alkyl, C₁-C₆ alkyl, or C₁-C₄ alkyl). In embodiments, R^{15A} is substituted (e.g., with a substituent group, a size-limited substituent group or a lower substituent group) alkyl (e.g., C₁-C₈ alkyl, C₁-C₆ alkyl, or C₁-C₄ alkyl). In embodiments, R^{15B} is hydrogen or substituted or unsubstituted alkyl. In embodiments, R^{15B} is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{15B} is hydrogen. In embodiments, R^{15B} is unsubstituted alkyl (e.g., C₁-C₈ alkyl, C₁-C₆ alkyl, or C₁-C₄ alkyl). In embodiments, R^{15B} is substituted (e.g., with a substituent group, a size-limited substituent group or a lower substituent group) alkyl (e.g., C₁-C₈ alkyl, C₁-C₆ alkyl, or C₁-C₄ alkyl).

In embodiments, R^{15A} is hydrogen, methyl, ethyl, propyl, isopropyl, butyl, isobutyl, t-butyl, pentyl, isopentyl, hexyl. In embodiments, R^{15A} is hydrogen. In embodiments, R^{15A} is methyl. In embodiments, R^{15A} is ethyl. In embodiments, R^{15A} is propyl. In embodiments, R^{15A} is isopropyl. In embodiments, R^{15A} is butyl. In embodiments, R^{15A} is isobutyl. In embodiments, R^{15A} is t-butyl. In embodiments, R^{15A} is pentyl. In embodiments, R^{15A} is hexyl. In embodiments, R^{15B} is hydrogen, methyl, ethyl, propyl, isopropyl, butyl, isobutyl, t-butyl, pentyl, isopentyl, hexyl. In embodiments, R^{15B} is hydrogen. In embodiments, R^{15B} is methyl. In embodiments, R^{15B} is ethyl. In embodiments, R^{15B} is propyl. In embodiments, R^{15B} is isopropyl. In embodiments, R^{15B} is butyl. In embodiments, R^{15B} is isobutyl. In embodiments, R^{15B} is t-butyl. In embodiments, R^{15B} is pentyl. In embodiments, R^{15B} is hexyl.

In embodiments, R¹⁶ is independently hydrogen, —C(O)R^{16A}, —C(O)OR^{16A}, —OR^{16A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁶ (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R¹⁶ is independently substituted with a plurality of groups selected from substituent groups, size-limited substituent groups, and lower substituent groups; each substituent group, size-limited substituent group, and/or lower substituent group may optionally be different. In embodiments, when R¹⁶ is independently substituted, it is substituted with at least one substituent group. In embodiments, when R¹⁶ is independently substituted, it is substituted with at least one size-limited substituent group. In embodiments, when R¹⁶ is independently substituted, it is substituted with at least one lower substituent group.

In embodiments, R¹⁶ is independently hydrogen or substituted or unsubstituted alkyl. In embodiments, R¹⁶ is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R¹⁶ is independently hydrogen. In embodiments, R¹⁶ is independently unsubstituted alkyl (e.g., C₁-C₈ alkyl, C₁-C₆ alkyl, or C₁-C₄ alkyl). In embodiments, R¹⁶ is independently substituted (e.g. with a substituent group, a size-limited substituent group or a lower substituent group) alkyl (e.g., C₁-C₈alkyl, C₁-C₆ alkyl, or C₁-C₄ alkyl).

In embodiments, R¹⁶ is independently hydrogen, methyl, ethyl, propyl, isopropyl, butyl, isobutyl, t-butyl, pentyl, isopentyl, or hexyl.

In embodiments, R¹⁶ is independently hydrogen. In embodiments, R¹⁶ is independently methyl. In embodiments, R¹⁶ is independently ethyl. In embodiments, R¹⁶ is independently propyl. In embodiments, R¹⁶ is independently isopropyl. In embodiments, R¹⁶ is independently butyl. In embodiments, R¹⁶ is independently isobutyl. In embodiments, R¹⁶ is independently t-butyl. In embodiments, R¹⁶ is independently pentyl. In embodiments, R¹⁶ is independently isopentyl. In embodiments, R¹⁶ is independently hexyl.

In embodiments, each R^{15A}, R^{15B}, and R^{16A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H,

—SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{15A}, R^{15B}, and R^{16A} is independently substituted with one or more substituent groups. In embodiments, each R^{15A}, R^{15B}, and R^{16A} is independently substituted with one or more size-limited substituent groups. In embodiments, each R^{15A}, R^{15B}, and R^{16A} is independently substituted with one or more lower substituent groups.

In embodiments, R^{15A} and R^{15B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{15A} and R^{15B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{15A} and R^{15B} substituents bonded to the same nitrogen atom is substituted, it is substituted with at least one substituent group. In embodiments, when a heterocycloalkyl formed by the joining of R^{15A} and R^{15B} substituents bonded to the same nitrogen atom is substituted, it is substituted with at least one size-limited substituent group. In embodiments, when a heterocycloalkyl formed by the joining of R^{15A} and R^{15B} substituents bonded to the same nitrogen atom is substituted, it is substituted with at least one lower substituent group. In embodiments, when a heteroaryl formed by the joining of R^{15A} and R^{15B} substituents bonded to the same nitrogen atom is substituted, it is substituted with at least one substituent group. In embodiments, when a heteroaryl formed by the joining of R^{15A} and R^{15B} substituents bonded to the same nitrogen atom is substituted, it is substituted with at least one size-limited substituent group. In embodiments, when a heteroaryl formed by the joining of R^{15A} and R^{15B} substituents bonded to the same nitrogen atom is substituted, it is substituted with at least one lower substituent group.

In embodiments, each X is independently —Cl, —Br, —I or —F. In embodiments, X is independently —Cl. In embodiments, X is independently —Br. In embodiments, X is independently —I. In embodiments, X is independently —F.

In embodiments, provided herein is a structure of formula (IH):

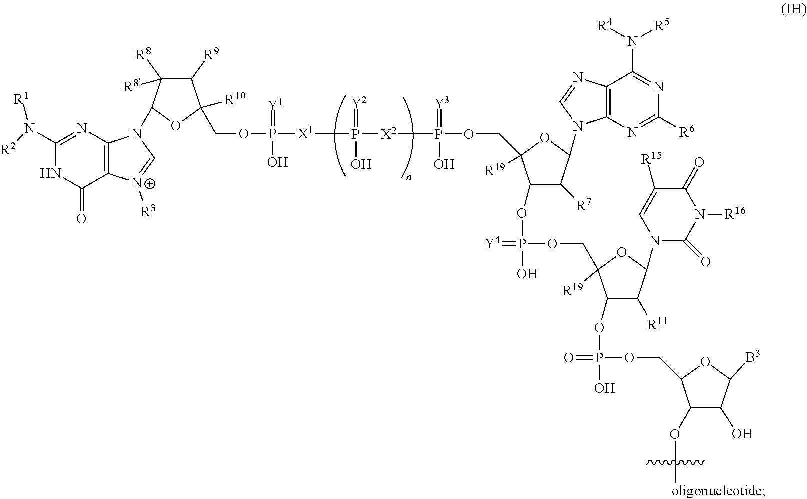

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁴, R⁵, R⁶, R⁷, R⁸, R^{8′}, R⁹, R¹⁰, R¹¹, R¹⁵, R¹⁶, R¹⁹, X¹, X², Y¹, Y², Y³, Y⁴, Y⁵, B³, and n, are each as defined herein, including in embodiments.

In embodiments, provided herein is a structure of formula (IJ):

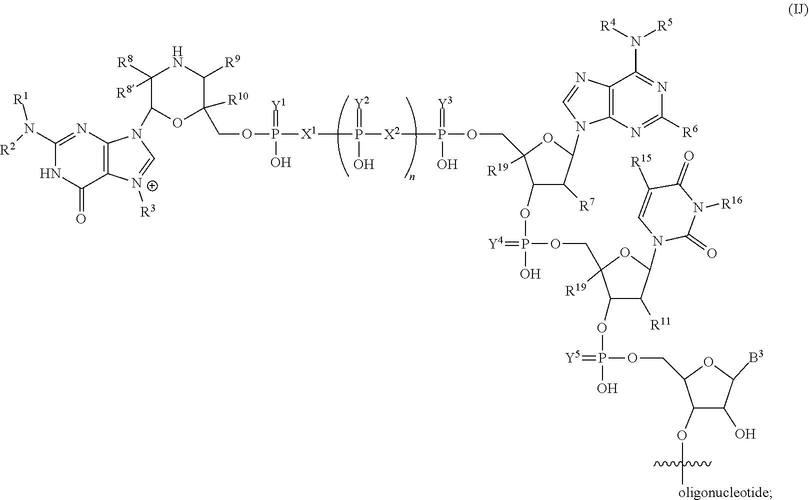

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁴, R⁵, R⁶, R⁷, R⁸, R^{8′}, R⁹, R¹⁰, R¹¹, R¹⁵, R¹⁶, R¹⁹, X¹, X², Y¹, Y², Y³, Y⁴, Y⁵, B³, and n, are each as defined herein, including in embodiments.

In embodiments, provided herein is a structure of formula (IK):

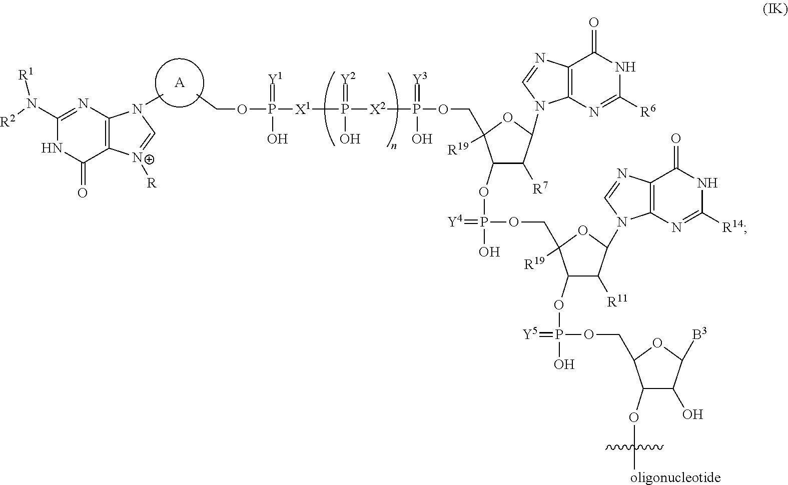

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁶, R⁷, R¹¹, R¹⁴, R¹⁹, X¹, X², Y¹, Y², Y³, Y⁴, Y⁵, B³, Ring A, and n, are each as defined herein, including in embodiments.

In embodiments, provided herein is a structure of formula (IL):

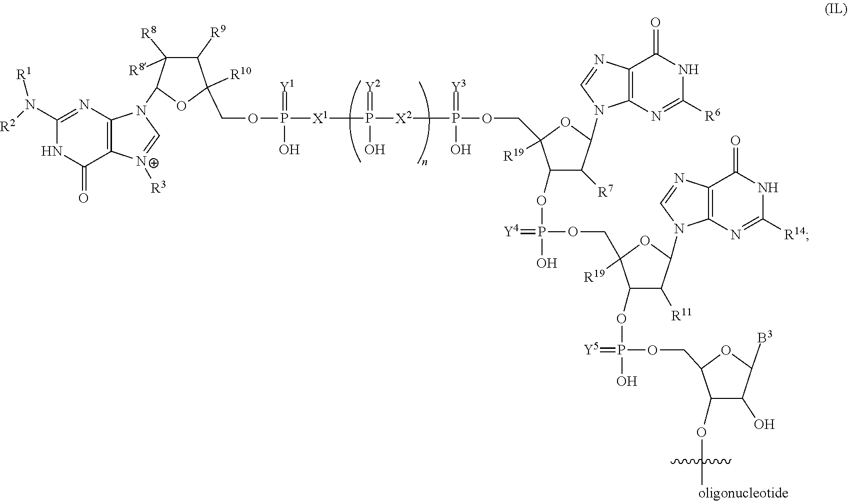

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁶, R⁷, R⁸, R^{8′}, R⁹, R¹⁰, R¹¹, R¹⁴, R¹⁹, X¹, X², Y¹, Y², Y³, Y⁴, Y⁵, B³, and n, are each as defined herein, including in embodiments.

In embodiments, provided herein is a structure of formula (IM):

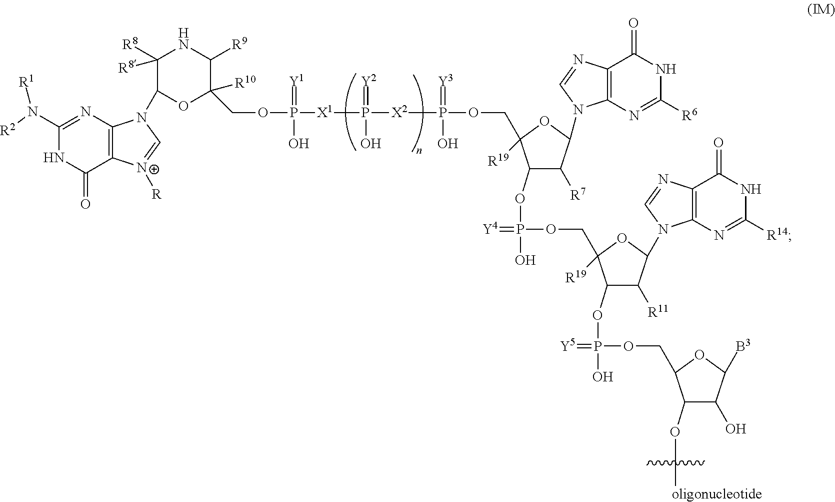

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁶, R⁷, R⁸, R^{8′}, R⁹, R¹⁰, R¹¹, R¹⁴, R¹⁹, X¹, X², Y¹, Y², Y³, Y⁴, Y⁵, B³, and n, are each as defined herein, including in embodiments.

In embodiments, provided herein is a structure of formula (IN):

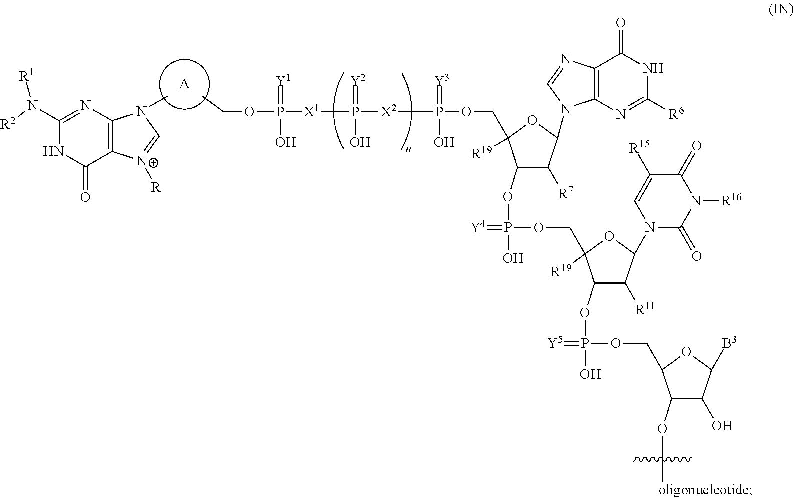

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein: R¹, R², R³, R⁶, R⁷, R¹¹, R¹⁵, R¹⁶, R¹⁹, X¹, X², Y¹, Y², Y³, Y⁴, Y⁵, B³, Ring A, and n, are each as defined herein, including in embodiments.

In embodiments, provided herein is a structure of formula (IO):

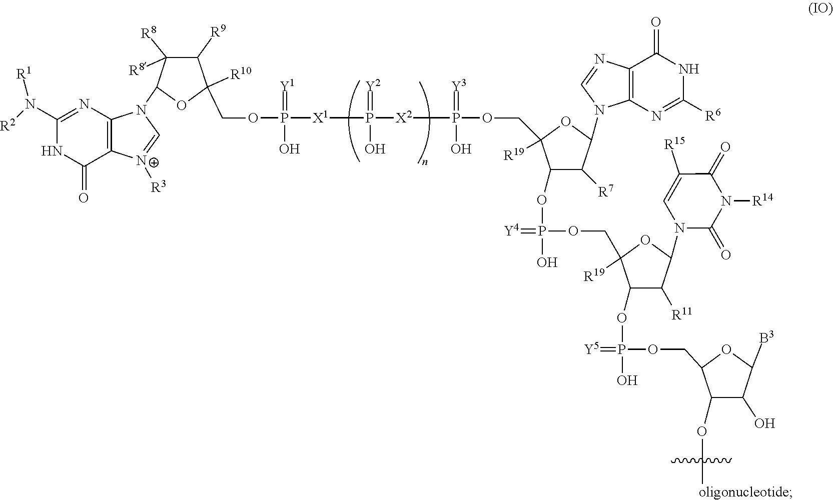

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁶, R⁷, R⁸, R^{8′}, R⁹, R¹⁰, R¹¹, R¹⁵, R¹⁶, R¹⁹, X¹, X², Y¹, Y², Y³, Y⁴, Y⁵, B³, and n, are each as defined herein, including in embodiments.

In embodiments, provided herein is a structure of formula (IP):

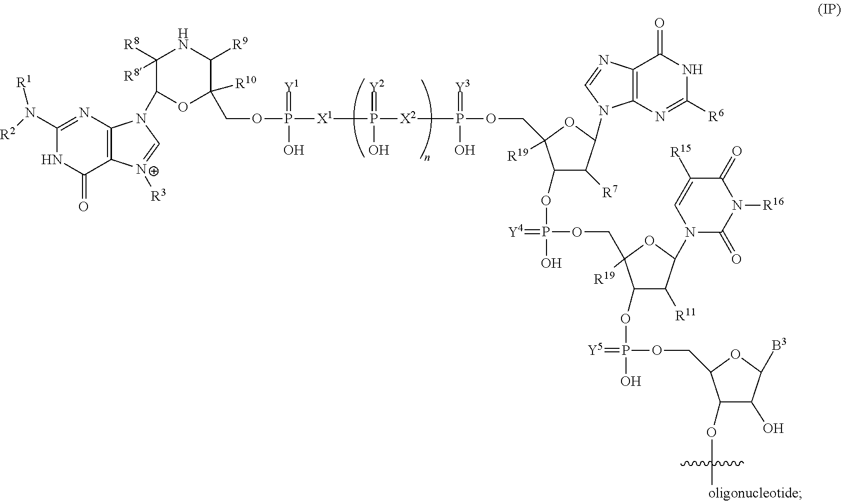

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁶, R⁷, R⁸, R^{8′}, R⁹, R¹⁰, R¹¹, R¹⁵, R¹⁶, R¹⁹, X¹, X², Y¹, Y², Y³, Y⁴, Y⁵, B³, and n, are each as defined herein, including in embodiments.

In embodiments, provided herein is a structure of formula (IQ):

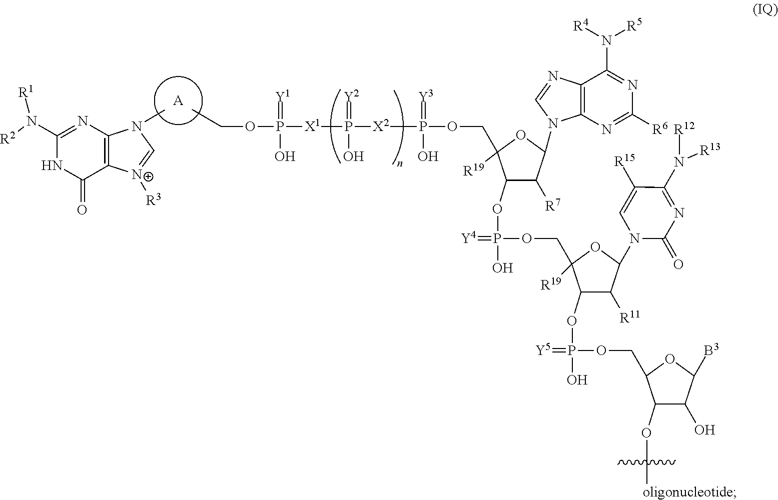

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein: R¹, R², R³, R⁴, R⁵, R⁶, R⁷, R¹¹, R¹², R¹³, R¹⁵, R¹⁹, X¹, X², Y¹, Y², Y³, Y⁴, Y⁵, B³, Ring A, and n, are each as defined herein, including in embodiments.

In embodiments, provided herein is a structure of formula (IR):

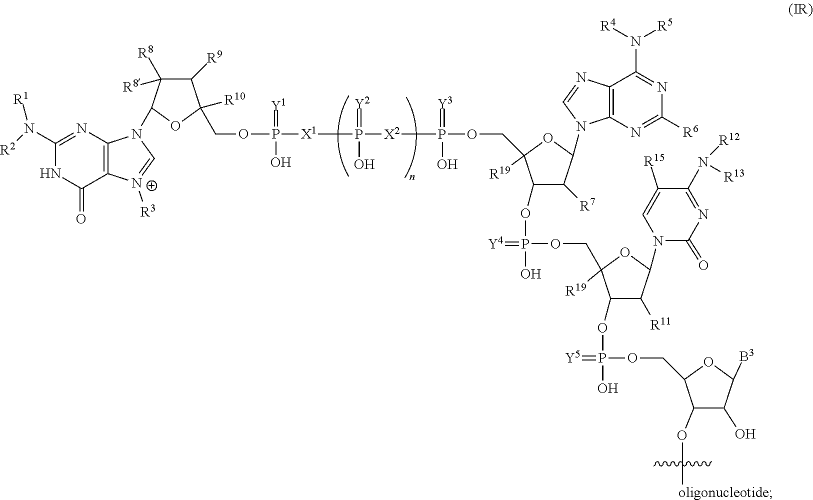

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁴, R⁵, R⁶, R⁷, R⁸, R^{8′}, R⁹, R¹⁰, R¹¹, R¹², R¹³, R¹⁵, R¹⁹, X¹, X², Y¹, Y², Y³, Y⁴, Y⁵, B³, and n, are each as defined herein, including in embodiments.

In embodiments, provided herein is a structure of formula (IS):

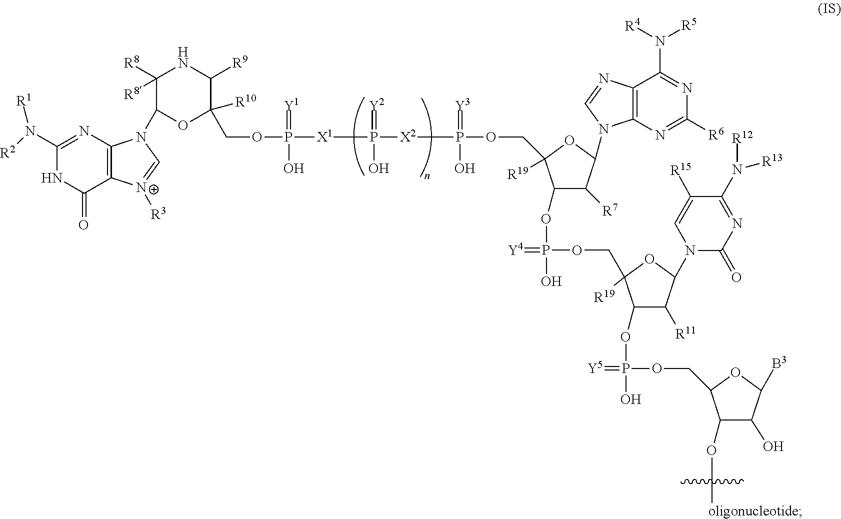

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein: R¹, R², R³, R⁴, R⁵, R⁶, R⁷, R⁸, R^{8′}, R⁹, R¹⁰, R¹¹, R¹², R¹³, R¹⁵, R¹⁹, X¹, X², Y¹, Y², Y³, Y⁴, Y⁵, B³, and n, are each as defined herein, including in embodiments.

In embodiments, provided herein is a structure of formula (IT):

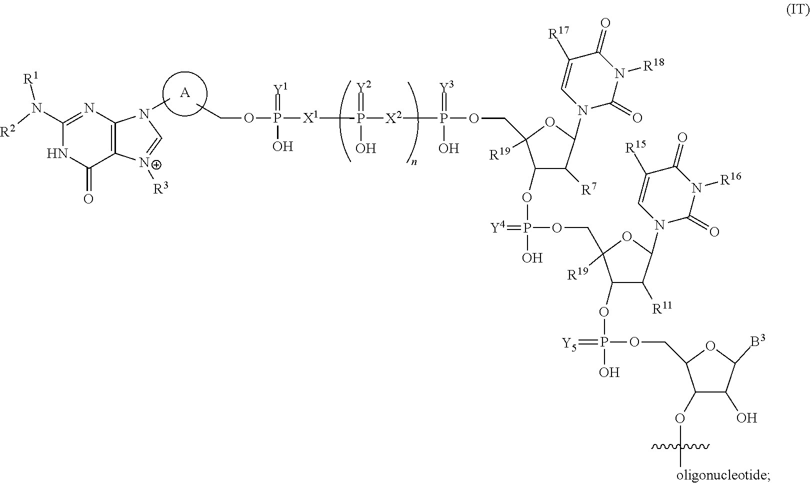

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof,
wherein:
R¹⁷ is hydrogen, halogen, —CCl₃, —CBr₃, —CF₃, —CI₃, —CHCl₂, —CHBr₂, —CHF₂, —CHI₂, CH₂Cl, —CH₂Br, —CH₂F, —CH₂I, —CN, —OR^{17A}, —NR^{17A}R^{17B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁸ is independently hydrogen, —C(O)R^{18A}, —C(O)OR^{18A}, —OR^{18A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{17A}, R^{17B}, and R^{18A}, is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
—OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{17A} and R^{17B} substituents bonded to the same nitrogen atom may optionally be joined to form a substituted or unsubstituted heterocycloalkyl or substituted or unsubstituted heteroaryl.
wherein: R¹, R², R³, R⁷, R¹¹, R¹⁵, R¹⁶, R¹⁹, X¹, X², Y¹, Y², Y³, Y⁴, Y³, B³, Ring A, and n, are each as defined herein, including in embodiments.

In embodiments, R¹⁷ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{17A}, —NR^{17A}R^{17B}, —NO₂, —S H, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁷ is hydrogen, halogen, or —NR^{17A}R^{17B}. In embodiments, R¹⁷ is hydrogen. In embodiments, R¹⁷ is halogen. In embodiments, R¹⁷ is —NR^{17A}R^{17B}. In embodiments, R¹⁷ is —Cl. In embodiments, R¹⁷ is —Br. In embodiments, R¹⁷ is —I. In embodiments, R¹⁷ is —F. In embodiments, R¹⁷ is —NHMe. In embodiments, R¹⁷ is —NH₂. In embodiments, R¹⁷ is —NMe₂.

In embodiments, R¹⁷ is hydrogen, —F, —NHMe, —NH₂, or —NMe₂. In embodiments, R¹⁷ is hydrogen, —NHMe, or —NH₂.

In embodiments, R^{17A} is hydrogen or substituted or unsubstituted alkyl. In embodiments, R^{17A} is hydrogen. In embodiments, R^{17A} is substituted alkyl. In embodiments, R^{17A} is an unsubstituted alkyl. In embodiments, R^{17B} is hydrogen or substituted or unsubstituted alkyl. In embodiments, R^{17B} is hydrogen. In embodiments, R^{17B} is substituted alkyl. In embodiments, R^{17B} is an unsubstituted alkyl.

In embodiments, R^{17A} is hydrogen, substituted (e.g., substituted with at least one substituent group, size-limited substituent group, or lower substituent group) or unsubstituted alkyl (e.g., C₁-C₈alkyl, C₁-C₆ alkyl, or C₁-C₄ alkyl). In embodiments, R^{17B} is hydrogen, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl).

In embodiments, a substituted R^{17A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17A} is substituted with a plurality of groups selected from substituent groups, size-limited substituent groups, and lower substituent groups; each substituent group, size-limited substituent group, and/or lower substituent group may optionally be different. In embodiments, when R^{17A} is substituted, it is substituted with at least one substituent group. In embodiments, when R^{17A} is substituted, it is substituted with at least one size-limited substituent group. In embodiments, when R^{17A} is substituted, it is substituted with at least one lower substituent group. In embodiments, a substituted R^{17B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17B} is substituted with a plurality of groups selected from substituent groups, size-limited substituent groups, and lower substituent groups; each substituent group, size-limited substituent group, and/or lower substituent group may optionally be different. In embodiments, when R^{17B} is substituted, it is substituted with at least one substituent group. In embodiments, when R^{17B} is substituted, it is substituted with at least one size-limited substituent group. In embodiments, when R^{17B} is substituted, it is substituted with at least one lower substituent group.

In embodiments, R^{17A} is hydrogen or substituted or unsubstituted alkyl. [0021] In embodiments, R^{17A} is hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{17A} is hydrogen. In embodiments, R^{17A} is unsubstituted alkyl (e.g., C₁-C₈alkyl, C₁-C₆ alkyl, or C₁-C₄ alkyl). In embodiments, R^{17A} is substituted (e.g., with a substituent group, a size-limited substituent group or a lower substituent group) alkyl (e.g., C₁-C₈ alkyl, C₁-C₆ alkyl, or C₁-C₄ alkyl). In embodiments, R^{17B} is hydrogen or substituted or unsubstituted alkyl. In embodiments, R^{17B} is hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{17B} is hydrogen. In embodiments, R^{17B} is unsubstituted alkyl (e.g., C₁-C₈alkyl, C₁-C₆ alkyl, or C₁-C₄ alkyl). In embodiments, R^{17B} is substituted (e.g., with a substituent group, a size-limited substituent group or a lower substituent group) alkyl (e.g., C₁-C₈ alkyl, C₁-C₆ alkyl, or C₁-C₄ alkyl).

In embodiments, R^{17A} is hydrogen, methyl, ethyl, propyl, isopropyl, butyl, isobutyl, t-butyl, pentyl, isopentyl, hexyl. In embodiments, R^{17A} is hydrogen. In embodiments, R^{17A} is methyl. In embodiments, R^{17A} is ethyl. In embodiments, R^{17A} is propyl. In embodiments, R^{17A} is isopropyl. In embodiments, R^{17A} is butyl. In embodiments, R^{17A} is isobutyl. In embodiments, R^{17A} is t-butyl. In embodiments, R^{17A} is pentyl. In embodiments, R^{17A} is hexyl. In embodiments, R^{17B} is hydrogen, methyl, ethyl, propyl, isopropyl, butyl, isobutyl, t-butyl, pentyl, isopentyl, hexyl. In embodiments, R^{17B} is hydrogen. In embodiments, R^{17B} is methyl. In embodiments, R^{17B} is ethyl. In embodiments, R^{17B} is propyl. In embodiments, R^{17B} is isopropyl. In embodiments, R^{17B} is butyl. In embodiments, R^{17B} is isobutyl. In embodiments, R^{17B} is t-butyl. In embodiments, R^{17B} is pentyl. In embodiments, R^{17B} is hexyl.

In embodiments, R¹⁸ is independently hydrogen, —C(O)R^{18A}, —C(O)OR^{18A}, —OR^{18A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, a substituted R¹⁸ (e.g., substituted alkyl, substituted heteroalkyl, substituted cycloalkyl, substituted heterocycloalkyl, substituted aryl, and/or substituted heteroaryl) is independently substituted with at least one substituent group, size-limited substituent group, or lower substituent group; wherein if the substituted R¹⁸ is independently substituted with a plurality of groups selected from substituent groups, size-limited substituent groups, and lower substituent groups; each substituent group, size-limited substituent group, and/or lower substituent group may optionally be different. In embodiments, when R¹⁸ is independently substituted, it is substituted with at least one substituent group. In embodiments, when R¹⁸ is independently substituted, it is substituted with at least one size-limited substituent group. In embodiments, when R¹⁸ is independently substituted, it is substituted with at least one lower substituent group.

In embodiments, R¹⁸ is independently hydrogen or substituted or unsubstituted alkyl. In embodiments, R¹⁸ is independently hydrogen or substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, R¹⁸ is independently hydrogen. In embodiments, R¹⁸ is independently unsubstituted alkyl (e.g., C₁-C₈ alkyl, C₁-C₆ alkyl, or C₁-C₄ alkyl). In embodiments, R¹⁸ is independently substituted (e.g. with a substituent group, a size-limited substituent group or a lower substituent group) alkyl (e.g., C₁-C₈alkyl, C₁-C₆ alkyl, or C₁-C₄ alkyl).

In embodiments, R¹⁸ is independently hydrogen, methyl, ethyl, propyl, isopropyl, butyl, isobutyl, t-butyl, pentyl, isopentyl, or hexyl.

In embodiments, R¹⁸ is independently hydrogen. In embodiments, R¹⁸ is independently methyl. In embodiments, R¹⁸ is independently ethyl. In embodiments, R¹⁸ is independently propyl. In embodiments, R¹⁸ is independently isopropyl. In embodiments, R¹⁸ is independently butyl. In embodiments, R¹⁸ is independently isobutyl. In embodiments, R¹⁸ is independently t-butyl. In embodiments, R¹⁸ is independently pentyl. In embodiments, R¹⁸ is independently isopentyl. In embodiments, R¹⁸ is independently hexyl.

In embodiments, each R^{17A}, R^{17B}, and R^{18A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H,

—SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{17A}, R^{17B}, and R^{18A} is independently substituted with one or more substituent groups. In embodiments, each R^{17A}, R^{17B}, and R^{18A} is independently substituted with one or more size-limited substituent groups. In embodiments, each R^{17A}, R^{17B}, and R^{18A} is independently substituted with one or more lower substituent groups.

In embodiments, R^{17A} and R^{17B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{17A} and R^{17B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{17A} and R^{17B} substituents bonded to the same nitrogen atom is substituted, it is substituted with at least one substituent group. In embodiments, when a heterocycloalkyl formed by the joining of R^{17A} and R^{17B} substituents bonded to the same nitrogen atom is substituted, it is substituted with at least one size-limited substituent group. In embodiments, when a heterocycloalkyl formed by the joining of R^{17A} and R^{17B} substituents bonded to the same nitrogen atom is substituted, it is substituted with at least one lower substituent group. In embodiments, when a heteroaryl formed by the joining of R^{17A} and R^{17B} substituents bonded to the same nitrogen atom is substituted, it is substituted with at least one substituent group. In embodiments, when a heteroaryl formed by the joining of R^{17A} and R^{17B} substituents bonded to the same nitrogen atom is substituted, it is substituted with at least one size-limited substituent group. In embodiments, when a heteroaryl formed by the joining of R^{17A} and R^{17B} substituents bonded to the same nitrogen atom is substituted, it is substituted with at least one lower substituent group.

In embodiments, each X is independently —Cl, —Br, —I or —F. In embodiments, X is independently —Cl. In embodiments, X is independently —Br. In embodiments, X is independently —I. In embodiments, X is independently —F.

In embodiments, provided herein is a structure of formula (IU):

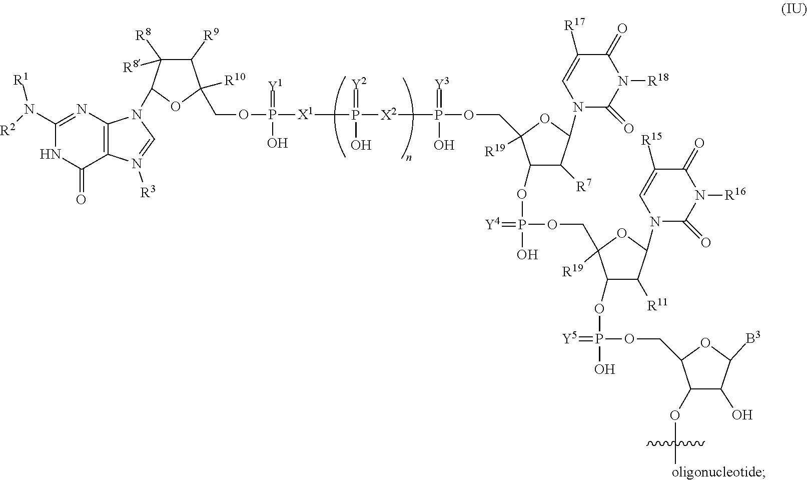

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein R¹, R², R³, R⁷, R⁸, R^{8′}, R⁹, R¹⁰, R¹¹, R¹⁵, R¹⁶, R¹⁷, R¹⁸, R¹⁹, X¹, X², Y¹, Y², Y³, Y⁴, Y⁵, B³, and n, are each as defined herein, including in embodiments.

In embodiments, provided herein is a structure of formula (IV):

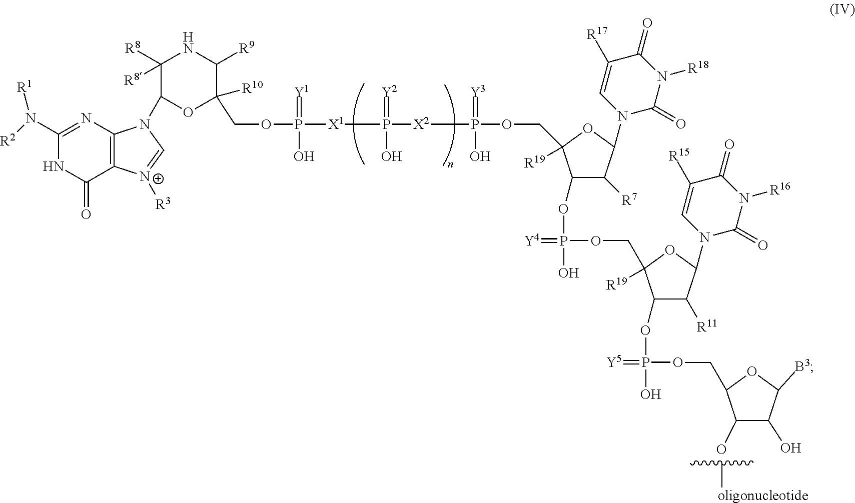

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁷, R⁸, R^{8′}, R⁹, R¹⁰, R¹¹, R¹⁵, R¹⁶, R¹⁷, R¹⁸, R¹⁹, X¹, X², Y¹, Y², Y³, Y⁴, Y⁵, B³, and n, are each as defined herein, including in embodiments.

In embodiments, provided herein is a structure of formula (IW):

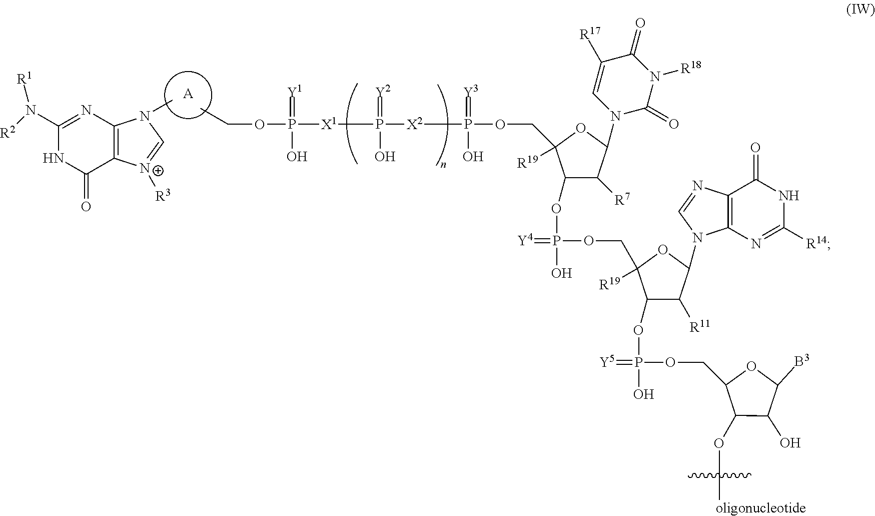

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁷, R¹¹, R¹⁴, R¹⁷, R¹⁸, R¹⁹, X¹, X², Y¹, Y², Y³, Y⁴, Y⁵, B³, Ring A, and n, are each as defined herein, including in embodiments.

In embodiments, provided herein is a structure of formula (IX):

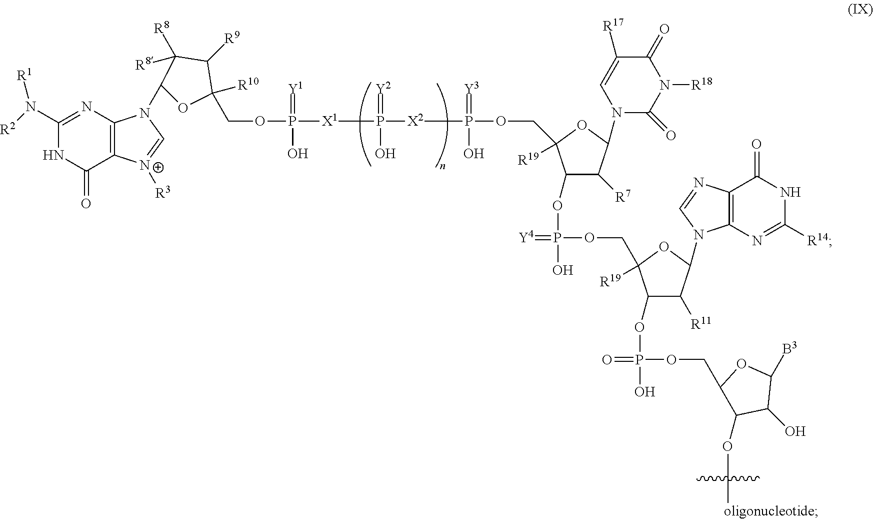

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁷, R⁸, R^{8′}, R⁹, R¹⁰, R¹¹, R¹⁴, R¹⁷, R¹⁸, R¹⁹, X¹, X², Y¹, Y², Y³, Y⁴, Y⁵, B³, and n, are each as defined herein, including in embodiments.

In embodiments, provided herein is a structure of formula (IY):

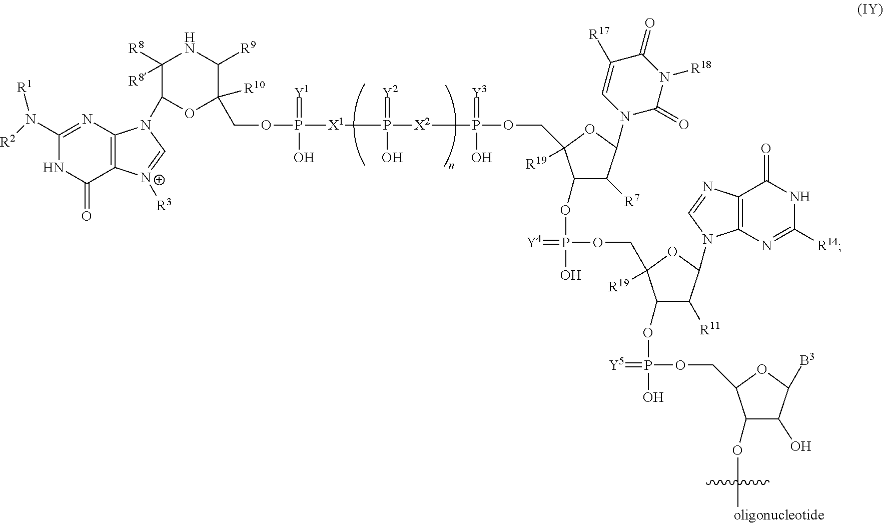

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁷, R⁸, R^{8′}, R⁹, R¹⁰, R¹¹, R¹⁴, R¹⁷, R¹⁸, R¹⁹, X¹, X², Y¹, Y², Y³, Y⁴, Y⁵, B³, and n, are each as defined herein, including in embodiments.

In embodiments, provided herein is a structure of formula (IZ):

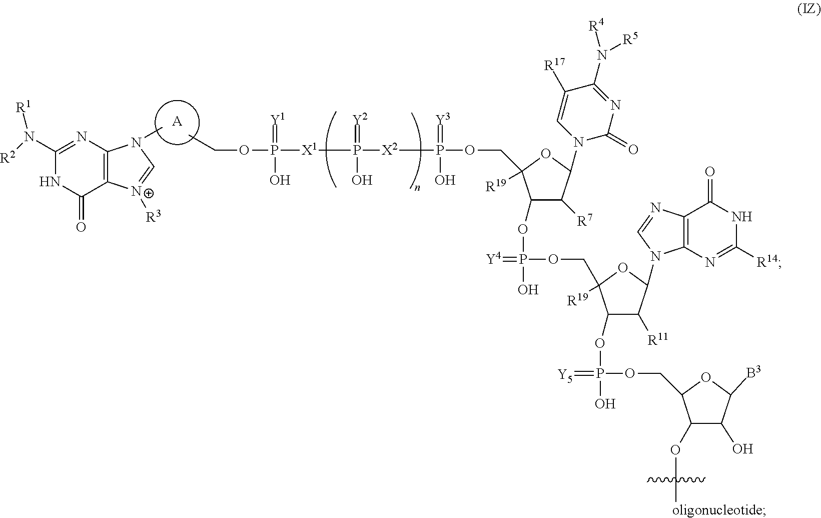

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁷, R¹¹, R¹⁴, R¹⁷, R¹⁹, X¹, X², Y¹, Y², Y³, Y⁴, Y⁵, B³, Ring A, and n, are each as defined herein, including in embodiments.

In embodiments, provided herein is a structure of formula (IAA):

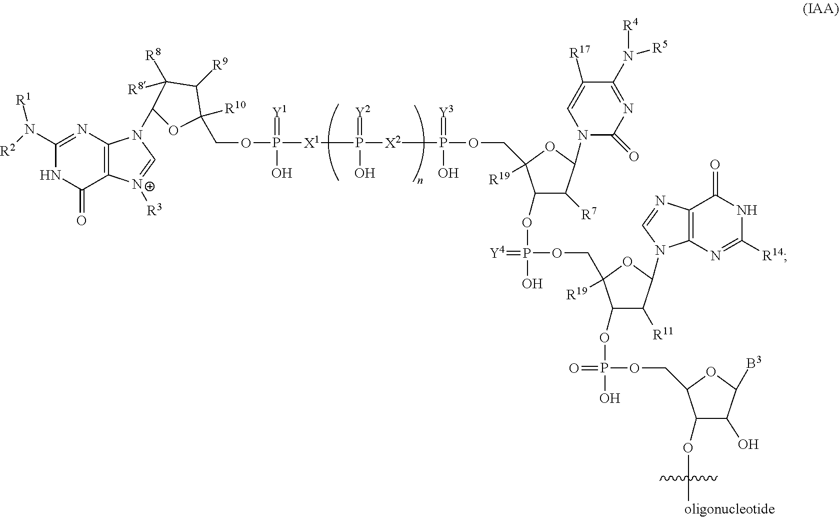

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁷, R⁸, R^{8′}, R⁹, R¹⁰, R¹¹, R¹⁴, R¹⁷, R¹⁹, X¹, X², Y¹, Y², Y³, Y⁴, Y⁵, B³, and n, are each as defined herein, including in embodiments.

In embodiments, provided herein is a structure of formula (IBB):

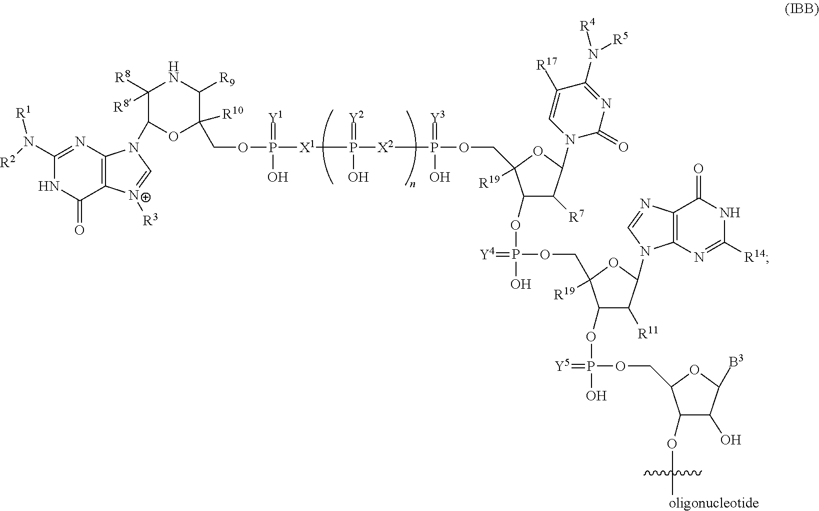

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁷, R⁸, R^{8′}, R⁹, R¹⁰, R¹¹, R¹⁴, R¹⁷, R¹⁹, X¹, X², Y¹, Y², Y³, Y⁴, Y⁵, B³, and n, are each as defined herein, including in embodiments.

In embodiments, provided herein is a structure of formula (ICC):

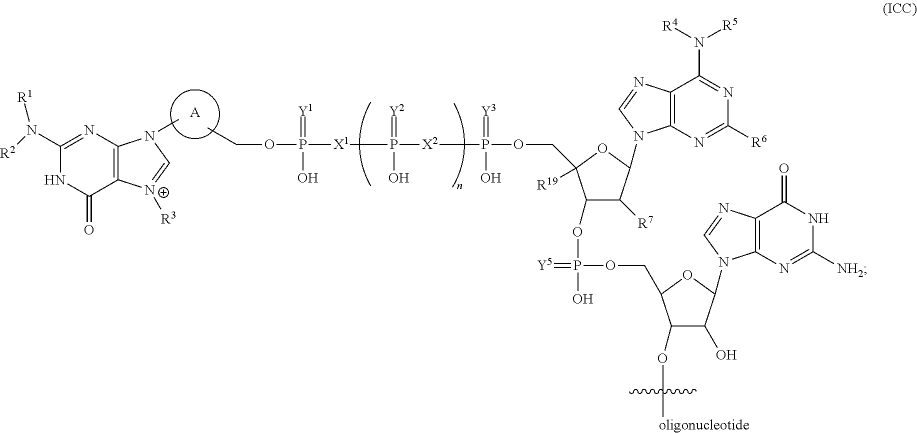

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein R¹, R², R³, R⁴, R⁵, R⁶, R⁷, R¹⁹, X¹, X², Y¹, Y², Y³, Y⁵, Ring A, and n, are each as defined herein, including in embodiments.

In embodiments, provided herein is a structure of formula (IDD):

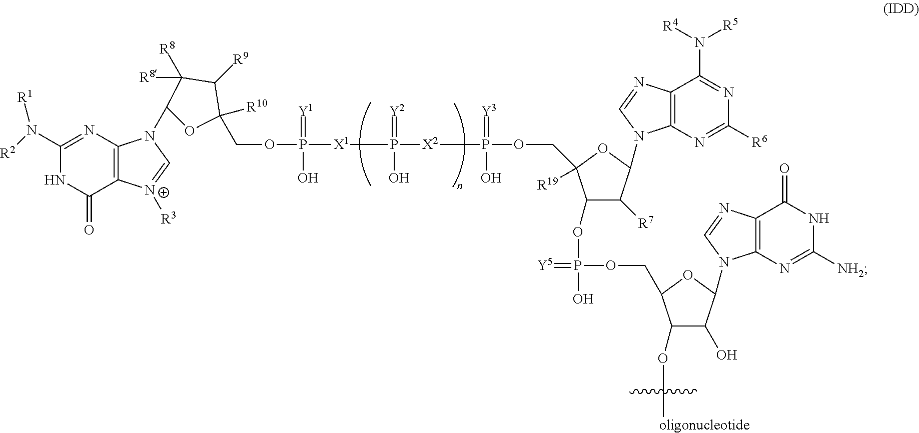

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁶, R⁷, R⁸, R^{8′}, R⁹, R¹⁰, R¹⁹, X¹, X², Y¹, Y², Y³, Y⁵, and n, are each as defined herein, including in embodiments.

In embodiments, provided herein is a structure of formula (IEE):

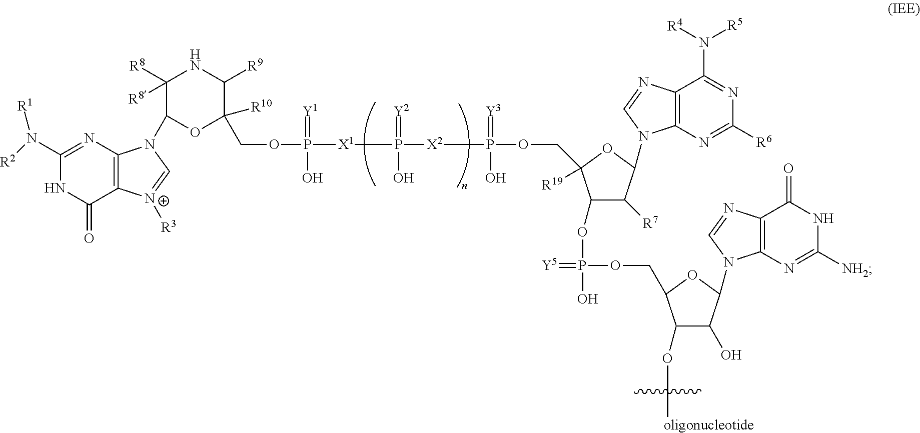

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁶, R⁷, R⁸, R^{8′}, R⁹, R¹⁰, R¹⁹, X¹, X², Y¹, Y², Y³, Y⁵, and n, are each as defined herein, including in embodiments.

In embodiments, provided herein is a structure of formula (IFF):

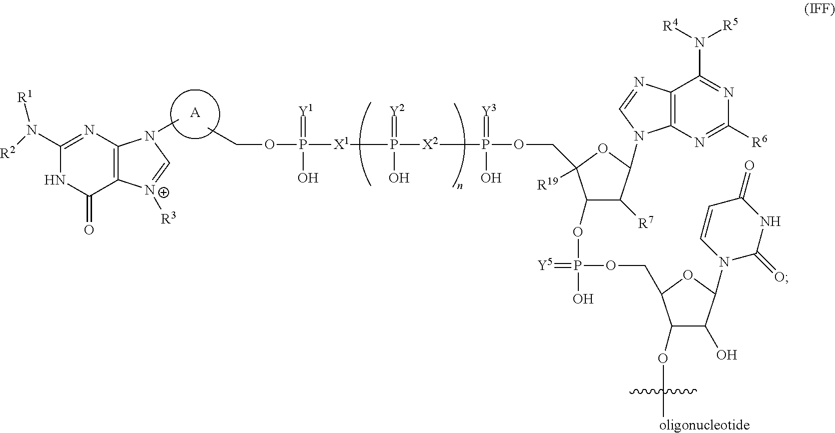

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein R¹, R², R³, R⁴, R⁵, R⁶, R⁷, R¹⁹, X¹, X², Y¹, Y², Y³, Y⁵, Ring A, and n, are each as defined herein, including in embodiments.

In embodiments, provided herein is a structure of formula (IGG):

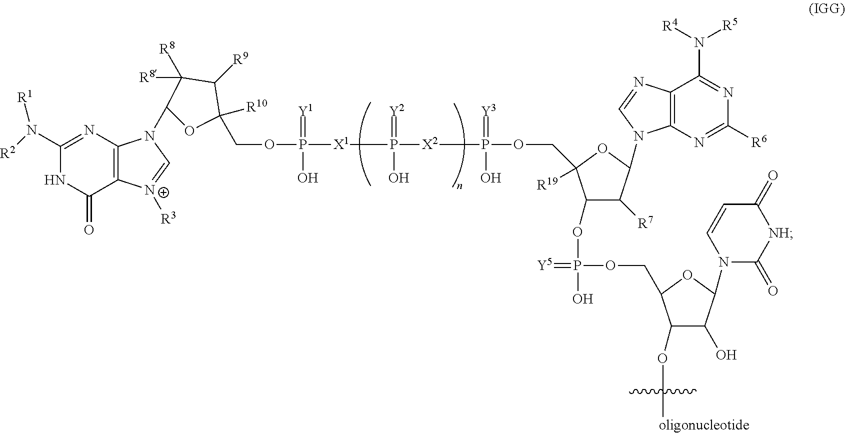

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁶, R⁷, R⁸, R^{8′}, R⁹, R¹⁰, R¹⁹, X¹, X², Y¹, Y², Y³, Y⁵, and n, are each as defined herein, including in embodiments.

In embodiments, provided herein is a structure of formula (IHH):

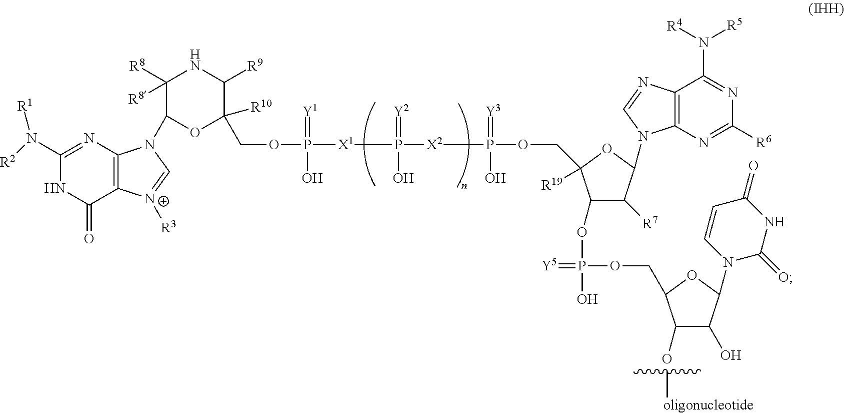

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, R⁶, R⁷, R⁸, R^{8′}, R⁹, R¹⁰, R¹⁹, X¹, X², Y¹, Y², Y³, Y⁵, and n, are each as defined herein, including in embodiments.

In embodiments, provided herein is a structure of formula (IJJ):

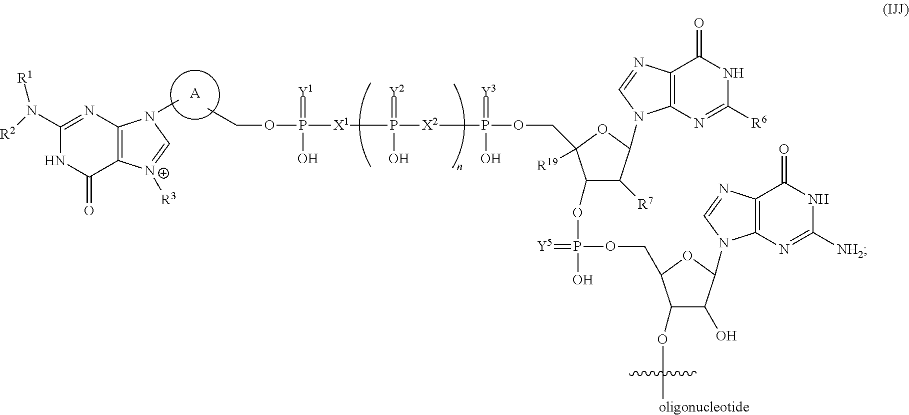

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein R¹, R², R³, R⁶, R⁷, R¹⁹, X¹, X², Y¹, Y², Y³, Y⁵, Ring A, and n, are each as defined herein, including in embodiments.

In embodiments, provided herein is a structure of formula (IKK):

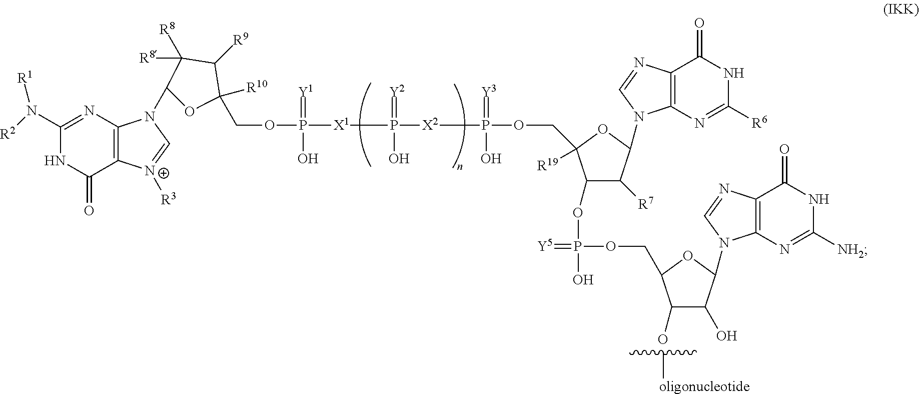

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁶, R⁷, R⁸, R^{8′}, R⁹, R¹⁰, R¹⁹, X¹, X², Y¹, Y², Y³, Y⁵, and n, are each as defined herein, including in embodiments.

In embodiments, provided herein is a structure of formula (ILL):

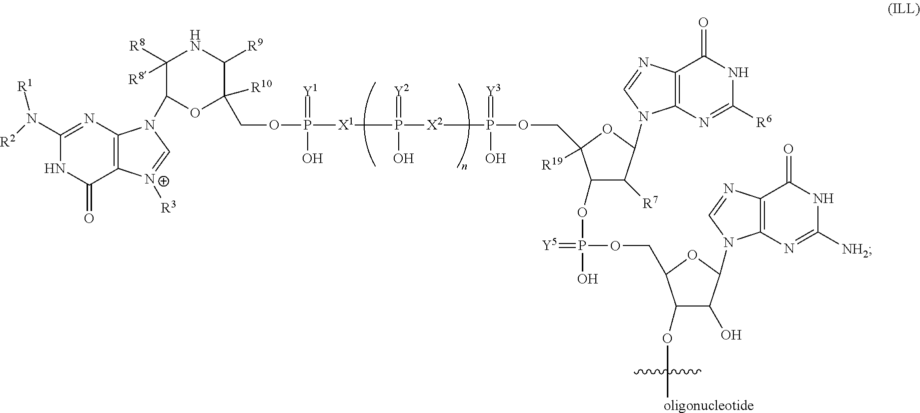

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁶, R⁷, R⁸, R^{8′}, R⁹, R¹⁰, R¹⁹, X¹, X², Y¹, Y², Y³, Y⁵, and n, are each as defined herein, including in embodiments.

In an aspect, provided herein is an oligonucleotide comprising 50-12000 nucleotides, whose 5′ end comprises a structure of formula (II):

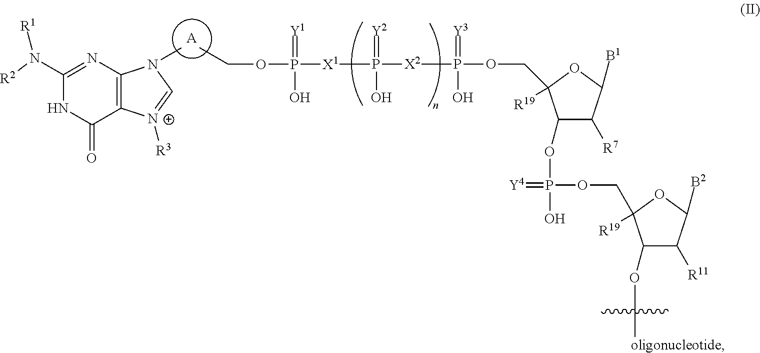

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   B¹ and B² are each independently a natural, modified, or unnatural nucleoside base;
   Ring A is a substituted or unsubstituted cycloalkylene, substituted or unsubstituted heterocycloalkylene, substituted or unsubstituted arylene, or substituted or unsubstituted heteroarylene;
   X¹ and X² are each independently —O—, —CH₂—, —CX₂—, —N(R¹⁰¹)—, —BH—, or —S—;
   Y¹, Y², Y³, and Y⁴ are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁷ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{7A}, —NR^{7A}R^{7B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R¹⁹ is independently hydrogen,
      halogen, —CCl₃, —CBr₃, —CF₃, —CI₃, —CHCl₂, —CHBr₂,
      —CHF₂, —CHI₂, —CH₂Cl, —CH₂Br, —CH₂F, —CH₂I, —CN, —OR^{19A}, —NR^{19A}R^{19B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁷ and R¹⁹ together with the carbon atoms to which they are connected form a substituted or unsubstituted cycloalkylene or substituted or unsubstituted heterocycloalkylene;
each R¹¹ is independently hydrogen,
   halogen, —CCl₃, —CBr₃, —CF₃, —CI₃, —CHCl₂, —CHBr₂,
   —CHF₂, —CHI₂, —
   CH₂Cl, —CH₂Br, —CH₂F, —CH₂I, —CN, —OR^{11A}, —NR^{11A}R^{11B}, —COOH, —CONH₂,
   —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R¹¹ and R¹⁹ together with the carbon atoms to which they are connected form a substituted or unsubstituted cycloalkylene or substituted or unsubstituted heterocycloalkylene;
each R^{1A}, R^{2A}, R^{3A}, R^{7A}, R^{7B}, R^{11A}, R^{11B}, R^{19A}, and R^{19B} is independently
   hydrogen, —CX₃,
   —CHX₂, —CH₂X, —C(O)OH, —C(O)NH₂, —CN, —OH, —NH₂, —COOH, —CONH₂, —NO₂, —SH, —SO₃H,
   —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R^{7A} and R^{7B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer from 0 to 3; and
each X is independently —Cl, —Br, —I or —F;
   with the proviso that R¹¹ is not OH.

In embodiments, provided herein is a structure of formula (IIA):

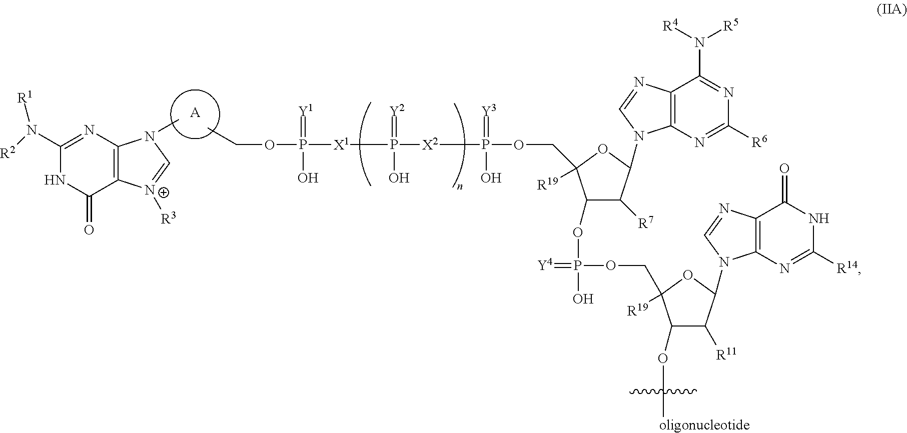

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
R⁴ is independently hydrogen, —C(O)R^{4A}, —C(O)OR^{4A}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently hydrogen, —C(O)R^{5A}, —C(O)OR^{5A}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R⁴ and R⁵ together with the nitrogen atom to which they are connected form a substituted or unsubstituted heteroaryl or a substituted or unsubstituted heterocyclyl;
R⁶ is hydrogen, halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{6A}, —NR^{6A}R^{6B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ is hydrogen, halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{14A}, —NR^{14A}R^{14B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{4A}, R^{5A}, R^{6A}, R^{6B}, R^{14B}, and R^{14B} is independently hydrogen, —CX₃, —CHX₂, —CH₂X,
   —C(O)OH, —C(O)NH₂, —CN, —OH, —NH₂, —COOH, —CONH₂, —NO₂, —SH, —SO₃H, —SO₄H, —SO₂NH₂,
   —NHNH₂, —ONH₂, —NHC═(O)NHNH₂, —NHC═(O)NH₂, —NHSO₂H, —NHC═(O)H, —NHC(O)OH,
   —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{6A} and R^{9B} substituents bonded to the same nitrogen atom may optionally be joined to form a substituted or unsubstituted heterocycloalkyl or substituted or unsubstituted heteroaryl; and R^{14A} and R^{14B} substituents bonded to the same nitrogen atom may optionally be joined to form a substituted or unsubstituted heterocycloalkyl or substituted or unsubstituted heteroaryl;
   R¹, R², R³, R⁷, R¹¹, R¹⁹, X¹, X², Y¹, Y², Y³, Y⁴, Ring A, and n, are each as defined herein, including in embodiments.

In embodiments, provided herein is a structure of formula (IIB):

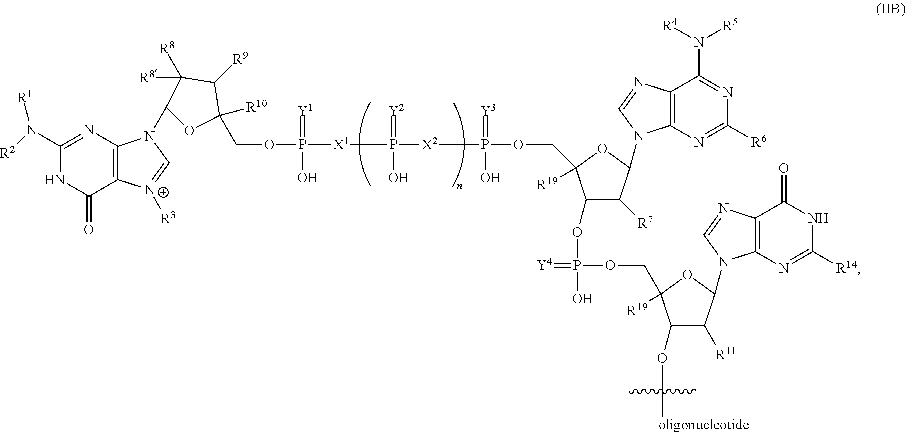

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
R⁸ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{8A}, —NR^{8A}R^{8B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8′} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{8′A}, —NR^{8′A}R^{8′B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8′A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R⁹ is independently hydrogen,
   halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{9A}, —NR^{9A}R^{9B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{9A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁸ and R⁹ or R^{8′} and R⁹ together with the carbon atoms to which they are connected form a substituted or unsubstituted heterocycloalkylene;
R¹⁰ is hydrogen, halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{10A}, —NR^{10A}R^{10B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁸ and R¹⁰ or R^{8′} and R¹⁰ together with the carbon atoms to which they are connected form a substituted or unsubstituted cycloalkylene or substituted or unsubstituted heterocycloalkylene;
each R^{8A}, R^{8B}, R^{8′A}, R^{8′B}, R^{9A}, R^{9B}, R^{10A}, and R^{10B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H,
   —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8A} and R^{8B} substituents bonded to the same nitrogen atom may optionally be joined to form a substituted or unsubstituted heterocycloalkyl or substituted or unsubstituted heteroaryl; R^{8′A} and R^{8′B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl.
   R¹, R², R³, R⁴, R⁵, R⁶, R⁷, R¹¹, R¹⁴, R¹⁹, X¹, X², Y¹, Y², Y³, Y⁴, and n, are each as defined herein, including in embodiments.

In embodiments, provided herein is a structure of formula (IIC):

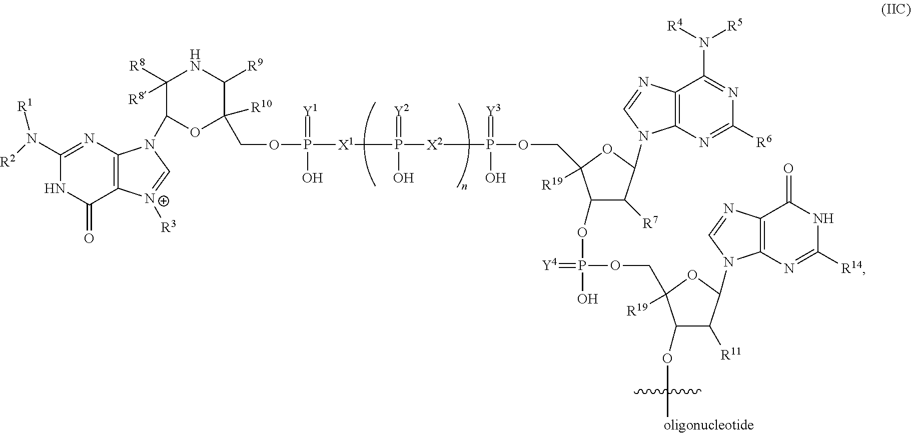

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof, wherein R¹, R², R³, R⁴, R⁵, R⁶, R⁷, R¹¹, R¹⁴, R¹⁹, X¹, X², Y¹, Y², Y³, Y⁴, and n, are each as defined herein, including in embodiments.

In embodiments, provided herein is an oligonucleotide whose 5′ end comprises a compound selected from:

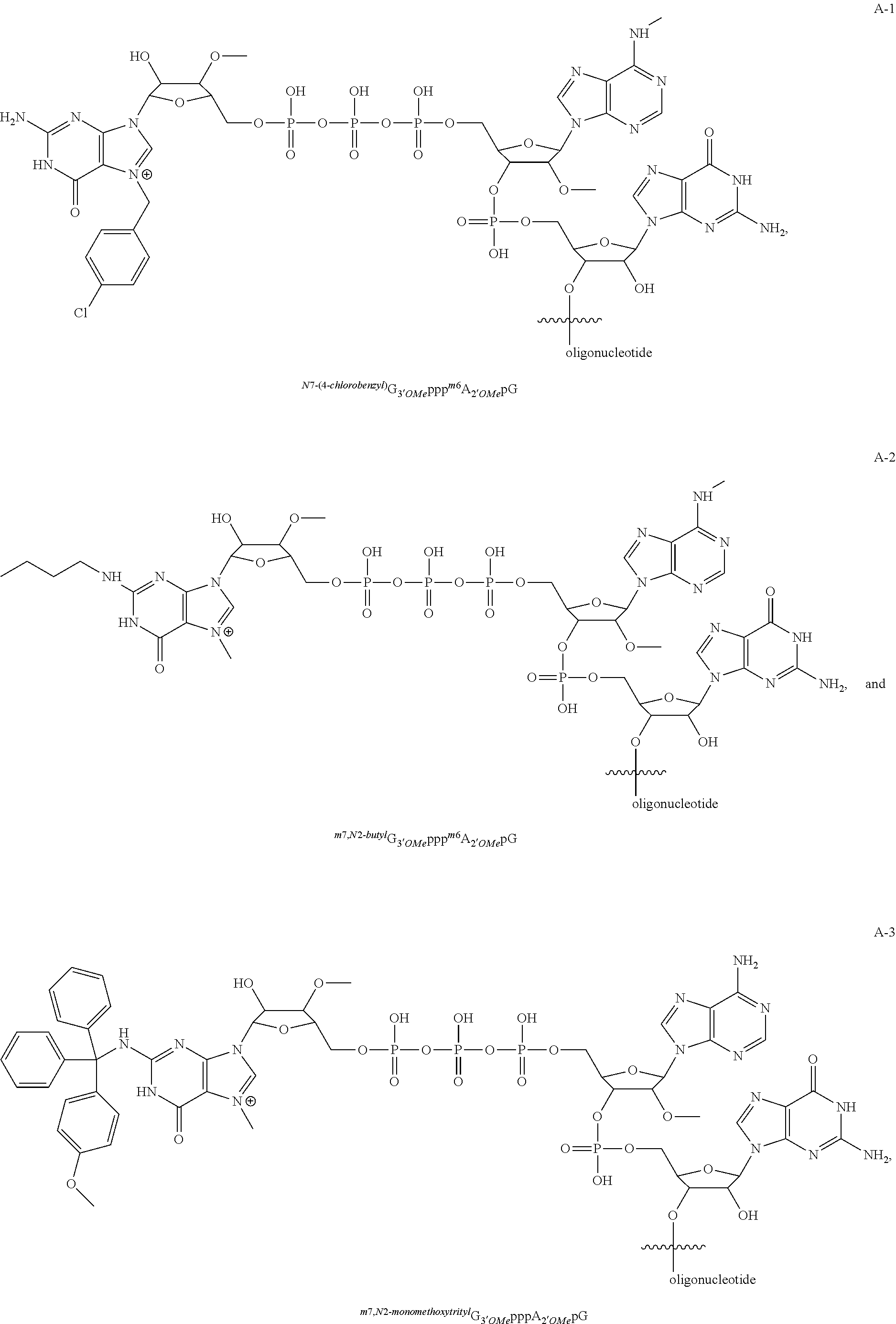

and an enantiomer, a mixture of enantiomers, a mixture of two or more diastereomers, a tautomer, a mixture of two or more tautomers, or an isotopic variant thereof; or a pharmaceutically acceptable salt, solvate, or hydrate, thereof.

In embodiments, provided herein is an oligonucleotide whose 5′ end comprises a compound of the following structure:

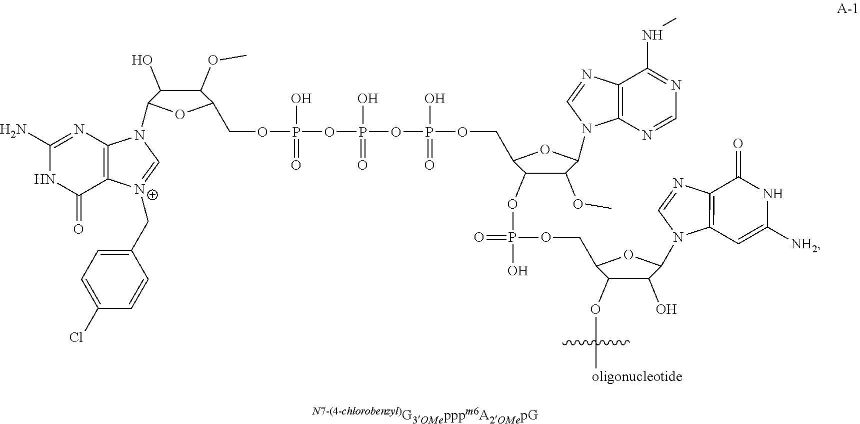

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate, thereof.

In embodiments, provided herein is an oligonucleotide whose 5′ end comprises a compound of the following structure:

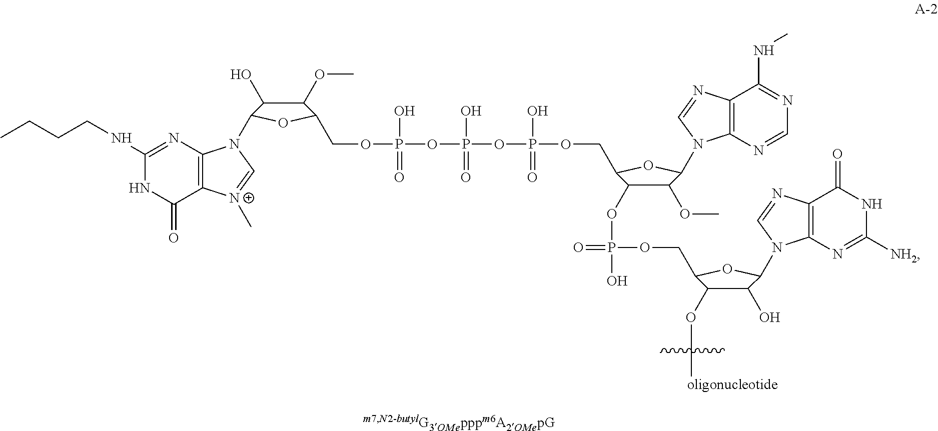

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate, thereof.

In embodiments, provided herein is an oligonucleotide whose 5′ end comprises a compound of the following structure:

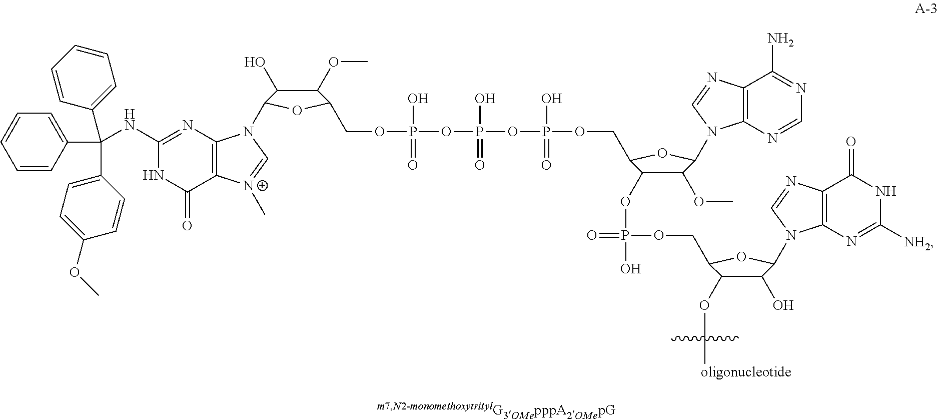

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate, thereof.

As understood by those of skill in the art, the structures shown of the compounds described herein are representations of one form of the compound. Although such compounds may be drawn or described in protonated (free acid) form, in ionized (anionic) form, or ionized and in association with one or more cations (salt form), aqueous solutions of such compounds exist in equilibrium among such forms. For example, a phosphate linkage of a compound described herein, in aqueous solution, exists in equilibrium among free acid, anion, and salt forms.

In embodiments, a structure of formula (I) can be depicted in protonated form (free acid):

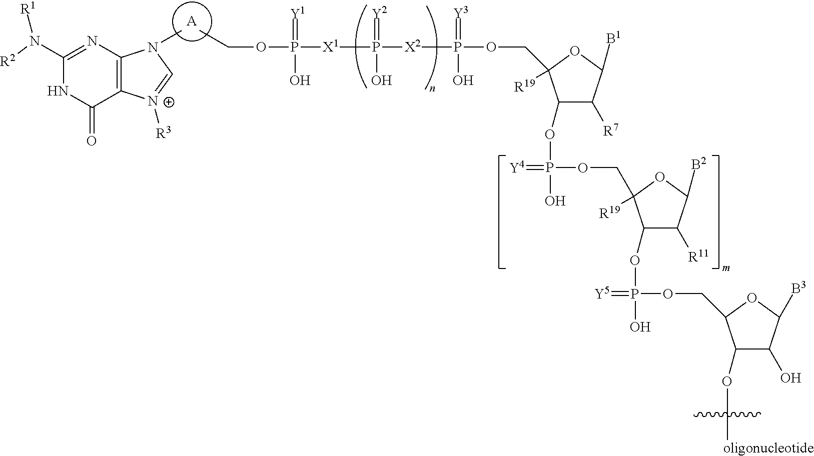

In embodiments, a structure of formula (I) can be depicted in ionized (anionic) form:

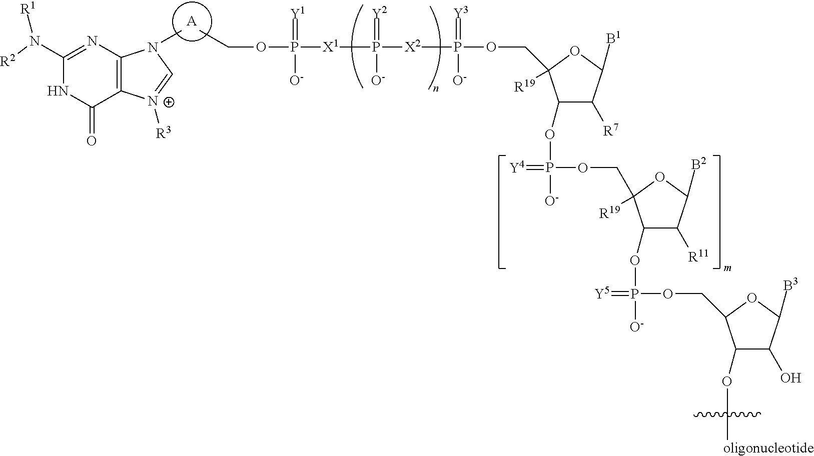

In embodiments, a structure of formula (I) can be depicted in ionized and in association with one or more cations (salt) form:

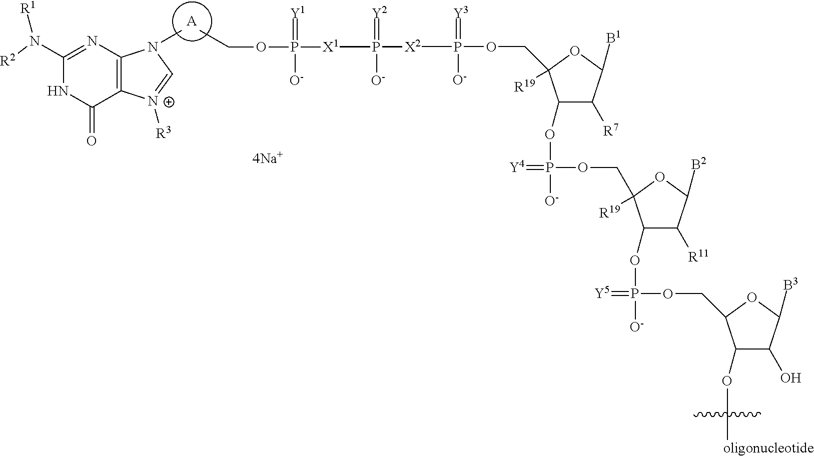

As understood by those of skill in the art, the cation salt form may exist in which one, two, three, four, or five cations are present. Unless otherwise indicated, compounds described herein are intended to include all such forms. Moreover, certain compounds have several such linkages, each of which is in equilibrium. Thus, compounds in solution exist in an ensemble of forms at multiple positions all at equilibrium. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation.

In embodiments, provided herein is a 5′-capped oligonucleotide as described herein including in embodiments, wherein the pharmaceutically acceptable salt is a sodium salt, a lithium salt, or a potassium salt. In embodiments, provided herein is a 5′-capped oligonucleotide as described herein including in embodiments, wherein the pharmaceutically acceptable salt is a sodium salt. In embodiments, provided herein is a 5′-capped oligonucleotide as described herein including in embodiments, wherein the pharmaceutically acceptable salt is a lithium salt. In embodiments, provided herein is a 5′-capped oligonucleotide as described herein including in embodiments, wherein the pharmaceutically acceptable salt is a potassium salt.

In embodiments, provided herein is an initiating capped oligonucleotide primer selected from: ^{m7, N2-monomethoxytrityl}G_{3′OMe}pppA_{2′OMe}pG, ^{N7-(4-chlorobenzyl)}G_{3′OMe}ppp^{m6}A_{2′OMe}pG, and ^{m7, N2-butyl}G_{3′OMe}ppp^{m6}A_{2′OMe}pG.

In embodiments, provided herein is an initiating capped oligonucleotide primer of the following formula: ^{m7, N2-monomethoxytrityl}G_{3′OMe}pppA_{2′OMe}pG.

In embodiments, provided herein is an initiating capped oligonucleotide primer of the following formula: ^{N7-(4-chlorobenzyl)}G_{3′OMe}ppp^{m6}A_{2′OMe}pG.

In embodiments, provided herein is an initiating capped oligonucleotide primer of the following formula: ^{m7, N2-butyl}G_{3′OMe}ppp^{m6}A_{2′OMe}pG.

III. Synthesis of Purified 5′-Capped mRNAs and Oligonucleotides

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

The compounds described herein can be prepared via a two step process, including an activation step followed by a coupling step. During the activation step, a TEA salt of a desired (modified) guanosine-5′-diphosphate is dissolved in a solvent mixture (usually water/DMSO) and activated with an activating reagent such as 1-(3-dimethylaminopropyl)-3-ethylcarbodiimide hydrochloride salt (EDC·HCl salt), followed by addition of imidazole. After a period of time, an activated intermediate (imidazolide) is formed and prepared for use in a coupling step. The activated intermediate is then coupled with the desired (modified) dinucleotide.

Alternatively, a TEA salt of a diphosphate of the desired (modified) dinucleotide is dissolved in a solvent mixture (usually water/DMSO) and activated with an activating reagent such as 1-(3-dimethylaminopropyl)-3-ethylcarbodiimide hydrochloride salt (EDC·HCl salt), followed by addition of imidazole. After a period of time, an activated intermediate (imidazolide) is formed and prepared for use in a coupling step. The activated intermediate is then coupled with the TEA salt of the modified guanosine-5′-monophosphate.

Both general synthesis routes are depicted below in the Examples section as Scheme 1, Scheme 2, Scheme 3, and Scheme 4. Scheme 1, Scheme 2, Scheme 3, and Scheme 4 are provided for representative purposes only, and those of ordinary skill in the art will understand that the schemes can be applied, with modifications within the purview of those of skill in the art along with the disclosures in the Examples section, to any of the compounds described herein (i.e., compounds A-1 to A-16).

The synthetic methods described herein can be performed as a one-pot synthesis, such that all steps are performed in a single reactor with no isolation of intermediate products during the course of the synthetic method. Further description of these general synthetic methods can be found in International Patent Application No. PCT/US2023/061255 which is incorporated herein by reference in its entirety.

In embodiments, provided herein is a method for preparing a protected 5′-capped oligonucleotide by reacting compound of formula (III):

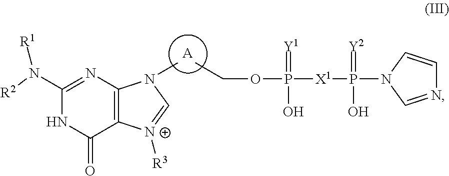

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
Ring A is a substituted or unsubstituted cycloalkylene, substituted or unsubstituted heterocycloalkylene, substituted or unsubstituted arylene, or substituted or unsubstituted heteroarylene;
X¹ is independently —O—, —CH₂—, —CX₂—, —N(R¹⁰¹)—, —BH—, or —S—;
R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{1A}, R^{2A}, and R^{3A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
—OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each X is independently —Cl, —Br, —I or —F;
with a 5′-phosphate-oligonucleotide.

In embodiments, provided herein is a method for preparing a protected 5′-capped oligonucleotide by reacting compound of formula (IIIA):

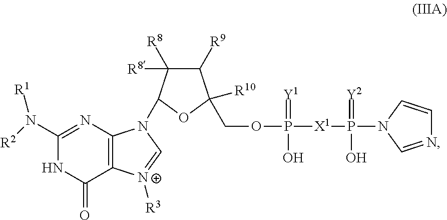

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
R⁸ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{8A}, —NR^{8A}R^{8B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8′} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{8′A}, —NR^{8′A}R^{8′B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8′A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R⁹ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{9A}, —NR^{9A}R^{9B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{9A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁸ and R⁹ or R^{8′} and R⁹ together with the carbon atoms to which they are connected form a substituted or unsubstituted heterocycloalkylene;
R¹⁰ is hydrogen, halogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OR^{10A}, —NR^{10A}R^{10B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁸ and R¹⁰ or R^{8′} and R¹⁰ together with the carbon atoms to which they are connected form a substituted or unsubstituted cycloalkylene or substituted or unsubstituted heterocycloalkylene;
each R^{8A}, R^{8B}, R^{8′A}, R^{8′B}, R^{9A}, R^{9B}, R^{10A}, and R^{10B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H,
—SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8A} and R^{8B} substituents bonded to the same nitrogen atom may optionally be joined to form a substituted or unsubstituted heterocycloalkyl or substituted or unsubstituted heteroaryl; R^{8′A} and R^{8′B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; and R^{10A} and R^{10B} substituents bonded to the same nitrogen atom may optionally be joined to form a substituted or unsubstituted heterocycloalkyl or substituted or unsubstituted heteroaryl; and
R¹, R², R³, X¹, Y¹, and Y² are as defined herein, including in embodiments;
with a 5′-phosphate-oligonucleotide.

In embodiments, provided herein is a method for preparing a protected 5′-capped oligonucleotide by reacting compound of formula (IIIB):

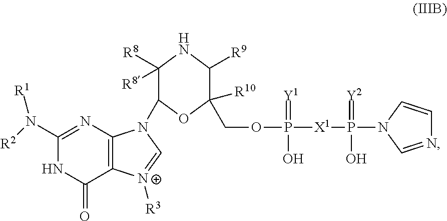

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
R¹, R², R³, R⁸, R^{8′}, R⁹, R¹⁰, X¹, Y¹, and Y² are as defined herein, including in embodiments; with a 5′-phosphate-oligonucleotide.

In embodiments, provided herein is a 5′-capped oligonucleotide, as described herein including in embodiments, wherein a 5′-phosphate-oligonucleotide is prepared via chemical synthesis. In embodiments, provided herein is a protected 5′-capped oligonucleotide, as described herein including in embodiments, wherein a 5′-phosphate-oligonucleotide is prepared via chemical synthesis.

In embodiments, a protected 5′-capped oligonucleotide described herein, including in embodiments, comprises hydrophobic groups R¹, R², and R³. In embodiments, R¹, R², and R³ are each independently a removable hydrophobic group. In embodiments, R¹ is independently a removable hydrophobic group. In embodiments, R² is independently a removable hydrophobic group. In embodiments, R³ is a removable hydrophobic group.

In embodiments, provided herein is a 5′-capped oligonucleotide, as described herein including in embodiments, wherein the capping may be performed chemically or enzymatically. In embodiments, provided herein is a 5′-capped oligonucleotide, as described herein including in embodiments, the capping may be performed chemically. In embodiments, provided herein is a 5′-capped oligonucleotide, as described herein including in embodiments, the capping may be performed enzymatically.

In embodiments, provided herein is a modified 5′-capped oligonucleotide. In embodiments, the 5′-cappped oligonucleotide is modified at its 3′ end. In embodiments, the 3′ end refers to the first 10 nucleotides at the 3′ end. In embodiments, the 3′ end refers to the first 10 internucleotide linkages at the 3′ end.

In embodiments, the 5′-capped oligonucleotide modified at its 3′ end includes 1 or more modified nucleosides. In embodiments, the 5′-capped oligonucleotide modified at its 3′ end includes 2 or more modified nucleosides. In embodiments, the 5′-capped oligonucleotide modified at its 3′ end includes 3 or more modified nucleosides. In embodiments, the 5′-capped oligonucleotide modified at its 3′ end includes 1 modified nucleoside. In embodiments, the 5′-capped oligonucleotide modified at its 3′ end includes 2 modified nucleosides. In embodiments, the 5′-capped oligonucleotide modified at its 3′ end includes 3 modified nucleosides. In embodiments, the 5′-capped oligonucleotide modified at its 3′ end includes 4 modified nucleosides. In embodiments, the 5′-capped oligonucleotide modified at its 3′ end includes 5 modified nucleosides. In embodiments, the 5′-capped oligonucleotide modified at its 3′ end includes 6 modified nucleosides. In embodiments, the 5′-capped oligonucleotide modified at its 3′ end includes 7 modified nucleosides. In embodiments, the 5′-capped oligonucleotide modified at its 3′ end includes 8 modified nucleosides. In embodiments, the 5′-capped oligonucleotide modified at its 3′ end includes 9 modified nucleosides. In embodiments, the 5′-capped oligonucleotide modified at its 3′ end includes 10 modified nucleosides.

In embodiments, the 5′-capped oligonucleotide modified at its 3′ end includes 2 or more nucleotides that are linked together by a modified internucleotide linkage. In embodiments, the 5′-capped oligonucleotide modified at its 3′ end includes 2 nucleotides that are linked together by a modified internucleotide linkage. In embodiments, the 5′-capped oligonucleotide modified at its 3′ end includes 3 nucleotides that are linked together by a modified internucleotide linkage. In embodiments, the 5′-capped oligonucleotide modified at its 3′ end includes 4 nucleotides that are linked together by a modified internucleotide linkage. In embodiments, the 5′-capped oligonucleotide modified at its 3′ end includes 5 nucleotides that are linked together by a modified internucleotide linkage. In embodiments, the 5′-capped oligonucleotide modified at its 3′ end includes 6 nucleotides that are linked together by a modified internucleotide linkage. In embodiments, the 5′-capped oligonucleotide modified at its 3′ end includes 7 nucleotides that are linked together by a modified internucleotide linkage. In embodiments, the 5′-capped oligonucleotide modified at its 3′ end includes 8 nucleotides that are linked together by a modified internucleotide linkage. In embodiments, the 5′-capped oligonucleotide modified at its 3′ end includes 9 nucleotides that are linked together by a modified internucleotide linkage. In embodiments, the 5′-capped oligonucleotide modified at its 3′ end includes 10 nucleotides that are linked together by a modified internucleotide linkage. In embodiments, 4 or more nucleotides that are linked together by a modified internucleotide linkage are all adjacent to each other. In embodiments, when 4 or more nucleotides are linked by modified internucleotide linkages only each 2 nucleotides must be adjacent. For example, when 4 nucleotides are linked by modified internucleotide linkages, 2 nucleotides must be adjacent but the other two nucleotides (although adjacent to each other) may be separate from the first two nucleotides.

In embodiments, the 5′-capped oligonucleotide modified at its 3′ end includes 1 or more modified nucleosides and 2 or more nucleotides that are linked together by a modified internucleotide linkage. In embodiments, the 5′-capped oligonucleotide modified at its 3′ end includes 2 or more modified nucleosides and 2 or more nucleotides that are linked together by a modified internucleotide linkage. In embodiments, the 5′-capped oligonucleotide modified at its 3′ end includes 3 or more modified nucleosides and 2 or more nucleotides that are linked together by a modified internucleotide linkage. In embodiments, the 5′-capped oligonucleotide modified at its 3′ end includes 4 or more modified nucleosides and 2 or more nucleotides that are linked together by a modified internucleotide linkage. In embodiments, the 5′-capped oligonucleotide modified at its 3′ end includes 5 or more modified nucleosides and 2 or more nucleotides that are linked together by a modified internucleotide linkage. In embodiments, the 5′-capped oligonucleotide modified at its 3′ end includes 6 or more modified nucleosides and 2 or more nucleotides that are linked together by a modified internucleotide linkage. In embodiments, the 5′-capped oligonucleotide modified at its 3′ end includes 7 or more modified nucleosides and 2 or more nucleotides that are linked together by a modified internucleotide linkage. In embodiments, the 5′-capped oligonucleotide modified at its 3′ end includes 8 or more modified nucleosides and 2 or more nucleotides that are linked together by a modified internucleotide linkage. In embodiments, the 5′-capped oligonucleotide modified at its 3′ end includes 9 or more modified nucleosides and 2 or more nucleotides that are linked together by a modified internucleotide linkage. In embodiments, the 5′-capped oligonucleotide modified at its 3′ end includes 10 or more modified nucleosides and 2 or more nucleotides that are linked together by a modified internucleotide linkage.

In embodiments, a structure of formula (I) or formula (II) comprises one or more removable hydrophobic group(s). In embodiments, a structure of formula (I) or formula (II) comprises one removable hydrophobic group. In embodiments, a structure of formula (I) or formula (II) comprises two removable hydrophobic groups. In embodiments, a structure of formula (I) or formula (II) comprises three removable hydrophobic groups. In embodiments, a structure of formula (I) or formula (II) comprises four removable hydrophobic groups. In embodiments, a structure of formula (I) or formula (II) comprises five removable hydrophobic groups.

In embodiments, a structure of formula (I) or formula (II) comprises one or more non-removable hydrophobic group(s). In embodiments, a structure of formula (I) or formula (II) comprises one non-removable hydrophobic group. In embodiments, a structure of formula (I) or formula (II) comprises two non-removable hydrophobic groups. In embodiments, a structure of formula (I) or formula (II) comprises three non-removable hydrophobic groups. In embodiments, a structure of formula (I) or formula (II) comprises four non-removable hydrophobic groups. In embodiments, a structure of formula (I) or formula (II) comprises five non-removable hydrophobic groups.

In embodiments, one or more removable hydrophobic group(s) are attached to the G nucleobase

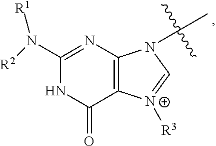

and/or Ring A, and/or nucleobase B¹ of structure of formula (I) or formula (II). In embodiments, one or more removable hydrophobic group(s) are attached to the G nucleobase

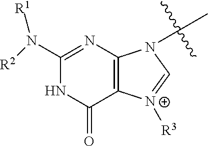

of structure of formula (I) or formula (II). In embodiments, one or more removable hydrophobic group(s) are attached to Ring A of structure of formula (I) or formula (II). In embodiments, one or more removable hydrophobic group(s) are attached to nucleobase B¹ of structure of formula (I) or formula (II).

In embodiments, one removable hydrophobic group is attached to the G nucleobase

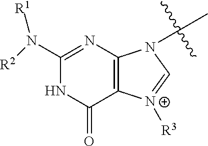

of structure of formula (I) or formula (II). In embodiments, two removable hydrophobic groups are attached to the G nucleobase

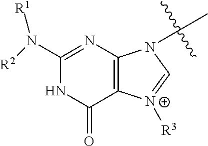

of structure of formula (I) or formula (II). In embodiments, three removable hydrophobic groups are attached to the G nucleobase

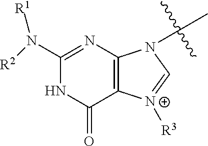

of structure of formula (I) or formula (II).

In embodiments, one removable hydrophobic group is attached to Ring A of structure of formula (I) or formula (II). In embodiments, two removable hydrophobic groups is attached to Ring A of structure of formula (I) or formula (II).

In embodiments, one removable hydrophobic group is attached to nucleobase B¹ of structure of formula (I) or formula (II). In embodiments, two removable hydrophobic groups are attached to nucleobase B¹ of structure of formula (I) or formula (II). In embodiments, three removable hydrophobic groups are attached to nucleobase B¹ of structure of formula (I) or formula (II).

In embodiments, Ring A is a ribose ring. In embodiments, Ring A is a morpholino ring.

In embodiments, each removable hydrophobic group is independently silyl, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, each removable hydrophobic group is independently substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, each removable hydrophobic group (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the removable hydrophobic group is substituted with a plurality of groups selected from substituent groups, size-limited substituent groups, and lower substituent groups; each substituent group, size-limited substituent group, and/or lower substituent group may optionally be different. In embodiments, when each removable hydrophobic group is substituted, it is substituted with at least one substituent group. In embodiments, when each removable hydrophobic group is substituted, it is substituted with at least one size-limited substituent group. In embodiments, when each removable hydrophobic group is substituted, it is substituted with at least one lower substituent group.

In embodiments, each removable hydrophobic group is independently silyl, trityl, alkyl, heterocycloalkyl, or aryl substituted ethyl ether, fluorenylmethyloxycarbonyl (Fmoc), t-butyldimethyl silyl (TBDMS), t-butyldiphenyl silyl (TBDPS), dimethoxytrityl (DMT), monomethoxytrityl (MMT), or a modified trityl.

In embodiments, each removable hydrophobic group is independently silyl. In embodiments, each removable hydrophobic group is independently trityl. In embodiments, each removable hydrophobic group is independently alkyl, heterocycloalkyl, or aryl substituted ethyl ether. In embodiments, each removable hydrophobic group is independently fluorenylmethyloxycarbonyl (Fmoc). In embodiments, each removable hydrophobic group is independently t-butyldimethyl silyl (TBDMS). In embodiments, each removable hydrophobic group is independently t-butyldiphenyl silyl (TBDPS). In embodiments, each removable hydrophobic group is independently dimethoxytrityl (DMT). In embodiments, each removable hydrophobic group is independently monomethoxytrityl (MMT). In embodiments, each removable hydrophobic group is independently a modified trityl.

In embodiments, one or more non-removable hydrophobic group(s) are attached to the G nucleobase

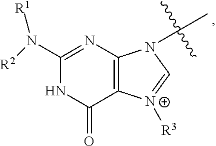

and/or Ring A of structure of formula (I) or formula (II). In embodiments, one or more non-removable hydrophobic group(s) are attached to the G nucleobase

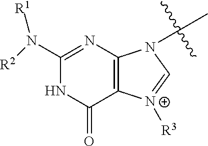

of structure of formula (I) or formula (II). In embodiments, one or more non-removable hydrophobic group(s) are attached to Ring A of structure of formula (I) or formula (II).

In embodiments, one non-removable hydrophobic group is attached to the G nucleobase

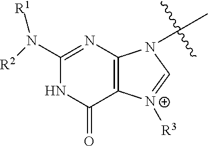

of structure of formula (I) or formula (II). In embodiments, two non-removable hydrophobic groups are attached to the G nucleobase

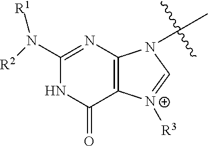

of structure of formula (I) or formula (II). In embodiments, three non-removable hydrophobic groups are attached to the G nucleobase

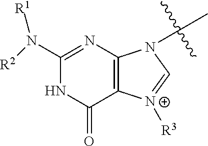

of structure of formula (I) or formula (II).

In embodiments, one non-removable hydrophobic group is attached to Ring A of structure of formula (I) or formula (II). In embodiments, two non-removable hydrophobic groups are attached to Ring A of structure of formula (I) or formula (II).

In embodiments, each non-removable hydrophobic group attached to the G nucleobase

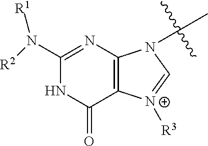

and/or Ring A of structure of formula (I) or formula (II) is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, each non-removable hydrophobic group attached to the G nucleobase

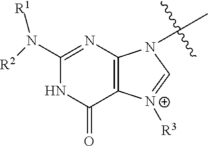

and/or Ring A of structure of formula (I) or formula (II) is independently substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl).

In embodiments, each non-removable hydrophobic group attached to the G nucleobase

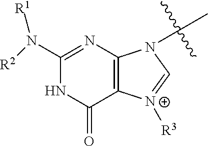

and/or Ring A of structure of formula (I) or formula (II) (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the non-removable hydrophobic group is substituted with a plurality of groups selected from substituent groups, size-limited substituent groups, and lower substituent groups; each substituent group, size-limited substituent group, and/or lower substituent group may optionally be different. In embodiments, when each non-removable hydrophobic group is substituted, it is substituted with at least one substituent group. In embodiments, when each non-removable hydrophobic group is substituted, it is substituted with at least one size-limited substituent group. In embodiments, when each non-removable hydrophobic group is substituted, it is substituted with at least one lower substituent group.

In embodiments, each non-removable hydrophobic group attached to the G nucleobase

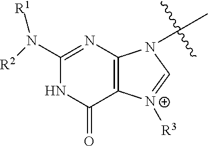

and/or Ring A of structure of formula (I) or formula (II) is independently ethyl, propyl, butyl, 4-chlorobenzyl, benzyl, iso-propyl, or acetyl.

In embodiments, one or more non-removable hydrophobic group(s) are attached to nucleobase B¹ of structure of formula (I) or formula (II). In embodiments, one or more non-removable hydrophobic group(s) are attached to nucleobase B¹ of structure of formula (I) or formula (II).

In embodiments, one non-removable hydrophobic group is attached to nucleobase B¹ of structure of formula (I) or formula (II). In embodiments, two non-removable hydrophobic groups are attached to nucleobase B¹ of structure of formula (I) or formula (II). In embodiments, three non-removable hydrophobic groups are attached to nucleobase B¹ of structure of formula (I) or formula (II).

In embodiments, each non-removable hydrophobic group attached to nucleobase B¹ of structure of formula (I) or formula (II) is independently substituted or unsubstituted alkyl, substituted or unsubstituted heteroalkyl, substituted or unsubstituted cycloalkyl, or substituted or unsubstituted heterocycloalkyl.

In embodiments, each non-removable hydrophobic group attached to nucleobase B¹ of structure of formula (I) or formula (II) is independently substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), or substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl).

In embodiments, each non-removable hydrophobic group attached to nucleobase B¹ of structure of formula (I) or formula (II) (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the non-removable hydrophobic group is substituted with a plurality of groups selected from substituent groups, size-limited substituent groups, and lower substituent groups; each substituent group, size-limited substituent group, and/or lower substituent group may optionally be different. In embodiments, when each non-removable hydrophobic group is substituted, it is substituted with at least one substituent group. In embodiments, when each non-removable hydrophobic group is substituted, it is substituted with at least one size-limited substituent group. In embodiments, when each non-removable hydrophobic group is substituted, it is substituted with at least one lower substituent group.

In embodiments, each non-removable hydrophobic group attached to nucleobase B¹ of structure of formula (I) or formula (II) is independently substituted or unsubstituted alkyl. In embodiments, each non-removable hydrophobic group attached to nucleobase B¹ of structure of formula (I) or formula (II) is independently substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, each non-removable hydrophobic group attached to nucleobase B¹ of structure of formula (I) or formula (II) is independently substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, each non-removable hydrophobic group attached to nucleobase B¹ of structure of formula (I) or formula (II) is independently unsubstituted alkyl (e.g., C₁-C₈alkyl, C₁-C₆ alkyl, or C₁-C₄ alkyl).

In embodiments, each non-removable hydrophobic group attached to nucleobase B¹ of structure of formula (I) or formula (II) is independently ethyl, propyl, butyl, iso-propyl, or acetyl.

In embodiments, each non-removable hydrophobic group is independently ethyl. In embodiments, each non-removable hydrophobic group is independently propyl. In embodiments, each non-removable hydrophobic group is independently butyl. In embodiments, each non-removable hydrophobic group is independently benzyl. In embodiments, each non-removable hydrophobic group is independently 4-chlorobenzyl. In embodiments, each non-removable hydrophobic group is independently iso-propyl. In embodiments, each non-removable hydrophobic group is independently acetyl.

In embodiments, each removable hydrophobic group is not a photocleavable hydrophobic group. In embodiments, each removable hydrophobic group is acid labile. In embodiments, each removable hydrophobic group may be removed under mild acid conditions. In embodiments, each removable hydrophobic group is base labile. In embodiments, each removable hydrophobic group may be removed under mild base conditions. In embodiments, each removable hydrophobic group is thermolabile. In embodiments, each removable hydrophobic group may be removed with mild heating.

In embodiments, each removable hydrophobic group is a removable purification handle. In embodiments, each non-removable hydrophobic group is a non-removable purification handle.

Embodiments, translation permissible hydrophobic group may act as a purification handle. In embodiments, removable hydrophobic group may be translation permissible. In embodiments, any translation permissible group can be kept (e.g., not removed) even if it is a removable hydrophobic group.

In embodiments a removable or non-removable purification handle covalently attached to the 5′-capped oligonucleotide may help separate 5′-capped oligonucleotides from the uncapped oligonucleotides and other impurities in the reaction mixture. In embodiments a removable purification handle covalently attached to the 5′-capped oligonucleotide may help separate 5′-capped oligonucleotides from the uncapped oligonucleotides and other impurities in the reaction mixture. In embodiments a non-removable purification handle covalently attached to the 5′-capped oligonucleotide may help separate 5′-capped oligonucleotides from the uncapped oligonucleotides and other impurities in the reaction mixture.

In an aspect, provided herein is a protected 5′-capped oligonucleotide prepared by the reaction of compound of formula (III):

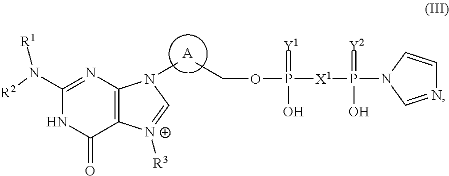

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   Ring A is a substituted or unsubstituted cycloalkylene, substituted or unsubstituted heterocycloalkylene, substituted or unsubstituted arylene, or substituted or unsubstituted heteroarylene;
   X¹ is independently —O—, —CH₂—, —CX₂—, —N(R¹⁰¹)—, —BH—, or —S—;
   Y¹ and Y² are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR³⁴, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R^{1A}, R^{2A}, and R^{3A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
      —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each X is independently —Cl, —Br, —I or —F;
   with a 5′-phosphate-oligonucleotide

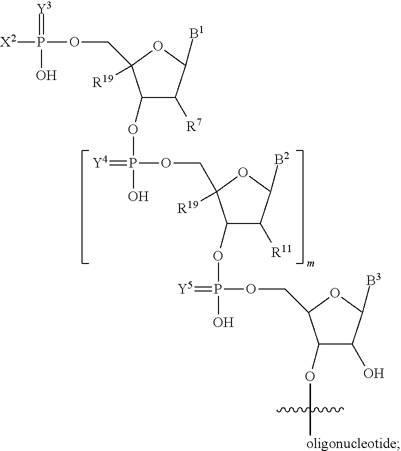

wherein R⁷, R¹¹, R¹⁹, Y³, Y⁴, Y⁵, B¹, B², B³, X², and m are as defined herein, including in embodiments.

In an aspect, provided herein is a protected 5′-capped oligonucleotide prepared by the reaction of compound of formula (IIIA):

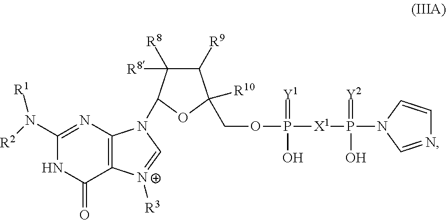

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   R⁸ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{8A}, —NR^{8A}R^{8B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R^{8′} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{8′A}, —NR^{8′A}R^{8′B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{8′A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R⁹ is independently hydrogen,
      halogen, —CCl₃, —CBr₃, —CF₃, —CI₃, —CHCl₂, —CHBr₂,
      —CHF₂, —CHI₂, —CH₂Cl, —CH₂Br, —CH₂F, —CH₂I, —CN, —OR⁹⁴, —NR^{9A}R^{9B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)R^{9A}, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁸ and R⁹ or R^{8′} and R⁹ together with the carbon atoms to which they are connected form a substituted or unsubstituted heterocycloalkylene;
R¹⁰ is hydrogen, halogen, —CCl₃, —CBr₃, —CF₃, —CI₃, —CHCl₂, —CHBr₂,
   —CHF₂, —CHI₂, —CH₂Cl, —CH₂Br, —CH₂F, —CH₂I, —CN, —OR^{10A}, —NR^{10A}R^{10B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁸ and R¹⁰ or R^{8′} and R¹⁰ together with the carbon atoms to which they are connected form a substituted or unsubstituted cycloalkylene or substituted or unsubstituted heterocycloalkylene;
each R^{8A}, R^{8B}, R^{8′A}, R^{8′B}, R^{9A}, R^{9B}, R^{10A}, and R^{10B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H,
   —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{8A} and R^{8B} substituents bonded to the same nitrogen atom may optionally be joined to form a substituted or unsubstituted heterocycloalkyl or substituted or unsubstituted heteroaryl; R^{8′A} and R^{8′B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; and R^{10A} and R^{10B} substituents bonded to the same nitrogen atom may optionally be joined to form a substituted or unsubstituted heterocycloalkyl or substituted or unsubstituted heteroaryl;
   with a 5′-phosphate-oligonucleotide

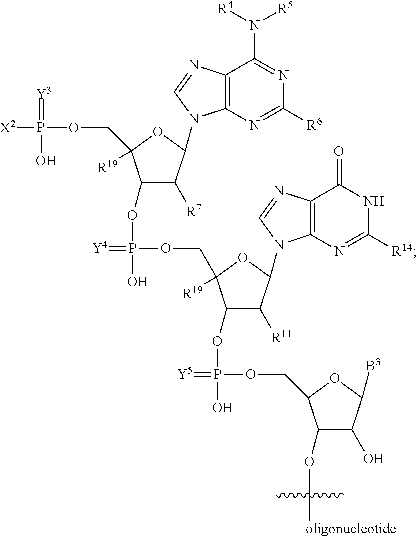

wherein R¹, R², R³, R⁴, R⁵, R⁶, R⁷, R¹¹, R¹⁴, R¹⁹, Y¹, Y², Y³, Y⁴, Y⁵, B³, X¹, and X² are as defined herein, including in embodiments.

In an aspect, provided herein is a protected 5′-capped oligonucleotide prepared by the reaction of compound of formula (IIIB):

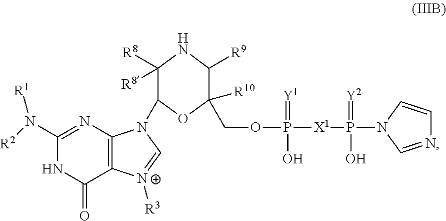

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
with a 5′-phosphate-oligonucleotide

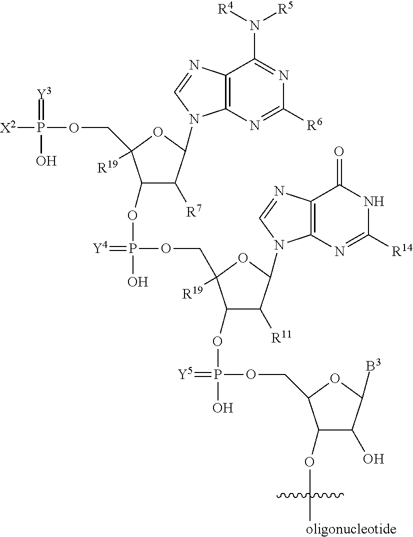

wherein R¹, R², R³, R⁴, R⁵, R⁶, R⁷, R⁸, R^{8′}, R⁹, R¹⁰, R¹¹, R¹⁴, R¹⁹, Y¹, Y², Y³, Y⁴, Y⁵, B³, X¹, and X² are as defined herein, including in embodiments.

In embodiments, the 5′-phosphate-oligonucleotide, described herein including in embodiments, is prepared via chemical synthesis.

In embodiments, provided herein is a protected 5′-capped oligonucleotide. In embodiments, provided herein is a 5′-capped oligonucleotide prepared by the removal of the protecting group(s) of the protected 5′-capped oligonucleotide, described herein including in embodiments. In embodiments, the deprotection (removal of the protecting group(s)) includes treating the protected 5′-capped oligonucleotide with acid, base, or heat. In embodiments, the deprotection includes treating the protected 5′-capped oligonucleotide with a mild acid, a mild base, or mild heat. In embodiments, the removable hydrophobic group(s) or removable purification handle(s) on the 5′-capped oligonucleotide are acid labile, base labile, or thermolabile (as described in the definition section). In embodiments, the removable hydrophobic group(s) or removable purification handle(s) on the 5′-capped oligonucleotide are acid labile. In embodiments, the removable hydrophobic group(s) or removable purification handle(s) on the 5′-capped oligonucleotide are base labile. In embodiments, the removable hydrophobic group(s) or removable purification handle(s) on the 5′-capped oligonucleotide are thermolabile.

In embodiments, the 5′-capped oligonucleotides described herein including in embodiments, are substantially free of enzymatic byproducts. In embodiments, the 5′-capped oligonucleotides described herein including in embodiments, are substantially free of double stranded RNA (dsRNA). In embodiments, the 5′-capped oligonucleotides described herein including in embodiments, are substantially free of truncated oligonucleotides. In embodiments, the 5′-capped oligonucleotides described herein including in embodiments, are substantially free of uncapped oligonucleotides.

In an aspect, provided herein is a composition comprising a chemically synthesized oligonucleotide wherein the composition comprises less than 1% by weight of oligonucleotide whose 5′ end does not comprise a structure of formula (I) or formula (II). In an aspect, provided herein is a composition comprising a chemically synthesized oligonucleotide wherein the composition comprises less than 0.5% by weight of oligonucleotide whose 5′ end does not comprise a structure of formula (I) or formula (II). In an aspect, provided herein is a composition comprising a chemically synthesized oligonucleotide wherein the composition comprises less than 0.25% by weight of oligonucleotide whose 5′ end does not comprise a structure of formula (I) or formula (II). In an aspect, provided herein is a composition comprising a chemically synthesized oligonucleotide wherein the composition comprises less than 0.1% by weight of oligonucleotide whose 5′ end does not comprise a structure of formula (I) or formula (II). In an aspect, provided herein is a composition comprising a chemically synthesized oligonucleotide wherein the composition comprises less than 0.05% by weight of oligonucleotide whose 5′ end does not comprise a structure of formula (I) or formula (II).

In embodiments, provided herein is a composition comprising a chemically synthesized oligonucleotide wherein the composition comprises less than about 0.01%, less than about 0.02%, less than about 0.03%, less than about 0.04%, less than about 0.05%, less than about 0.06%, less than about 0.07%, less than about 0.08%, less than about 0.09%, less than about 0.1%, less than about 0.15%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10%, by weight of oligonucleotide whose 5′ end does not comprise a structure of formula (I) or formula (II).

In embodiments, provided herein is a composition comprising a chemically synthesized oligonucleotide wherein the composition is substantially free of oligonucleotide whose 5′ end does not comprise a structure of formula (I) or formula (II).

In embodiments, provided herein is a composition comprising a chemically synthesized oligonucleotide wherein the composition is substantially free of enzymatic byproducts. In embodiments, provided herein is a composition comprising a chemically synthesized oligonucleotide wherein the composition is substantially free of dsRNA. In embodiments, provided herein is a composition comprising a chemically synthesized oligonucleotide wherein the composition is substantially free of truncated oligonucleotides. In embodiments, provided herein is a composition comprising a chemically synthesized oligonucleotide wherein the composition is substantially free of uncapped oligonucleotides.

In embodiments, provided herein is a composition comprising (a) more than 90% of 5′-capped oligonucleotide described herein, including in embodiments; (b) less than 10% of 5′-capped oligonucleotide described herein, including in embodiments; and optionally (c) less than 1% of oligonucleotide whose 5′ end does not comprise a structure of formula (I) or formula (II).

In embodiments, provided herein is a composition comprising (a) more than 95% of 5′-capped oligonucleotide described herein, including in embodiments; (b) less than 5% of 5′-capped oligonucleotide described herein, including in embodiments; and optionally (c) less than 1% of oligonucleotide whose 5′ end does not comprise a structure of formula (I) or formula (II).

In embodiments, provided herein is a composition comprising (a) more than 98% of 5′-capped oligonucleotide described herein, including in embodiments; (b) less than 2% of 5′-capped oligonucleotide described herein, including in embodiments; and optionally (c) less than 1% of oligonucleotide whose 5′ end does not comprise a structure of formula (I) or formula (II).

In embodiments, provided herein is a composition comprising (a) more than 99% of 5′-capped oligonucleotide described herein, including in embodiments; (b) less than 1% of 5′-capped oligonucleotide described herein, including in embodiments; and optionally (c) less than 1% of oligonucleotide whose 5′ end does not comprise a structure of formula (I) or formula (II).

In embodiments, provided herein is a composition comprising (a) more than 99% of 5′-capped oligonucleotide described herein, including in embodiments; and (b) less than 1% of 5′-capped oligonucleotide described herein, including in embodiments

In an aspect, provided herein is a process for preparing an oligonucleotide comprising 50-12000 nucleotides, whose 5′ end comprises a structure of formula (I):

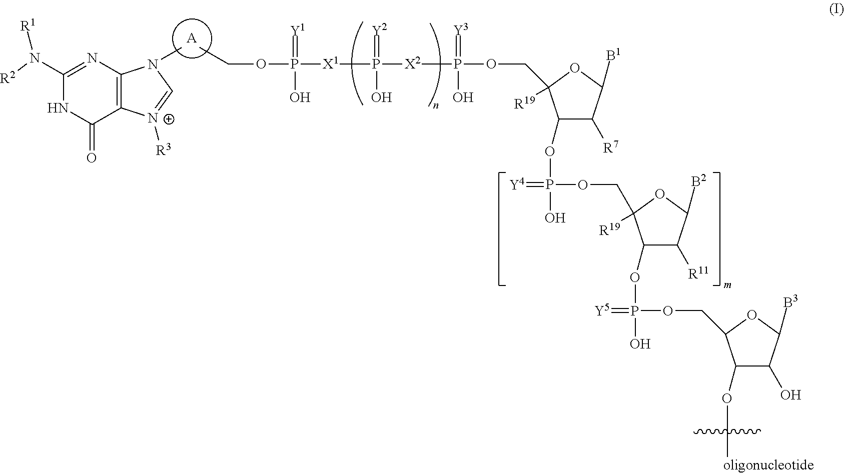

or
formula (II):

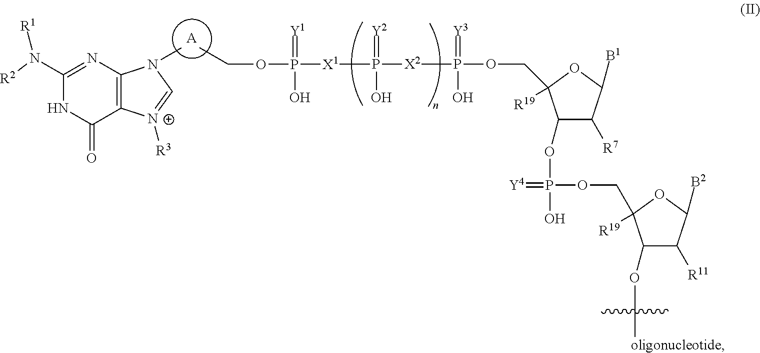

or an enantiomer, a mixture of enantiomers, a mixture of two or more diastereomers, a tautomer, a mixture of two or more tautomers, or an isotopic variant thereof, or a pharmaceutically acceptable salt, solvate, or hydrate thereof;
comprising (a) reacting an imidazolide of formula (III)

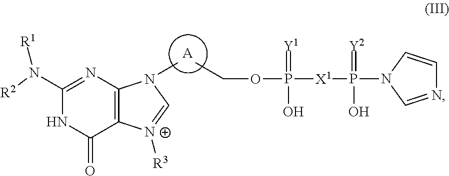

or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
   Ring A is a substituted or unsubstituted cycloalkylene, substituted or unsubstituted heterocycloalkylene, substituted or unsubstituted arylene, or substituted or unsubstituted heteroarylene;
   X¹ is independently —O—, —CH₂—, —CX₂—, —N(R¹⁰¹)—, —BH—, or —S—;
   Y¹ or Y² are each independently O, S, or Se;
   R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
   R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   each R^{1A}, R^{2A}, and R^{3A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH,
      —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each X is independently —Cl, —Br, —I or —F;
   with a 5′-phosphate-oligonucleotide

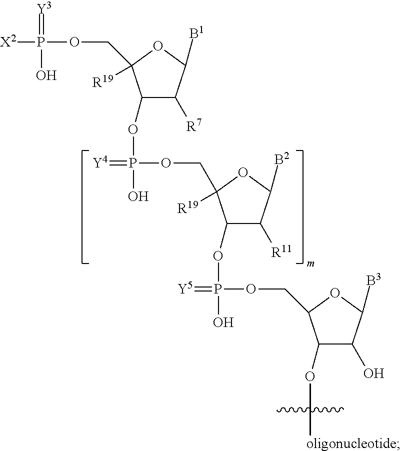

wherein R⁷, R¹¹, R¹⁹, Y³, Y⁴, Y³, B¹, B², B³, X², and m are as defined herein including in embodiments, and (b) optionally removing the removable hydrophobic group(s).

In embodiments, provided herein is a 5′-capped oligonucleotide wherein the oligonucleotide comprises 30 to 15000, 30 to 14000, 30 to 13000, 30 to 12000, 40 to 13000, 40 to 12000, 50 to 13000, 50 to 12000, 50 to 11000, 50 to 10000, 50 to 9000, 50 to 8000, 50 to 7000, 50 to 6000, 50 to 5000, 50 to 4000, 50 to 3000, 50 to 2000, 50 to 1000, 50 to 900, 50 to 800, 50 to 700, 50 to 600, 50 to 500, 50 to 400, 50 to 300, 50 to 200, or 50 to 100 nucleotides.

In embodiments, provided herein is a 5′-capped oligonucleotide wherein the oligonucleotide comprises fewer than 500, fewer than 400, fewer than 300, fewer than 250, fewer than 200, fewer than 150, fewer than 100, fewer than 75 nucleotides. In embodiments, provided herein is a 5′-capped oligonucleotide wherein the oligonucleotide comprises fewer than 200, fewer than 150, fewer than 100, fewer than 75 nucleotides. In embodiments, provided herein is a 5′-capped oligonucleotide wherein the oligonucleotide comprises from about 50 to about 100 nucleotides.

Non-enzymatic methods of making mRNAs can potentially be more cost effective and more amenable to scale-up than traditional approaches involving enzymes. In a recently published non-enzymatic method (Abe et al., ACS Chem. Biol. 2022, 17, 1308-1314), a 5′-phosphate RNA molecule was synthesized on solid phase using an automated oligo synthesizer and cleaved from the solid phase. Following cleavage from the solid support, the 5′-phosphate RNA was coupled with N7-methylated GDP imidazole (Im-m⁷GDP) to form 5′-capped-mRNA. However, the Im-m⁷GDP coupling yield could be variable and it is difficult to purify the target 5′-capped mRNA. The present disclosure relates to non-enzymatic processes to produce mRNAs (oligonucleotides) with high efficiency and purity.

In one aspect, the disclosure provides modified Im-m⁷GDP compounds that can react with 5′-phosphate-mRNA molecules to produce 5′-capped mRNA molecules (also referred to as 5′-capped oligonucleotides). The modified 5′-capped mRNAs comprise protecting groups that can be removed under mild reaction conditions. The modified 5′-capped mRNA molecule can then be readily separated from impurities in the reaction mixture, including uncapped mRNA molecules. Thereafter, the removable protecting group or groups can be chemically or thermally removed from the purified 5′-capped mRNA generating a highly pure and readily translatable 5′-capped mRNA molecule. Alternatively, the modified 5′-capped mRNAs can comprise protecting groups that may be non-removable. The non-removable groups may not be easily removable under mild reaction conditions, or they may be simply not removed because they do not interfere with the translation of the mRNA. Skipping the removal step allows for a smaller number of overall reaction steps and purification steps. The modified 5′-capped mRNA molecule, comprising non-removable protecting groups, can then be readily separated from impurities in the reaction mixture, including uncapped mRNA molecules, generating a highly pure and readily translatable 5′-capped mRNA molecule.

In certain embodiments, the modified Im-m⁷GDP of the disclosure has the structure of Formula (III′D)

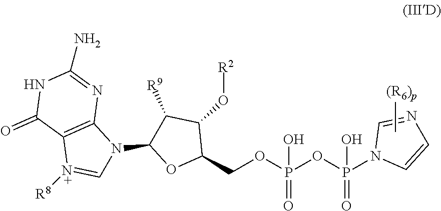

or a salt thereof, wherein R² is a removable hydrophobic group, R⁶ is a substituent other than hydrogen (e.g., halo, amino, C₁₋₆alkyl, C₁₋₆alkoxy, —OH, or —CN), R⁸ is selected from the group consisting of hydrogen, C₂₋₁₂ alkyl, (C₂₋₁₂alkyl)halo, (C₂₋₁₂alkyl)NH₂, (C₂₋₁₂alkyl)OH, cyclic alkyl group optionally substituted with one or more NH₂, NHC₁₋₆ alkyl, halo or OH group, or a 5 or 6 membered aromatic or heteroaromatic ring optionally substituted with one or more NH₂, NHC₁₋₆ alkyl, halo or OH group, R⁹ is selected from the group consisting of hydrogen, OH, NR¹¹R¹², C₁₋₆ alkyl, OC₁₋₆ alkyl, halo (e.g., F, Cl, Br or I), O(C₁₋₆ alkyl)O C₁₋₆ alkyl, (C₁₋₆ alkyl)N₃, O(C₁₋₆ alkyl)N₃, CH₂halo, CH(halo)₂, C(halo)₃, O(C₁₋₆alkyl)halo, O(C₁₋₆alkyl)CH₂halo, O(C₁₋₆alkyl)CH(halo)₂, O(C₁₋₆alkyl)C(halo)₃, OCH₂—CH═CH₂, O(CH₂)₂—OCH₃, O(CH₂)₂—SCH₃, O(CH₂)₂—OCF₃, O(C₁₋₆alkyl)-N(R¹¹)(R¹²), O(C₁₋₆alkyl)-O(C₁₋₆alkyl)N(R¹¹)(R¹²), and OCH₂C(═O)—N(R¹¹)(R¹²), R¹¹ and R¹² are each independently H or C₁₋₆alkyl, and p is an integer from 0-3.

In certain embodiments R² in the compound of Formula (III′D) is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs). In some embodiments, the compound of Formula (III′D) is a disodium salt. In some embodiments, the compound of Formula (III′D) is a monosodium salt.

In other embodiments, R² in the compound of Formula (III′D) is a silyl group. In some embodiments, R² is a triphenylsilyl group. In some embodiments, R² is a triphenylsilyl group, wherein one or more of the phenyl rings is substituted. Particular substituents include, but are not limited to C₁₋₆alkyl (e.g., CH₃ or isopropyl), OC₁₋₆alkyl (e.g., OCH₃), and halo. In other embodiments, R² is an (alkyl)diphenylsilyl group. In some such embodiments, one of the phenyl groups is substituted. Particular substituents include, but are not limited to C₁₋₆alkyl (e.g., CH₃ or isopropyl), OC₁₋₆alkyl (e.g., OCH₃), and halo. In other embodiments, R² is an (alkyl)(napththalen-2-yl)(phenyl). In other embodiments, R² is a (dialkyl)phenylsilyl group. In some such embodiments, the phenyl groups is substituted. Particular substituents include, but are not limited to C₁₋₆alkyl (e.g., CH₃ or isopropyl), OC₁₋₆alkyl (e.g., OCH₃), and halo.

In some embodiments, R² in the compound of Formula (III′D) is one of the following groups.

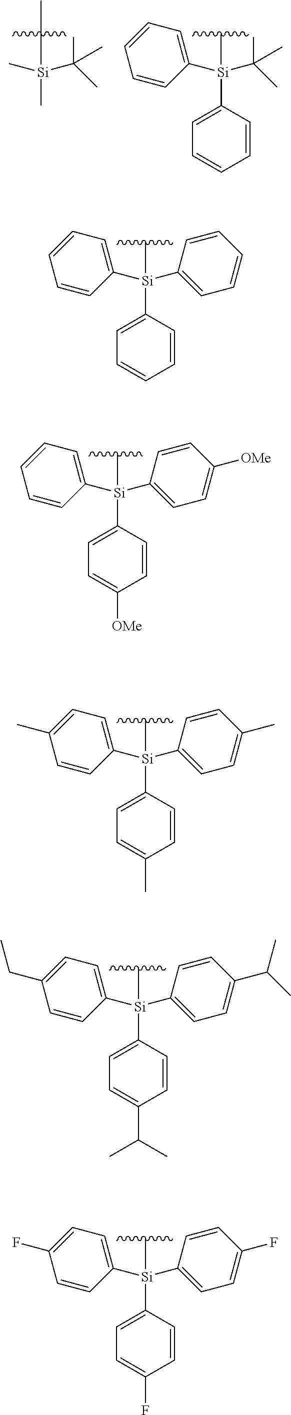

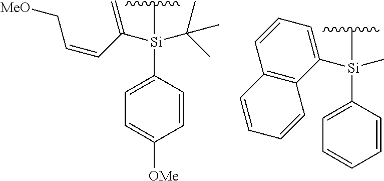

In some embodiments of the compound of Formula (III′D), R⁸ is C₁₋₆alkyl. In some such embodiments R⁸ is CH₃, In other embodiments R⁸ is ethyl.

In some embodiments of the compound of Formula (III′D), R⁸ is (C₂₋₆alkyl)halo. In other embodiments of the compound of Formula (III′D), R⁸ is (C₂₋₆alkyl)NH₂. In other embodiments of the compound of Formula (III′D), R⁸ is (C₂₋₆alkyl)OH.

In some embodiments of the compound of Formula (III′D), R⁸ is a cyclopentyl or cyclohexyl group. In some embodiments, the cyclopentyl or cyclohexyl group is substituted with one or more NH₂, NHC₁₋₆ alkyl, halo or OH group

In some embodiments of the compound of Formula (III′D), R⁹ is halo. In some such embodiments, R⁹ is F. In other embodiments of the compound of Formula (III′D), R⁹ is OH. In other embodiments of the compound of Formula (III′D), R⁹ is OCH₃. In other embodiments of the compound of Formula (III′D), R⁹ is OCH₂N₃. In other embodiments of the compound of Formula (III′D), R⁹ is OCH₂CH₂OCH₃. In other embodiments of the compound of Formula (III′D), R⁹ is selected from the group consisting of OCH₂F, OCHF₂, OCF₃, OCH₂CH₃, O(CH₂)₂F, OCH₂CHF₂, OCH₂CF₃, OCH₂—CH═CH₂, and O(CH₂)₂—OCH₃. In any of the foregoing embodiments, R⁸ can be CH₃. In any of the foregoing embodiments, p can be 0. In any of the foregoing embodiments, R² can be trityl (Trt), monomethoxytrityl or dimethoxytrityl (DMT).

In certain embodiments, the modified Im-m⁷GDP of the disclosure has the structure of Formula (III″D)

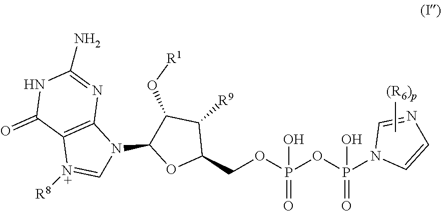

or a salt thereof, wherein R¹ is a removable hydrophobic group, and the variables R⁶, R⁸, R⁹ and p are defined as above.

In certain embodiments R¹ in the compound of Formula (III″D) is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs). In some embodiments, the compound of Formula (III″D) is a disodium salt. In some embodiments, the compound of Formula (III″D) is a monosodium salt.

In other embodiments, R¹ in the compound of Formula (III″D) is a silyl group. In some embodiments, R¹ is a triphenylsilyl group. In some embodiments, R¹ is a triphenylsilyl group, wherein one or more of the phenyl rings is substituted. Particular substituents include, but are not limited to C₁₋₆alkyl (e.g., CH₃ or isopropyl), OC₁₋₆alkyl (e.g., OCH₃), and halo. In other embodiments, R¹ is an (alkyl)diphenylsilyl group. In some such embodiments, one of the phenyl groups is substituted. Particular substituents include, but are not limited to C₁₋₆alkyl (e.g., CH₃ or isopropyl), OC₁₋₆alkyl (e.g., OCH₃), and halo. In other embodiments, R¹ is an (alkyl)(napththalen-2-yl)(phenyl). In other embodiments, R¹ is a (dialkyl)phenylsilyl group. In some such embodiments, the phenyl groups is substituted. Particular substituents include, but are not limited to C₁₋₆alkyl (e.g., CH₃ or isopropyl), OC₁₋₆alkyl (e.g., OCH₃), and halo.

In other embodiments, R¹ in the compound of Formula (III″D) is one of the following groups.

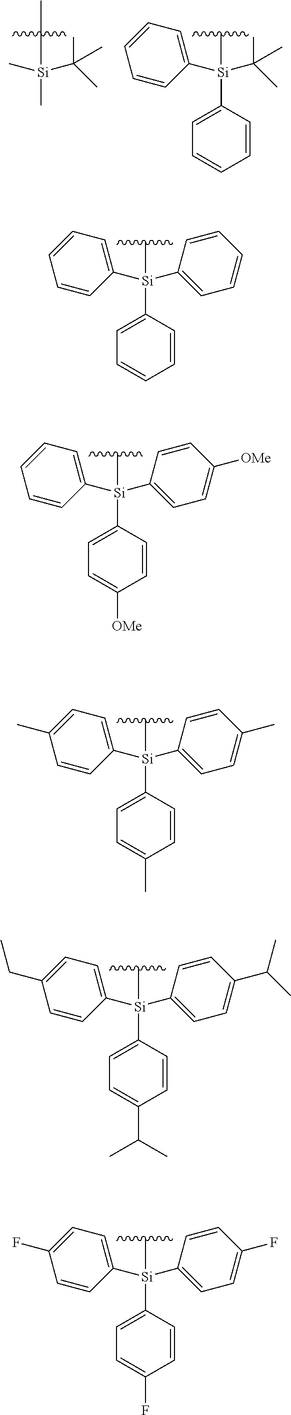

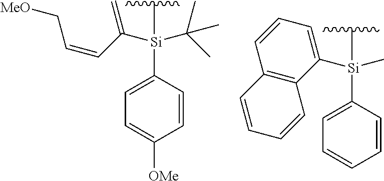

In some embodiments of the compound of Formula (III″D), R⁸ is C₁₋₆alkyl. In some such embodiments R⁸ is CH₃, In other embodiments R⁸ is ethyl.

In some embodiments of the compound of Formula (III″D), R⁸ is (C₂₋₆alkyl)halo. In other embodiments of the compound of Formula (III″D), R⁸ is (C₂₋₆alkyl)NH₂. In other embodiments of the compound of Formula (III″D), R⁸ is (C₂₋₆alkyl)OH.

In some embodiments of the compound of Formula (III″D), R⁸ is a cyclopentyl or cyclohexyl group. In some embodiments, the cyclopentyl or cyclohexyl group is substituted with one or more NH₂, NHC₁₋₆ alkyl, halo or OH group

In some embodiments of the compound of Formula (III″D), R⁹ is halo. In some such embodiments, R⁹ is F. In other embodiments of the compound of Formula (III″D), R⁹ is OH. In other embodiments of the compound of Formula (III″D), R⁹ is OCH₃. In other embodiments of the compound of Formula (III″D), R⁹ is OCH₂N₃. In other embodiments of the compound of Formula (III″D), R⁹ is OCH₂CH₂OCH₃. In other embodiments of the compound of Formula (III″D), R⁹ is selected from the group consisting of OCH₂F, OCHF₂, OCF₃, OCH₂CH₃, O(CH₂)₂F, OCH₂CHF₂, OCH₂CF₃, OCH₂—CH═CH₂, and O(CH₂)₂—OCH₃. In any of the foregoing embodiments, R⁸ can be CH₃. In any of the foregoing embodiments, p can be 0. In any of the foregoing embodiments, R¹ can be trityl (Trt), monomethoxytrityl or dimethoxytrityl (DMT).

In certain embodiments, the modified Im-m⁷GDP of the disclosure has the structure of Formula (IIID′)

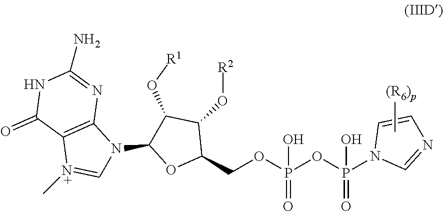

or a salt thereof, wherein one of R¹ or R² is a removable hydrophobic group and the other R¹ or R² is hydrogen or C₁₋₃ alkyl, R⁶ is a substituent other than hydrogen (e.g., halo, amino, C₁₋₆alkyl, C₁₋₆alkoxy, —OH, or —CN), and p is an integer from 0-3. In some embodiments, the compound of Formula (IIID′) is a compound of Formula (IIID) (p=0). In certain embodiments R¹ or R² in the compound of Formula (IIID′) is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs). In some embodiments of the compound of Formula (IIID′) where R¹ is a removable hydrophobic group, R² is H. In other embodiments of the compound of Formula (IIID′) where R¹ is a removable hydrophobic group, R² is CH₃. In some embodiments of the compound of Formula (IIID′) where R² is a removable hydrophobic group, R¹ is H. In other embodiments of the compound of Formula (IIID′) where R² is a removable hydrophobic group, R¹ is CH₃. In some embodiments, the compound of Formula (IIID′) is a disodium salt. In some embodiments, the compound of Formula (IIID′) is a monosodium salt.

In some embodiments, the modified Im-m⁷GDP has the structure of Formula (XI):

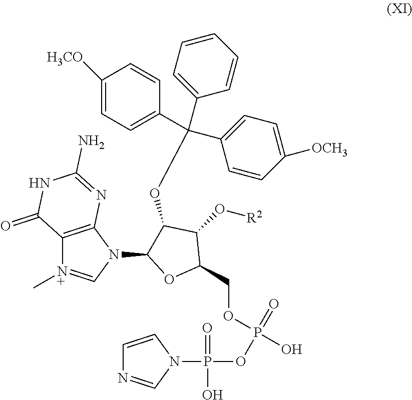

or a salt thereof, wherein R² is hydrogen or C₁₋₃ alkyl. In some such embodiments, R² is H. In other such embodiments, R² is CH₃. In some embodiments, the compound of Formula (XI) is a disodium salt. In some embodiments, the compound of Formula (XI) is a monosodium salt.

In some embodiments, the modified Im-m⁷GDP has the structure of Formula (XII):

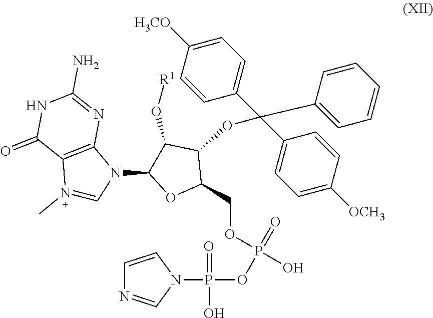

or a salt thereof, wherein R¹ is hydrogen or C₁₋₃ alkyl. In some such embodiments, R¹ is H. In other such embodiments, R¹ is CH₃. In some embodiments, the compound of Formula (XII) is a disodium salt. In some embodiments, the compound of Formula (XII) is a monosodium salt.

In some embodiments, the modified Im-m⁷GDP has the structure of Formula (XIII):

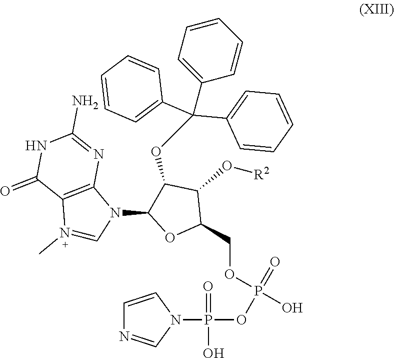

or a salt thereof, wherein R² is hydrogen or C₁₋₃ alkyl. In some such embodiments, R² is H. In some embodiments, R² is CH₃. In other of the forgoing embodiments, R² is CH₃. In some embodiments, the compound of Formula (XIII) is a disodium salt. In some embodiments, the compound of Formula (XIII) is a monosodium salt.

In some embodiments, the modified Im-m⁷GDP has the structure of Formula (XIV):

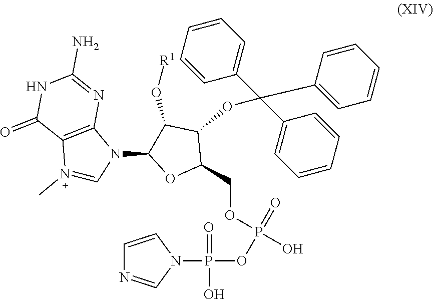

or a salt thereof, wherein R¹ is hydrogen or C₁₋₃ alkyl. In some such embodiments, R¹ is H. In some embodiments, R¹ is CH₃. In other of the forgoing embodiments, R¹ is CH₃. In some embodiments, the compound of Formula (XIV) is a disodium salt. In some embodiments, the compound of Formula (XIV) is a monosodium salt.

A compound of Formula (III′D), Formula (III″D) or Formula (IIID′) can be produced using standard synthetic methods known in the art. As an example, Scheme 1 depicts an exemplary synthesis of a precursor (compound A) of a specific compound of Formula (IIID′), whereon the 2′-hydroxy substituent is protected with a trityl (Trt) protecting group.

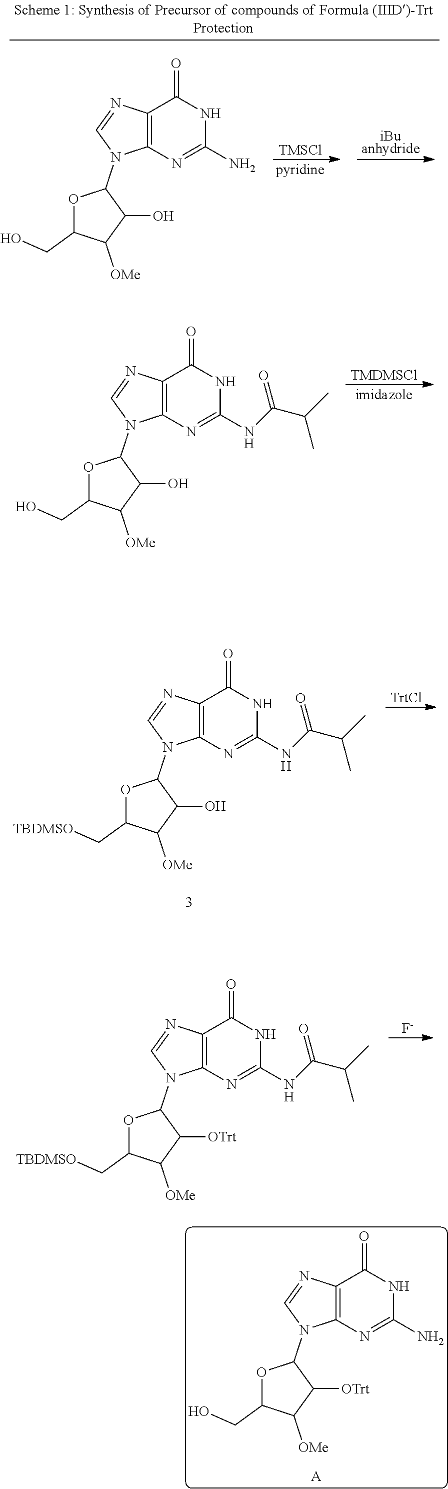

Scheme 1A depicts an exemplary embodiment of introducing a silyl protecting group.

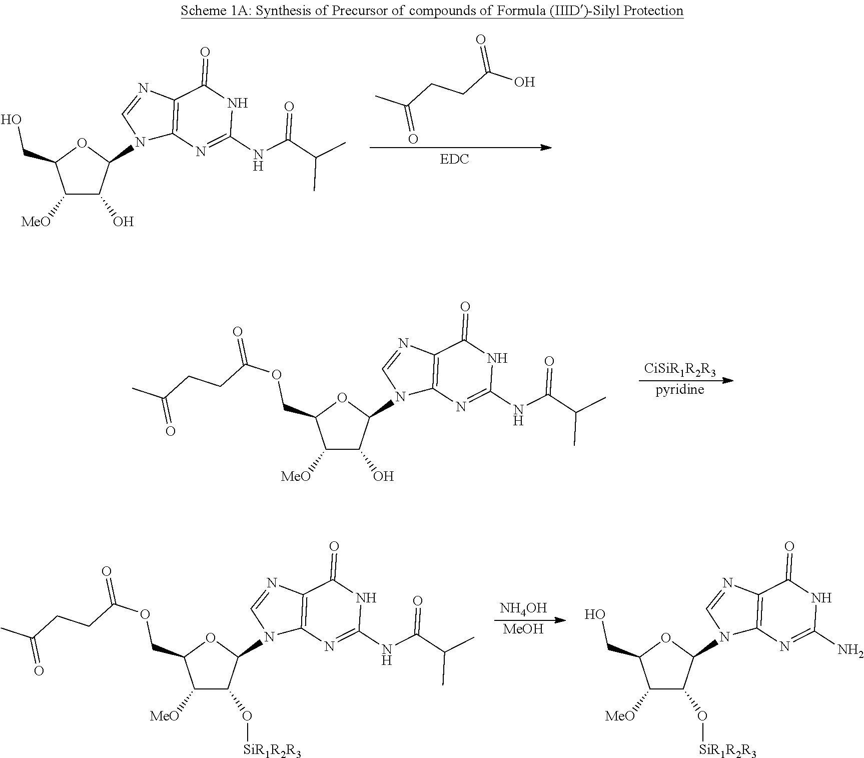

In some embodiments, —SiR₁R₂R₃ in Scheme 1A can be one of the following compounds:

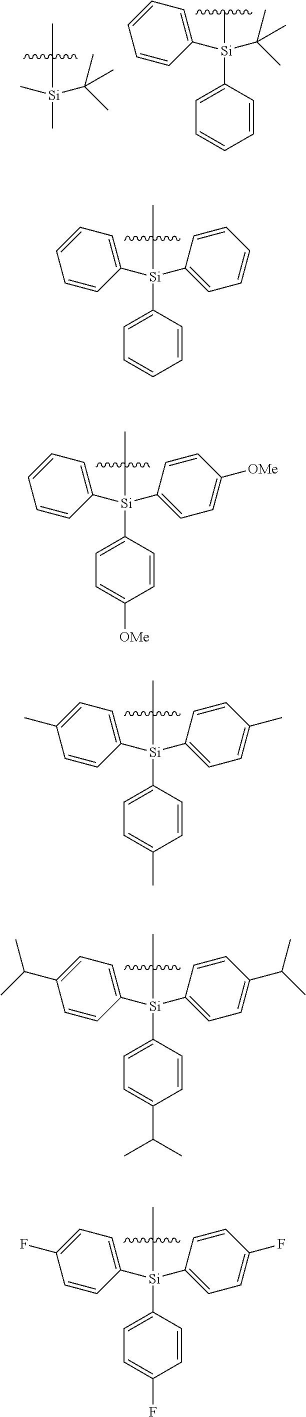

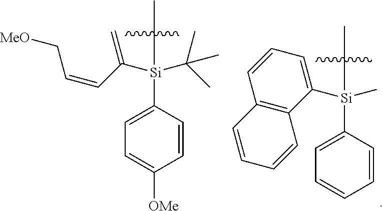

The silyl protecting group can be removed by methods known in the art, such as acids or fluorides (e.g. tetra-n-butylammonium fluoride (TBAF).

Scheme 2 depicts the synthesis of a specific compound of Formula (IIID′) from the precursor (compound A).

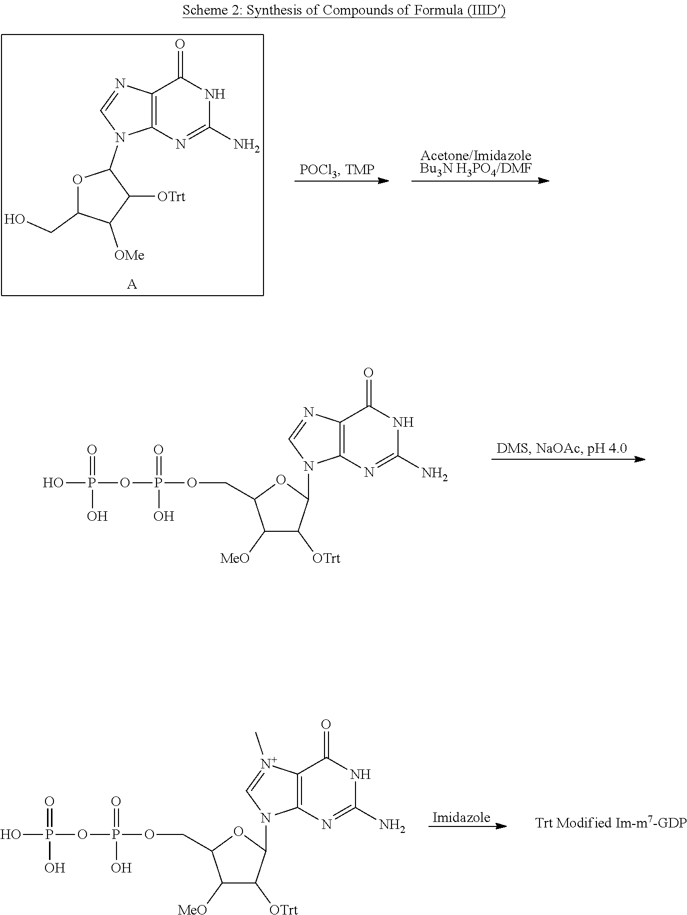

In some embodiments, R¹ or R² of the compound of Formula (III′D), Formula (III″D) or Formula (IIID′) can be a photolabile protecting group. Examples of photolabile protecting groups include but are not limited to arylcarbonylmethyl groups (e.g., phenacyl, o-alkylphenacyl, p-hydroxyphenacyl and benzoin groups), nitroaryl groups, coumarin-4-yl groups, arylmethyl groups, meta-containing groups, and pivaloyl groups. A photolabile protected Im-m⁷GDP can be produced using standard synthetic methods known in the art. As an example, Scheme 3 depicts an exemplary synthesis of a precursor (compound B) of a specific compound of Formula (IIID′), whereon the 2′-hydroxy substituent is protected with a photolabile protecting group.

In some embodiments, R¹ or R² of the compound of Formula (III′D), Formula (III″D) or Formula (IIID′) can be an alkyl, heterocycloalkyl, or aryl substituted ethyl ether protecting group. Examples of substituted alkyl, heterocycloalkyl, or aryl ethyl ether protecting groups include but are not limited to tetrahydrofuranyl, t-butyl ethyl ether, isopropyl ethyl ether, and the like. These protecting groups can be removed, for example, with mild acids, TBAF, or heat (thermolabile).

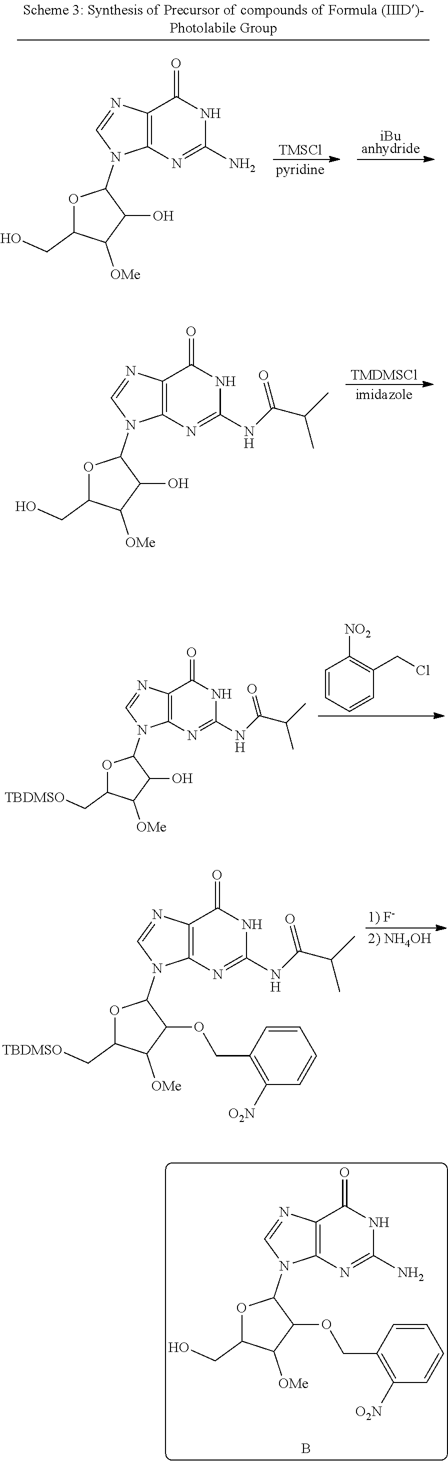

In some embodiments, the amino group of the N7-methylated GDP moiety of the Im-m⁷GDP is modified with a removable hydrophobic group. For instance, in particular embodiments, the modified Im-m⁷GDP has the structure of Formula (III′E):

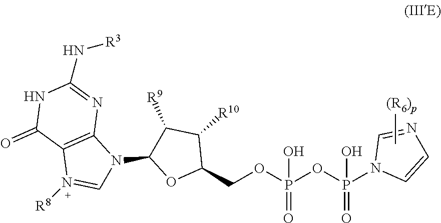

or a salt thereof, wherein R³ is a removable hydrophobic group, R⁶ is a substituent other than hydrogen (e.g., halo, amino, C₁₋₆alkyl, C₁₋₆alkoxy, —OH, or —CN), R⁸ is selected from the group consisting of hydrogen, C₂₋₁₂ alkyl, (C₂₋₁₂alkyl)halo, (C₂₋₁₂alkyl)NH₂, (C₂₋₁₂alkyl)OH, cyclic alkyl group optionally substituted with one or more NH₂, NHC₁₋₆ alkyl, halo or OH group, or a 5 or 6 membered aromatic or heteroaromatic ring optionally substituted with one or more NH₂, NHC₁₋₆ alkyl, halo or OH group, R⁹ and R¹⁰ are each independently selected from the group consisting of hydrogen, OH, NR¹¹R¹², C₁₋₆ alkyl, OC₁₋₆ alkyl, halo (e.g., F, Cl, Br or I), O(C₁₋₆ alkyl)O C₁₋₆ alkyl, (C₁₋₆ alkyl)N₃, O(C₁₋₆ alkyl)N₃, CH₂halo, CH(halo)₂, C(halo)₃, O(C₁₋₆alkyl)halo, O(C₁₋₆alkyl)CH₂halo, O(C₁₋₆alkyl)CH(halo)₂, O(C₁₋₆alkyl)C(halo)₃, OCH₂—CH═CH₂, O(CH₂)₂—OCH₃, O(CH₂)₂—SCH₃, O(CH₂)₂—OCF₃, O(C₁₋₆alkyl)-N(R¹⁰)(R¹¹), O(C₁₋₆alkyl)-O(C₁₋₆alkyl)N(R¹¹)(R¹²), and OCH₂C(═O)—N(R¹¹)(R¹²), R¹¹ and R¹² are each independently H or C₁₋₆alkyl, and p is an integer from 0-3. In some embodiments, the compound of Formula (III′E) is a compound of Formula (IIIE) (p=0). In some embodiments, R³ is a 9-fluorenylmethoxycarbonyl (Fmoc) group or a substituted Fmoc group. Examples of substituted Fmoc groups include, but are not limited to, 2,7-di-tert-butyl-Fmoc, 2-fluro-Fmoc, and 2-monoisooctyl-Fmoc. In other embodiments, R³ is an acyl. In one such embodiment, the acyl has the formula

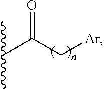

wherein Ar is a substituted or unsubstituted aromatic moiety and n is an integer from 0 to 5. In a particular embodiments, the acyl has the formula

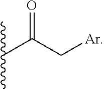

Particular acyls include, but are not limited to

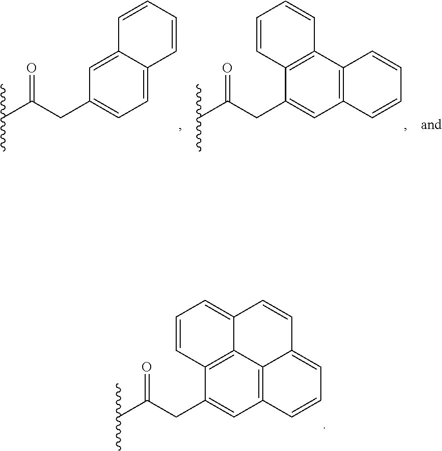 [SMILES: *C(=O)Cc1ccc2ccccc2c1 ; *C(=O)Cc1cc2ccccc2c2ccccc12 ; *C(=O)Cc1cc2cccc3ccc4cccc1c4c32]

In some embodiments, R³ is 2-(4-nitrophenylsulfonyl)ethoxycarbonyl (Nsc), 1,1-dioxobenzo[b]thiophene-2-ylmethoxycarbonyl (Bsmoc), (1,1,dioxonaphtho[1,2-b]thiophene-2-yl)methylcarbonyl (α-Nsmoc), 1-(4,4-dimethyl-2,6-dioxocyclohex-1-ylidene)-e-methylbutyl (ivDde), 2-phenyl(methyl)sulfonio)ethoxycarbonyl tetrafluoroborate (Pms), wthanesulfonylethoxycarbonyl (Esc) 9-fluorenylmethyl (Fm), 4-(N-[1-(4,4-dimethyl-2,6-dioxocyclohexylidene)-3-methylbutyl]-amino)benzyl (Dmab) or 2-(4-sulfophenylsulfonyl)ethoxycarbonyl (Sps). In some of the foregoing embodiments R⁴ is H and R⁵ is H. In other of the forgoing embodiments, R⁴ is CH₃ and R⁵ is H. In other of the forgoing embodiments, R⁴ is CH₃ and R⁵ is CH₃. In other of the forgoing embodiments, R⁴ is Hand R⁵ is H. In some embodiments, the compound of Formula (III′E) is a disodium salt. In some embodiments, the compound of Formula (III′E) is a monosodium salt.

In some embodiments of the compound of Formula (III′E), R⁹ is halo. In some such embodiments, R⁹ is F. In other embodiments of the compound of Formula (III′E), R⁹ is OH. In other embodiments of the compound of Formula (III′E), R⁹ is OCH₃. In other embodiments of the compound of Formula (III′E), R⁹ is OCH₂N₃. In other embodiments of the compound of Formula (III′E), R⁹ is OCH₂CH₂OCH₃. In other embodiments of the compound of Formula (III′E), R⁹ is selected from the group consisting of OCH₂F, OCHF₂, OCF₃, OCH₂CH₃, O(CH₂)₂F, OCH₂CHF₂, OCH₂CF₃, OCH₂—CH═CH₂, and O(CH₂)₂—OCH₃. In any of the foregoing embodiments, R⁸ can be C₁₋₆alkyl. In some such embodiments, R⁸ is CH₃. In any of the foregoing embodiments, p can be 0. In any of the foregoing embodiments, R¹⁰ can be H, OH, OCH₃, or F.

In some embodiments of the compound of Formula (III′E), R⁹ is halo. In some such embodiments, R¹⁰ is F. In other embodiments of the compound of Formula (III′E), R¹⁰ is OH. In other embodiments of the compound of Formula (III′E), R¹⁰ is OCH₃. In other embodiments of the compound of Formula (III′E), R¹⁰ is OCH₂N₃. In other embodiments of the compound of Formula (III′E), R¹⁰ is OCH₂CH₂OCH₃. In other embodiments of the compound of Formula (III′E), R¹⁰ is selected from the group consisting of OCH₂F, OCHF₂, OCF₃, OCH₂CH₃, O(CH₂)₂F, OCH₂CHF₂, OCH₂CF₃, OCH₂—CH═CH₂, and O(CH₂)₂—OCH₃. In any of the foregoing embodiments, R⁸ can be C₁₋₆alkyl. In some such embodiments, R⁸ is CH₃. In any of the foregoing embodiments, p can be 0. In any of the foregoing embodiments, R⁹ can be H, OH, OCH₃, or F.

In some embodiments of the compound of Formula (III′E), R⁸ is C₁₋₆alkyl. In some such embodiments R⁸ is CH₃. In other embodiments R⁸ is ethyl.

In some embodiments of the compound of Formula (III′E), R⁸ is (C₂₋₆alkyl)halo. In other embodiments of the compound of Formula (III′E), R⁸ is (C₂₋₆alkyl)NH₂. In other embodiments of the compound of Formula (III′E), R⁸ is (C₂₋₆alkyl)OH.

In some embodiments of the compound of Formula (III′E), R⁸ is a cyclopentyl or cyclohexyl group. In some embodiments, the cyclopentyl or cyclohexyl group is substituted with one or more NH₂, NHC₁₋₆ alkyl, halo or OH group.

In some embodiments, the amino group of the N7-methylated GDP moiety of the Im-m⁷GDP is modified with a removable hydrophobic group. For instance, in particular embodiments, the modified Im-m⁷GDP has the structure of Formula (IIIE′):

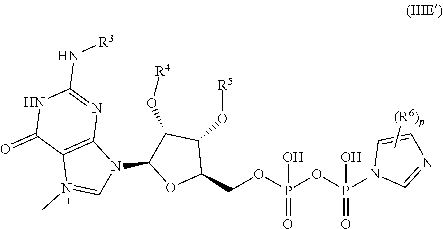

or a salt thereof, wherein R³ is a removable hydrophobic group, R⁴ is hydrogen or C₁₋₃ alkyl. R⁵ is hydrogen or C₁₋₃ alkyl, R⁶ is a substituent other than hydrogen (e.g., halo, amino, C₁₋₆alkyl, C₁₋₆alkoxy, —OH, or —CN), and p is an integer from 0-3. In some embodiments, the compound of Formula (IIIE′) is a compound of Formula (IIIE) (p=0). In some embodiments, R³ is a silyl protecting group. In embodiments R³ can be, for example,

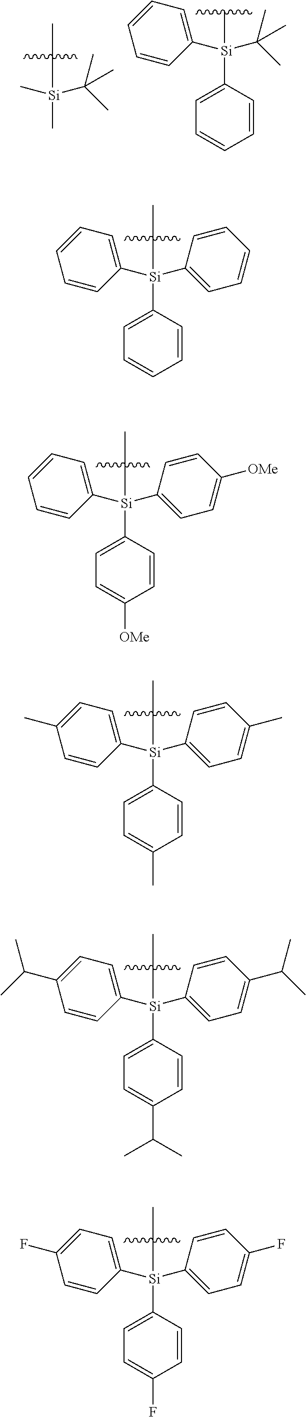

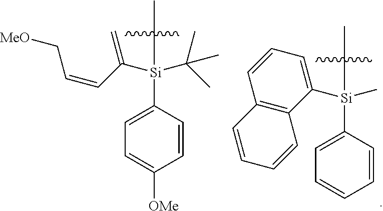

In some embodiments, R³ is a 9-fluorenylmethoxycarbonyl (Fmoc) group or a substituted Fmoc group. Examples of substituted Fmoc groups include, but are not limited to, 2,7-di-tert-butyl-Fmoc, 2-fluro-Fmoc, and 2-monoisooctyl-Fmoc. In other embodiments, R³ is an acyl. In one such embodiment, the acyl has the formula

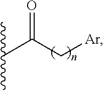

wherein Ar is a substituted or unsubstituted aromatic moiety and n is an integer from 0 to 5. In a particular embodiments, the acyl has the formula

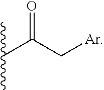

Particular acyl include, but are not limited to

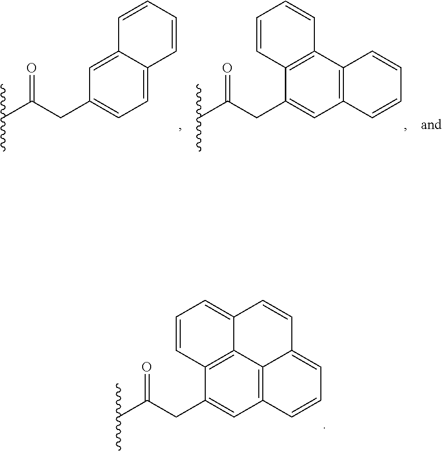 [SMILES: *C(=O)Cc1ccc2ccccc2c1 ; *C(=O)Cc1cc2ccccc2c2ccccc12 ; *C(=O)Cc1cc2cccc3ccc4cccc1c4c32]

In some embodiments, R³ is 2-(4-nitrophenylsulfonyl)ethoxycarbonyl (Nsc), 1,1-dioxobenzo[b]thiophene-2-ylmethoxycarbonyl (Bsmoc), (1,1,dioxonaphtho[1,2-b]thiophene-2-yl)methylcarbonyl (α-Nsmoc), 1-(4,4-dimethyl-2,6-dioxocyclohex-1-ylidene)-e-methylbutyl (ivDde), 2-phenyl(methyl)sulfonio)ethoxycarbonyl tetrafluoroborate (Pms), wthanesulfonylethoxycarbonyl (Esc) 9-fluorenylmethyl (Fm), 4-(N-[1-(4,4-dimethyl-2,6-dioxocyclohexylidene)-3-methylbutyl]-amino)benzyl (Dmab) or 2-(4-sulfophenylsulfonyl)ethoxycarbonyl (Sps). In some of the foregoing embodiments R⁴ is H and R⁵ is H. In other of the forgoing embodiments, R⁴ is CH₃ and R⁵ is H. In other of the forgoing embodiments, R⁴ is CH₃ and R⁵ is CH₃. In other of the forgoing embodiments, R⁴ is H and R⁵ is H. In some embodiments, the compound of Formula (IIIE′) is a disodium salt. In some embodiments, the compound of Formula (IIIE′) is a monosodium salt.

A compound of Formula (III′E) or (IIIE′) can be produced using standard synthetic methods known in the art. As an example, Scheme 4 depicts an exemplary synthesis of a precursor of a specific compound of Formula (IIIE′).

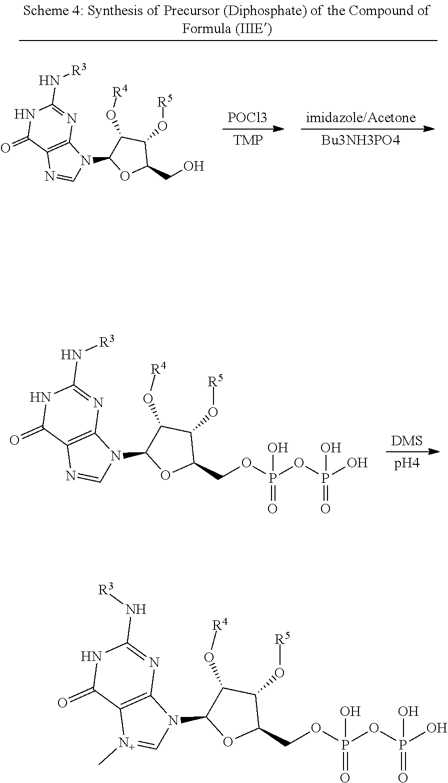

In other embodiments, the modified Im-m⁷GDP has a structure wherein the free amino group on the 7-methylguanosine moiety of the cap can be converted to a phthalimide moiety. For instance, in some embodiments, the protected capping agent can have the structure of Formula (III′F) or Formula (III′G):

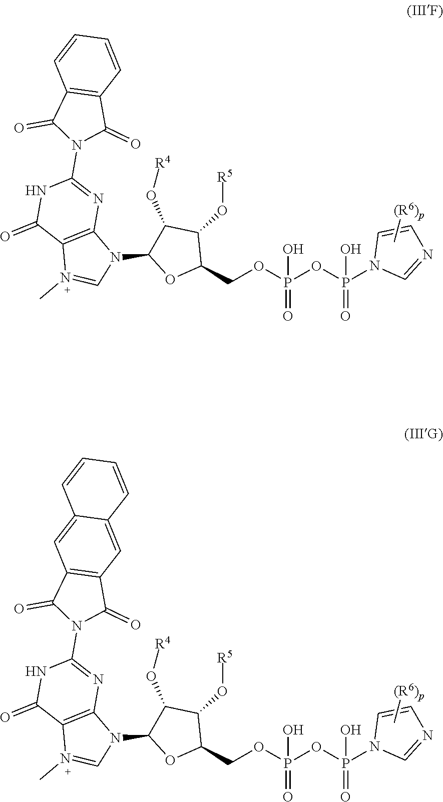

or a salt thereof, wherein R⁴, R⁵, R⁶ and p are defined as above. In some embodiments, the compound of Formula (III′F) is a compound of Formula (IIIF) (p-0). In some embodiments, the compound of Formula (III′G) is a compound of Formula (IIIG) (p=0). In some embodiments of the compound of Formula (III′F) or Formula (III′G), R⁴ is H and R⁵ is H. In other embodiments, R⁴ is CH₃ and R⁵ is H. In other embodiments, R⁴ is CH₃ and R⁵ is CH₃. In other embodiments, R⁴ is Hand R⁵ is CH₃. In some embodiments, the compound of Formula (III′F) or Formula (III′G) is a disodium salt. In some embodiments, the compound of Formula (III′F) or Formula (III′G) is a monosodium salt.

A compound of Formula (III′F) or Formula (III′G) can be produced using standard synthetic methods known in the art. As an example, Scheme 5 depicts an exemplary synthesis of a precursor of a specific compound of Formula (III′F).

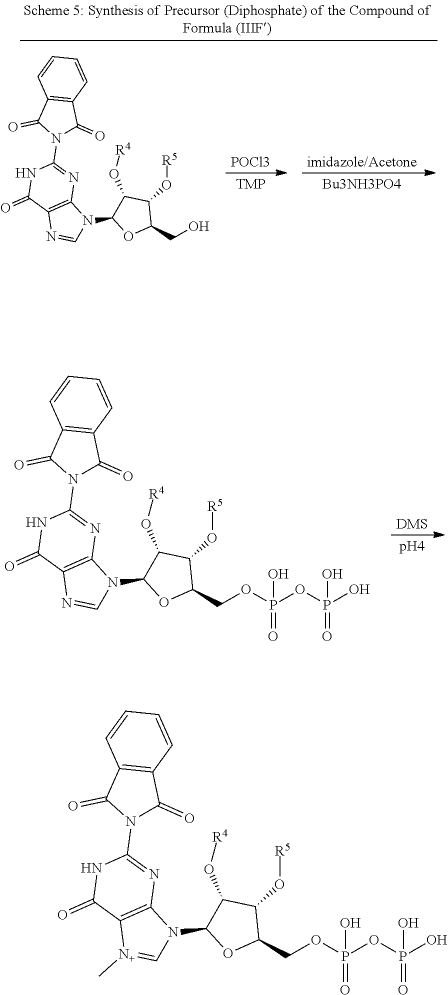

In some embodiments, the modified Im-m⁷GDP compounds that can react with 5′-phosphate-mRNA molecules to produce 5′-capped mRNA molecules that are protected at both the 2′-position (or the 3′-position) and the N7-methylated GDP moiety of the Im-m⁷GDP with a removable hydrophobic group. In certain embodiments, the modified Im-m⁷GDP has the structure of Formula (III′H):

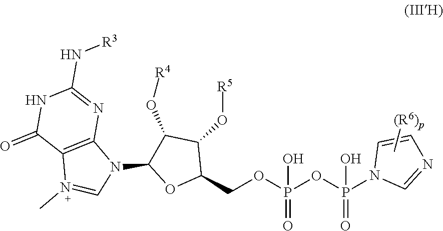

or a salt thereof, wherein R¹, R², R³, R⁶ and p are defined as above. In some embodiments, the compound of Formula (III′H) is a compound of Formula (IIIH) (p=0). In some embodiments, R¹ or R² is trityl (Trt), monomethoxytrityl, or dimethoxytrityl (DMT). In other embodiments, R³ is an Fmoc group or a substituted Fmoc group. In other such embodiments, R³ is an acyl. In one such embodiment, the acyl has the formula

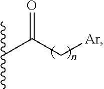

wherein Ar is a substituted or unsubstituted aromatic moiety and n is an integer from 0 to 5. In a particular embodiment, the acyl has the formula

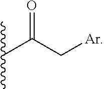

Particular acyls include, but are not limited to

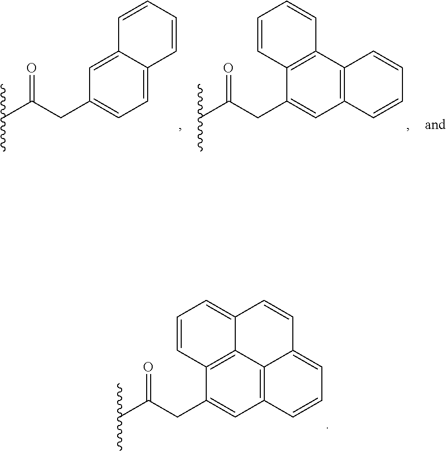 [SMILES: *C(=O)Cc1ccc2ccccc2c1 ; *C(=O)Cc1cc2ccccc2c2ccccc12 ; *C(=O)Cc1cc2cccc3ccc4cccc1c4c32]

In some embodiments, R¹ is trityl (Trt), monomethoxytrityl, or dimethoxytrityl (DMT) and R² is an Fmoc or substituted Fmoc group. In some such embodiments, R² is CH₃. In some embodiments, R² is trityl (Trt), monomethoxytrityl, or dimethoxytrityl (DMT) and R² is an Fmoc or substituted Fmoc group. In some such embodiments, R¹ is CH₃.

A modified Im-m⁷GDP compound of the disclosure (e.g. a compound of Formula (IIID), (III′D), (III″D), (IIID′), (IIIE), (III′E), (IIIE′). (IIIF), (III′F), (IIIG), (III′G), (IIIH), (III′H), (XI), (XII), or (XIII)) can be reacted with a 5′-phosphate-mRNA to form a protected 5′-capped mRNA, wherein the 2′-position or 3′-position of the cap and/or N7-methylated GDP moiety of the cap are protected with one or more removable hydrophobic groups. The 5′-phosphate-mRNA can be prepared non-enzymatically using an automated synthesizer, as described in Abe et al., ACS Chem. Biol. 2022, 17, 1308-1314. As set forth below, a variety of different 5′-phosphate-mRNA molecules can be prepared that react with the modified Im-m⁷GDP compounds of the disclosure. For instance, 5′-phosphate-mRNA molecules with modified nucleotides at the 5′-end can be prepared non-enzymatically and reacted with the modified Im-m⁷GDP compound of the disclosure. Following reaction between a modified Im-m⁷GDP compound of the disclosure and a 5′-phosphate-mRNA, the protecting group(s) can be removed using methods known in the art. For instance, protecting groups can be removed through the addition of a weak acid or a weak base, or by heating.

In certain embodiments, the capping methods of the disclosure (i.e., reacting modified Im-m⁷GDP compounds with 5′-phosphate-mRNA molecules) are performed in solution (e.g., an organic solvent) following removal of the 5′-phosphate-mRNA from a solid support. For instance, in particular embodiments, the capping methods of the disclosure can be performed in DMSO, DMF, THF or methylene chloride. In some embodiments, the capping methods of the disclosure are performed in the presence of a salt that includes a divalent cation. In some such embodiments, the divalent salt is CaCl₂, ZnCl₂, MgCl₂, or CuCl₂. In some embodiments, the capping methods of the disclosure are performed in the presence of 1-methylimidazole.

Different salt forms of the 5′-phosphate-mRNA molecules can be reacted with modified Im-m⁷GDP compounds of the disclosure to generate 5′-capped mRNA molecules. In some embodiments, the 5′-phosphate-mRNA undergoing the capping reaction is a sodium salt. In other embodiments, the 5′-phosphate-mRNA undergoing the capping reaction is an ammonium salt (e.g., a trimethylammonium salt).

In one embodiment, modified Im-m⁷GDP compounds of the disclosure can have a structure having one of the formulas depicted below:

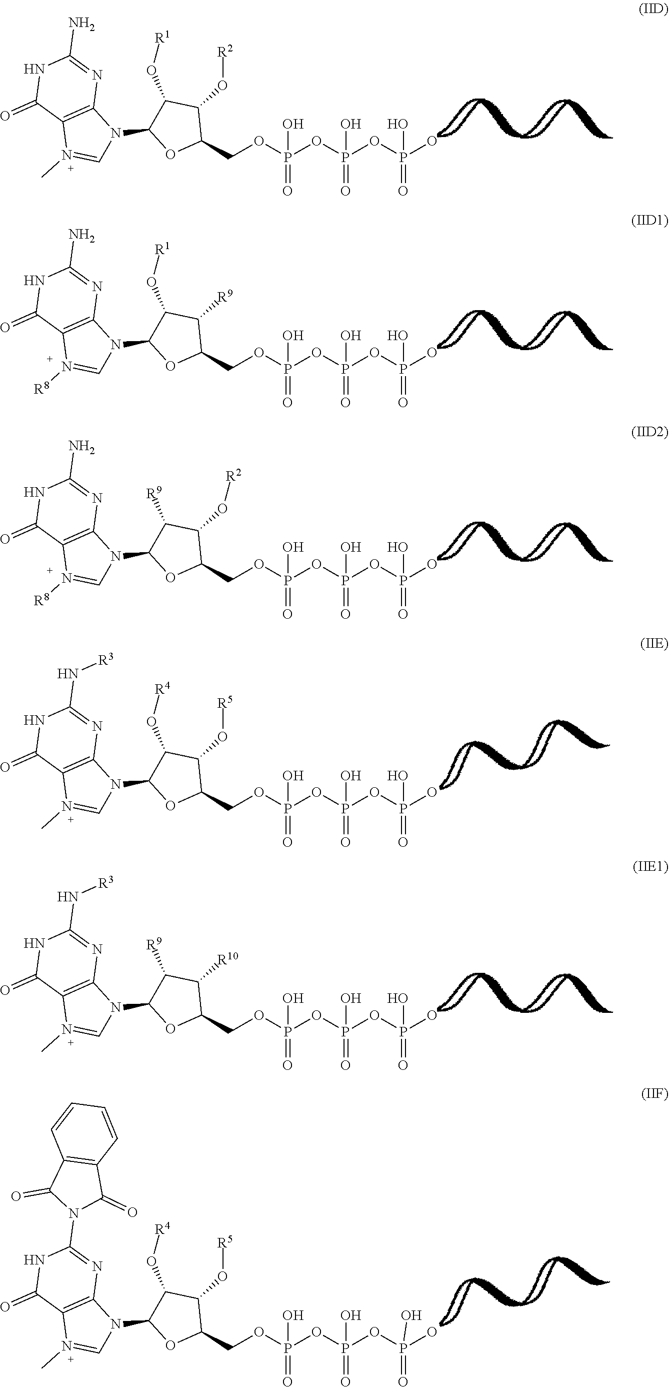

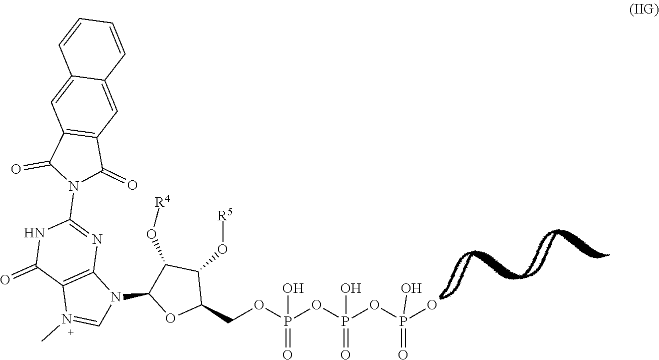

or a salt thereof, wherein R¹, R², R³, R⁴, R⁵, R⁸, and R⁹ are defined as above, and the helical structure represents the mRNA portion of the protected 5′-capped mRNA. Following purification of the compound of Formula IID, IID1, IID2, IIE, IIE1, IIF or IIG, including the removal of any uncapped mRNAs, the protecting group can be chemically removed, yielding the purified 5′-capped mRNA.

In one embodiment, modified Im-m⁷GDP compounds of the disclosure can have a structure having one of the formulas depicted below:

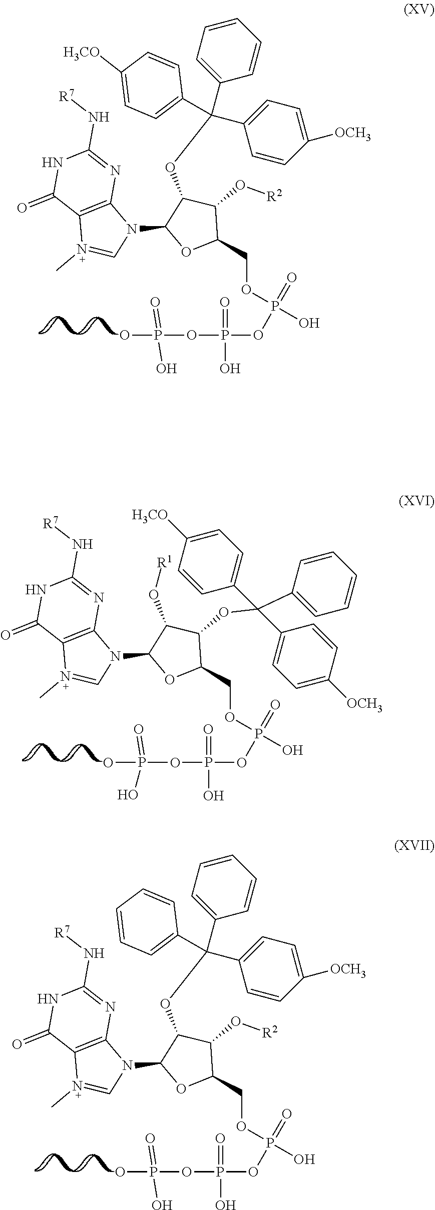

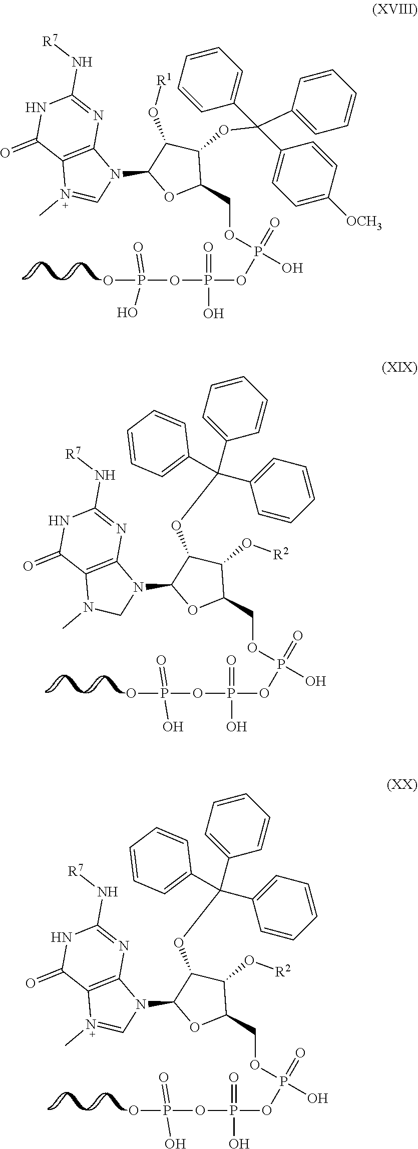

or a salt thereof, wherein R¹ and R² is defined as above, R⁷ is H or R³ (as defined above), and the helical structure represents the mRNA portion of the protected 5′-capped mRNA. In some embodiments, R⁷ is H. In some embodiments, R⁷ is an Fmoc group or a substituted Fmoc group. In some such embodiments, R² is H. In some embodiments, R² is CH₃. In some such embodiments, R¹ is H. In some embodiments, R¹ is CH₃. Following purification of the compound of any of Formula (XV) to (XX), including the removal of any uncapped mRNAs, the protecting group can be chemically or thermally removed, yielding the purified 5′-capped mRNA. For instance, in some embodiments the trityl group can be removed by reacting the compound of any one of Formulas (XV) to (XX) with a mild acid such as trifluoroacetic acid (TFA).

In some embodiments, the purified 5′-capped mRNAs (following removal of the protecting group(s)) of the disclosure comprise fewer than 200 nucleotide bases. In some embodiments, the purified 5′-capped mRNAs of the disclosure comprise fewer than 150 nucleotide bases. In some embodiments, the purified 5′-capped mRNAs of the disclosure comprise fewer than 130 nucleotide bases. In some embodiments, the purified 5′-capped mRNAs of the disclosure comprise fewer than 120 nucleotide bases. In some embodiments, the purified 5′-capped mRNAs of the disclosure comprise fewer than 110 nucleotide bases. In some embodiments, the purified 5′-capped mRNAs of the disclosure comprise fewer than 100 nucleotide bases. In some embodiments, the purified 5′-capped mRNAs of the disclosure comprise fewer than 100 nucleotide bases. In some embodiments, the purified 5′-capped mRNAs of the disclosure comprise fewer than 50 nucleotide bases. In some embodiments, the purified 5′-capped mRNAs of the disclosure comprise from about 30 nucleotide bases to about 75 nucleotide bases. In some embodiments, the purified 5′-capped mRNAs of the disclosure comprise from about 50 nucleotide bases to about 100 nucleotide bases. In some embodiments, the purified 5′-capped mRNAs of the disclosure comprise from about 50 nucleotide bases to about 150 nucleotide bases. In some embodiments, the purified 5′-capped mRNAs of the disclosure comprise from about 75 nucleotide bases to about 150 nucleotide bases. In some embodiments, the purified 5′-capped mRNAs of the disclosure comprise from about 100 nucleotide bases to about 150 nucleotide bases. In some embodiments, the purified 5′-capped mRNAs of the disclosure comprise from about 100 nucleotide bases to about 125 nucleotide bases. In some embodiments, the purified 5′-capped mRNAs of the disclosure comprise from about 120 nucleotide bases to about 150 nucleotide bases.

Following separation of the modified 5′-capped mRNAs of the disclosure from impurities (e.g., uncapped mRNAs), the protecting group(s) of modified 5′-capped mRNAs are removed to yield highly pure 5′-capped mRNAs. Alternatively, where the protecting group(s) are non-removable, following separation of the modified 5′-capped mRNAs of the disclosure from impurities (e.g., uncapped mRNAs), the highly pure modified 5′-capped mRNAs are ready to be translated. In some embodiments, the disclosure provides compositions comprising chemically synthesized 5′-capped mRNA molecules that comprise less than about 2% by weight of uncapped 5′-phosphate-mRNA molecules. In some embodiments, the compositions comprise less than 1% by weight of uncapped 5′-phosphate-mRNA molecules. In some embodiments, the compositions comprise less than 0.5% by weight of uncapped 5′-phosphate-mRNA molecules. In some embodiments, the compositions comprise less than 0.25% by weight of uncapped 5′-phosphate-mRNA molecules. In some embodiments, the compositions comprise less than 0.1% by weight of uncapped 5′-phosphate-mRNA molecules. In some embodiments, the compositions are substantially free of uncapped 5′-phosphate-mRNA molecules. In some embodiments, the compositions comprise from about 0.1% to about 1% by weight of uncapped 5′-phosphate-mRNA molecules. In some embodiments, the compositions comprise from about 0.1% to about 0.7% by weight of uncapped 5′-phosphate-mRNA molecules. In some embodiments, the compositions comprise from about 0.1% to about 0.5% by weight of uncapped 5′-phosphate-mRNA molecules. In some embodiments, the compositions comprise from about 0.1% to about 0.3% by weight of uncapped 5′-phosphate-mRNA molecules. In some embodiments, the compositions comprise from about 0.05% to about 1% by weight of uncapped 5′-phosphate-mRNA molecules. In some embodiments, the compositions comprise from about 0.05% to about 0.8% by weight of uncapped 5′-phosphate-mRNA molecules. In some embodiments, the compositions comprise from about 0.05% to about 0.5% by weight of uncapped 5′-phosphate-mRNA molecules. In some embodiments, the compositions comprise from about 0.05% to about 0.2% by weight of uncapped 5′-phosphate-mRNA molecules. In some embodiments, the compositions comprise from about 0.05% to about 0.1% by weight of uncapped 5′-phosphate-mRNA molecules. In any of the foregoing embodiments, the compositions are substantially free of any enzymatic byproducts.

IV. Hybrid mRNAs

In another aspect, the disclosure provides hybrid mRNAs produced using ligation methods as disclosed herein. In embodiments, the disclosure provides hybrid 5′-capped oligonucleotides comprising 50-12000 nucleotides.

In some embodiments, a 5′-capped mRNAs produced in accordance with the methods of the disclosure, as described in Section III, can be ligated to another RNA molecule to increase the size (i.e., the number of nucleotide bases) of the mRNA. For instance, a 5′-capped mRNA produced in accordance with the disclosure can be ligated to another RNA transcript that comprises at least 50 nucleotide bases, at least 100 nucleotide bases, at least 200 nucleotide bases, at least 300 nucleotide bases, at least 400 nucleotide bases, at least 500 nucleotide bases, at least 600 nucleotide bases, at least 700 nucleotide bases, at least 800 nucleotide bases, at least 900 nucleotide bases, at least 1,000 nucleotide bases, at least 1,200 nucleotide bases, at least 1,500 nucleotide bases, at least 2,000 nucleotide bases, at least 2,500 nucleotide bases, at least 3,000 nucleotide bases, at least 4,000 nucleotide bases, at least 5,000 nucleotide bases, at least 6,000 nucleotide bases, at least 7,000 nucleotide bases, at least 8,000 nucleotide bases, at least 9,000 nucleotide bases, at least 10,000 nucleotide bases, at least 11,000 nucleotide bases, or at least 12,000 nucleotide bases, to produce an elongated mRNA transcript. For instance, if a 5′-capped mRNA produced in accordance with the disclosure, as described in Section III, that comprises 100 nucleotide bases is ligated to a second RNA transcript that comprises 400 nucleotide bases, the resultant transcript would comprise 500 nucleotide bases.

In some embodiments, a 5′-capped mRNA produced in accordance with the methods of the disclosure can be ligated to another RNA molecule comprising at least about 500 nucleotide bases and no more than 12,000 nucleotide bases. In some embodiments, a 5′-capped mRNA produced in accordance with the methods of the disclosure can be ligated to another RNA molecule comprising at least about 500 nucleotide bases and no more than 8,000 nucleotide bases. In some embodiments, a 5′-capped mRNAs produced in accordance with the methods of the disclosure can be ligated to another RNA molecule comprising at least about 1,000 nucleotide bases and no more than 5,000 nucleotide bases.

In some embodiments, the protecting group (e.g., Trt, DMT or Fmoc) of the 5′-capped mRNA produced in accordance with the disclosure is not removed prior to reaction with the second mRNA transcript. In such embodiments, the protecting group is removed following the ligation transcript, hence generating the final elongated transcript.

In other embodiments, the protecting group of the 5′-capped mRNA produced in accordance with the disclosure is removed prior to the ligation reaction. In such embodiments, the ligation reaction produces the final elongated transcript.

A general schematic for ligating a 5′-capped mRNA produced by methods of the disclosure with a second (uncapped) mRNA to produce an elongated 5′-capped mRNA is shown in Scheme 6.

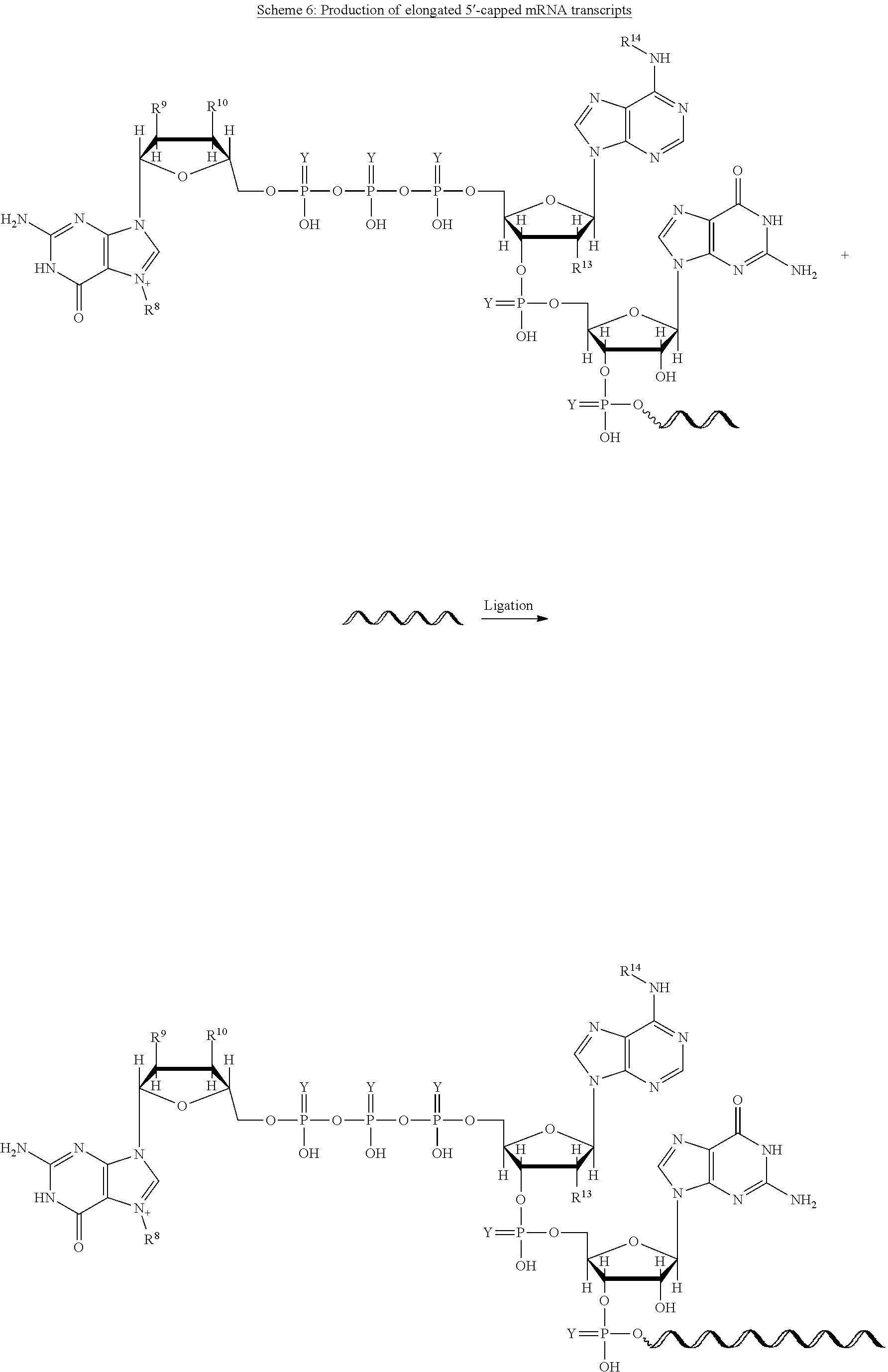

In Scheme 6, variables R⁸, R⁹, and R¹⁰ are defined as above, each Y is O or S, R¹³ is selected from the group consisting of hydrogen, OH, NR¹⁵R¹⁶, C₁₋₆ alkyl, OC₁₋₆ alkyl, halo (e.g., F, Cl, Br or I), O(C₁₋₆ alkyl)O C₁₋₆ alkyl, (C₁₋₆ alkyl)N₃, O(C₁₋₆ alkyl)N₃, CH₂halo, CH(halo)₂, C(halo)₃, O(C₁₋₆alkyl)halo, O(C₁₋₆alkyl)CH₂halo, O(C₁₋₆alkyl)CH(halo)₂, O(C₁₋₆alkyl)C(halo)₃, OCH₂—CH═CH₂, O(CH₂)₂—OCH₃, O(CH₂)₂—SCH₃, O(CH₂)₂—OCF₃, O(C₁₋₆alkyl)-N(R¹⁵)(R¹⁶), O(C₁₋₆alkyl)-O(C₁₋₆alkyl)N(R¹⁵)(R¹⁶), and OCH₂C(═O)—N(R¹⁵)(R¹⁶), R¹⁴ is H or C₁₋₆ alkyl, and R¹⁵ and R¹⁶ are each independently H or C₁₋₆alkyl. The 5′-capped mRNA transcript produced in accordance with the methods above is ligated to a second (uncapped) RNA transcript [symbol] produced independently. In some embodiments, the second RNA transcript can be produced through an in vitro transcription process. In other embodiments, the second RNA transcript can be produced through synthetic means. In embodiments, where the RNA transcript can be produced through synthetic means, the RNA may have modified nucleobase(s), modified sugar(s), and/or modified internucleotide linkage(s). In embodiments, a nucleobase or a sugar may be modified with a hydrophobic group. In some embodiments, the second RNA transcript is longer than the 5′-capped mRNA transcript. In some embodiments, the 5′-capped mRNA transcript is not polyadenylated at the 3′-end prior to reaction with the second RNA transcript. In some embodiments, the second RNA transcript is polyadenylated at the 3′-end.

In some embodiments, the 5′-capped mRNA transcript and the second (uncapped) transcript are ligated enzymatically. The RNA ligase catalyzes the formation of a 3′→5′ phosphodiester bond between the 3′-OH group on capped mRNA and the 5′-phosphate group on the second (uncapped) transcript. In some embodiments, the ligation reaction is a template-independent ligation reaction. In other embodiments, the ligation reaction is a template-dependent ligation reaction. In such embodiments, the template for the modified 5′-capped mRNA transcript and the second (uncapped) transcript can be any nucleic acid or nucleic acid analog that is capable of mediating a template-directed nucleic acid synthesis. In some embodiments, the template nucleic acid is attached to a solid support.

In some embodiments, the RNA ligase is a T4 RNA ligase 1. A general schematic of a ligation catalyzed by T4 RNA ligase is shown in Scheme 7. In Scheme 7, variables R⁸, R⁹, and R¹⁰, R¹³, R¹⁴ and Y are defined as above. Scheme 7 shows exemplary embodiments of the first two nucleotide bases that are linked to the 5′-cap of the mRNA transcript. It will be understood that the first two nucleotides can comprise other modified sugar moieties or modified nucleobases, such as those described in Section VI, below. Furthermore, Scheme 7 shows exemplary embodiments of the first two internucleoside linkages of the mRNA transcript after the 5′-cap. It will be understood that other modified internucleoside linkages can be used to link these nucleosides together, such as those described in Section VII, below.

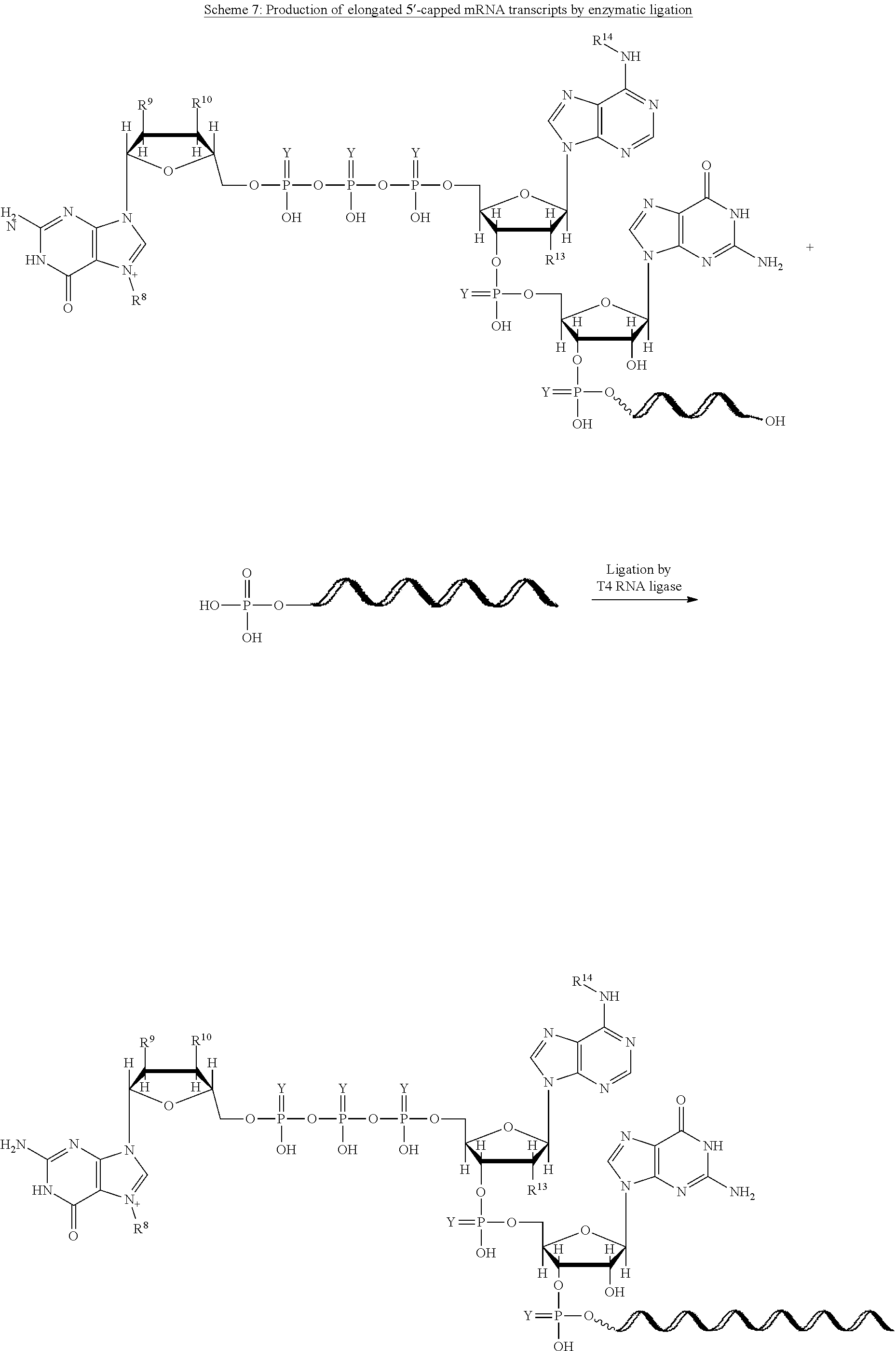

In some embodiments, the 5′-capped mRNA transcript and the second (uncapped) transcript are ligated chemically. In some such embodiments, a first click handle [symbol] is installed at the 3′-end of the 5′-capped mRNA transcript and a complementary second click handle [symbol] is installed at the 5′-end of the second (uncapped) mRNA transcript. A click reaction between the first click handle and the second click handle covalently links the 5′-capped mRNA transcript and the second (uncapped) transcript.

A general schematic of a click ligation is shown in Scheme 8. In Scheme 8, variables R⁸, R⁹, and R¹⁰, R¹³, R¹⁴ and Y are defined as above. The reaction between the first click handle and the second click handle produces a click product [symbol] that chemically links the two transcripts. Scheme 8 shows exemplary embodiments of the first two nucleotide bases that are linked to the 5′-cap of the mRNA transcript. It will be understood that the first two nucleotides can comprise other modified sugar moieties or modified nucleobases, such as those described in Section VI, below. Furthermore, Scheme 8 shows exemplary embodiments of the first two internucleoside linkages of the mRNA transcript after the 5′-cap. In will be understood that other modified internucleoside linkages can be used to link these nucleosides together, such as those described in Section VII, below.

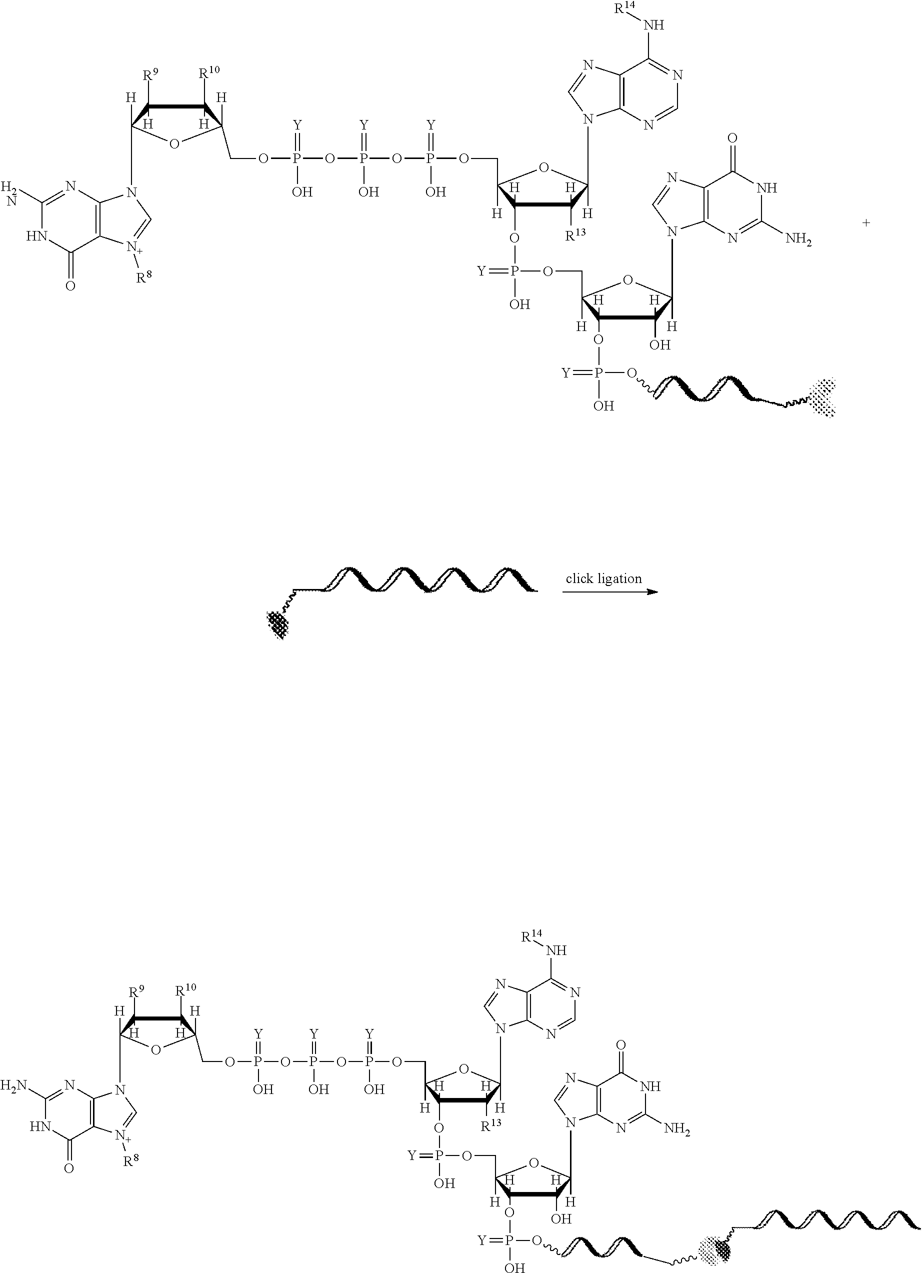

Scheme 9 shows an exemplary embodiment of a click reaction to chemically ligate RNA transcripts, where click₁ represents click handle 1 and click₂ represents click handle 2. The reaction between click handle 1 and click handle 2 produces a click product that ligates the 5′-capped mRNA transcript and the second (uncapped) transcript.

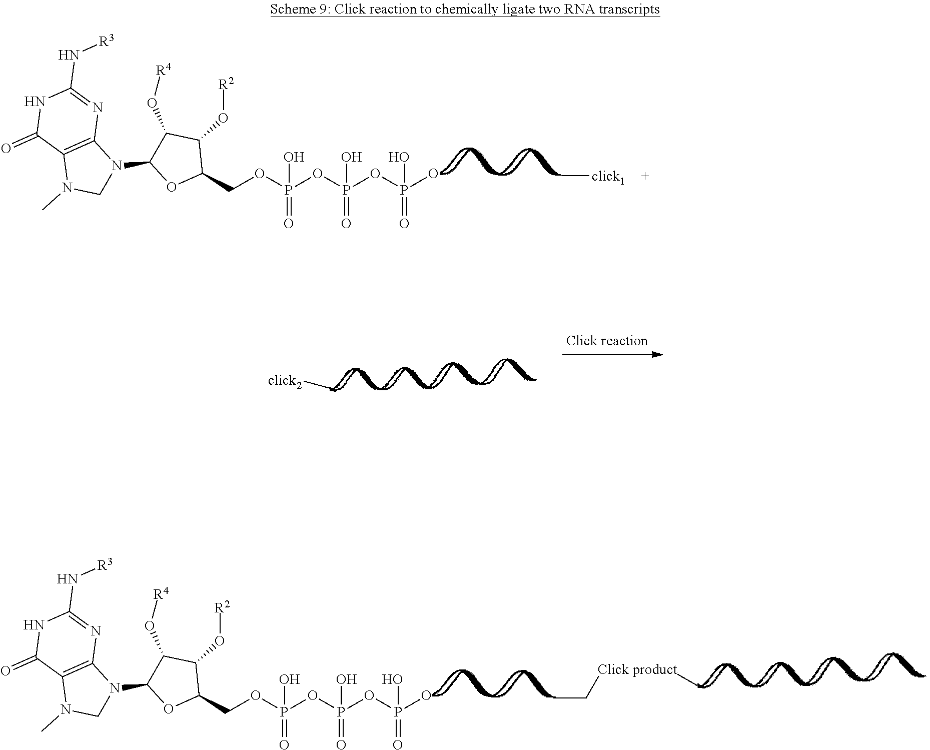

In some embodiments, the first click handle comprises an alkyne or azido group. In some embodiments, the second click handle comprises an alkyne or azido group. Methods of installing azido and alkyne click handles on RNA transcripts are described in Paredes and Das, ChemBioChem, 2010, 12 (1), 125-131. In some embodiments, the alkyne (or azido) group on the first click handle and the azido (or alkyne) group on the second click handle can be ligated using a copper-catalyzed Click reaction, thereby forming a triazole. In one embodiment, the Click reaction is a copper(I)-promoted azide-alkyne cycloaddition reaction. One such copper-catalyzed Click reaction is a Huisgen 1,3-dipolar cycloaddition (CuAAC) between an azide and an alkyne. The triazole-linked backbone does not compromise the function of the mRNA.

Scheme 10 shows an exemplary embodiment of a copper catalyzed Click reaction to chemically ligate two RNA transcripts. The click reaction between click handle 1 (alkyne) and click handle 2 (azide) produces a click product (triazole moiety) that ligates the 5′-capped mRNA transcript and the second (uncapped) transcript.

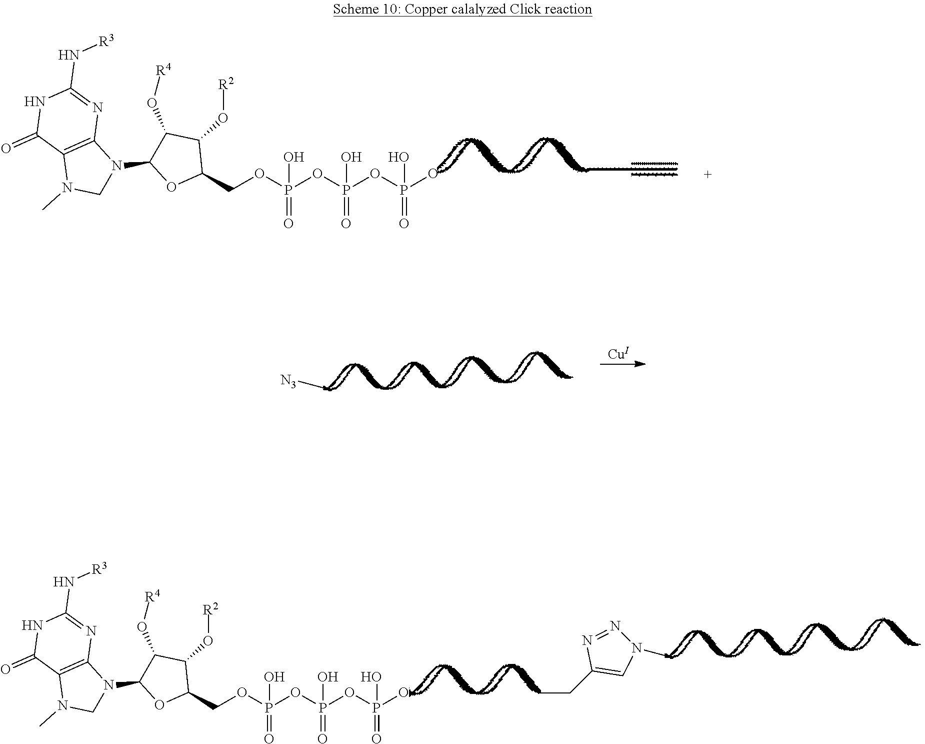

In another embodiment, the click product can be formed using copper-free Click chemistry. For example, the click product can be formed between an azide on the 5′-capped mRNA or second (uncapped) mRNA transcript and a dibenzocyclooctene (DBCO) moiety on the 5′-capped mRNA or second (uncapped) mRNA transcript. Alternatively, the click product can be formed using a Staudinger reaction between an azide and a phosphine, thus producing an aza-ylide.

In particular embodiments, the Click product can be formed from an inverse electron demand Diels-Alder reaction between a trans-cyclooctene (TCO) moiety on the first or second click handle and a tetrazine ring on the first or second click handle.

In some embodiments, the ligation methods disclosed herein (e.g., RNA ligase mediated ligation or click ligation) can produce hybrid RNA transcripts that have modified (i.e., unnatural) nucleosides or modified internucleoside linkages at the 5′-end of the transcript and unmodified (i.e., naturally occurring) nucleosides that include phosphodiester linkages at the 3′-end of the transcript. In some embodiments, the ligation methods disclosed herein (e.g., RNA ligase mediated ligation or click ligation) can produce hybrid RNA transcripts that have modified (i.e., unnatural) nucleosides or modified internucleoside linkages at the 3′-end of the transcript and unmodified (i.e., naturally occurring) nucleosides that include phosphodiester linkages at the 5′-end of the transcript. Modified nucleosides can include modified sugar moieties and/or modified nucleobases. Specific embodiments of modified nucleosides are discussed in Section VI, below. Specific embodiments of modified internucleoside linkages are discussed in Section VII, below.

In some embodiments, the first 50 to 150 nucleotide bases (along with the 5′-cap) of a transcript are generated using synthetic methods disclosed herein and are then ligated to a longer transcript produced by in vitro transcription. The longer transcript will include naturally occurring nucleotide bases and a phosphodiester backbone. However, the 5′-capped RNA transcript produced in accordance with the disclosure can include at least one modified nucleoside and/or at least one modified internucleoside linkage. For instance, in some embodiments, at least 2%, at least 5%, at least 10%, at least 20%, at least 30%, at least 40%, at least 50%, at least 60%, at least 70%, at least 80% at least 90% or 100% of the nucleosides are modified nucleosides. These modified nucleosides can comprise modifications to the sugar moiety or to the nucleoside base. In some embodiments at least 2%, at least 5%, at least 10%, at least 20%, at least 30%, at least 40%, at least 50%, at least 60%, at least 70%, at least 80%, at least 90%, or 100% of the phosphodiester linkages are modified. In some such embodiments, the modified linkages are thiophosphodiester linkages. In embodiments, the hybrid mRNA transcripts of the disclosure would only have a first portion (5′-end) of the final mRNA transcript with modified nucleotide bases or linkages, these modifications will be sufficient to confer increased stability and/or translation efficiency to the overall transcript.

Accordingly, the disclosure provides highly pure 5′-capped mRNA transcripts comprising a first portion and a second portion, wherein the first portion comprises one or more modified nucleosides and/or one or more modified internucleoside linkages and the second portion includes only of natural (unmodified) nucleosides linked by natural phosphodiester linkages. Additionally, the disclosure provides highly pure 5′-capped mRNA transcripts comprising a first portion and a second portion, wherein the second portion comprises one or more modified nucleosides and/or one or more modified internucleoside linkages and the first portion includes only of natural (unmodified) nucleosides linked by natural phosphodiester linkages. In some embodiments, at least 2%, at least 5%, at least 10%, at least 20%, at least 30%, at least 40%, at least 50%, at least 60%, at least 70%, at least 80% at least 90% or 100% of the nucleosides in the first portion are unnatural nucleosides. In some embodiments, at least 2%, at least 5%, at least 10%, at least 20%, at least 30%, at least 40%, at least 50%, at least 60%, at least 70%, at least 80% at least 90% or 100% of the nucleosides in the second portion are unnatural nucleosides. In some embodiments, at least 2%, at least 5%, at least 10%, at least 20%, at least 30%, at least 40%, at least 50%, at least 60%, at least 70%, at least 80%, at least 90% or 100% of the linkages between the nucleotide bases in the first portion are modified internucleoside linkages. In some embodiments, at least 2%, at least 5%, at least 10%, at least 20%, at least 30%, at least 40%, at least 50%, at least 60%, at least 70%, at least 80%, at least 90% or 100% of the linkages between the nucleotide bases in the second portion are modified internucleoside linkages. In some such embodiments, the modified internucleoside linkages are thiophosphodiester linkages. In some embodiments, the first portion and the second portion of the mRNA transcript is separated by a click product. In some such embodiments, the click product is a triazole ring.

The methods of ligating two mRNA transcripts as disclosed herein provide specific advantages that are not yet realized with current techniques used to synthetically prepare mRNA transcripts such as in vitro transcription. For example, the disclosed method allows for the production of long mRNA transcripts (e.g., longer than 1,000 nucleotide bases) that can have a variety of different 5′-cap analogs. Some of the different 5′-cap analogs are shown in Table 1, below. Additionally, the methodology enables the incorporation of modifications to the nucleosides or phosphate backbone into the target mRNA. Such modifications could potentially improve the stability and/or translation of the mRNA transcripts following delivery to a target cell.

In another embodiment, two or more purified 5′-capped mRNAs produced in accordance with the disclosure can be ligated to generate larger 5′-capped mRNA molecules. For instance, two purified 5′-capped mRNAs each comprising 125 nucleotide bases can be ligated together to generate a 5′-capped mRNA comprising 250 nucleotide bases. The larger mRNA transcript can be readily purified by traditional methods (e.g., HPLC). Advantageously, the longer 5′-capped mRNA transcripts can be produced on large scales using the methods of the disclosure.

V. Purified 5′-Capped mRNA Analogs

The disclosed methods can also be used to prepare translatable 5′-capped mRNA molecules with site-specific modifications in the 5′-capped mRNA molecules. These site-specific modifications can potentially increase translation activity and reduce immunogenicity. In some embodiments, a modification can be introduced at the 5′-end of the mRNA directly bonded to the cap. In some embodiments, the modification involves substituting a CH₃ group for a hydrogen at the 2′-position of the sugar of the first and/or second nucleotide at the 5′-end of the mRNA. In some embodiments, the 5′-end of the capped mRNA has a sequence as shown in Table 1 of U.S. Pat. No. 10,494,399, the contents of which are hereby incorporated by reference. Other mRNA molecules with modifications of the 5′-end of the mRNA are depicted in Table 1. In embodiments, a modification can be introduced at the 3′-end of the mRNA. In embodiments, the modification includes substituting a CH₃ group for a hydrogen at the 2′-position of the sugar of any of the first 10 nucleotides at the 3′-end of the mRNA. In embodiments, the modification includes 2 or more modified internucleotide linkages between the first 10 nucleotides at the 3′-end of the mRNA.

TABLE 1
Synthesized mRNAs

| Cap Analog | mRNA Structure |
|---|---|
| ^{m7}GpppA_{2′OMe}pG | 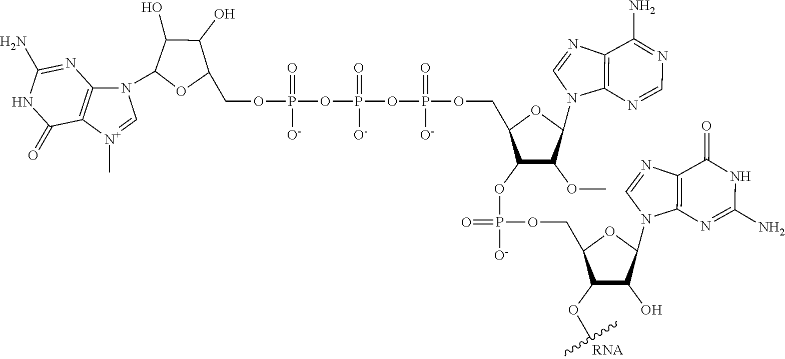 |
| m⁷G_{3′OMe}pppA_{2′OMe}pG | 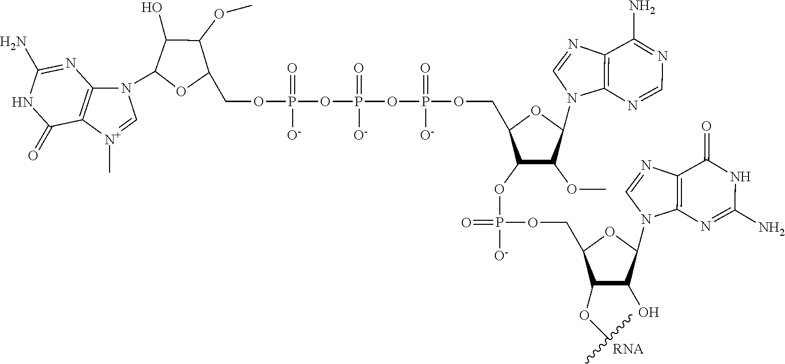 |
| m⁷Gppp(N6- methyladenine)_{2′OMe}pG | 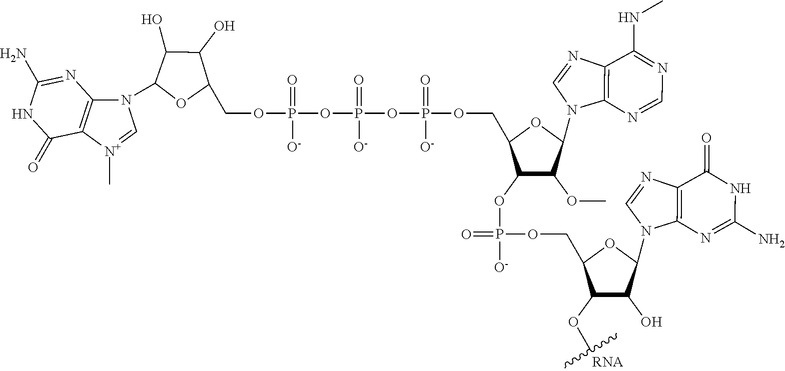 |
| m⁷G_{3′OMe}ppp(N6- methyladenine)_{2′OMe}pG | 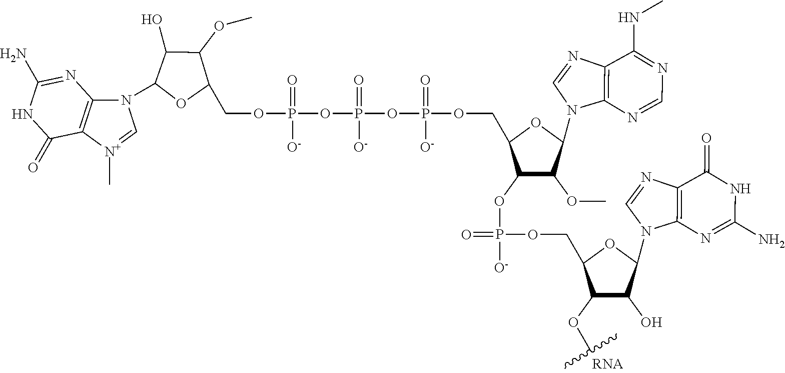 |
| m⁷GpppA_{2′,4′-LNA}pG | 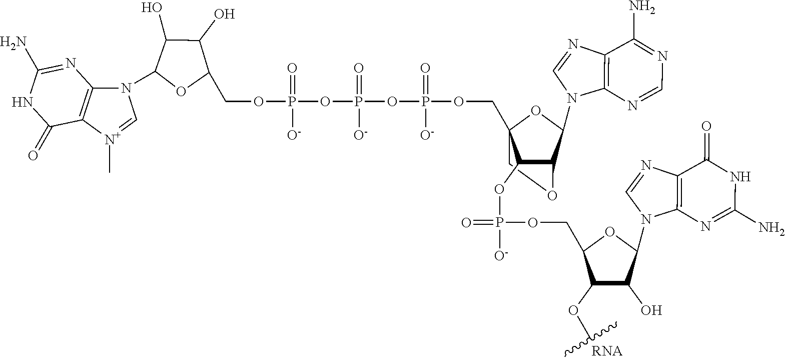 |
| m⁷G_{3′OMe}pppA_{2′,4′-} _{LNA}pG | 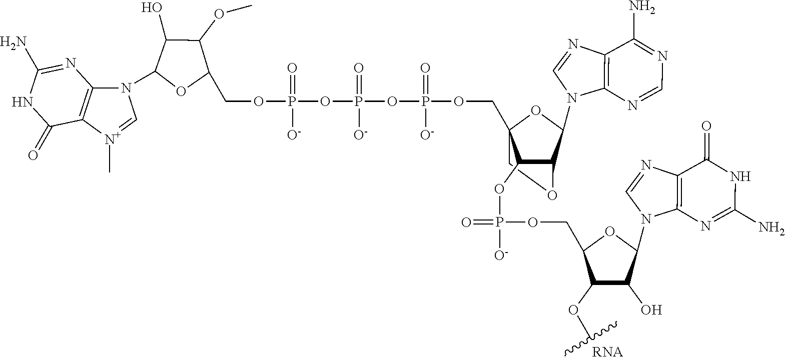 |
| m⁷Gppp(diamino- purine)_{2′OMe}pG | 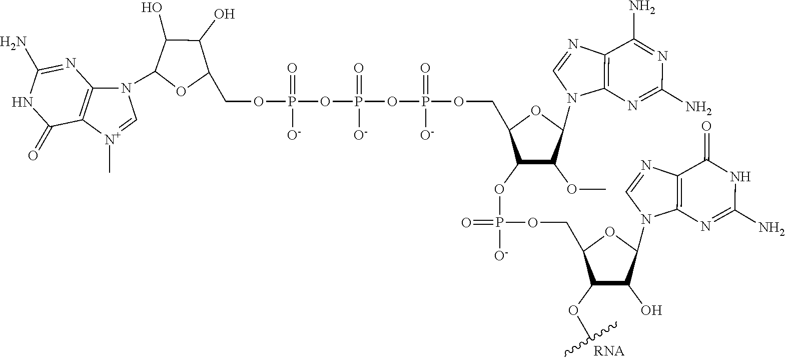 |
| m⁷G_{3′OMe}ppp(diamino- purine)_{2′OMe}pG | 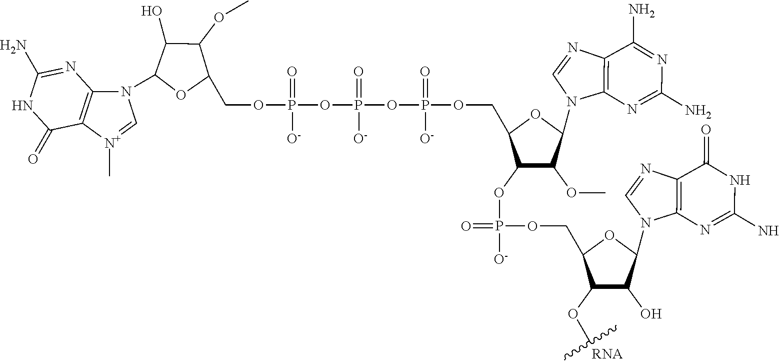 |
| m⁷G_{2′4′-} _{LNA}pppA_{2′OMe}pG | 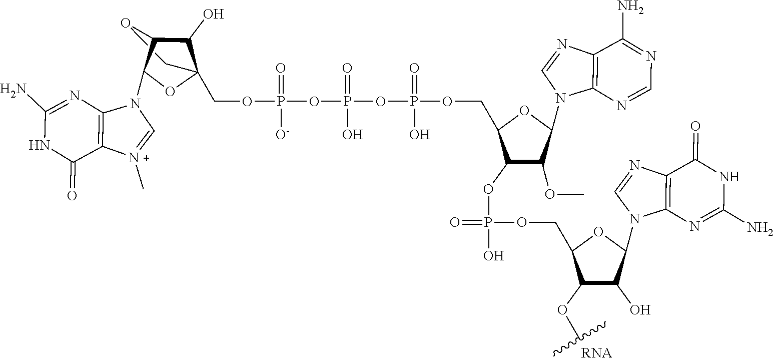 |

### VI. Modified Nucleosides

Modified nucleosides comprise a modified sugar moiety or a modified nucleobase or both a modified sugar moiety and a modified nucleobase.

### Modified Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH (“OMe” or ‘O-methyl”), and 2′-O(CH₂)₂OCH₃ (“MOE” or “O-methoxyethyl”), and T-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6. A “2′-O—N-methyl acetamide nucleoside” or “2′-NMA nucleoside” is shown below:

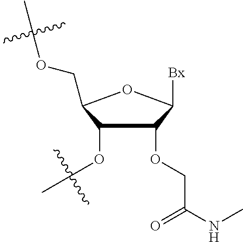

In certain embodiments, 2′-substituent groups are selected from among: halo, allyl, amino, azido, SH, CN, OCN, CF, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et ah, U.S. Pat. No. 5,859,221; and Cook et ah, U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al, WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et ah, WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂), ON(CH₃)₂, O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl, for example, OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and) CH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, and OCH₂C(═O)—N(H)CH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et ah, U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C1-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25 (22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., Org. Chem., 1998, 63, 10035-10039; Srivastava et al., J. Am. Chem. Soc., 2007, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et ak, U.S. Pat. No. 8,268,980; Seth et ak, U.S. Pat. No. 8,546,556; Seth et al, U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

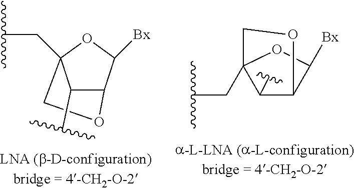

α-L-methyleneoxy (4′-CH₂—O-2′) or α-L-LNA bicyclic nucleosides have been incorporated into oligonucleotides that showed antisense activity (Frieden et al, Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

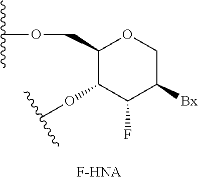

(“F-HNA”, see e.g. Swayze et al., U.S. Pat. No. 8,088,904; Swayze et al., U.S. Pat. No. 8,440,803; Swayze et al., U.S. Pat. No. 8,796,437; and

Swayze et al., U.S. Pat. No. 9,005,906; F-HNA can also be referred to as a F-THP or 3′-fluoro tetrahydropyran), and nucleosides comprising additional modified THP compounds having the formula:

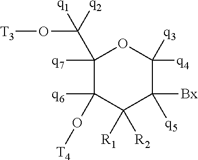

wherein, independently, for each of said modified THP nucleoside: Bx is a nucleobase moiety;

T₃ and T₄ are each, independently, an internucleoside linking group linking the modified THP nucleoside to the remainder of a nucleic acid compound or one of T₃ and T₄ is an internucleoside linking group linking the modified THP nucleoside to the remainder of a nucleic acid compound and the other of T₃ and T₄ is H, a hydroxyl protecting group, a linked conjugate group, or a 5′ or 3′-terminal group; q₁, q₂, q₃, q₄, q₅. q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R¹ and R² is independently selected from among: hydrogen, halogen, substituted or unsubstituted alkoxy, NJ₁J₂, SJ₁, N₃, OC(═X)J₁, OC(═X)NJ₁J₂, NJ₃C(═X) NJ₁J₂, and CN, wherein X is O, S or NJ₁, and each of J₂, and J₃ is, independently, H or C₁-C₆ alkyl.

In certain embodiments, modified THP nucleosides are provided wherein qi. q₂, q₃, q₄, qs, q₆ and q₇ are each H. In certain embodiments, at least one of q₁, q₂, q₃, q₄, q₅. q₆ and q₇ is other than H. In certain embodiments, at least one of q₁, q₂, q₃, q₄, q₅. q₆ and q₇ is methyl. In certain embodiments, modified THP nucleosides are provided wherein one of R¹ and R² is F. In certain embodiments, R¹ is F and R² is H, in certain embodiments, R¹ is methoxy and R² is H, and in certain embodiments, R¹ is methoxyethoxy and R² is H.

In certain embodiments, sugar surrogates comprise rings having more than 5 atoms and more than one heteroatom. For example, nucleosides comprising morpholino sugar moieties and their use in nucleic acid compounds have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et ak, U.S. Pat. No. 5,185,444; and Summerton et al., U.S. Pat. No. 5,034,506). As used here, the term “morpholino” means a sugar surrogate having the following structure:

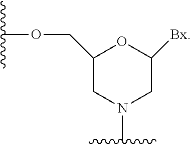

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides comprising such acyclic sugar surrogates include but are not limited to: peptide nucleic acid (“PNA”), acyclic butyl nucleic acid (see, e.g., Kumar et al, Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### Modified Nucleobases

In certain embodiments, modified nucleic acid compounds comprise one or more nucleosides comprising an unmodified nucleobase. In certain embodiments, modified nucleic acid compounds comprise one or more nucleosides comprising a modified nucleobase. In certain embodiments, modified nucleic acid compounds comprise one or more nucleosides that does not comprise a nucleobase, referred to as a basic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyl adenine, 2-thiouracil, 2-thiothymine and 2-thiocytosine, 5-propynyl (—CDC—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al, U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### VII. Modified Internucleoside Linkages

In certain embodiments, nucleosides of nucleic acid compounds may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond, P(O₂)═O, (also referred to as unmodified or naturally occurring linkages); phosphotriesters; methylphosphonates; methoxypropylphosphonates (“MOP”); phosphoramidates; mesyl phosphoramidates; phosphorothioates (P(O₂)═S); and phosphorodithioates (HS—P═S). Representative non-phosphorus containing internucleoside linking groups include but are not limited to methylenemethylimino (—CH₂—N(CH)—O—CH₂—); thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of nucleic acid compounds. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Nucleic acid compounds comprising internucleoside linkages having a chiral center can be prepared as populations of nucleic acid compounds comprising stereorandom internucleoside linkages, or as populations of modified nucleic acid compounds comprising phosphorothioate internucleoside linkages in particular stereochemical configurations. In certain embodiments, populations of nucleic acid compounds comprise phosphorothioate internucleoside linkages wherein all of the phosphorothioate internucleoside linkages are stereorandom. Such nucleic acid compounds can be generated using synthetic methods that result in random selection of the stereochemical configuration of each phosphorothioate internucleoside linkage. Nonetheless, as is well understood by those of skill in the art, each individual phosphorothioate of each individual nucleic acid compound has a defined stereoconfiguration. In certain embodiments, populations of nucleic acid compounds are enriched for nucleic acid compounds comprising one or more particular phosphorothioate internucleoside linkages in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of nucleic acid compounds can be generated using synthetic methods known in the art, e.g., methods described in Oka et al., JACS, 2003, 125, 8307, Wan et al. Nuc. Acid. Res., 2014, 42, 13456, and WO 2017/015555. In certain embodiments, a population of nucleic acid compounds is enriched for nucleic acid compounds having at least one indicated phosphorothioate in the (Sp) configuration. In certain embodiments, a population of nucleic acid compounds is enriched for modified oligonucleotides having at least one phosphorothioate in the (Rp) configuration. In certain embodiments, nucleic acid compounds comprising (Rp) and/or (Sp) phosphorothioates comprise one or more of the following formulas, respectively, wherein “B” indicates a nucleobase:

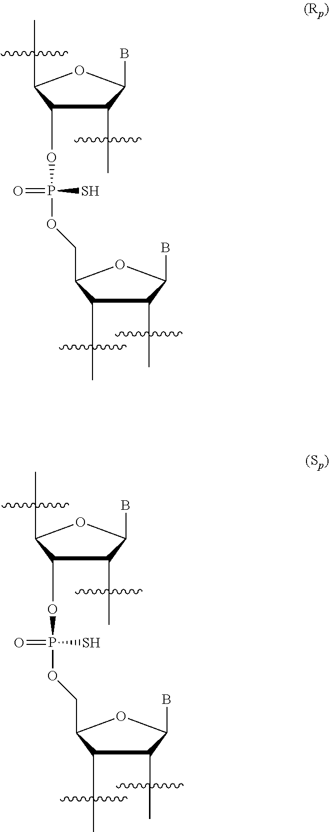

Unless otherwise indicated, chiral internucleoside linkages of nucleic acid compounds described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (see e.g., Carbohydrate Modifications in Antisense Research, Y. S. Sanghvi and P. D. Cook, Eds., ACS Symposium Series 580; Chapters 3 and 4, 40-65). Further neutral intemucleoside linkages include nonionic linkages comprising mixed N, O, S and CH₂ component parts.

In certain embodiments, a modified internucleoside linkage is any of those described in WO 2021/030778, incorporated by reference herein.

### VIII. Pharmaceutical Formulations

The purified 5′-capped mRNA molecules described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the purified 5′-capped mRNA molecules described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the purified 5′-capped mRNA molecules described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the purified 5′-capped mRNA molecules described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the purified 5′-capped mRNA molecules described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the purified 5′-capped mRNA molecules described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

In addition, the purified 5′-capped mRNA molecules comprising compounds having formula (I) or formula (II) or any of the exemplary compounds disclosed herein or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof, may be administered alone or in combination with other therapeutic agents. Combination therapies according to the present invention comprise the administration of at least one exemplary purified 5′-capped mRNA molecule of the present disclosure and at least one other therapeutic agent in a pharmaceutical composition. The at least one exemplary purified 5′-capped mRNA molecule of the present disclosure and at least one other therapeutic agent(s) may be administered as a pharmaceutical composition separately or together. The amounts of the at least one exemplary purified 5′-capped mRNA molecule of the present disclosure and the at least one other therapeutic agent(s) and the relative timings of administration will be selected in order to achieve the desired combined therapeutic effect.

The purified 5′-capped mRNA molecules can include one or more of the compounds described herein and a pharmaceutically acceptable carrier. In embodiments, the purified 5′-capped mRNA molecules provided herein may be provided as a pharmaceutically acceptable salt (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002). As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.).

In embodiments, 5′-capped mRNA molecules described herein are in aqueous solution with sodium salt. In embodiments, 5′-capped mRNA molecules described herein are in aqueous solution with lithium salt. In embodiments, 5′-capped mRNA molecules described herein are in aqueous solution with potassium salt. In embodiments, 5′-capped mRNA molecules described herein are in aqueous solution with triethylammonium salt.

Administration of the purified 5′-capped mRNA molecules and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### IX. Methods of Treatment

Also described herein are methods for increasing in vivo translation of a polypeptide in a subject. The methods include administering to the subject a therapeutic dose unit comprising an effective amount of an mRNA encoding a polypeptide, wherein the mRNA comprises a purified 5′-cap having a formula according to any of the compounds as described herein. In some examples, the methods include administering to the subject a therapeutic dose unit comprising an effective amount of an mRNA encoding a polypeptide, wherein the mRNA is prepared in accordance with methods disclosed herein.

In embodiments, the methods include administering to the subject a therapeutic dose unit comprising an effective amount of a purified 5′-capped oligonucleotide as described herein, including in embodiments. In embodiments, provided herein is a pharmaceutical composition comprising the purified 5′-capped oligonucleotide as described herein, including in embodiments, and a pharmaceutically acceptable excipient. In embodiments, the pharmaceutical composition comprising the purified 5′-capped mRNA molecule described herein and a pharmaceutically acceptable excipient, may be used in vaccines. In embodiments, the vaccines may be vaccines against infectious diseases or cancer. In embodiments, the pharmaceutical composition comprising the purified 5′-capped mRNA molecule described herein and a pharmaceutically acceptable excipient, may be used in genetic vaccination, wherein an immune response is stimulated by introduction of a suitable mRNA, into a subject, which codes for an antigen or a fragment thereof. These vaccine strategies can require large quantities of purified capped RNA. The present methods facilitate such synthesis and subsequent purification of capped RNA so as to make these vaccines commercially feasible. The present methods also describe strategies to increase the percentage of full-length capped RNA in a transcription reaction leading to a more homogenous product.

In some embodiments, the 5′-capped mRNAs of the disclosure encode an antimicrobial peptide. In some embodiments, the 5′-capped mRNAs encode an antimicrobial peptide against antibiotic-resistant infections caused by methicillin-resistant Staphloococcus (MRSA).

In some embodiments, the 5′-capped mRNAs of the disclosure encode a hormone. In some such embodiments the hormone is a growth hormone.

### Methods for Making Compounds of the Invention.

As used herein, common organic chemistry abbreviations are defined as follows:
Ac Acetyl
ACN Acetonitrile
AcOH Acetic acid
aq. Aqueous
AX chromatography Anion exchange chromatography
DBU 1,8-Diazabicyclo[5.4.0]undec-7-ene
DCM dichloromethane
DI water Deionized water
DIAD Diisopropyl azodicarboxylate
DIEA or DIPEA Diisopropylethylamine
DMAP 4-dimethylaminopyridine
DMF N,N′-Dimethylformamide
DMS Dimethyl sulfate
DMSO Dimethyl sulfoxide
EDC or EDC·HCl 1-Ethyl-3-(3-dimethylaminopropyl)carbodiimide hydrochloride
EtOAc or EA Ethyl acetate
EtOH Ethanol
Fmoc Fluorenylmethyloxycarbonyl
g Gram(s)
hrs. Hour (hours)
HCl Hydrochloric acid
HPLC High-performance liquid chromatography
IPA Isopropyl alcohol
LC/MS Liquid chromatography-mass spectrometry
mg milligrams
MeOH Methanol
mL Milliliter(s)
μL/μL Microliter(s)
mmol millimoles
μmol/μmol micromoles
MMT monomethoxytrityl
MS mass spectrometry
NaH Sodium hydride
NaOAc Sodium acetate
NaOH Sodium hydroxide
Pyr Pyridine
RP-HPLC reverse phase HPLC
RT room temperature
t-Bu tert-Butyl
TEA Triethylamine
TEAB Tetraethylammonium bromide
Tert, t tertiary
TEA salt triethylammonium salt
TEAB triethylammonium bromide
TFA Trifluoracetic acid
TFAA Trifluoracetic anhydride
THF Tetrahydrofuran
TMP Trimethylphosphate
TPP Triphenylphosphine

General methods for making oligonucleotides with cap 1 or cap 2 analogs from a diphosphate and a 5′-monophosphate-oligonucleotide or a monophosphate and a 5′-diphosphate-oligonucleotide. The methods below may be a one-pot synthesis.

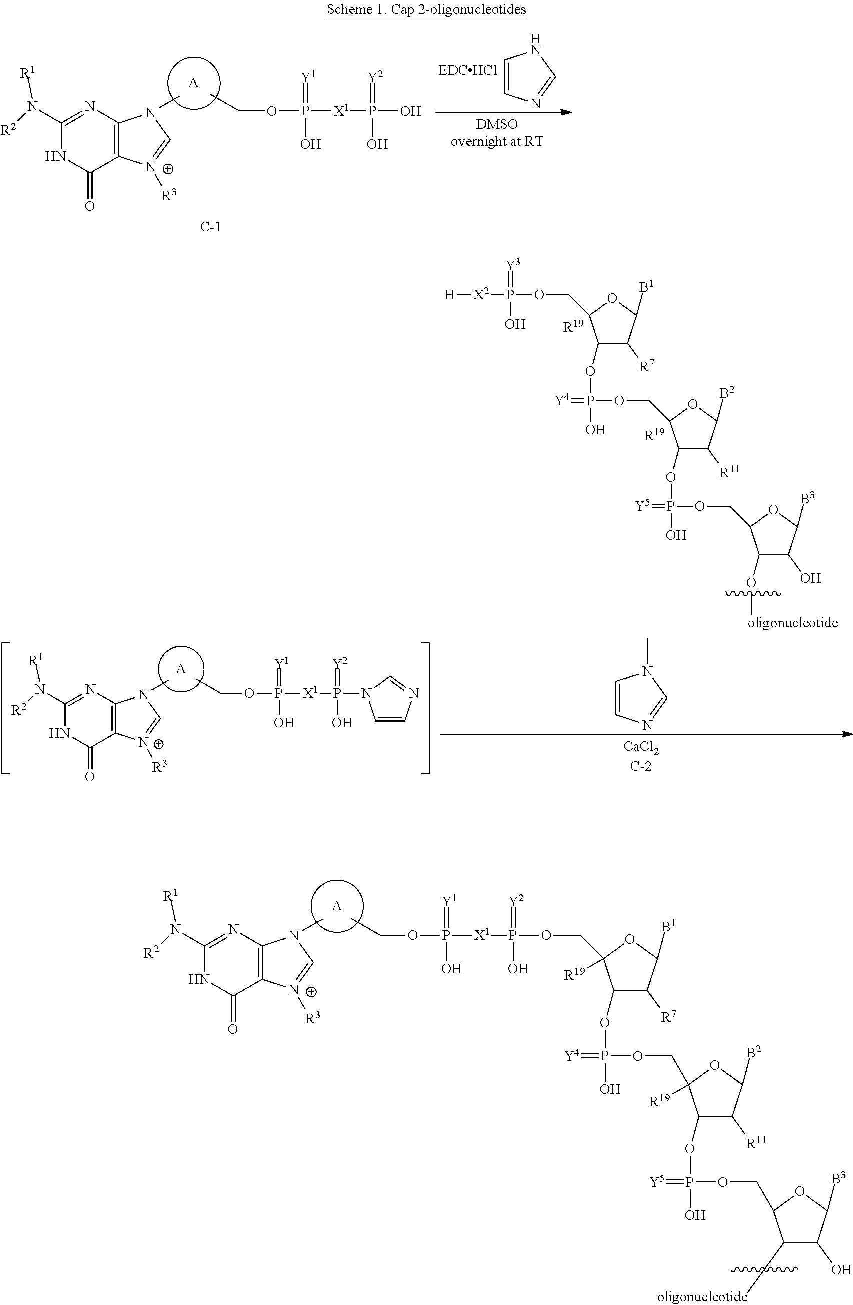

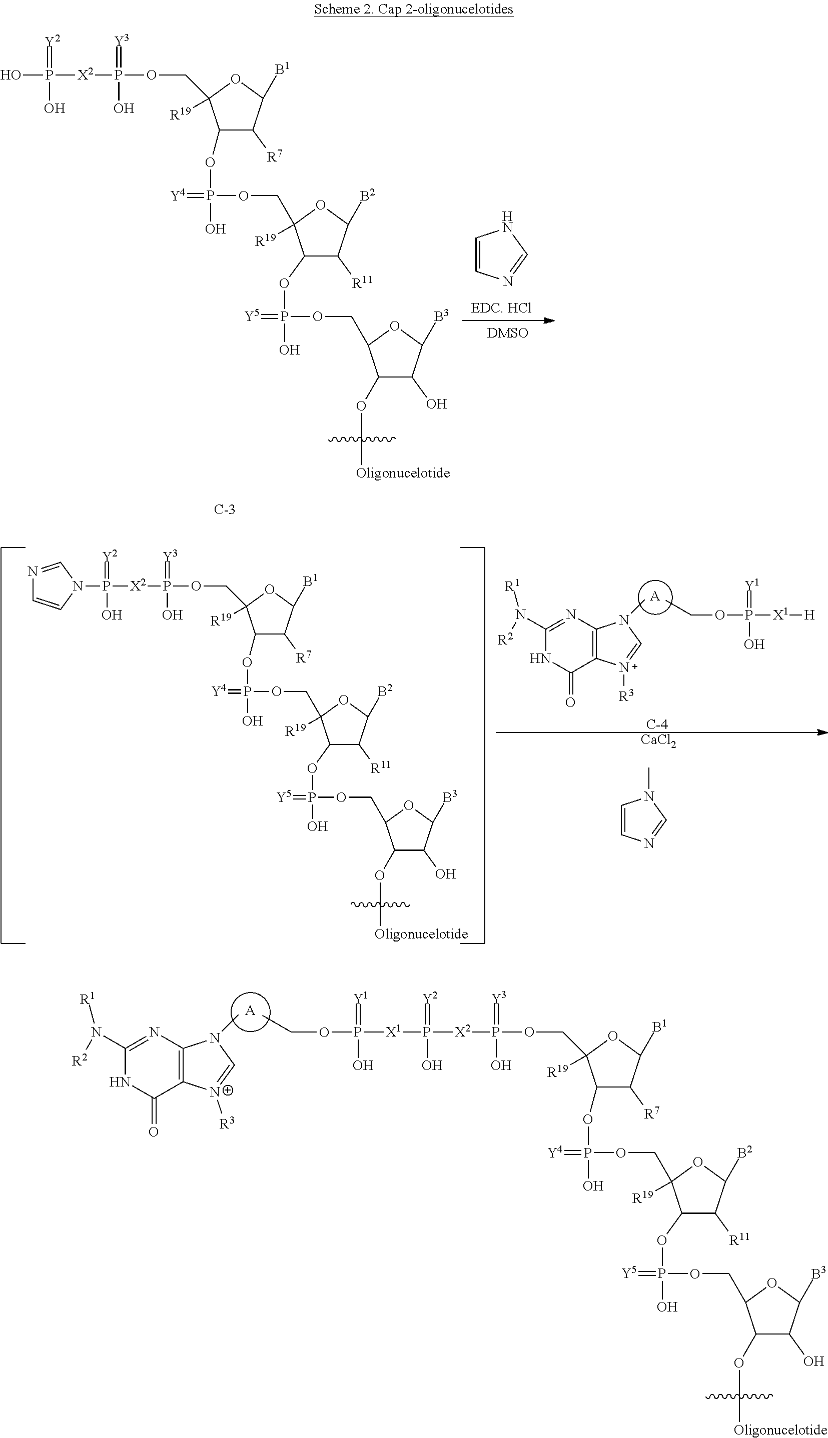

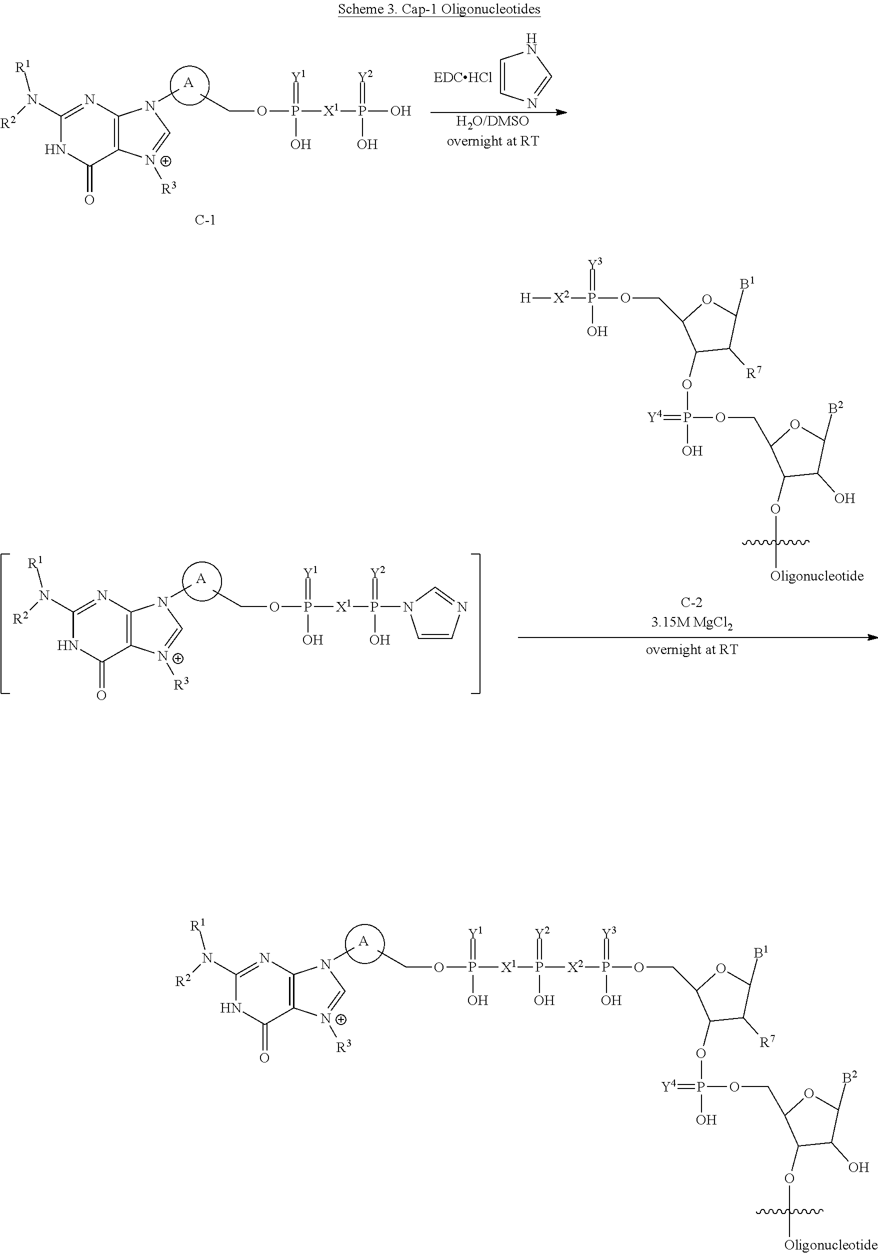

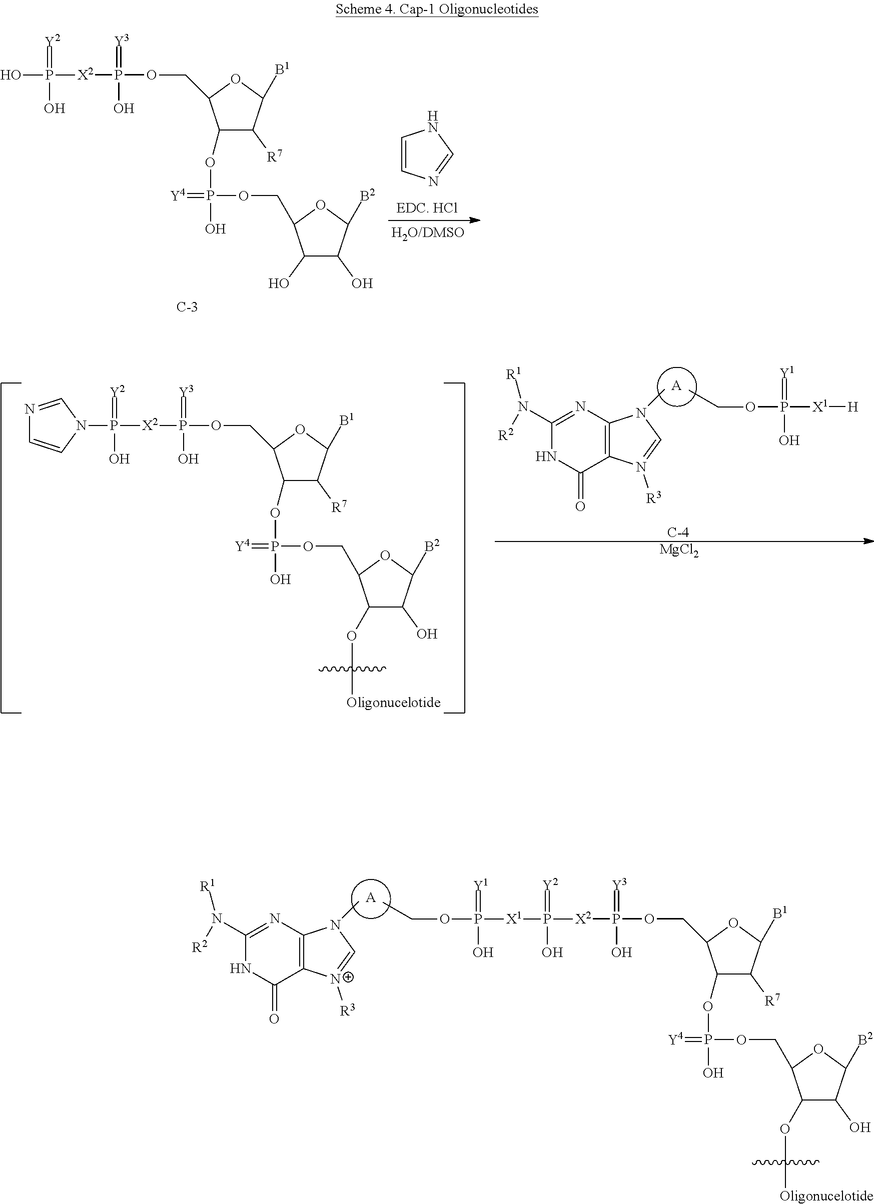

General procedures for Schemes 3 and 4 were previously described in International Patent Publication No. WO2023147352 which is incorporated herein by reference in its entirety. Procedures for specific examples of Schemes 3 and 4 are described below.

Procedures for In Vitro Transcription (IVT) reactions that use cap analogs depicted in Schemes 3 and 4 were previously described in International Patent Publication No. WO2017053297 which is incorporated herein by reference in its entirety.

### EXAMPLES

The following examples are meant to be illustrative and can be used to further understand embodiments of the present disclosure and should not be construed as limiting the scope of the present teachings in any way.

The chemical reactions described in the Examples can be readily adapted to prepare a number of other compounds of the present disclosure, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by utilizing other suitable reagents known in the art other than those described, or by making routing modifications of reaction conditions, reagents, and starting materials. Alternatively, other reactions disclosed herein or known in the art will be recognized as having applicability for preparing other compounds of the present disclosure.

### SYNTHETIC EXAMPLES

### Example S1: Synthesis of Compound A-1 ^{N7-(4-chlorobenzyl)}G_{3′OMe}ppp^{m6}A_{2′OMe}pG

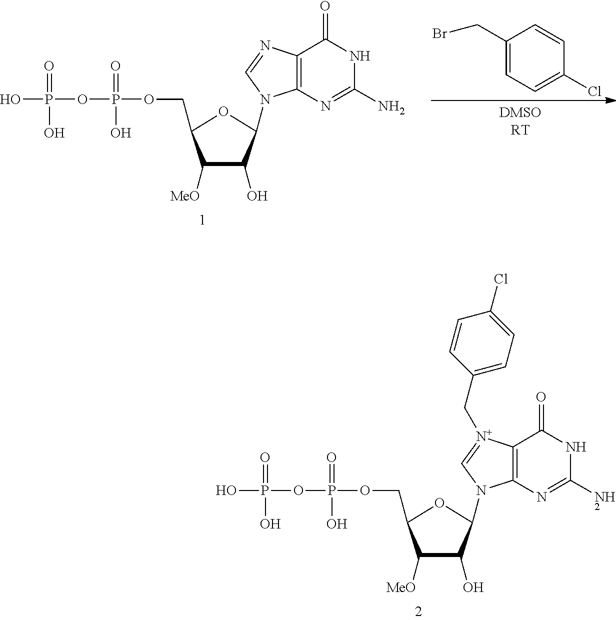

4-chlorobenzyl bromide (4.93 g, 24 mmol) was added to a stirred solution of compound 1 (2.05 g, 3.0 mmol) (which can be prepared according to methods described by Kore et al. (2009) J. Am. Chem. Soc. 131:6364-6365; custom order TriLink Biotechnologies) in DMSO (40 ml), and reaction was stirred overnight at room temperature. Reaction was stopped by dilution with 400 mL of water and extracted 3× with ethyl acetate. The crude product was purified by anion exchange chromatography. Then the product was desalted by Reverse Phase (Triart C18) resin. Fractions containing the product were collected and concentrated to afford compound 2 as an off-white solid (1.28 g, 80% yield).

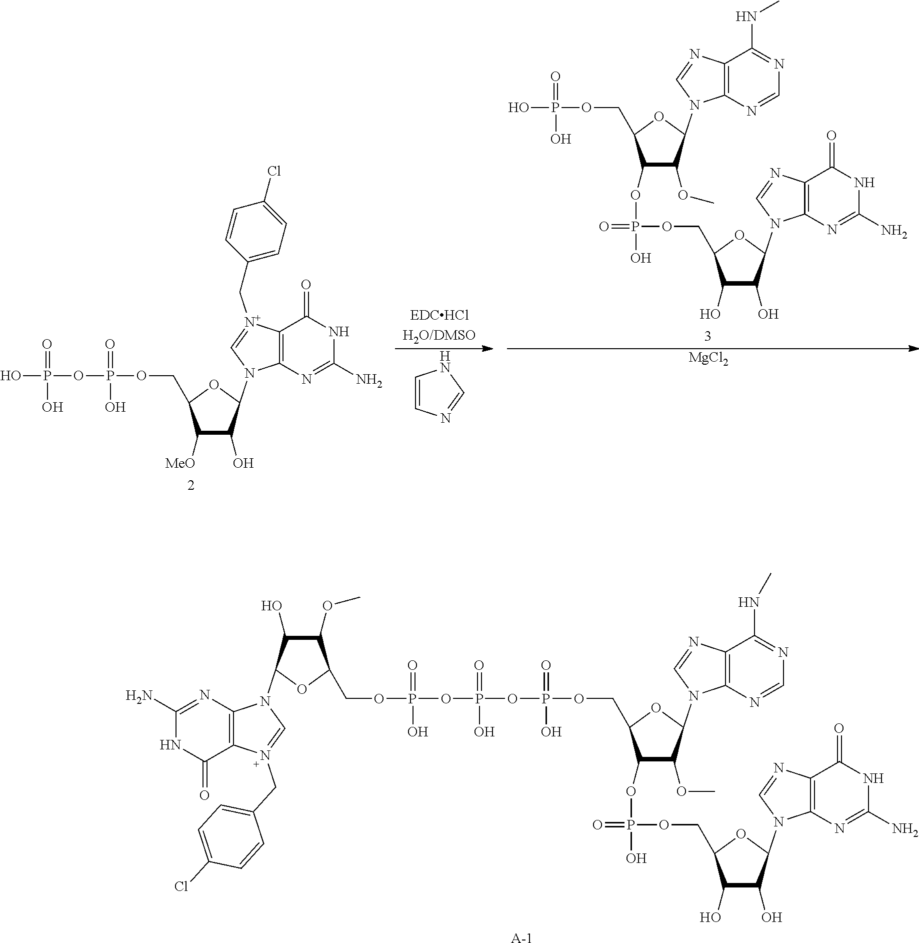

To a stirred solution of compound 2 (TEA salt, 0.526 g, 0.75 mmol) in 10% water/DMSO (4.40 mL), at room temperature, was added EDC hydrochloride salt (0.230 g, 1.2 mmol), followed by imidazole (0.153 g, 2.25 mmol). The resulting mixture was allowed to stir at room temperature overnight (8-16 hours). Upon completion of the reaction, 3.15M magnesium chloride (0.32 ml, 1 mmol) was added to the solution, followed by compound 3 (TEA salt, 0.460 g, 0.5 mmol) (custom order Wuxi App Tec). The resulting solution was allowed to stir at room temperature overnight. The crude reaction mixture was then diluted with 10× water and purified by anion exchange chromatography. Then the product was desalted by Reverse Phase (Triart C18) resin. Fractions containing the product were collected and concentrated to afford compound A-1 as a white powder (0.541 g, 25% yield).

1H NMR (500 MHz, D₂O): δ 9.47 (s, 1H), 8.31 (s, 1H), 8.31 (s, 1H), 8.10 (s, 1H), 7.93 (s, 1H), 7.13 (d, J=8.2 Hz, 2H), 6.99 (d, J=8.1 Hz, 2H), 5.94 (d, J=5.60 Hz, 1H), 5.89 (d, J=4.45 Hz, 1H), 5.80 (d, J=5.70 Hz, 1H), 5.48 (q, J=14.8 Hz, 2H), 4.95-4.92 (m, 1H), 4.49-4.47 (m, 2H), 4.45-4.41 (m, 3H), 4.40-4.32 (m, 3H), 4.24-4.14 (m, 5H), 3.48 (s, 3H), 3.43 (s, 3H), 3.07 (br s, 3H).

31P NMR (200 MHz, D₂O): δ −0.29 (1P), −10.68 (2P), −22.24 (1P).

MS m/z=1281.7 (M−3H).

### Example S2: Synthesis of Compound A-2 ^{m7, N2-butyl}G_{3′OMe}ppp^{m6}A_{2′OMe}pG

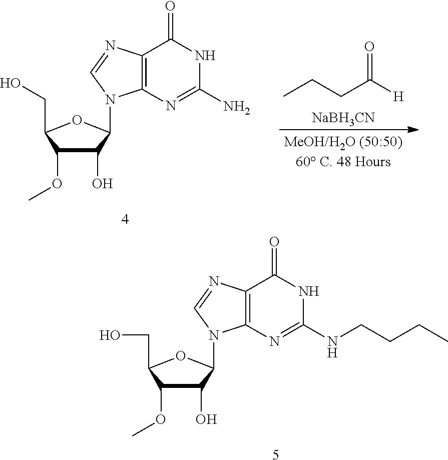

n-Butyraldehyde (45.1 mL, 50 mmol) was added to a solution of compound 4 (5.0 g, 17 mmol), and sodium cyanoborohydride (10.5 g, 170 mmol) in 50% methanol/water (50 mL) in a pressure flask. The mixture was stirred at 60° C. for 48 hours. The mixture was decanted and then concentrated under vacuum. The product was purified using flash chromatography. Compound 5 was obtained as a brown solid (2.23 g, 37% yield).

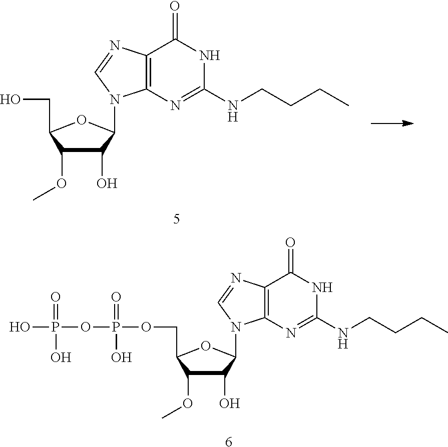

Compound 6 can be prepared from compound 5 according to methods described by Kore et al. (2009) J. Am. Chem. Soc. 131:6364-6365.

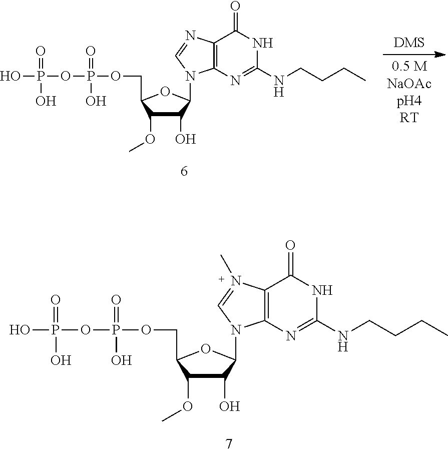

Compound 6 (1.53 g, 2.05 mmol) was dissolved in 0.5 M sodium acetate (pH4, 15.3 mL) and stirred vigorously at room temperature. Dimethyl sulfate (2.91 mL, 31 mmol) was added to the solution and stirring continued vigorously at room temperature for 2 hours. The pH was constantly monitored and adjusted to 3-4 with 1M NaOH. The reaction was then extracted 3× with ethyl acetate. The aqueous layer was collected, diluted 10× with water, and pH adjusted to 5.5 to 6.0 with 1M sodium hydroxide. The crude was then purified using anion exchange chromatography. Fractions containing the product were collected and dried to afford compound 7 as a brown residue (0.552 g, 40% Yield).

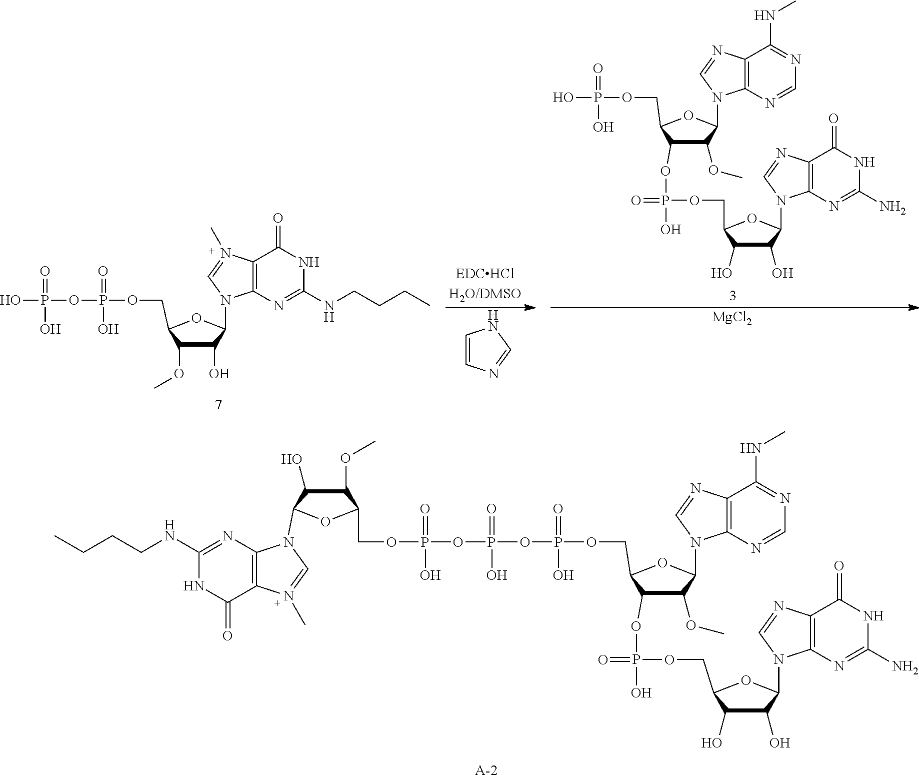

To a stirred solution of compound 7 (TEA salt, 0.504 g, 0.75 mmol) in 10% water/DMSO (4.41 mL), at room temperature, was added EDC hydrochloride salt (0.230 g, 1.2 mmol), followed by imidazole (0.153 g, 2.25 mmol). The resulting mixture was allowed to stir at room temperature overnight (8-16 hours). Upon completion of the reaction, 3.15M magnesium chloride (0.317 mL, 1 mmol) was added to the solution, followed by compound 3 (TEA salt, 0.460 g, 0.50 mmol). The resulting solution was allowed to stir at room temperature overnight. The crude reaction mixture was then diluted with 10× water and purified by anion exchange chromatography. Compound A-2 was pooled and concentrated under vacuum and precipitated as a sodium salt with sodium acetate and 95% absolute ethanol in water. Compound A-2 was obtained as a white solid (0.318 g, 46% yield).

1H NMR (500 MHz, D₂O): δ 8.21 (s, 1H), 8.04 (s, 1H), 7.87 (s, 1H), 5.89 (d, J=5 Hz, 1H), 5.78 (d, J=5 Hz, 2H), 4.86-4.83 (m, 2H), 4.72-4.70 (t, J=5 Hz, 2H), 4.62-4.61 (t, J=5 Hz, 1H), 4.47-4.44 (m, 2H), 4.39-4.36 (m, 2H), 4.34-4.29 (m, 3H), 4.23-4.17 (m, 4H), 4.05-4.03 (t, J=5 Hz, 1H), 3.98 (s, 3H), 3.44 (s, 6H), 3.30-3.27 (m, 1H), 3.18-3.13 (m, 1H), 3.05 (br s, 3H), 1.54-1.48 (m, 2H), 1.35-1.31 (m, 2H), 0.90-0.88 (t, J=5 Hz, 3H).

31P NMR (200 MHz, D₂O): δ −0.44 (1P), −10.97 (2P), −22.30 (1P).

MS m/z=1230.8 (M+H).

### Example S3: Synthesis of Oligonucleotide A-3 ^{m7, N2-MMT}G_{3′OMe}pppGpG-SEQ1A

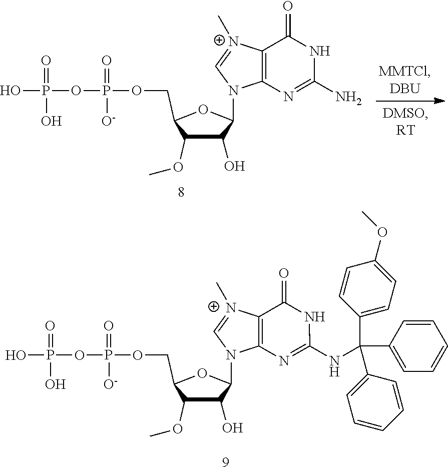

Compound 8 (1.0 g, 1.5 mmol) (Trilink Biotechnologies custom order) was dried on Hi-Vac for 48 hrs. and then suspended in anhydrous DMSO (15 mL). To the solution was added monomethoxy trityl chloride (MMTC1) (3.24 g, 10.5 mmol), followed by DBU (0.67 mL, 4.5 mmol) one equivalent at a time. The resulting mixture was allowed to stir at room temperature for 1 hr. Upon completion of the reaction, Hexanes was added (30 mL) to the solution, stirred 5 minutes, and decanted two separate times. A light stream of air was placed over the solution for 5 minutes to remove any Hexanes. The crude reaction mixture was then diluted with 10×20% acetonitrile in water and purified by anion exchange chromatography. Compound 9 was pooled and concentrated under vacuum. Compound 9 was obtained as an off-white powder (1.1 g, 80% yield).

1H NMR (500 MHz, D₂O): δ 7.41-7.39 (m, 4H), 7.34-7.29 (m, 2H), 7.27-7.25 (m, 4H), 7.24-7.22 (m, 2H), 5.40 (s, 1H), 4.32-4.25 (m, 3H), 4.08-4.02 (m, 4H), 3.95-3.93 (m, 1H), 3.72 (s, 1H), 3.36 (s, 3H).

31P NMR (200 MHz, D₂O): δ −7.46 (d, 1P), −10.78 (d, 1P).

MS m/z=742.0 [M−H].

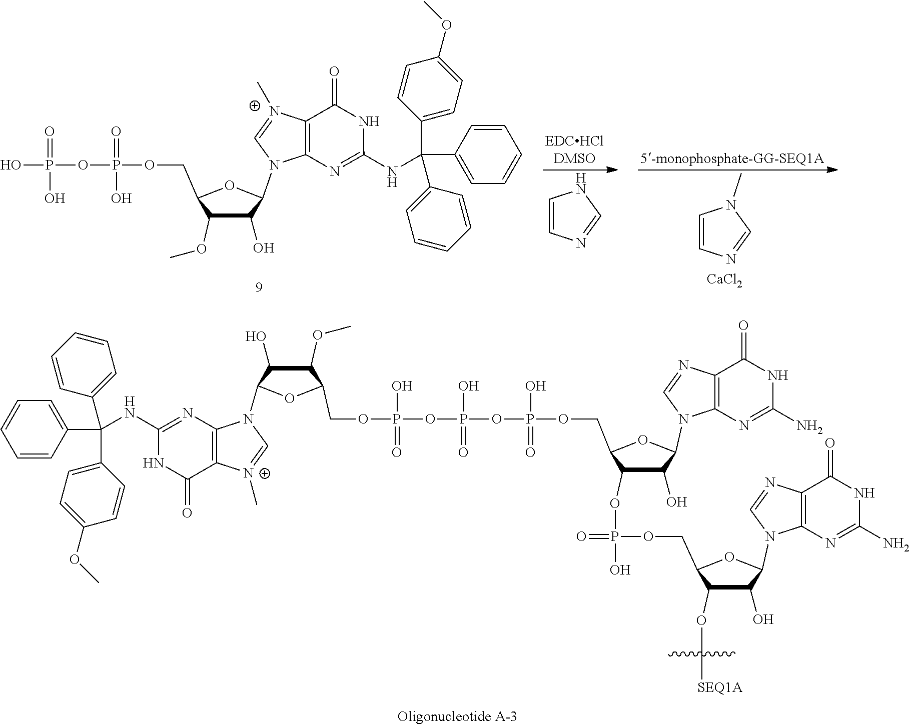

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-GG-SEQ1A (SEQ ID NO: 1) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-3 was pooled and concentrated under vacuum. Oligonucleotide A-3 was obtained as a clear film.

MS m/z=32994.6 [M+H].

### Example S4: Synthesis of Oligonucleotide A-4 ^{m7, N2-MMT}G_{3′OMe}pppA_{2′OMe}pG-SEQ3A

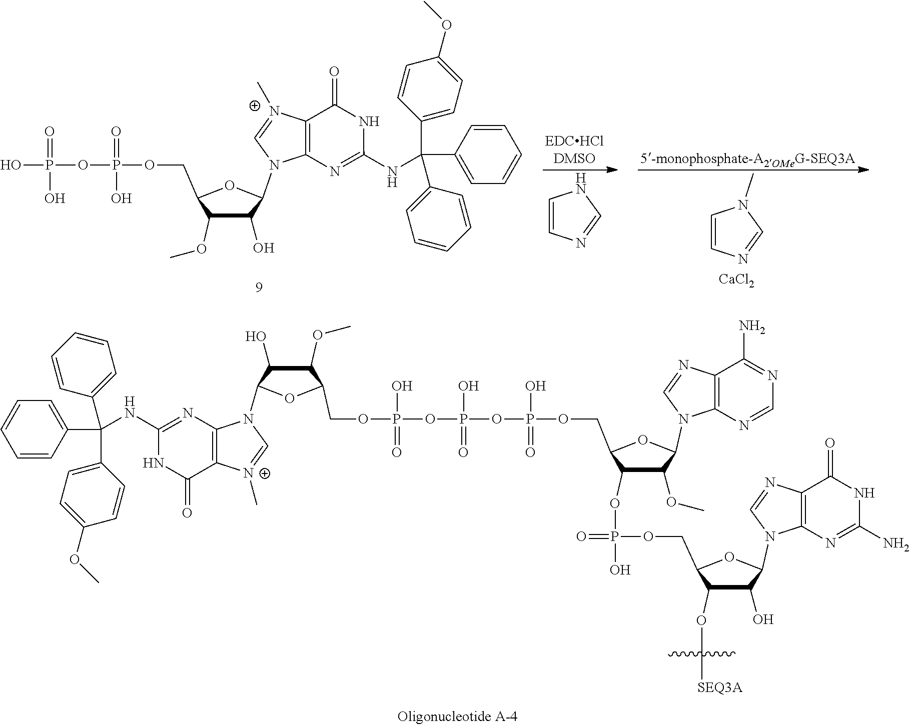

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ3A (SEQ ID NO: 3) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-4 was pooled and concentrated under vacuum. Oligonucleotide A-4 was obtained as a clear film.

MS m/z=32990.1 [M+H].

### Example S5: Synthesis of Oligonucleotide A-5 and A-6 ^{m7, N2-MMT}G_{3′OMe}pppGpG-SEQ2A and ^{m7}G_{3′OMe}pppGpG-SEQ2A

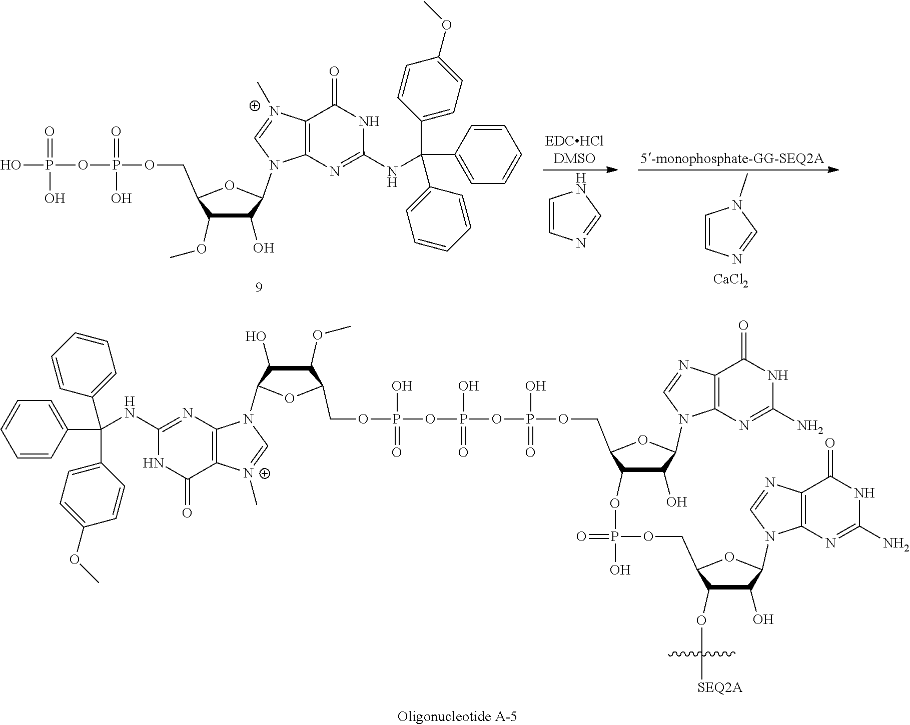

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-GG-SEQ2A (SEQ ID NO: 2) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-5 was pooled and concentrated under vacuum. Oligonucleotide A-5 was obtained as a clear film (40 nmol, 80% yield).

MS m/z=3943.6 [M+H].

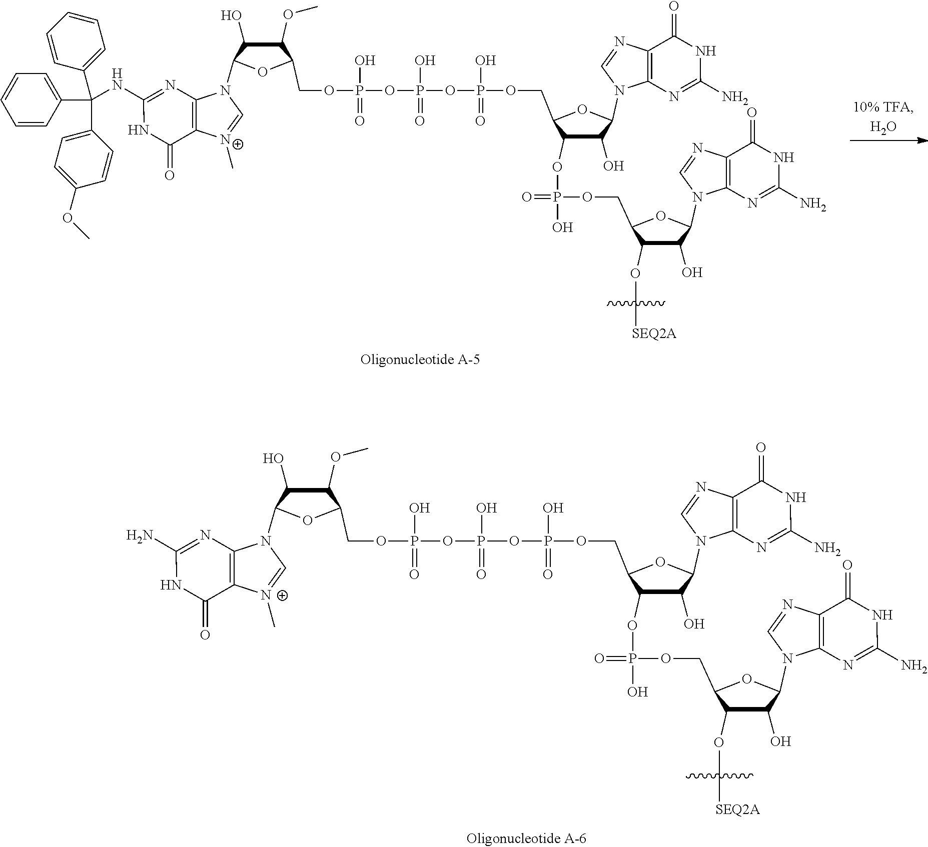

To a 0.2 mM solution of Oligonucleotide A-5 (TEA salt, 20 μL, 3.8 nmol) in DMSO, at room temperature, was added TFA (2.2 μL, 29 μmol). The solution was mixed with a pipette and allowed to incubate at room temperature for 30 min. The crude reaction mixture was then diluted in 50 mM TEAA, 2% acetonitrile in RNAse free water (5 mL) pre-chilled in an ice bath and purified by anion exchange chromatography. Oligonucleotide A-6 was pooled and concentrated under vacuum. Oligonucleotide A-6 was obtained as a clear film (1 nmol, in about 20% yield).

MS m/z=3671.3 [M+H].

### Example S6: Synthesis of Oligonucleotide A-7 ^{m7, N2-MMT}G_{3′OMe}pppA_{2′OMe}pG-SEQ4A

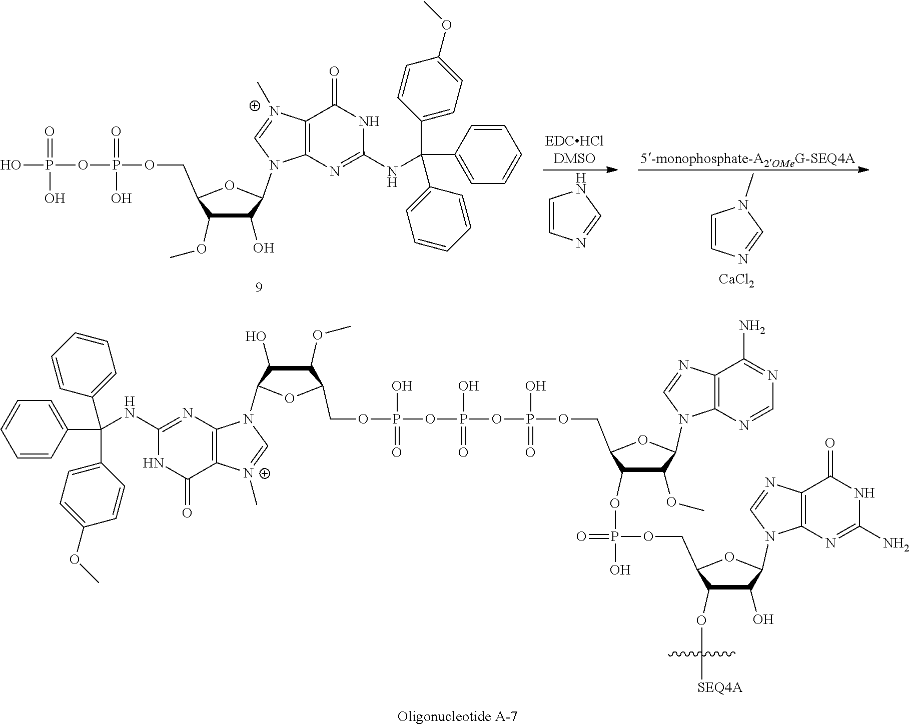

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ4A (SEQ ID NO: 4) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-7 was pooled and concentrated under vacuum. Oligonucleotide A-7 was obtained as a clear film.

MS m/z=29869.5 [M+H].

### Example S7: Synthesis of Oligonucleotide A-8 ^{m7, N2-MMT}G_{3′OMe}pppA_{2′OMe}pG-SEQ5A

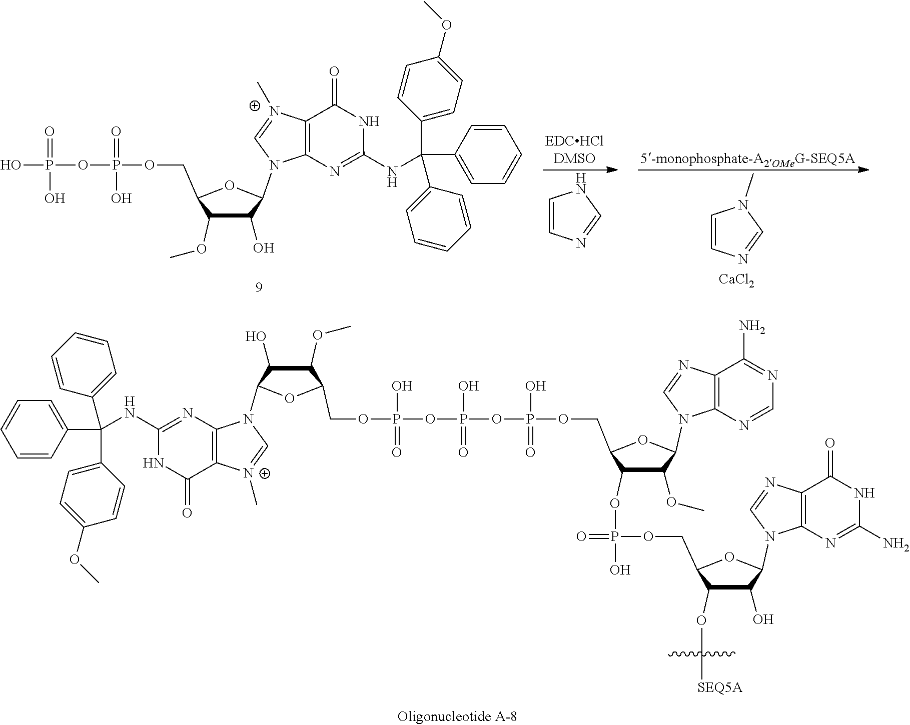

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ5A (SEQ ID NO: 5) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-8 was pooled and concentrated under vacuum. Oligonucleotide A-8 was obtained as a clear film.

MS m/z=30022.0 [M+H].

### Example S8: Synthesis of Oligonucleotide A-9 and A-10 ^{m7, N2-MMT}G_{3′OMe}pppA_{2′OMe}pG-SEQ6A and ^{m7}G_{3′OMe}pppA_{2′OMe}pG-SEQ5A

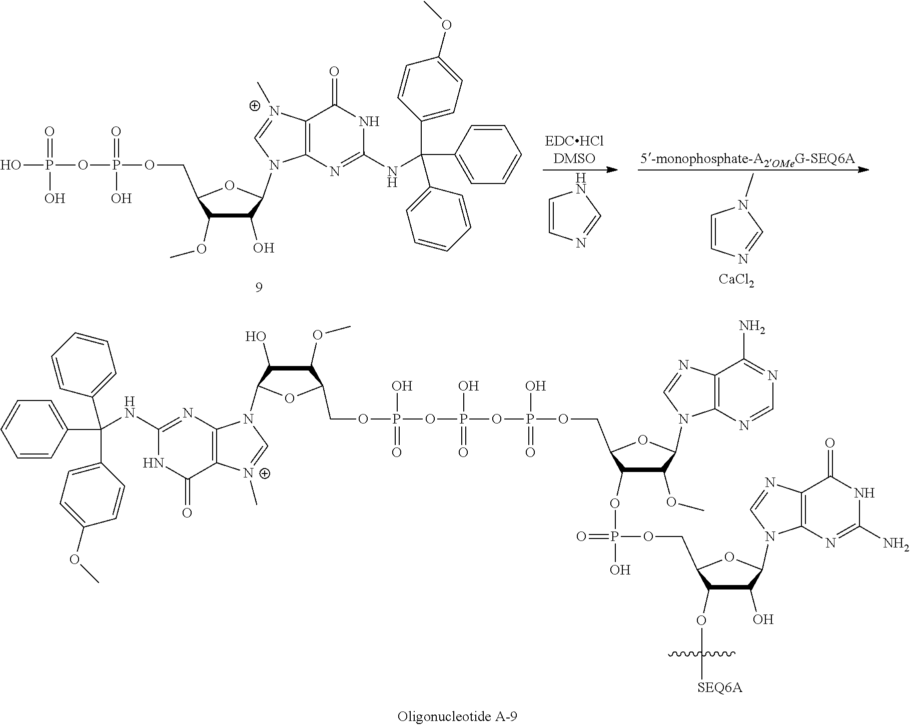

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (13.4 μL), 0.1 M calcium chloride (2 μL, 0.2 μmol), 1-methylimidazole (1.6 μL, 20 μmol), and 1 mM 5′-monophosphate-A_{2′OMe}G-SEQ6A (SEQ ID NO: 6) (custom ordered from Integrated DNA Technologies, 10 μL, 10 nmol) in DMSO were added to the crude reaction solution (2 μL). The resulting solutions were mixed with a pipette and incubated at 55° C. for 1 hr. The crude reaction mixture was then diluted in RNAse free water (5 mL) pre-chilled in an ice bath and purified by anion exchange chromatography. Oligonucleotide A-9 was pooled and concentrated under vacuum. Oligonucleotide A-9 was obtained as a clear film.

MS m/z=26726.6 [M+H].

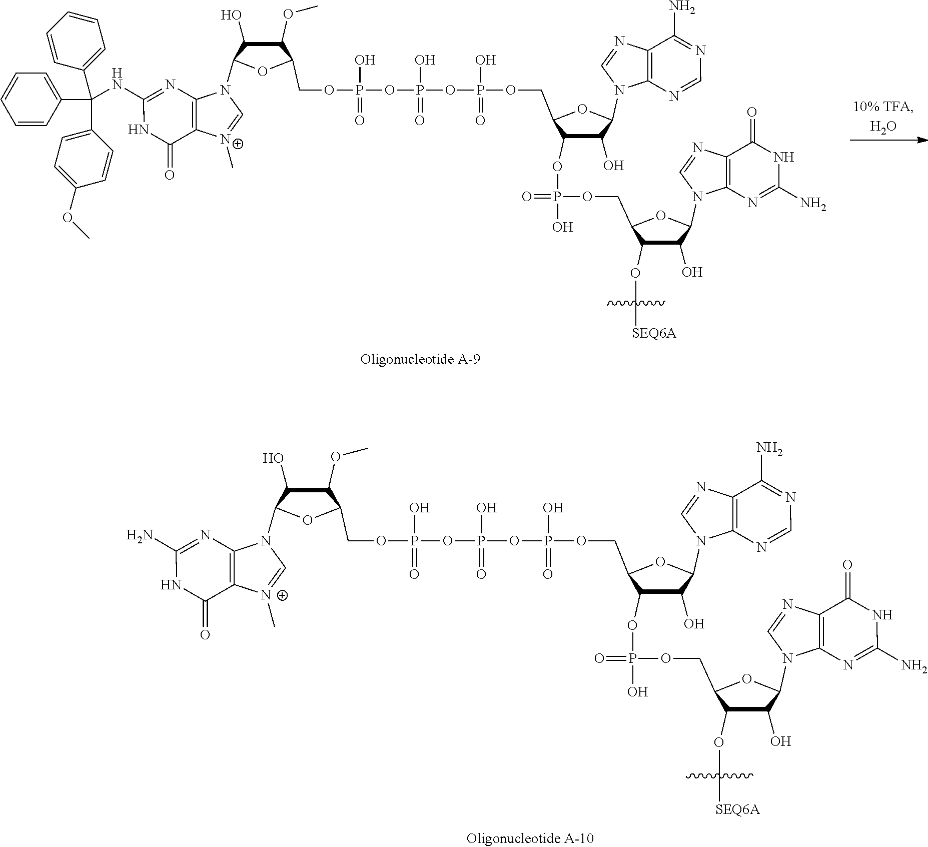

To a 0.2 mM solution of Oligonucleotide A-9 (TEA salt, 20 μL, 0.18 nmol) in DMSO, at room temperature, was added TFA (2.2 μL, 29 μmol). The solution was mixed with a pipette and allowed to incubate at room temperature for 30 min. The crude reaction mixture was then diluted in 50 mM TEAA, 2% acetonitrile in RNAse free water (5 mL) pre-chilled in an ice bath and purified by anion exchange chromatography. Oligonucleotide A-10 was pooled and concentrated under vacuum. Oligonucleotide A-10 was obtained as a clear film.

MS m/z=26452.7 [M+H].

### Example S9: Synthesis of Oligonucleotide A-11 and A-12 ^{m7, N2-MMT}G_{3′OMe}pppA_{2′OMe}pG-SEQ7A and ^{m7}G_{3′OMe}pppA_{2′OMe}pG-SEQ7A

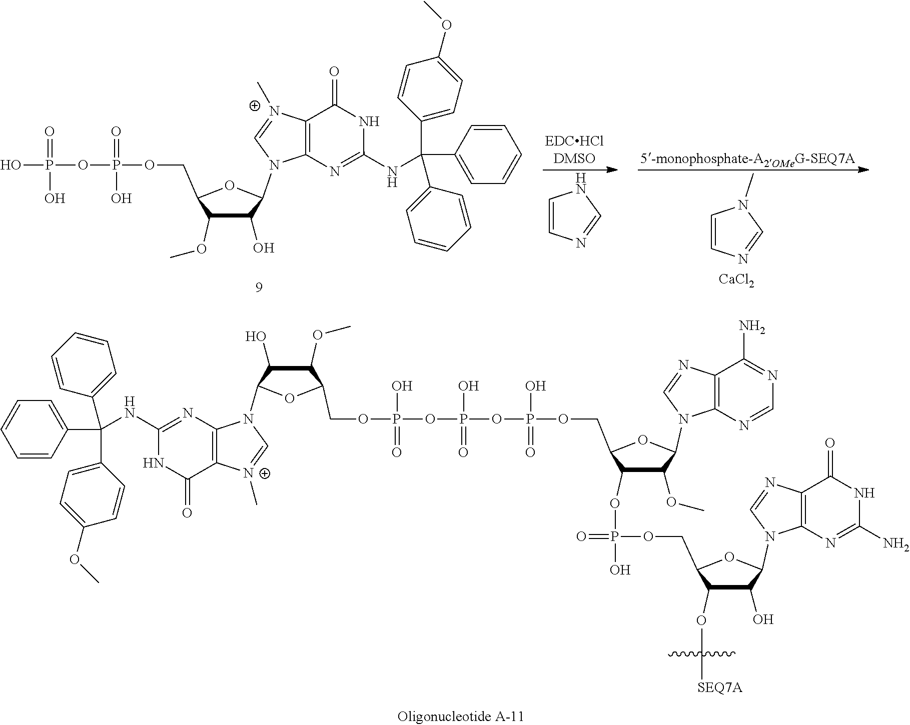

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (13.4 μL), 0.1 M calcium chloride (2 μL, 0.2 μmol), 1-methylimidazole (1.6 μL, 20 μmol), and 1 mM 5′-monophosphate-A_{2′OMe}G-SEQ7A (SEQ ID NO: 7) (custom ordered from Integrated DNA Technologies, 10 μL, 10 nmol) in DMSO were added to the crude reaction solution (2 μL). The resulting solutions were mixed with a pipette and incubated at 55° C. for 1 hr. The crude reaction mixture was then diluted in RNAse free water (5 mL) pre-chilled in an ice bath and purified by anion exchange chromatography. Oligonucleotide A-11 was pooled and concentrated under vacuum. Oligonucleotide A-11 was obtained as a clear film.

MS m/z=26876.2 [M+H].

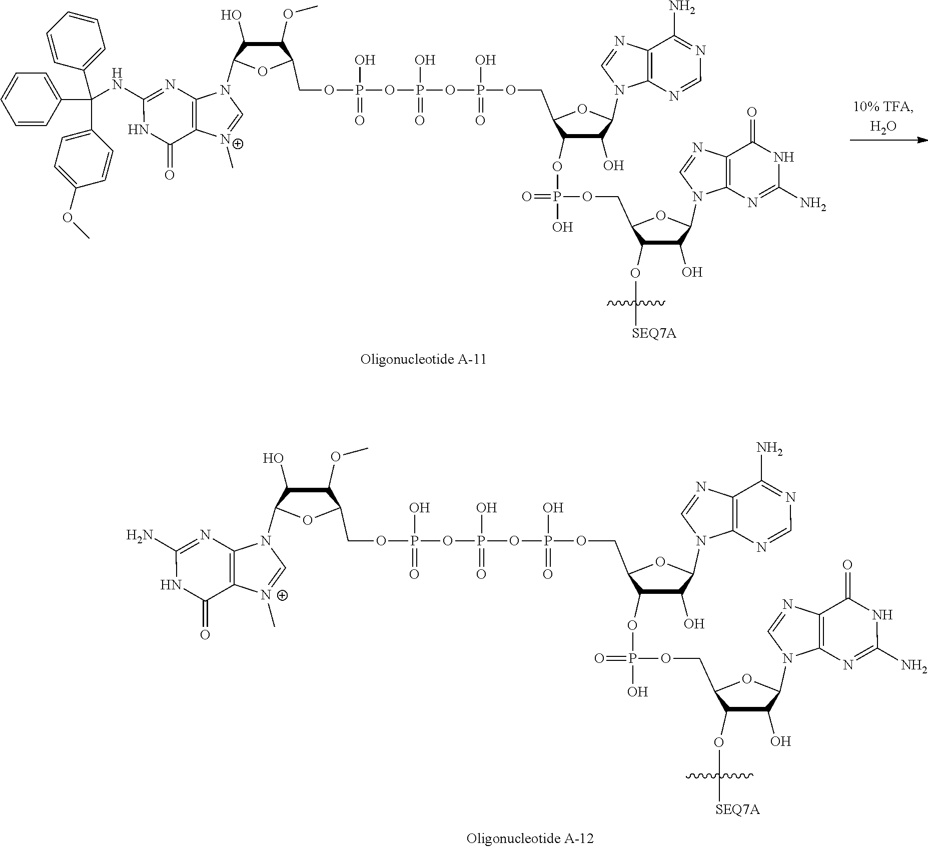

To a 0.2 mM solution of Oligonucleotide A-11 (TEA salt, 20 μL, 0.18 nmol) in DMSO, at room temperature, was added TFA (2.2 μL, 29 μmol). The solution was mixed with a pipette and allowed to incubate at room temperature for 30 min. The crude reaction mixture was then diluted in 50 mM TEAA, 2% acetonitrile in RNAse free water (5 mL) pre-chilled in an ice bath and purified by anion exchange chromatography. Oligonucleotide A-12 was pooled and concentrated under vacuum. Oligonucleotide A-12 was obtained as a clear film.

MS m/z=26553.8 [M+H].

### Example S10: Synthesis of Oligonucleotide A-13 ^{m7, N2-MMT}G_{3′OMe}pppA_{2′OMe}pG-SEQ8A

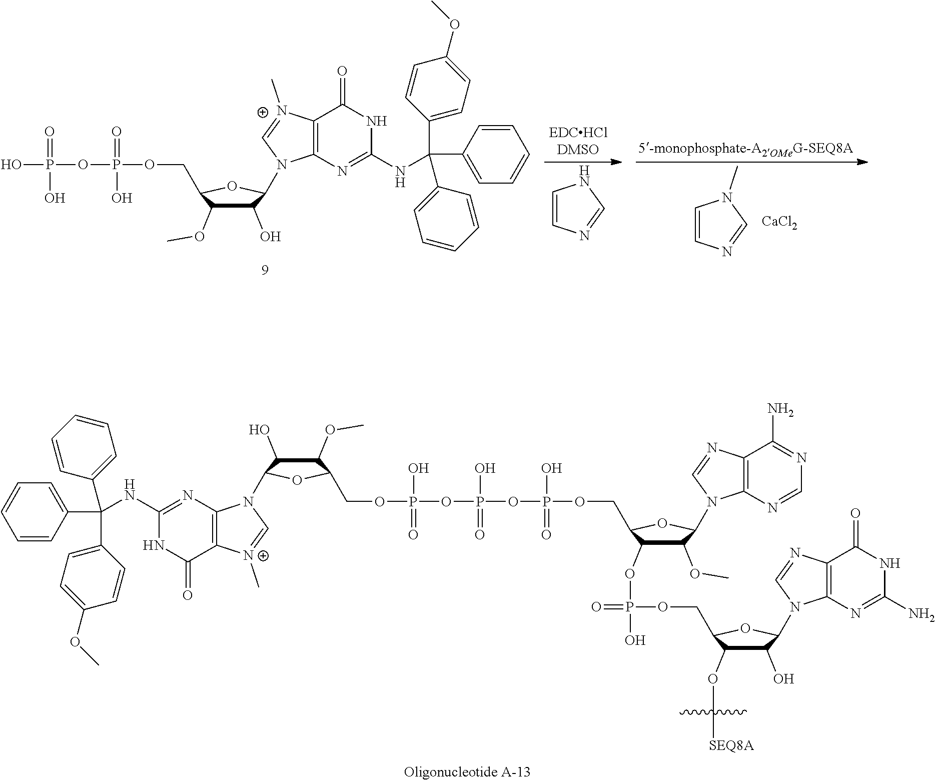

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ8A (SEQ ID NO: 8) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-13 was pooled and concentrated under vacuum. Oligonucleotide A-13 was obtained as a clear film.

MS m/z=23503.5 [M+H].

### Example S11: Synthesis of Oligonucleotide A-14 ^{m7, N2-MMT}G_{3′OMe}pppA_{2′OMe}pG-SEQ9A

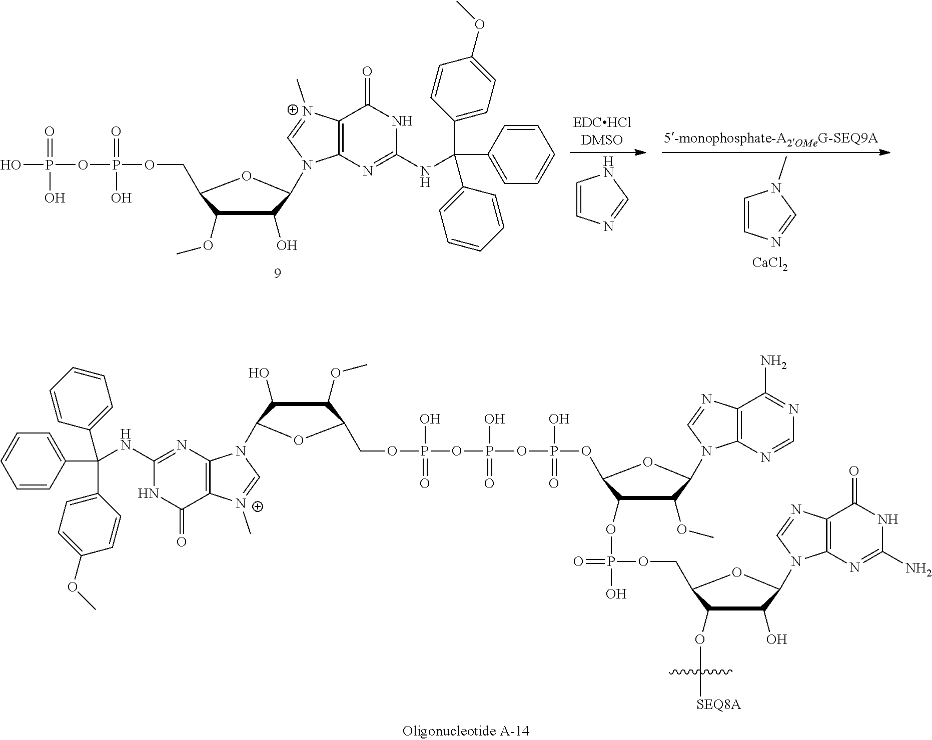

To a stirred solution of compound 9 (TEA salt, 12.4 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ9A (SEQ ID NO: 9) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-14 was pooled and concentrated under vacuum. Oligonucleotide A-14 was obtained as a clear film.

MS m/z=23655.8 [M+H].

### Example S12: Synthesis of Oligonucleotide A-15 ^{N7-(4-chlorobenzyl)}G_{3′OMe}ppp^{m6}A_{2′OMe}pG-SEQ5A

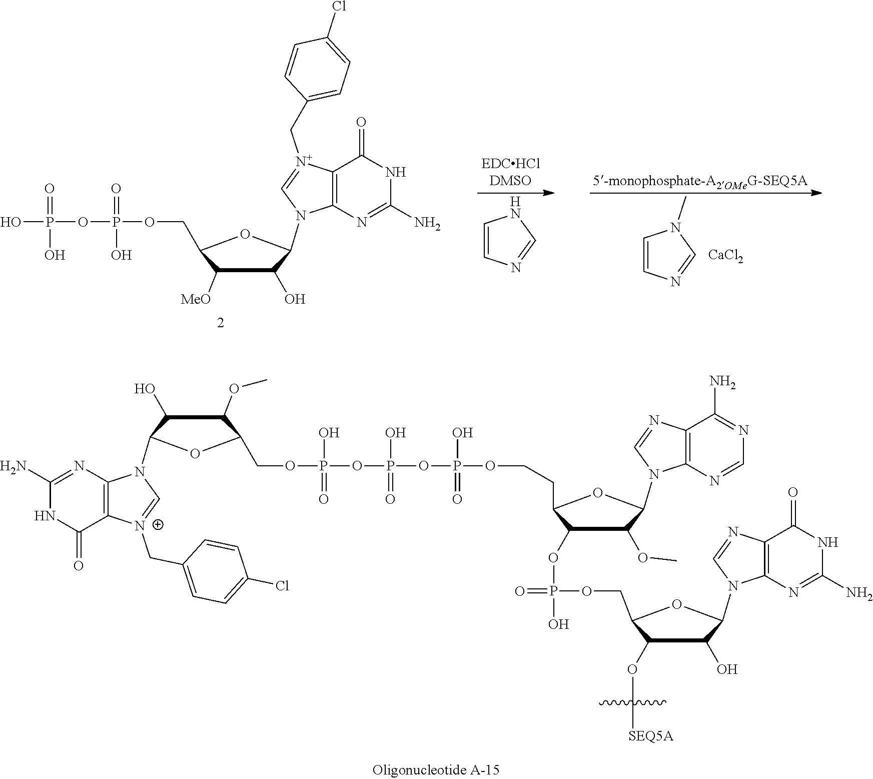

To a stirred solution of compound 2 (TEA salt, 10 mg, 0.013 mmol) in DMSO (0.13 mL), at room temperature, was added EDC hydrochloride salt (4.2 mg, 0.022 mmol), followed by imidazole (3.5 mg, 0.052 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ5A (SEQ ID NO: 5) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-15 was pooled and concentrated under vacuum. Oligonucleotide A-15 was obtained as a clear film.

MS m/z=29861.8 [M+H].

### Example S13: Synthesis of Oligonucleotide A-16 ^{m7, N2-butyl}G_{3′OMe}ppp^{m16}A_{2′OMe}pG-SEQ5A

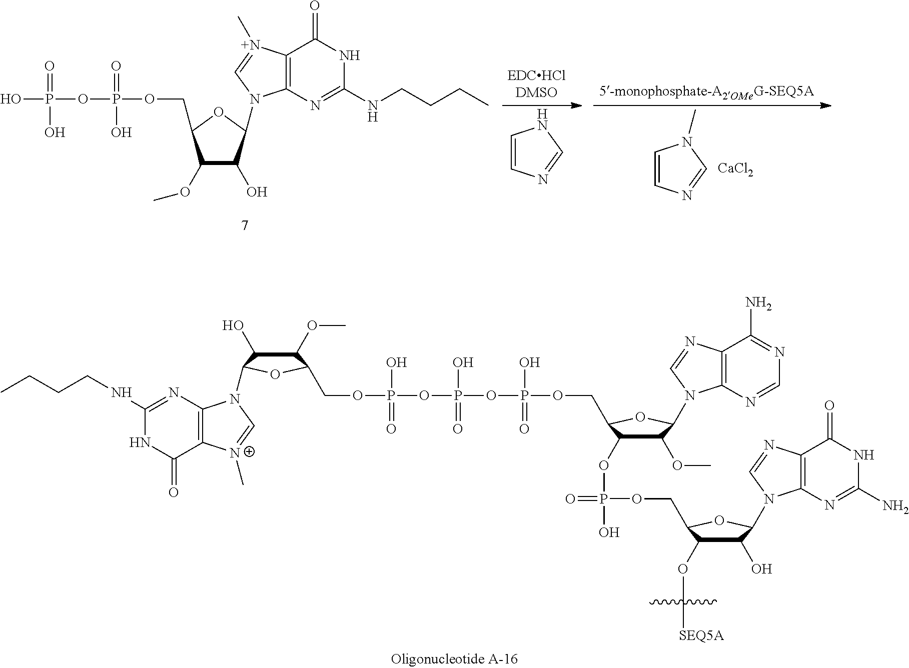

To a stirred solution of compound 7 (TEA salt, 10 mg, 0.014 mmol) in DMSO (0.14 mL), at room temperature, was added EDC hydrochloride salt (4.6 mg, 0.024 mmol), followed by imidazole (3.8 mg, 0.056 mmol). The resulting mixture was allowed to stir at room temperature overnight. Upon completion of the reaction, in a separate 1.5 mL Eppendorf tube, DMSO (62 μL), 0.1 M calcium chloride (10 μL, 1.0 μmol), 1-methylimidazole (8 μL, 100 μmol), and 1 mM 5′-monophosphate-A_{2′OMe}G-SEQ5A (SEQ ID NO: 5) (custom ordered from Integrated DNA Technologies, 10 μL, 10 nmol) in DMSO were added to the crude reaction solution (10 μL). The resulting solutions were mixed with a pipette and incubated at 55° C. for 1 hr. The crude reaction mixture was then diluted in RNAse free water (20 mL) pre-chilled in an ice bath and purified by anion exchange chromatography. Oligonucleotide A-16 was pooled and concentrated under vacuum. Oligonucleotide A-16 was obtained as a clear film.

MS m/z=29802.5 [M+H].

Each of the oligonucleotides SEQ ID NOs: 1, 3, 4, 5, 6, 7, 8, and 9 were custom ordered from Integrated DNA technologies, and were found to have significant amount of impurities (truncated species: oligonucleotides lacking 5′monophosphate), which distorts the true amount of oligonucleotide available for capping. Only SEQ ID NO: 2 (10 mer oligonucleotide) was determined to be pure.

### Example S14: mRNA Synthesis

mRNAs including each of the cap analogs (ARCA, A-1, A-2, A-control, and 3′OMe), including compounds as described herein, were synthesized for further testing. See Table 2 for synthesized analogs.

TABLE 2
Synthesized mRNAs

| Cap | |
|---|---|
| Analog | Synthesized mRNA Structure |
| ^{N7-} ⁽⁴⁻ ^{chlorobenzyl)} G_{3′OMe} ppp ^{m6}A_{2′OMe} pG Com- pound A-1 | 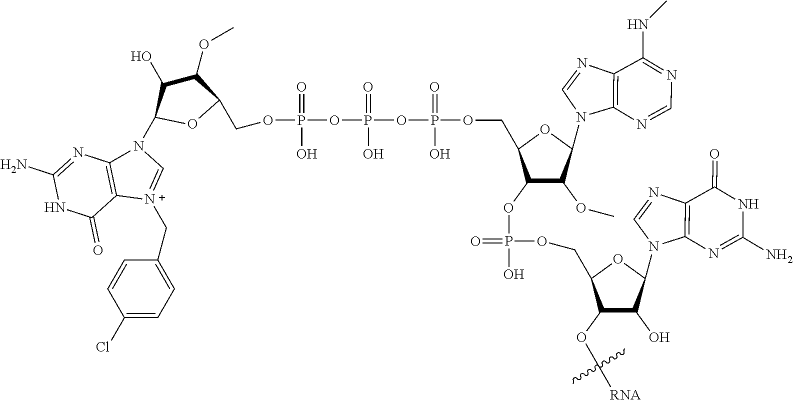 |
| ^{m7,} ^{N2-butyl} G_{3′OMe} ppp ^{m6}A_{2′OMe} pG Com- pound A-2 | 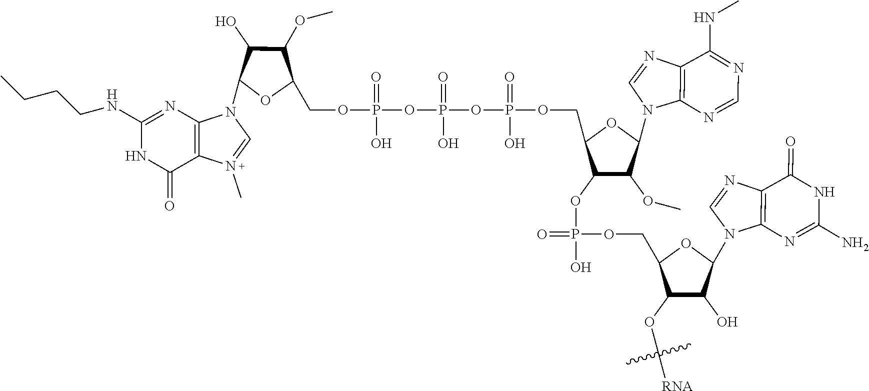 |
| ^{m7}G_{3′OMe} ppp ^{m6}A_{2′OMe} pG Com- pound A- control | 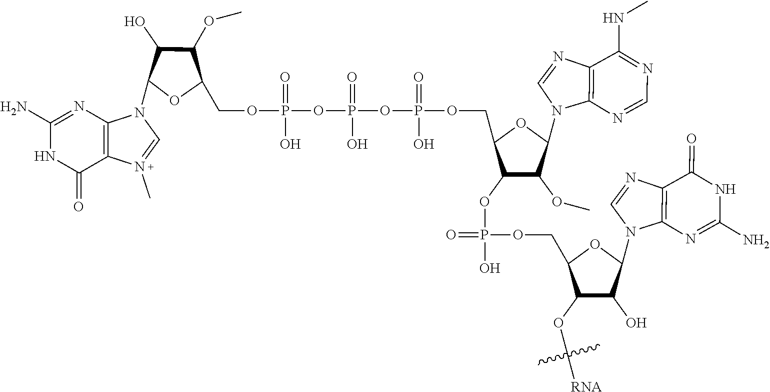 |
| ^{m7}G_{3′OMe} ppp A_{2′OMe} pG Com- pound 3′OMe | 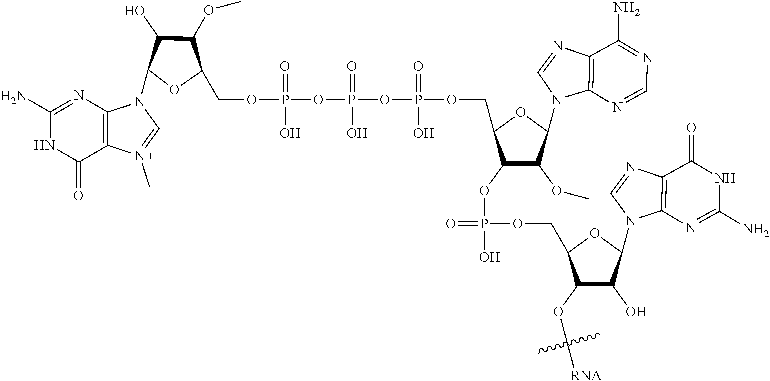 |
| ^{m7}G_{3′OMe} pppG ARCA | 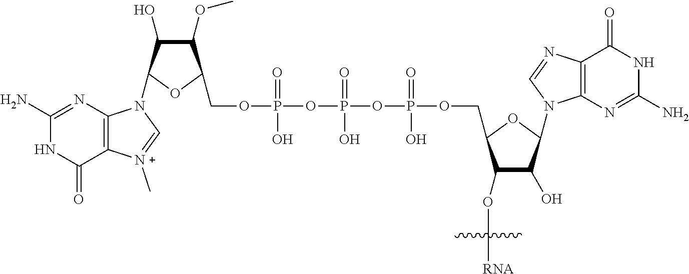 |

### Example B1: IP-RP HPLC Analysis of mRNA Capped with Cap Analog Bearing a Non-Removable Hydrophobic Group

Wasabi and FLuc mRNAs capped with cap analog ARCA, cap analog A-1, cap analog A-2, cap analog 3′OMe, or cap analog A-Control (see Table 2) were prepared as described in International Patent Publication Nos. WO2017053297 and WO2023147352 which are incorporated herein by reference in their entirety.

The IVT template was used to prepare mWasabi encoding mRNA (970 nucleotides) with cap analogs A-control, 3′OMe, A-1 or A-2 by in-vitro transcription. The mRNAs were purified prior to use in assays described below. Protein expression was measured by Allele Biotechnology in cell-based assays.

Alternatively, The IVT template was used to prepare FLuc encoding mRNA (1912 nucleotides) with cap analogs A-control or A-2 by in-vitro transcription.

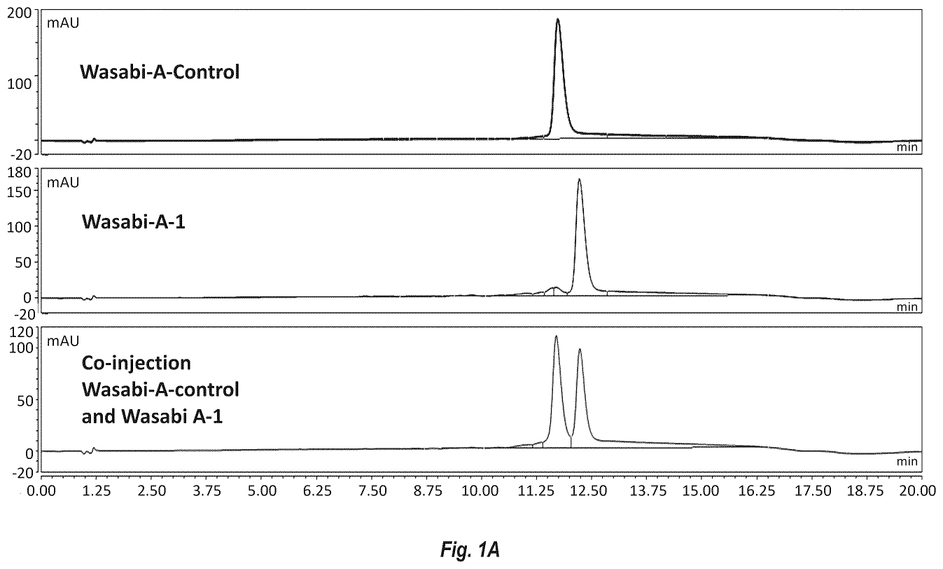

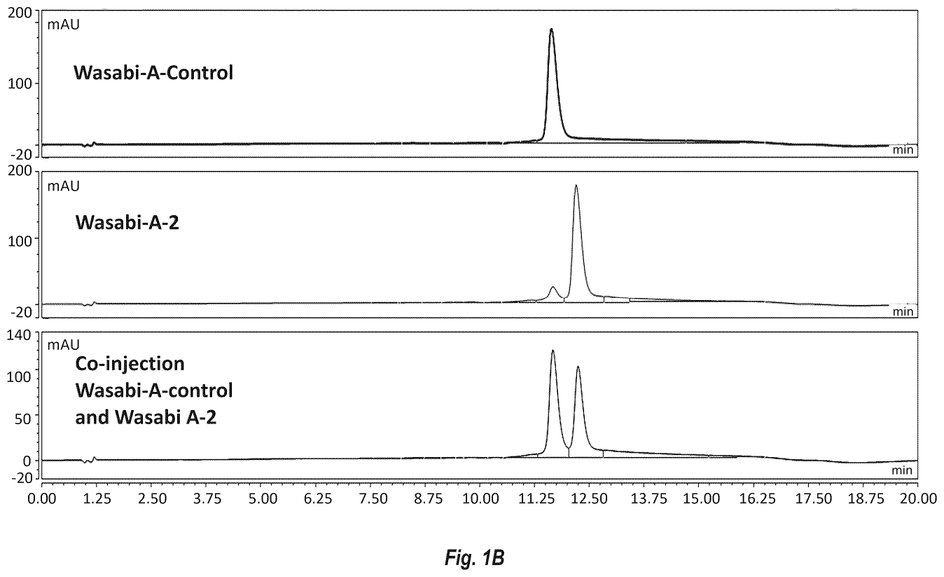

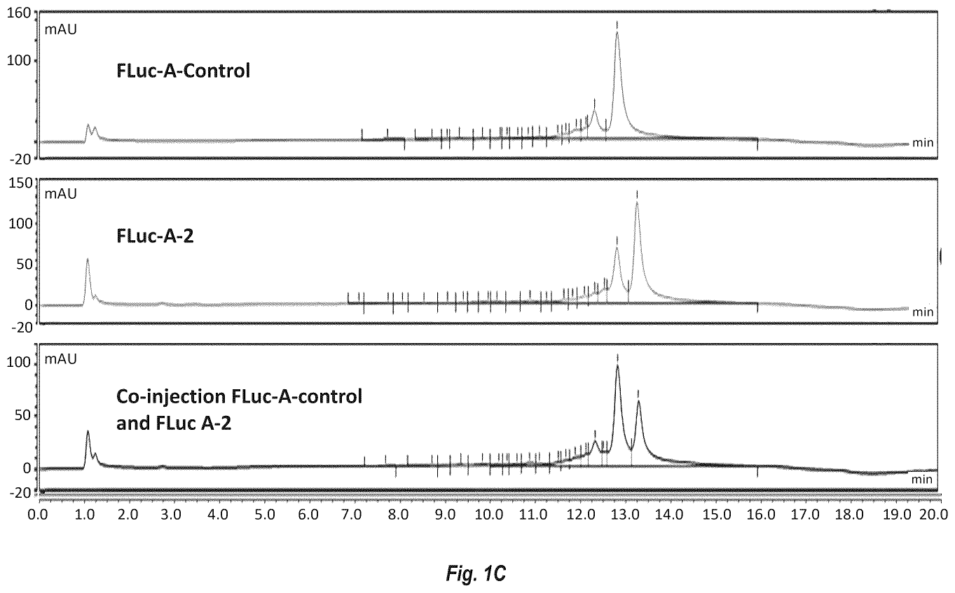

FIG. 1A shows three IP-RP HPLC chromatograms. The top trace is of Wasabi mRNA (about 970 nucleotides) capped with ^{m7}G_{3′OMe}ppp^{m6}A_{2′OMe}pG (A-control). The middle trace is of Wasabi mRNA capped with ^{N7-(4-chlorobenzyl)}G_{3′OMe}ppp^{m6}A_{2′OMe}pG (A-1). And the bottom trace is of a co-injection of Wasabi mRNA capped with ^{m7}G_{3′OMe}ppp^{m6} A_{2′OMe}pG (A-control) and Wasabi mRNA capped with N₇-(4-chlorobenzyl) G_{3′OMe}ppp^{m6}A_{2′OMe}pG (A-1). The difference between the control cap ^{m7}G_{3′OMe}ppp^{m6}A_{2′OMe}pG and cap A-1 ^{N7-(4-chlorobenzyl)}G_{3′OMe}ppp^{m6}A_{2′OMe}pG is a non-removable hydrophobic group (4-chlorobenzyl) at position N₇ of cap A-1. As demonstrated by the HPLC chromatograms, this small hydrophobic group is sufficient to allow for the separation of mRNAs that are 970 nucleotides long. This experiment demonstrates that a small hydrophobic group can solve the issue of mRNA purification.

FIG. 1B shows three IP-RP HPLC chromatograms. The top trace is of Wasabi mRNA (about 970 nucleotides) capped with ^{m7}G_{3′OMe}ppp^{m6}A_{2′OMe}pG (A-control). The middle trace is of Wasabi mRNA capped with ^{m7, N2-butyl}G_{3′OMe}ppp^{m6} A_{2′OMe}pG (A-2). And the bottom trace is of a co-injection of Wasabi mRNA capped with ^{m7}G_{3′OMe}ppp^{m6} A_{2′OMe}pG (A-control) and Wasabi mRNA capped with ^{m7, N2-butyl}G_{3′OMe}ppp^{m6}A_{2′OMe}pG (A-2). The difference between the control cap ^{m7}G_{3′OMe}ppp^{m6}A_{2′OMe}pG and cap A-2 ^{m7, N2-butyl}G_{3′OMe}ppp^{m6} A_{2′OMe}pG is a non-removable hydrophobic group (butyl) at position N2 of cap A-2. As demonstrated by the HPLC chromatograms, this small hydrophobic group is sufficient to allow for the separation of mRNAs that are 970 nucleotides long. This experiment demonstrates that a small hydrophobic group can solve the issue of mRNA purification.

FIG. 1C shows three IP-RP HPLC chromatograms. The top trace is of FLuc mRNA (about 1912 nucleotides) capped with ^{m7}G_{3′OMe}ppp^{m6}A_{2′OMe}pG (A-control). The middle trace is of Wasabi mRNA capped with ^{m7, N2-butyl}G_{3′OMe}ppp^{m6}A_{2′OMe}pG (A-2). And the bottom trace is of a co-injection of Wasabi mRNA capped with ^{m7}G_{3′OMe}ppp^{m6}A_{2′OMe}pG (A-control) and Wasabi mRNA capped with ^{m7, N2-butyl}G_{3′OMe}ppp^{m6}A_{2′OMe}pG (A-2). The difference between the control cap ^{m7}G_{3′OMe}ppp^{m6}A_{2′OMe}pG and cap A-2 ^{m7, N2-butyl}G_{3′OMe}ppp^{m6}A_{2′OMe}pG is a non-removable hydrophobic group (butyl) at position N2 of cap A-2. As demonstrated by the HPLC chromatograms, this small hydrophobic group is sufficient to allow for the separation of mRNAs that are 1912 nucleotides long. This experiment demonstrates that a small hydrophobic group can solve the issue of mRNA purification.

### Example B²: Protein Expression in Cell-Based Assays of mRNA Capped with Cap Analog Bearing a Non-Removable Hydrophobic Group

mWasabi Fluorescence Assay:

The day before transfection: cells were seeded as follows:
HEK293 cells: 6.25×10⁴/cm²
HeLa cells: 3.125×10⁴/cm²
The cells were dissociated with TrypLE
HEK293 cells were seeded in 96 well plates coated with Poly-D-Lysine (50 μL at 100 μg/mL per well).
HeLa cells were seeded in 96 well plates (no coating required).
The cells were seeded in 100 μL of growth media (Growth Media: DMEM+10% FBS supplemented with GlutaMAX and MEM Non-Essential Amino Acids).

The day of transfection: Opti-MEM and MessengerMax Transfection Reagent were allowed to reach room temperature. Two tubes were set up for each mRNA (i.e., for each cap analog).

Preparing tube 1:0.1 μL of MessengerMax was added to 5 μL Opti-MEM (these amounts were multiplied by the number of wells for each cap analog, in this case 6 repeats per cap analog were done), and the mixture was incubated at room temperature for 10 minutes.

Preparing tube 2: During the 10-minute incubation of mixture in tube 1, 12.5 ng of each mRNA (with different cap analog) was added to tubes containing 5 μL of Opti-MEM each (these amounts were multiplied by the number of wells for each cap analog, in this case 6 repeats per cap analog were done).

After the 10 min incubation of mixture in tube 1, mixture of tube 2 was added to mixture of tube 1, and the new mixture was incubated for 5 minutes.

96-well plates containing the cells, were washed once with 100 μL dPBS and the supernatant was aspirated and discarded. 10 μL of the mixture (mix of tube 1 and tube 2) was added to the cells within a 15-minute window (after the 5 min incubation period). 25 μL of Opti-MEM was added to each well, and the plates were incubated for 4 hours. After 4 hours, 100 μL of growth media was added to each well.

mWasabi fluorescent expression was quantified using Cytation 10 after 24 hours.

Cell Counts and viability readings were taken after 24 hrs using NC-200.

Structures of cap analogs used are shown in Table 2.

### Results

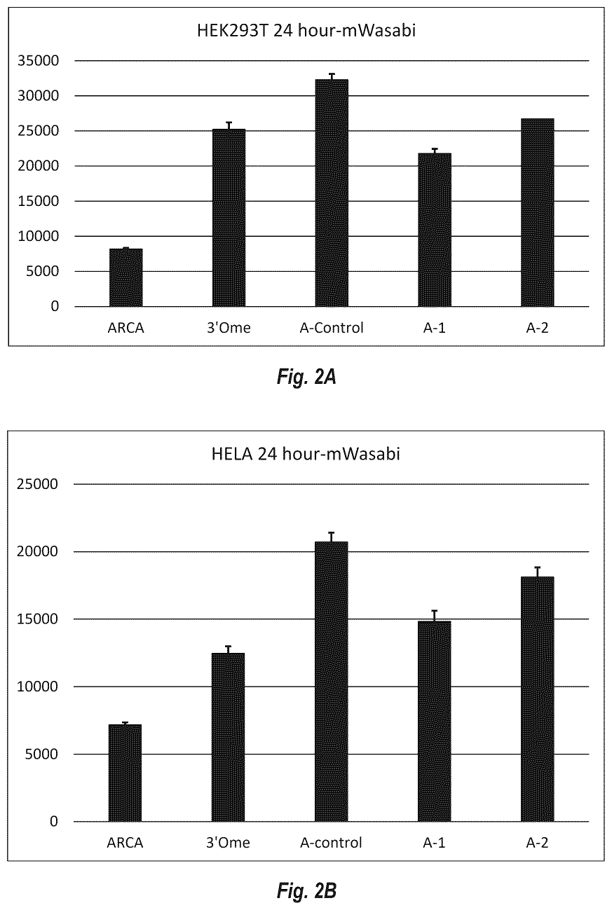

FIG. 2A shows fluorescence intensity (i.e., protein expression levels), 24 hours post transfection of mRNA capped with different cap analogs (see Table 2), in HEK293 cells. FIG. 2B shows fluorescence intensity (i.e., protein expression levels), 24 hours post transfection of mRNA capped with different cap analogs (see Table 2), in Hela cells. mRNA capped with ARCA was used as one of the controls.

In HEK293 cells, 24 hours post transfection, mRNAs capped with ^{N7-(4-chlorobenzyl)}G_{3′OMe}ppp^{m6}A_{2′OMe}pG (A-1) or ^{m7, N2-butyl}G_{3′OMe}ppp^{m6}A_{2′OMe}pG (A-2) displayed fluorescence intensity (i.e., highest protein expression) which was comparable to that of mRNA capped with Cap 3′OMe or mRNA capped with A-control, and much higher than that of mRNA capped with ARCA cap (FIG. 2A).

In HeLa cells, 24 hours post transfection, mRNAs capped with ^{N7-(4-chlorobenzyl)}G_{3′OMe}ppp^{m6}A_{2′OMe}pG (A-1) or ^{m7, N2-butyl}G_{3′OMe}ppp^{m6}A_{2′OMe}pG (A-2) displayed fluorescence intensity (i.e., highest protein expression) which was comparable to that of mRNA capped with Cap 3′OMe or mRNA capped with A-control, and much higher than that of mRNA capped with ARCA cap (FIG. 2B).

These results demonstrate that the translation of Wasabi mRNA was not affected by a small hydrophobic group at either position N2 or medium sized hydrophobic group at position N7 of G nucleotide.

Thus, using a cap analog with a small non-removable hydrophobic group (as small as n-butyl) can be a useful method for purifying full length mRNA, without affecting its translation.

### Example B3: IP-RP HPLC Analysis of Chemically Synthesized Oligonucleotides Capped with Cap Analog Bearing a Removable Hydrophobic Group

Oligonucleotide A-5 (5′-capped 10 mer oligo) was prepared as described above (Example S5) and used as a proof-of-concept model for longer oligonucleotides (100mer, 90mer and 80mer).

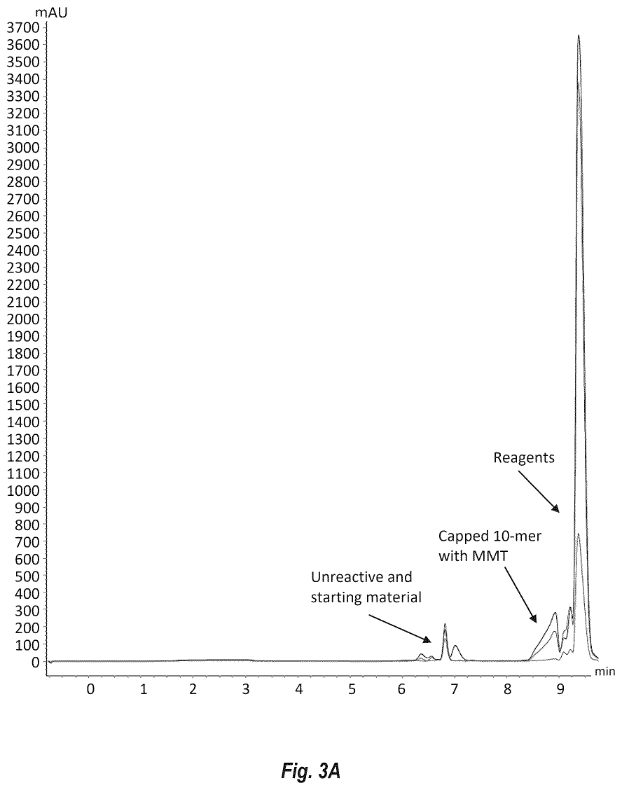

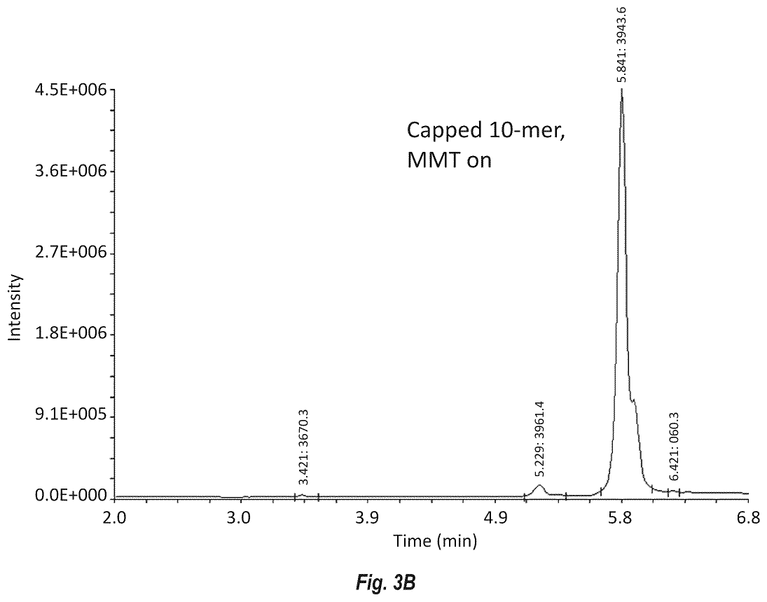

FIG. 3A shows the IP-RP HPLC chromatogram of 10-mer Oligonucleotide A-5 (SEQ ID NO: 2 capped with compound 9), whose cap comprises a removable hydrophobic group (MMT) at position N2. The fractions corresponding to the peak of Oligonucleotide A-5 (with retention time of ˜8.8 min.) were collected, concentrated, and analyzed by LC-MS (FIG. 3B).

The LC-MS chromatogram shows a single peak with expected molar mass of 3943.6 g/mol.

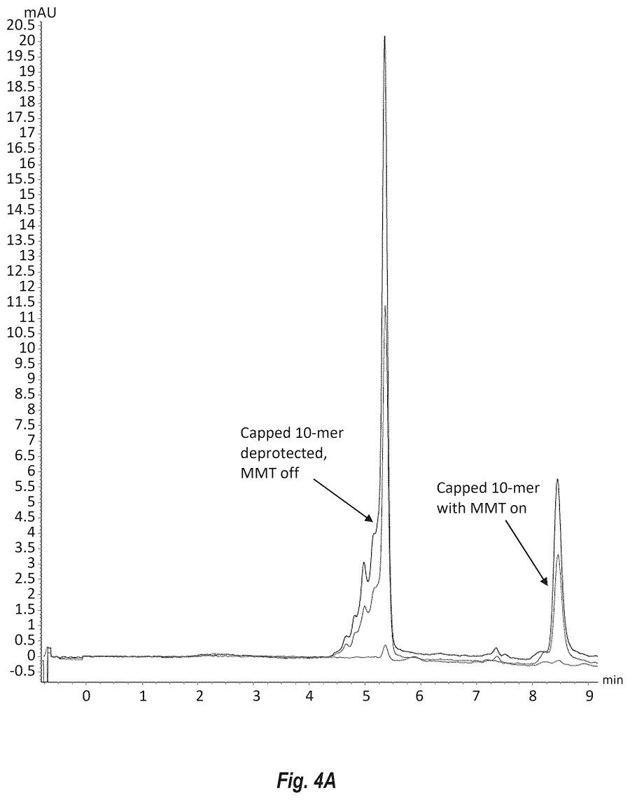

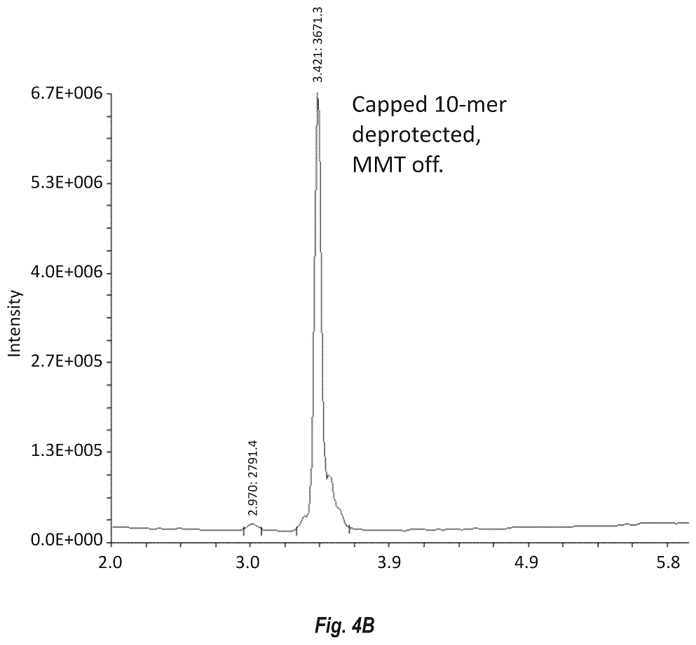

Oligonucleotide A-5 was deprotected as described above (Example S5). FIG. 4A shows the IP-RP HPLC chromatogram of Oligonucleotide A-5, whose cap comprises a removable hydrophobic group (MMT) at position N2, and the deprotected Oligonucleotide A-6 (MMT is removed). The fractions corresponding to the peak of Oligonucleotide A-6 (with retention time of ˜5.2 min.) were collected, concentrated, and analyzed by LC-MS (FIG. 4B).

The LC-MS chromatogram shows a single peak with expected molar mass of 3671.3 g/mol.

Thus, a cap analog with a removable hydrophobic group such as MMT can be used for purification of longer oligonucleotides (such as 50 mer to 4000 mer oligonucleotides), and then the hydrophobic group can be removed to allow translation without the large hydrophobic group.

This experiment was performed with 100mer oligonucleotide (Oligonucleotide A-3), 90mer oligonucleotides (Oligonucleotide A-7 and Oligonucleotide A-8), and 80mer oligonucleotides (Oligonucleotide A-9 and Oligonucleotide A-11).

Oligonucleotide A-3 (5′-capped 100 mer oligo) was prepared as described above (Example S3).

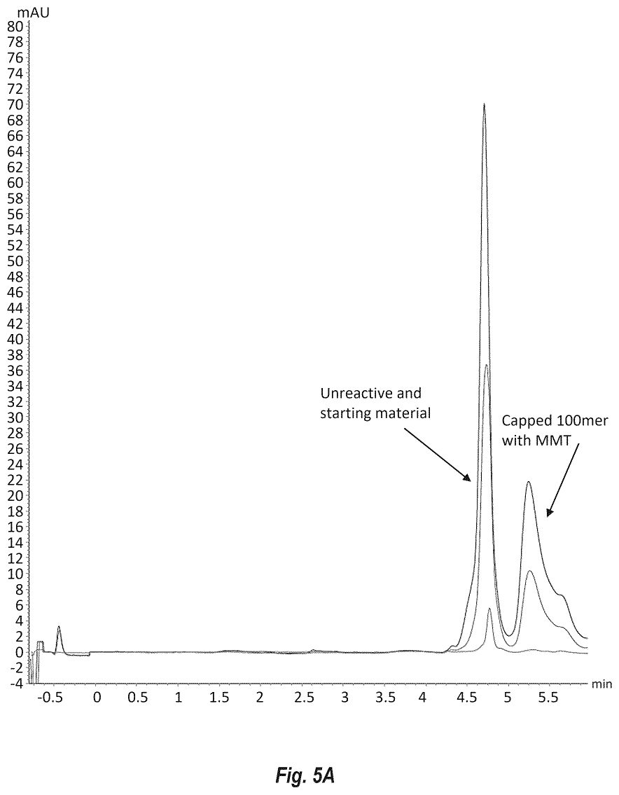

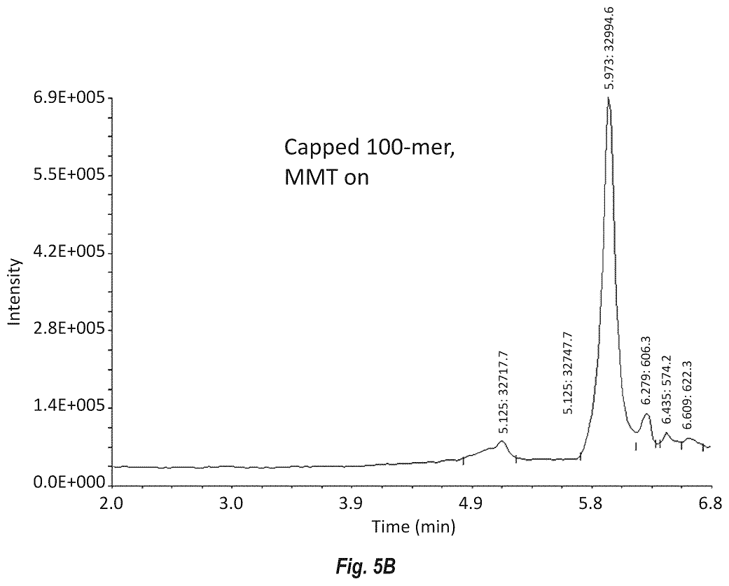

FIG. 5A shows the IP-RP HPLC chromatogram of 100-mer Oligonucleotide A-3 (SEQ ID NO: 1 capped with compound 9), whose cap comprises a removable hydrophobic group (MMT) at position N2. The fractions corresponding to the peak of Oligonucleotide A-3 (with retention time of ˜5.4 min.) were collected, concentrated, and analyzed by LC-MS (FIG. 5B).

The LC-MS chromatogram shows a single peak with expected molar mass of 32994.6 g/mol.

MMT was removed as described for Oligonucleotide-5 (results not shown).

Oligonucleotide A-3 (5′-capped 100 mer oligo) was prepared as described above (Example S3).

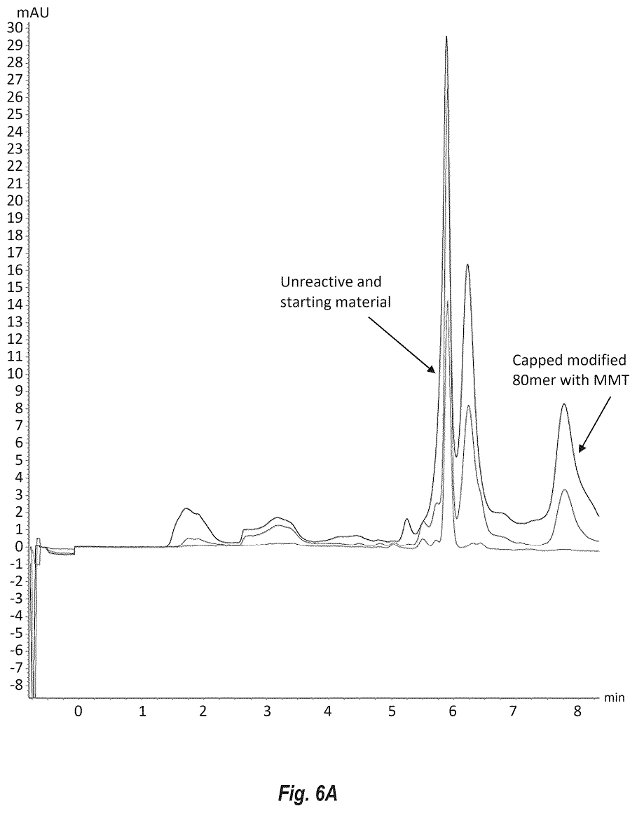

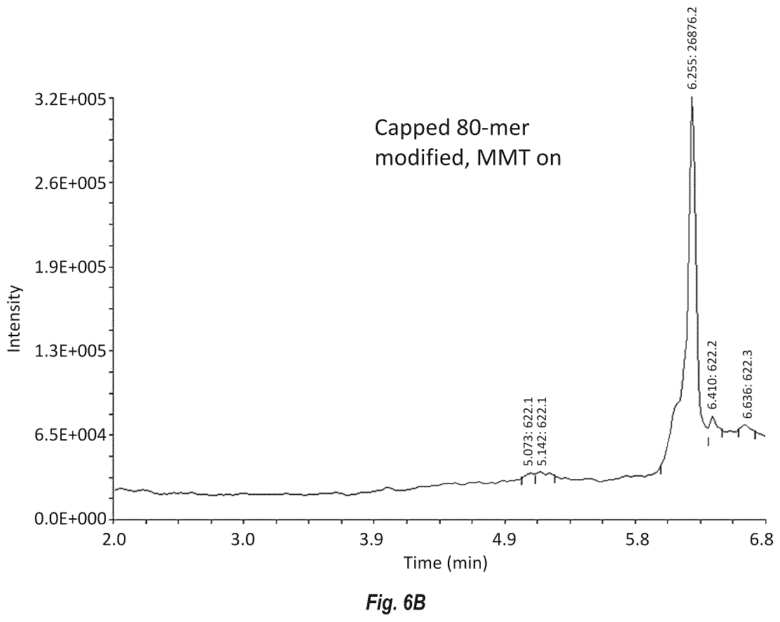

FIG. 6A shows the IP-RP HPLC chromatogram of 80-mer Oligonucleotide A-11 (SEQ ID NO: 7 capped with compound 9), whose cap comprises a removable hydrophobic group (MMT) at position N2. The fractions corresponding to the peak of Oligonucleotide A-11 (with retention time of ˜7.8 min.) were collected, concentrated, and analyzed by LC-MS (FIG. 6B).

The LC-MS chromatogram shows a single peak with expected molar mass of 26876.2 g/mol.

Oligonucleotide A-11 was deprotected as described above (Example S9) to yield the deprotected Oligonucleotide A-12 (MMT is removed). The fractions corresponding to the peak of Oligonucleotide A-12 were collected, concentrated, and analyzed by LC-MS. The LC-MS chromatogram shows a single peak with expected molar mass of 26553.8 g/mol (results not shown).

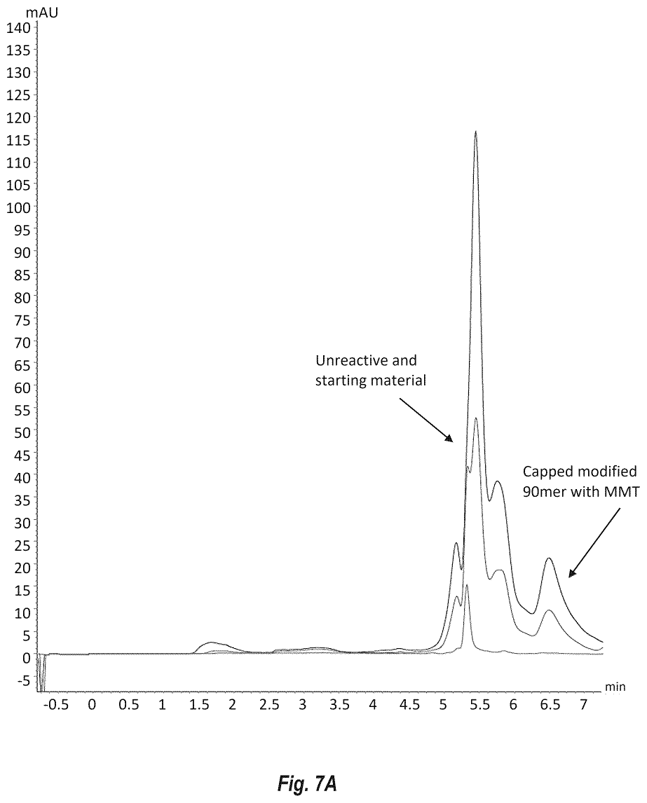

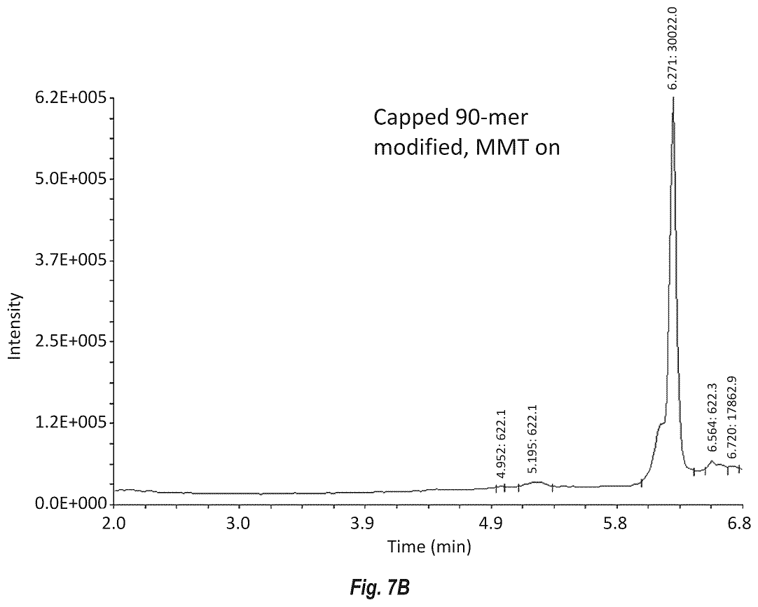

FIG. 7A shows the IP-RP HPLC chromatogram of 80-mer Oligonucleotide A-8 (SEQ ID NO: 5 capped with compound 9), whose cap comprises a removable hydrophobic group (MMT) at position N2. The fractions corresponding to the peak of Oligonucleotide A-8 (with retention time of ˜6.5 min.) were collected, concentrated, and analyzed by LC-MS (FIG. 7B).

The LC-MS chromatogram shows a single peak with expected molar mass of 30022.0 g/mol.

MMT was removed as described for Oligonucleotide-5 (results not shown).

These experiments show that a cap analog with a removable hydrophobic group such as MMT can be used for purification of longer oligonucleotides (80mer, 90mer, and 100mer), and then the hydrophobic group can be removed to allow translation without the large hydrophobic group.

### Example B4: LC-MS Analysis of Chemically Synthesized Modified 90-mer Oligonucleotide Capped with Cap Analog Bearing a Non-Removable Hydrophobic Group

Oligonucleotide A-15 and Oligonucleotide A-16 (5′-capped modified 90 mer oligos) were prepared as described above (Examples S12 and S13 respectively).

Each oligonucleotide (Oligonucleotide A-15 and Oligonucleotide A-16) was purified using LC-MS. The LC-MS chromatograms show separation of the capped oligonucleotides from the unreactive and starting materials.

For Oligonucleotide A-15 the retention time was 5.3 minutes. The retention time for the unreactive and starting materials was 5.0 minutes.

For Oligonucleotide A-16 the retention time was 5.4 minutes. The retention time for the unreactive and starting materials was 5.1 minutes.

This experiment demonstrates that cap analogs with non-removable hydrophobic groups (A-1 and A-2) can help with purification of long chemically synthesized oligonucleotides. Only full length strands contain 5′-monophosphate (aborted or partial strands would be capped with acetyl groups during the chemical synthesis) which can react with activated guanosine diphosphate (GDP) (i.e., imidazolide).

### Example B5: RNAse Digestion Assay of Modified and Unmodified 90-mer Oligonucleotide

Oligonucleotide SEQ ID NO: 4 is a 90-mer oligonucleotide. Oligonucleotide SEQ ID NO: 5 is a modified 90-mer oligonucleotide whose ribose sugars of the last 5 nucleotides, at the 3′end, were modified with methoxy at the 2′ position and the last 5 internucleotide linkages are phosphorothioates (P═S instead of P═O).

RNAse R and 10× RNAse R Buffer were purchased from Biosearch Technologies, Catalog No: RNR07250.

To a 1 mM solution of oligonucleotide SEQ ID NO: 4 in DMSO (1.5 μL, 1.5 nmol) was added RNAse free water (65.25 μL) and 10× RNAse R Buffer (0.2 M Tris-HCl, pH 8.0, 1 M KCl, 1 mM MgCl₂, 7.5 μL). An aliquot (10 μL) of the solution was removed as a time point 0 hours. RNAse R (20 U/μL, 0.75 μL) was added and the solution mixed with a pipette and incubated at room temperature for 8 hours. At 0, 2, 4, and 8 hours after addition of RNAse R an aliquot (10 μL) was placed in another container, 0.5 M EDTA (0.5 μL) was added, and then the solution incubated at 65° C. for 15 minutes to inactivate the enzyme. An aliquot (1 μL) of the solution taken at each time point was mixed with RNAse free water (19 μL) and then analyzed by LC-MS.

To a 1 mM solution of oligonucleotide SEQ ID NO: 5 in DMSO (1.5 μL, 1.5 nmol) was added RNAse free water (65.25 μL) and 10× RNAse R Buffer (0.2 M Tris-HCl, pH 8.0, 1 M KCl, 1 mM MgCl₂, 7.5 μL). An aliquot (10 μL) of the solution was removed as a time point 0 hours. RNAse R (20 U/μL, 0.75 μL) was added and the solution mixed with a pipette and incubated at room temperature for 8 hours. At 0, 2, 4, and 8 hours after addition of RNAse R an aliquot (10 μL) was placed in another container, 0.5 M EDTA (0.5 μL) was added, and then the solution incubated at 65° C. for 15 minutes to inactivate the enzyme. An aliquot (1 μL) of the solution taken at each time point was mixed with RNAse free water (19 μL) and then analyzed by LC-MS.

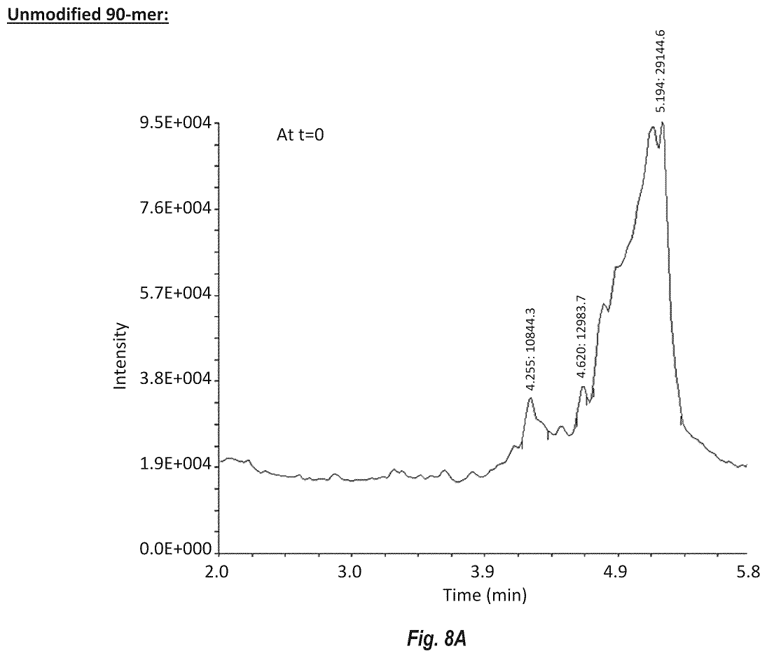

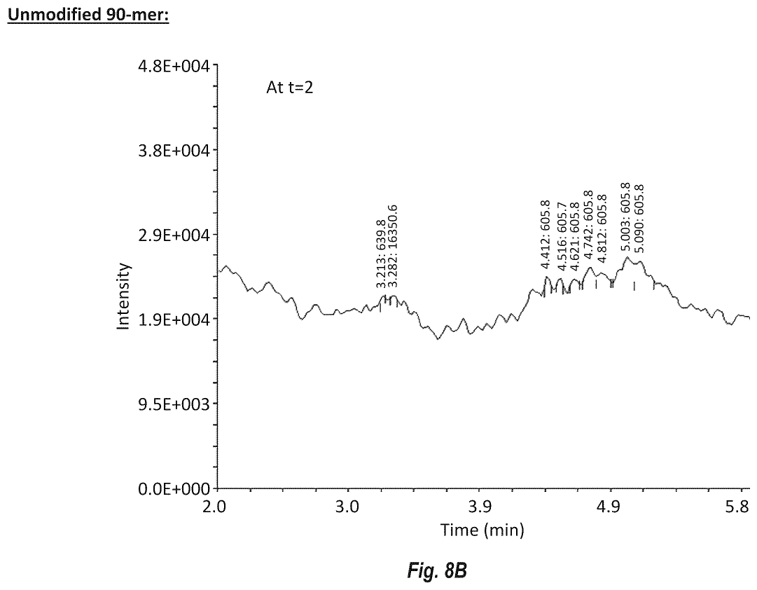

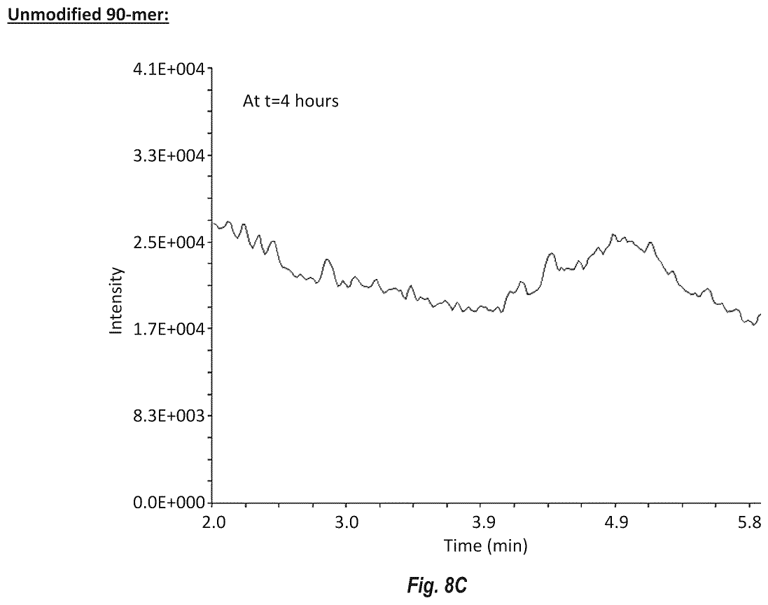

FIG. 8A shows an LC-MS chromatogram of crude oligonucleotide SEQ ID NO: 4 prior to the addition of RNAse R. FIG. 8B shows an LC-MS chromatogram of the oligonucleotide after 2 hours of digestion with RNAse R. FIG. 8C shows an LC-MS chromatogram of the oligonucleotide after 4 hours of digestion with RNAse R.

FIG. 8 demonstrates that unmodified 90-mer oligonucleotide is almost completely digested after 2 hrs, and completely digested after 4 hrs.

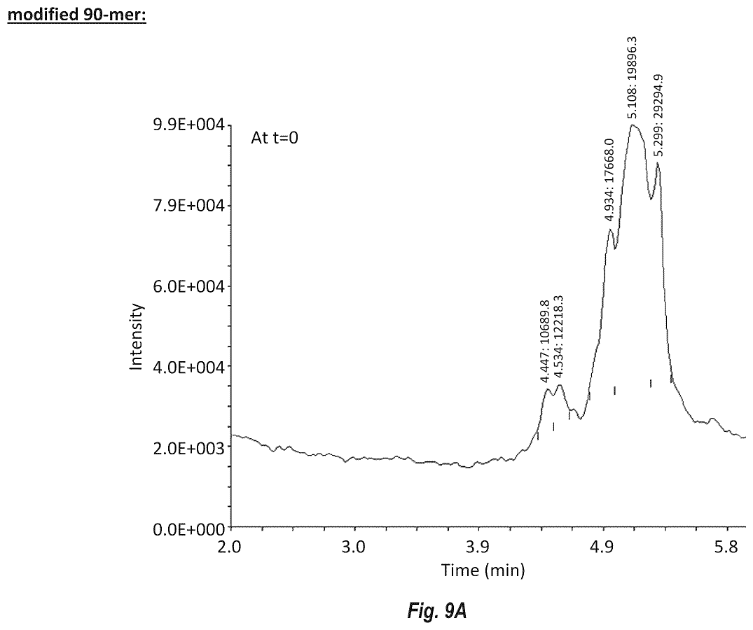

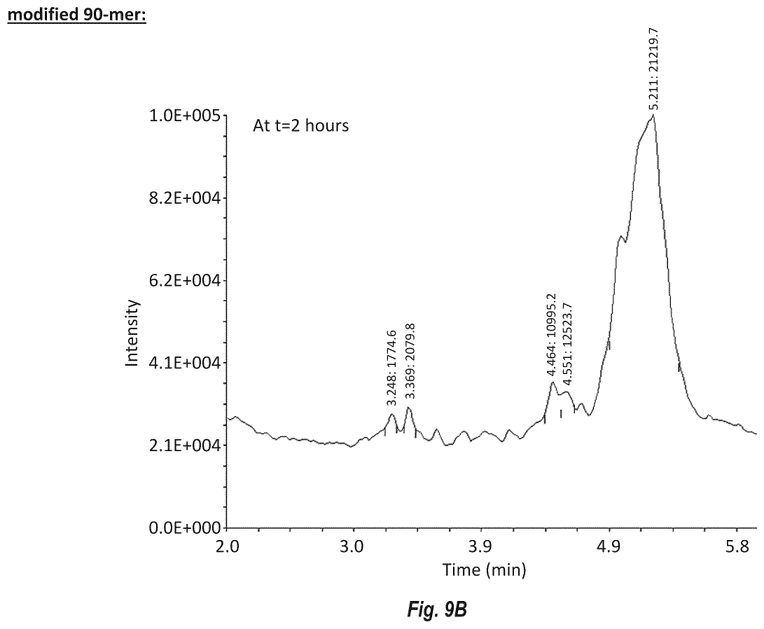

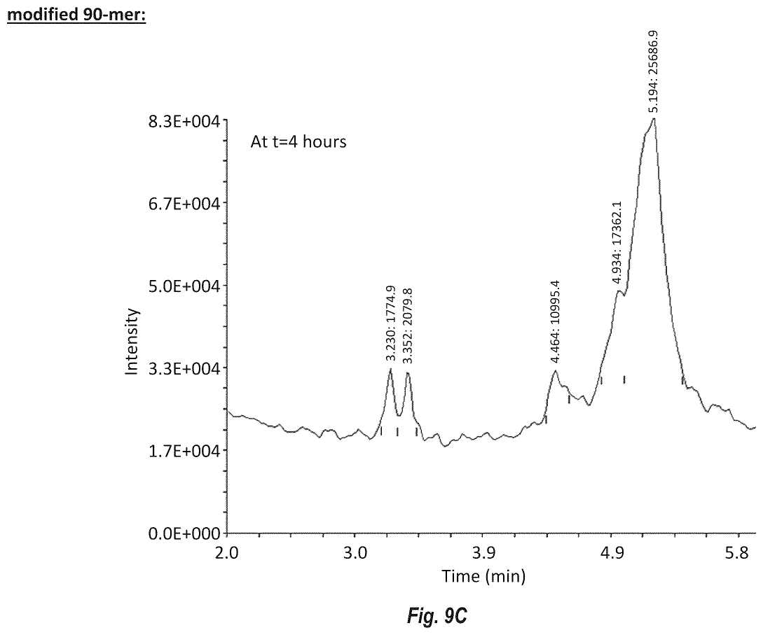

FIG. 9A shows an LC-MS chromatogram of crude oligonucleotide SEQ ID NO: 5 prior to the addition of RNAse R. FIG. 9B shows an LC-MS chromatogram of the oligonucleotide after 2 hours of digestion with RNAse R. FIG. 9C shows an LC-MS chromatogram of the oligonucleotide after 4 hours of digestion with RNAse R.

FIG. 9 demonstrates that modified 90-mer oligonucleotide is stable to digestion even after 4 hours.

This experiment demonstrates that small modifications at the 3′ end of an oligonucleotide greatly contribute to its stabilization. Accordingly, chemically synthesized modified oligonucleotides, which can be ligated together to provide a very long oligonucleotide, can provide more stable RNAs, which can then be purified using the methods described herein.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims. The description is to be read from the perspective of one of ordinary skill in the art; therefore, information well known to the skilled artisan is not necessarily included.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

The disclosure further includes the following numbered embodiments.

Embodiment 1 A compound of Formula (IIID):

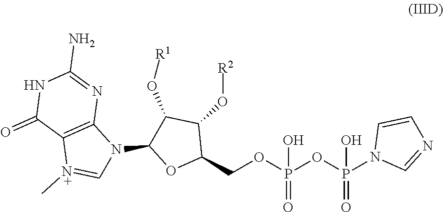

or a salt thereof, wherein one of R¹ or R² is a removable hydrophobic group and the other R¹ or R² is hydrogen or C₁₋₃ alkyl.

Embodiment 2 The compound of embodiment 1, wherein R¹ is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs).

Embodiment 3 The compound of embodiment 2, wherein R¹ is Trt.

Embodiment 4 The compound of embodiment 2, wherein R¹ is monomethoxytrityl.

Embodiment 5 The compound of embodiment 2, wherein R¹ is DMT.

Embodiment 6 The compound of embodiment 1, wherein R¹ is a photolabile protecting group.

Embodiment 7 The compound of embodiment, wherein R¹ is an arylcarbonylmethyl group, a nitroaryl group, a coumarin-4-yl group, an arylmethyl group, meta-containing group or a pivaloyl group.

Embodiment 8 The compound of embodiment, wherein R¹ is a nitroaryl group.

Embodiment 9 The compound of any one of embodiments 1-8, wherein R² is CH₃.

Embodiment 10 The compound of any one of embodiments 1-8, wherein R² is H.

Embodiment 11 The compound of embodiment 1, wherein R² is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs).

Embodiment 12 The compound of embodiment 11, wherein R² is Trt.

Embodiment 13 The compound of embodiment 11, wherein R² is monomethoxytrityl.

Embodiment 14 The compound of embodiment 11, wherein R² is DMT.

Embodiment 15 The compound of embodiment 1, wherein R² is a photolabile protecting group.

Embodiment 16 The compound of embodiment 15, wherein R² is an arylcarbonylmethyl group, a nitroaryl group, a coumarin-4-yl group, an arylmethyl group, meta-containing group or a pivaloyl group.

Embodiment 17 The compound of embodiment 16, wherein R² is a nitroaryl group.

Embodiment 18 The compound of any one of embodiments 1 and 11-17 wherein R¹ is CH₃.

Embodiment 19 The compound of any one of embodiments 1 and 11-17, wherein R¹ is H.

Embodiment 20 A compound of Formula (IIIE):

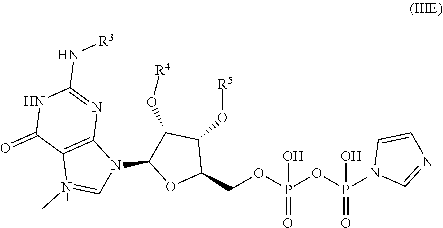

or a salt thereof, wherein R³ is a removable hydrophobic group, R⁴ is hydrogen or C₁₋₃ alkyl and R⁵ is hydrogen or C₁₋₃ alkyl.

Embodiment 21 The compound of embodiment 20, wherein R³ is a 9-fluorenylmethoxycarbonyl (Fmoc) group or a substituted Fmoc group.

Embodiment 22 The compound of embodiment 21, wherein R³ is 2,7-di-tert-butyl-Fmoc, 2-fluro-Fmoc or 2-monoisooctyl-Fmoc.

Embodiment 23 The compound of embodiment 21, wherein R³ is an acetyl group.

Embodiment 24 The compound of embodiment 23, wherein R³ is has the formula

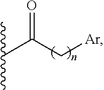

wherein Ar is a substituted or unsubstituted aromatic moiety and n is an integer from 0 to 5.

Embodiment 25 The compound of embodiment 24, wherein n is 1.

Embodiment 26 The compound of embodiment 25, wherein the acetyl group is

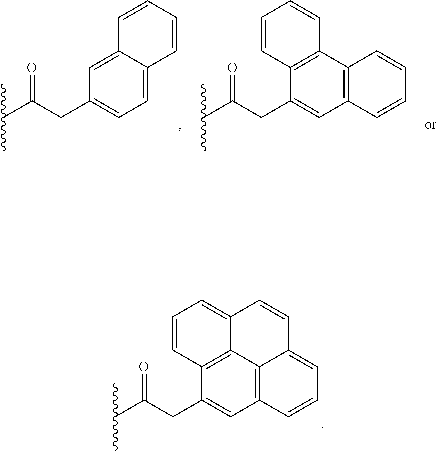 [SMILES: *C(=O)Cc1ccc2ccccc2c1 ; *C(=O)Cc1cc2ccccc2c2ccccc12 ; *C(=O)Cc1cc2cccc3ccc4cccc1c4c32]

Embodiment 27 The compound of embodiment 20, wherein R³ is 2-(4-nitrophenylsulfonyl)ethoxycarbonyl (Nsc), 1,1-dioxobenzo[b]thiophene-2-ylmethoxycarbonyl (Bsmoc), (1,1,dioxonaphtho[1,2-b]thiophene-2-yl)methylcarbonyl (α-Nsmoc), 1-(4,4-dimethyl-2,6-dioxocyclohex-1-ylidene)-e-methylbutyl (ivDde), 2-phenyl(methyl)sulfonio)ethoxycarbonyl tetrafluoroborate (Pms), wthanesulfonylethoxycarbonyl (Esc) 9-fluorenylmethyl (Fm), 4-(N-[1-(4,4-dimethyl-2,6-dioxocyclohexylidene)-3-methylbutyl]-amino)benzyl (Dmab) or 2-(4-sulfophenylsulfonyl)ethoxycarbonyl (Sps).

Embodiment 28 The compound of any one of embodiments 20-27, wherein R⁴ is CH₃ and R⁵ is H.

Embodiment 29 The compound of any one of embodiments 20-27, wherein R⁵ is CH₃ and R⁴ is H.

Embodiment 30 The compound of any one of embodiments 20-27, wherein R⁵ is CH₃ and R⁴ is CH₃.

Embodiment 31 A compound of Formula (IIIF):

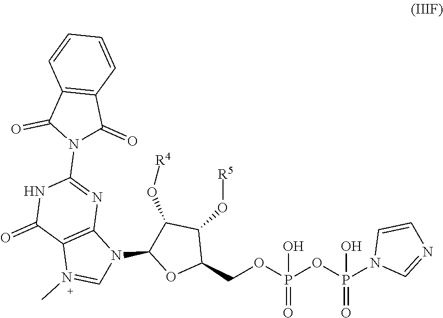

or a salt thereof, wherein R⁴ is hydrogen or C₁₋₃ alkyl and R⁵ is hydrogen or C₁₋₃ alkyl.

Embodiment 32 A compound of Formula (IIIG):

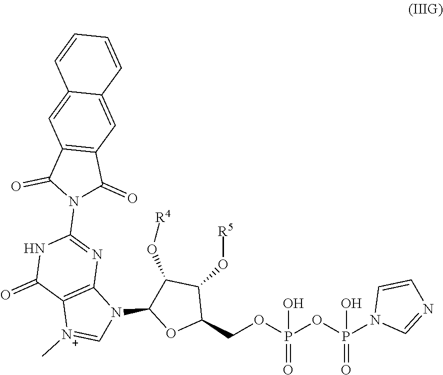

or a salt thereof, wherein R⁴ is hydrogen or C₁₋₃ alkyl and R⁵ is hydrogen or C₁₋₃ alkyl.

Embodiment 33 The compound of embodiment 31 or claim 32, wherein R⁴ is CH₃ and R⁵ is H.

Embodiment 34 The compound of embodiment 31 or claim 32, wherein R⁵ is CH₃ and R⁴ is H.

Embodiment 35 The compound of embodiment 31 or claim 32, wherein R⁵ is CH₃ and R⁴ is CH₃.

Embodiment 36 A compound of Formula (IIIH):

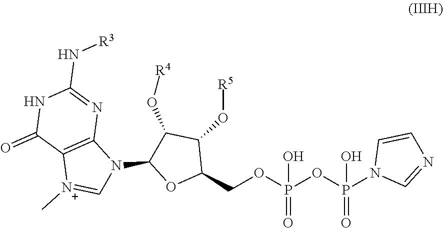

or a salt thereof, wherein one of R¹ or R² is a removable hydrophobic group and the other R¹ or R² is hydrogen or C₁₋₃ alkyl, and R³ is a removable hydrophobic group.

Embodiment 37 The compound of embodiment 36, wherein R¹ is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs).

Embodiment 38 The compound of embodiment 37, wherein R¹ is Trt.

Embodiment 39 The compound of embodiment 37, wherein R¹ is monomethoxytrityl.

Embodiment 40 The compound of embodiment 37, wherein R¹ is DMT.

Embodiment 41 The compound of embodiment 36, wherein R¹ is a photolabile protecting group.

Embodiment 42 The compound of embodiment 41, wherein R¹ is an arylcarbonylmethyl group, a nitroaryl group, a coumarin-4-yl group, an arylmethyl group, meta-containing group or a pivaloyl group.

Embodiment 43 The compound of embodiment 42, wherein R¹ is a nitroaryl group.

Embodiment 44 The compound of any one of embodiments 36-43, wherein R² is CH₃.

Embodiment 45 The compound of any one of embodiments 36-43, wherein R² is H.

Embodiment 46 The compound of embodiment 36, wherein R² is selected from the group consisting of trityl (Trt), monomethoxytrityl, dimethoxytrityl (DMT), p-methylbenzyl (Meb), trimethoxybenzyl (Tmob), 9-xanthenyl (Xan), 2,2,4,6,7-pentamthyl-5-dihydrobenzofuranylmethyl (Pmbf), benzyl (Bn), tert-butyl, a-Adamantyl (1-Ada), p-methoxybenzyl (Mob), tert-butyloxycarbonyl (Boc), 3,5-dimethoxyphenylisoproxycarbonyl (Ddz), 2-(4-biphenyl)isopropoxycarbonyl (BPoc) and 2-nitrophenylsulfenyl (NFs).

Embodiment 47 The compound of embodiment 46, wherein R² is Trt.

Embodiment 48 The compound of embodiment 46, wherein R² is monomethoxytrityl.

Embodiment 49 The compound of embodiment 46, wherein R² is DMT.

Embodiment 50 The compound of embodiment 46, wherein R² is a photolabile protecting group.

Embodiment 51 The compound of embodiment 50, wherein R² is an arylcarbonylmethyl group, a nitroaryl group, a coumarin-4-yl group, an arylmethyl group, meta-containing group or a pivaloyl group.

Embodiment 52 The compound of embodiment 51, wherein R² is a nitroaryl group.

Embodiment 53 The compound of any one of embodiments 46-52, wherein R¹ is CH₃.

Embodiment 54 The compound of any one of embodiments 46-52, wherein R¹ is H.

Embodiment 55 The compound of any one of embodiments 36-54, wherein the compound is a disodium salt.

Embodiment 56 The compound of any one of embodiments 36-54, wherein the compound is a monosodium salt.

Embodiment 57 The compound of any one of embodiments 1-56, wherein the imidazole group is substituted with 1-3 substituents.

Embodiment 58 The compound of embodiment 57, wherein the substituents on the imidazole are C₁₋₆ alkyl groups.

Embodiment 59 The compound of any one of embodiments 1-58, wherein the compound is a disodium salt.

Embodiment 60 The compound of any one of embodiments 1-58, wherein the compound is a monosodium salt.

Embodiment 61 A protected 5′-capped mRNA prepared by the reaction of a compound of any one of embodiments 1-60 and a 5′-phosphate-mRNA.

Embodiment 62 The protected 5′-capped mRNA of embodiment 61, wherein the 5′-phosphate-mRNA is prepared non-enzymatically.

Embodiment 63 The protected 5′-capped mRNA of embodiment 61 or embodiment 62, wherein the protected 5′-capped mRNA comprises fewer than 150 nucleotide bases.

Embodiment 64 The protected 5′-capped mRNA of embodiment 61 or embodiment 62, wherein the protected 5′-capped mRNA comprises fewer than 100 nucleotide bases.

Embodiment 65 The protected 5′-capped mRNA of embodiment 61 or embodiment 62, wherein the protected 5′-capped mRNA comprises fewer than 75 nucleotide bases.

Embodiment 66 The protected 5′-capped mRNA of embodiment 61 or embodiment 62, wherein the protected 5′-capped mRNA comprises from about 50 to about 100 nucleotide bases.

Embodiment 67 A 5′-capped mRNA prepared by removing the protecting group(s) of the protected 5′-capped mRNA of any one of embodiments 61-66.

Embodiment 68 A composition comprising a 5′-capped mRNA of embodiment 67, wherein the composition comprises less than 1% by weight of uncapped 5′-phosphate-mRNA molecules.

Embodiment 69 A composition comprising a 5′-capped mRNA of embodiment 67, wherein the composition comprises less than 0.5% by weight of uncapped 5′-phosphate-mRNA molecules.

Embodiment 70 A composition comprising a 5′-capped mRNA of embodiment 67, wherein the composition comprises less than 0.25% by weight of uncapped 5′-phosphate-mRNA molecules.

Embodiment 71 A composition comprising a 5′-capped mRNA of embodiment 67, wherein the composition comprises less than 0.1% by weight of uncapped 5′-phosphate-mRNA molecules.

Embodiment 72 A composition comprising a 5′-capped mRNA of embodiment 67, wherein the composition comprises less than 0.05% by weight of uncapped 5′-phosphate-mRNA molecules.

Embodiment 73 A composition comprising a 5′-capped mRNA of embodiment 67, wherein the composition is substantially free of 5′-phosphate-mRNA molecules.

Embodiment 74 A composition of any one of embodiments 68-73, wherein the composition is substantially free of enzymatic byproducts.

Embodiment 75 A protected 5′-capped mRNA of Formula (IID):

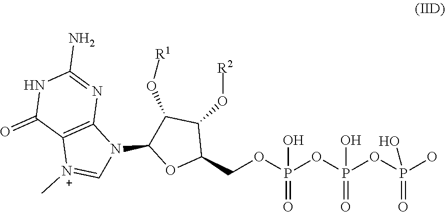

or a salt thereof, wherein one of R¹ or R² is a removable hydrophobic group and the other R¹ or R² is hydrogen or C₁₋₃ alkyl.

Embodiment 76 A 5′-capped mRNA prepared by removing the protecting group of a protected 5′-capped mRNA of embodiment 75.

Embodiment 77 An mRNA of Formula (IIE):

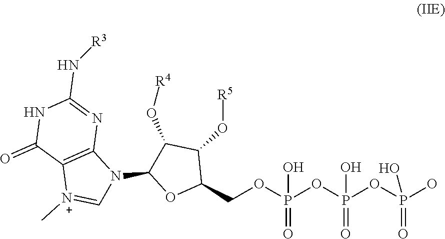

or a salt thereof, R³ is a removable hydrophobic group, R⁴ is hydrogen or C₁₋₃ alkyl and R⁵ is hydrogen or C₁₋₃ alkyl.

Embodiment 78 A 5′-capped mRNA prepared by removing the protecting group of a protected 5′-capped mRNA of embodiment 77.

Embodiment 79 An mRNA of Formula (IIF):

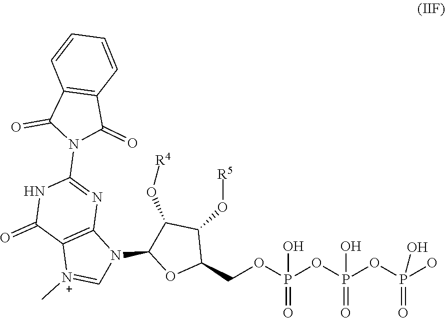

or a salt thereof, wherein R⁴ is hydrogen or C₁₋₃ alkyl and R⁵ is hydrogen or C₁₋₃ alkyl.

Embodiment 80 A 5′-capped mRNA prepared by removing the protecting group of a protected 5′-capped mRNA of embodiment 79.

Embodiment 81 An mRNA of Formula (IIG):

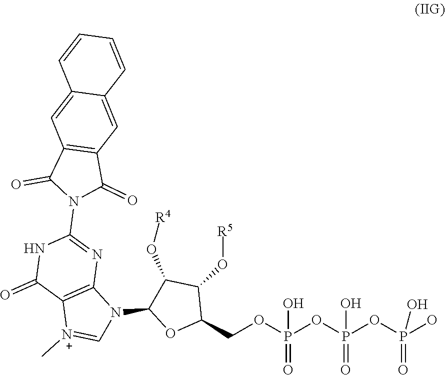

or a salt thereof, wherein R⁴ is hydrogen or C₁₋₃ alkyl and R⁵ is hydrogen or C₁₋₃ alkyl.

Embodiment 82 A 5′-capped mRNA prepared by removing the protecting group of a protected 5′-capped mRNA of embodiment 81.

Embodiment 83 An mRNA of Formula (IIH):

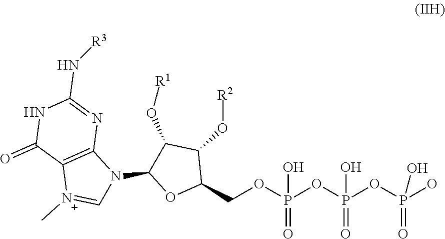

or a salt thereof, wherein one of R¹ or R² is a removable hydrophobic group and the other R¹ or R² is hydrogen or C₁₋₃ alkyl, and R³ is a removable hydrophobic group.

Embodiment 84 A 5′-capped mRNA prepared by removing the protecting group of a protected 5′-capped mRNA of embodiment 83.

Embodiment 85 A 5′-capped mRNA of any one of embodiments 76, 78, 80, 82 and 84 that is substantially free of 5′-phosphate-mRNA molecules.

Embodiment 86 A 5′-capped mRNA of any one of embodiments 76, 78, 80, 82 and 84 that is substantially free of enzymatic byproducts.

Embodiment 87 A composition comprising a chemically synthesized 5′-capped mRNA, wherein the composition comprises less than 1% by weight of uncapped 5′-phosphate-mRNA molecules.

Embodiment 88 A composition comprising a 5′-capped mRNA of embodiment 87, wherein the composition comprises less than 0.5% by weight of uncapped 5′-phosphate-mRNA molecules.

Embodiment 89 A composition comprising a 5′-capped mRNA of embodiment 87, wherein the composition comprises less than 0.25% by weight of uncapped 5′-phosphate-mRNA molecules.

Embodiment 90 A composition comprising a 5′-capped mRNA of embodiment 87, wherein the composition comprises less than 0.1% by weight of uncapped 5′-phosphate-mRNA molecules.

Embodiment 91 A composition comprising a 5′-capped mRNA of embodiment 87, wherein the composition comprises less than 0.05% by weight of uncapped 5′-phosphate-mRNA molecules.

Embodiment 92 A composition comprising a 5′-capped mRNA of embodiment 87, wherein the composition is substantially free of 5′-phosphate-mRNA molecules.

Embodiment 93 A composition of any one of embodiments 87-92, wherein the composition is substantially free of enzymatic byproducts.

Embodiment 94 A 5′-capped mRNA of any one of embodiments 76, 78, 80, 82 and 84, wherein the 5′-capped mRNA has a structure listed in Table 1.

Embodiment 95 A composition of any one of embodiments 68-74 and 87-93, wherein the 5′-capped mRNA has a structure listed in Table 1.

Embodiment 96 A composition of any one of embodiments 68-74 and 87-93, wherein the 5′-capped mRNA encodes an antimicrobial peptide.

Embodiment 97 The composition of embodiment 96, wherein 5′-capped mRNAs encode an antimicrobial peptide against antibiotic-resistant infections caused by methicillin-resistant Staphloococcus (MRSA).

Embodiment 98 A composition of any one of embodiments 68-74 and 87-93, wherein the 5′-capped mRNA encodes a hormone.

Embodiment 99 A method of treating an infection comprising administering to a subject in need thereof a composition of embodiment 96 or embodiment 97.

Embodiment 100 A process of preparing a 5′-capped mRNA comprising reacting a compound of any one of embodiments 1-64 with a 5′-phosphate-mRNA to form a protected 5′-capped mRNA, and deprotecting the protected 5′-capped mRNA.

Embodiment 101 The process of embodiment 100, wherein the 5′-phosphate-mRNA is prepared non-enzymatically.

Embodiment 102 The process of embodiment 101, wherein the 5′-phosphate-mRNA is prepared on a solid support.

Embodiment 103 The process pf any one of embodiments 100-102, wherein the reaction between the 5′-capped mRNA and the 5′-phosphate-mRNA is performed in an organic solvent.

Embodiment 104 The process of embodiment 103, wherein the organic solvent is DMSO, DMF, THF or methylene chloride.

Embodiment 105 The process of embodiment 104, wherein the organic solvent is DMSO.

Embodiment 106 The process of any one of embodiments 100-105, wherein the reaction is formed in the presence of a salt with a divalent cation.

Embodiment 107 The reaction of embodiment 106, wherein the divalent salt is CaCl₂, ZnCl₂, MgCl₂, or CuCl₂.

Embodiment 108 The process of any one of embodiments 100-107, wherein the reaction is performed in the presence of 1-methylimidazole.

1.-183. (canceled)

184. An oligonucleotide comprising 50-12000 nucleotides, whose 5′ end comprises a structure of formula
(I) 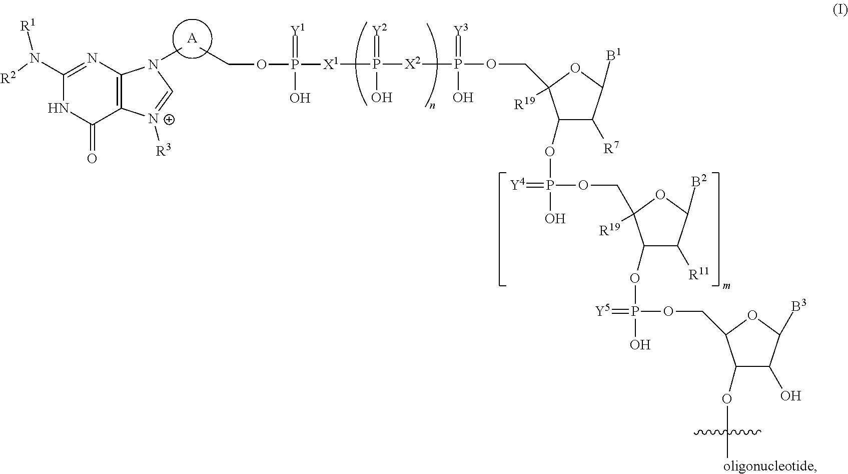
or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
B¹ and B³ are each independently a natural, modified, or unnatural nucleoside base;
each B² is independently a natural, modified, or unnatural nucleoside base;
Ring A is a substituted or unsubstituted cycloalkylene, substituted or unsubstituted heterocycloalkylene, substituted or unsubstituted arylene, or substituted or unsubstituted heteroarylene;
X¹ and X² are each independently —O—, —CH₂—, —CX₂—, —N(R¹⁰¹)—, —BH—, or —S—;
Y¹, Y², Y³, Y⁴, and Y⁵ are each independently O, S, or Se;
R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R⁷ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{7A}, —NR^{7A}R^{7B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁹ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{19A}, —NR^{19A}R^{19B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁷ and R¹⁹ together with the carbon atoms to which they are connected form a substituted or unsubstituted cycloalkylene or substituted or unsubstituted heterocycloalkylene;
each R¹¹ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{11A}, —NR^{11A}R^{11B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R¹¹ and R¹⁹ together with the carbon atoms to which they are connected form a substituted or unsubstituted cycloalkylene or substituted or unsubstituted heterocycloalkylene;
each R^{1A}, R^{2A}, R^{3A}, R^{7A}, R^{7B}, R^{11A}, R^{11B}, R^{19A}, and R^{19B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R^{7A} and R^{7B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
m is an integer from 0 to 8;
n is an integer from 0 to 3; and
each X is independently —Cl, —Br, —I or —F.

185. An oligonucleotide comprising 50-12000 nucleotides, whose 5′ end comprises a structure of formula (II): 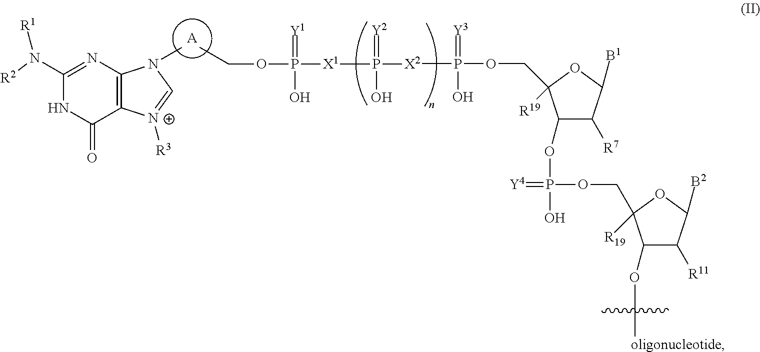
or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
B¹ and B² are each independently a natural, modified, or unnatural nucleoside base; Ring A is a substituted or unsubstituted cycloalkylene, substituted or unsubstituted heterocycloalkylene, substituted or unsubstituted arylene, or substituted or unsubstituted heteroarylene;
X¹ and X² are each independently —O—, —CH₂—, —CX₂—, —N(R¹⁰¹)—, —BH—, or —S—;
Y¹, Y², Y³, and Y⁴ are each independently O, S, or Se;
R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
R³ is hydrogen, —C(O)R^{3A}, C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{7A}, —NR^{7A}R^{7B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁹ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{19A}, —NR^{19A}R^{19B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R⁷ and R¹⁹ together with the carbon atoms to which they are connected form a substituted or unsubstituted cycloalkylene or substituted or unsubstituted heterocycloalkylene; each R¹¹ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{11A}, —NR^{11A}R^{11B}, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R¹¹ and R¹⁹ together with the carbon atoms to which they are connected form a substituted or unsubstituted cycloalkylene or substituted or unsubstituted heterocycloalkylene;
each R^{1A}, R^{2A}, R^{3A}, R^{7A}, R^{7B}, R^{11A}, R^{11B}, R^{19A}, and R^{19B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or R^{7A} and R^{7B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer from 0 to 3; and
each X is independently —Cl, —Br, —I or —F;
with the proviso that R¹¹ is not OH.

186. The oligonucleotide of claim 185, wherein the oligonucleotide comprises 3 or more modified nucleosides and 2 or more nucleotides that are linked together by a modified internucleotide linkage, at its 3′ end.

187. The oligonucleotide of claim 185, wherein the structure of formula (I) or formula (II) comprises one or more removable hydrophobic group(s).

188. The oligonucleotide of claim 185, wherein the structure of formula (I) or formula (II) comprises one or more non-removable hydrophobic group(s).

189. The oligonucleotide of claim 185, wherein Ring A is a substituted or unsubstituted heterocycloalkylene.

190. The oligonucleotide of claim 185, wherein R¹ is independently hydrogen, methyl, ethyl, propyl, isopropyl, butyl, isobutyl, t-butyl, pentyl, isopentyl, hexyl, phenyl, benzyl, 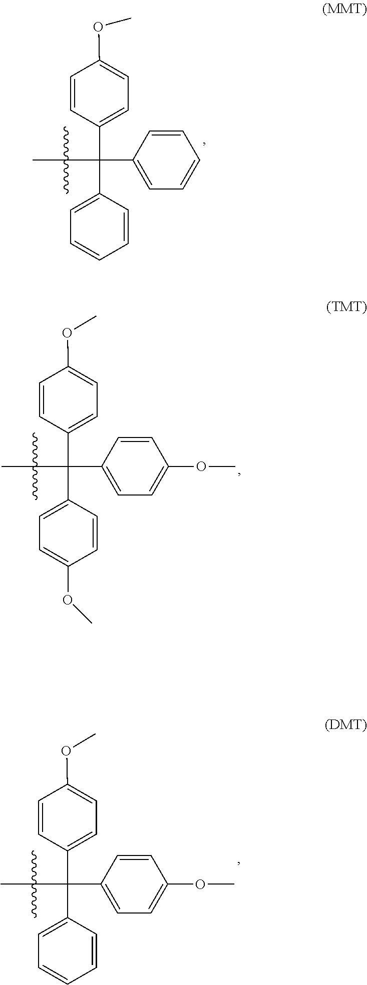 [SMILES: *C(c1ccccc1)(c1ccccc1)c1ccc(OC)cc1 ; *C(c1ccc(OC)cc1)(c1ccc(OC)cc1)c1ccc(OC)cc1 ; *C(c1ccccc1)(c1ccc(OC)cc1)c1ccc(OC)cc1]
or modified trityl.

191. The oligonucleotide of claim 185, wherein R² is independently hydrogen, methyl, ethyl, propyl, isopropyl, butyl, isobutyl, t-butyl, pentyl, isopentyl, hexyl, phenyl, benzyl, 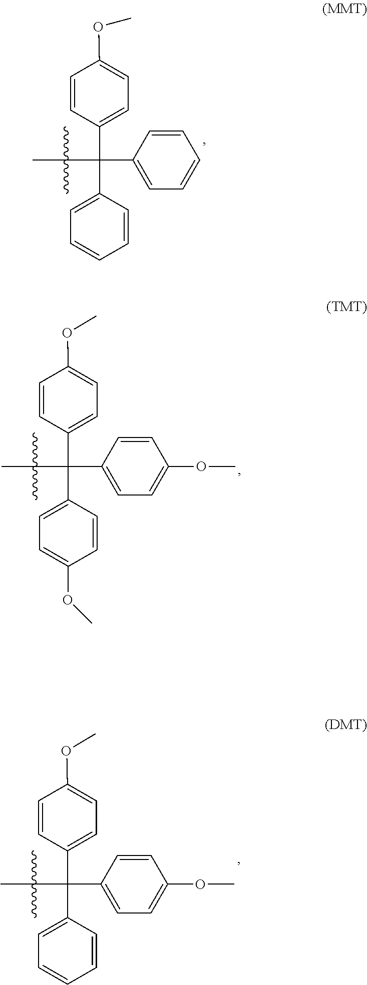 [SMILES: *C(c1ccccc1)(c1ccccc1)c1ccc(OC)cc1 ; *C(c1ccc(OC)cc1)(c1ccc(OC)cc1)c1ccc(OC)cc1 ; *C(c1ccccc1)(c1ccc(OC)cc1)c1ccc(OC)cc1]
or modified trityl and/or wherein R³ is independently hydrogen, methyl, ethyl, propyl, isopropyl, butyl, isobutyl, t-butyl, pentyl, isopentyl, hexyl, phenyl, benzyl, or 4-chlorobenzyl.

192. The oligonucleotide of claim 185, wherein R¹, R² and R³ are independently hydrogen, substituted or unsubstituted alkyl, or substituted or unsubstituted aryl.

193. The oligonucleotide of claim 185, wherein R⁷ is independently hydrogen, halogen, or —OR^{7A}.

194. The oligonucleotide of claim 185, wherein R⁷ and R¹¹ are independently hydrogen, hydroxy, methoxy, ethoxy, propoxy, butoxy, or t-butoxy.

195. The oligonucleotide of claim 185, wherein R¹⁹ is independently hydrogen and/or wherein R¹¹ is independently hydrogen, halogen, or —OR^{11A}.

196. The oligonucleotide of claim 185, wherein X¹ is —O—, —CH₂—, or —CX₂— and/or wherein X² is —O—, —CH₂—, —CX₂—, —N(R¹⁰¹)—, or —BH—.

197. The oligonucleotide of claim 185, wherein Y¹, Y², Y³, and Y⁴ are each independently O or S.

198. The oligonucleotide of claim 185, wherein n is 1 or 2.

199. An oligonucleotide whose 5′ end comprises a compound selected from the group consisting of: 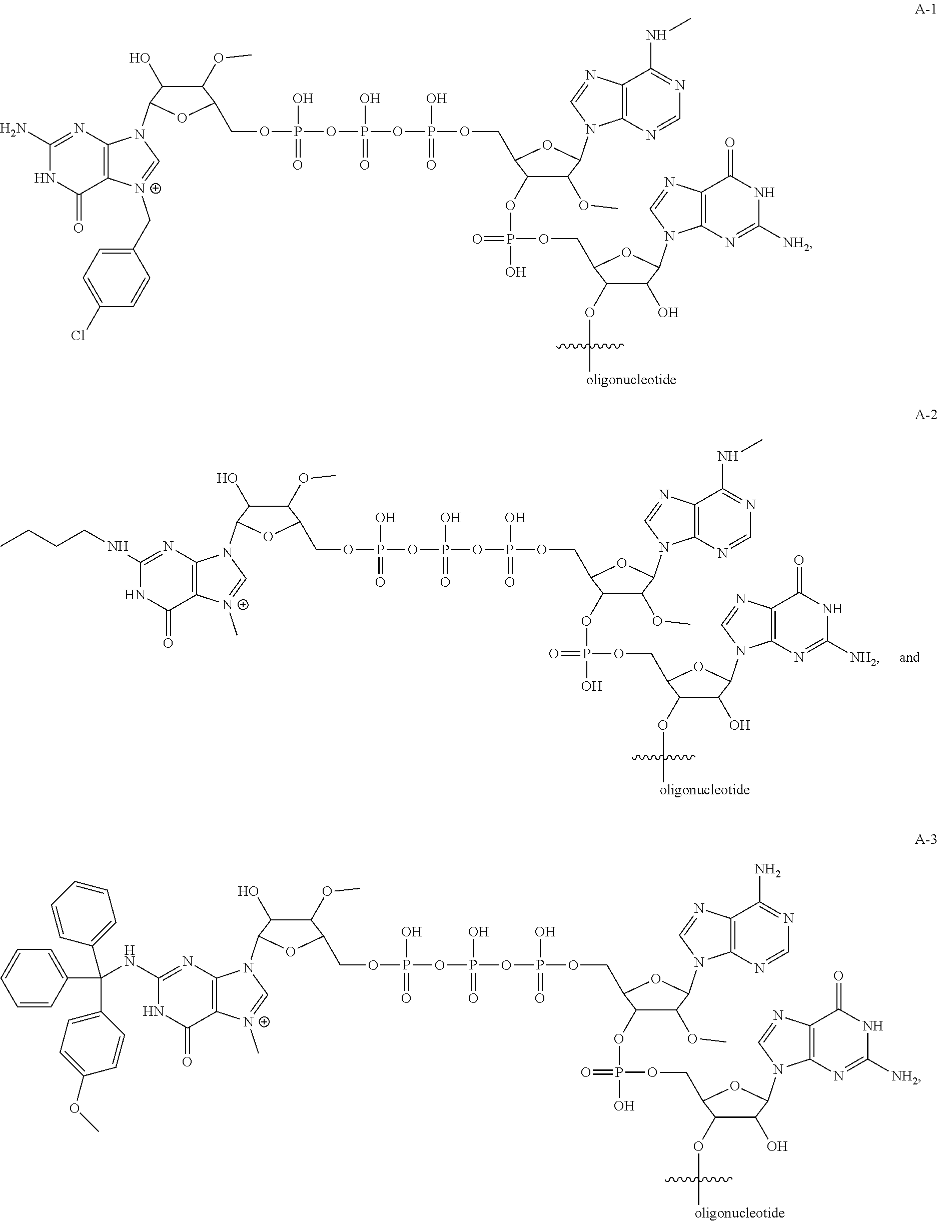
or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof.

200. The oligonucleotide of claim 185, wherein each non-removable hydrophobic group is a non-removable purification handle.

201. A 5′-capped oligonucleotide prepared by removing the protecting group(s) of the protected 5′-capped oligonucleotide of claim 185.

202. A composition comprising the chemically synthesized oligonucleotide of claim 185, wherein the composition comprises less than 1% by weight of oligonucleotide whose 5′ end does not comprise a structure of formula (I) or formula (II).

203. A process for preparing an oligonucleotide comprising 50-12000 nucleotides, whose 5′ end comprises a structure of formula (I): 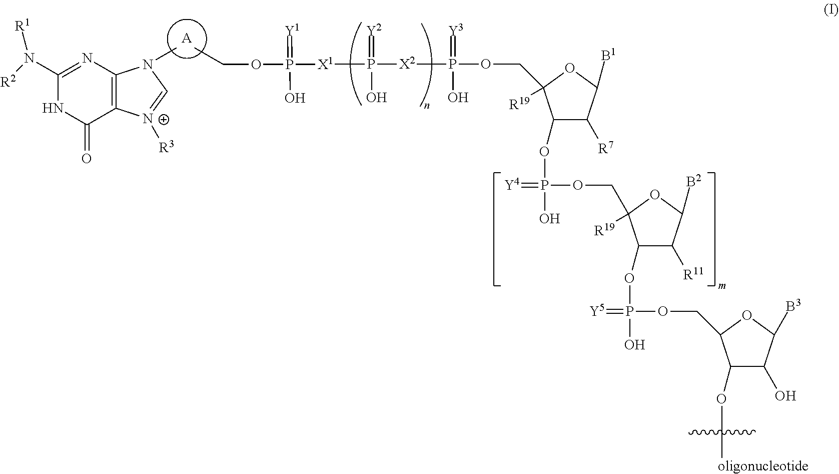
or formula (II): 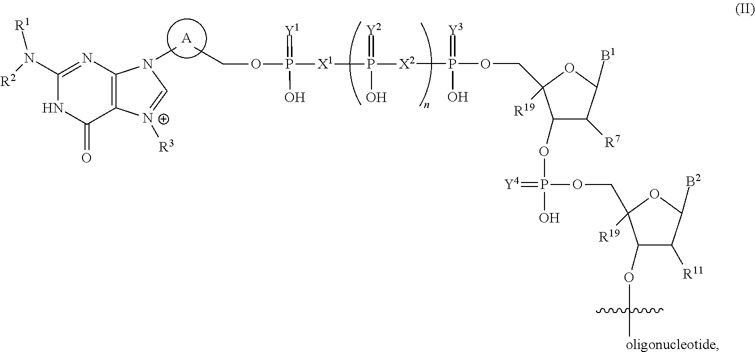
or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
comprising (a) reacting an imidazolide of formula (III) 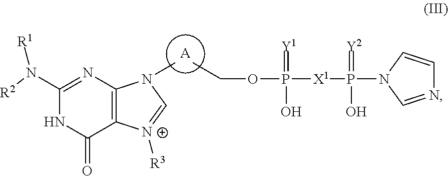
or an enantiomer, a mixture of enantiomers, a mixture of two or more diastereomers, a tautomer, a mixture of two or more tautomers, or an isotopic variant thereof; or a pharmaceutically acceptable salt, solvate, or hydrate thereof;
wherein:
Ring A is a substituted or unsubstituted cycloalkylene, substituted or unsubstituted heterocycloalkylene, substituted or unsubstituted arylene, or substituted or unsubstituted heteroarylene;
X¹ is independently —O—, —CH₂—, —CX₂—, —N(R¹⁰¹)—, —BH—, or —S—;
Y¹ and Y² are each independently O, S, or Se;
R¹ is independently hydrogen, —C(O)R^{1A}, —C(O)OR^{1A}, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, —C(O)R^{2A}, —C(O)OR^{2A}, —OR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R¹ and R² together with the nitrogen atom to which they are connected form a substituted or unsubstituted heteroaryl or a substituted or unsubstituted heterocyclyl;
R³ is hydrogen, —C(O)R^{3A}, —C(O)OR^{3A}, —OR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{1A}, R^{2A}, and R^{3A} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each X is independently —Cl, —Br, —I or —F;
with a 5′-phosphate-oligonucleotide 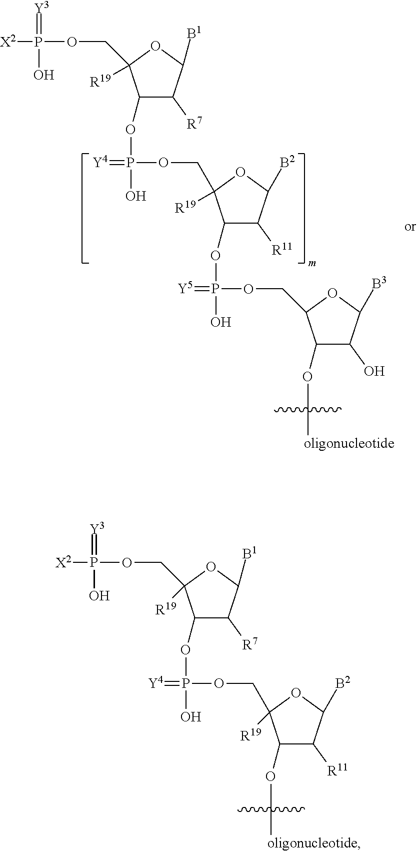
and (b) optionally removing the removable hydrophobic group(s).


SEQUENCE LISTING (9 sequences)

SEQ ID NO: 1 - RNA, 100 nt, synthetic construct
  source 1..100: mol_type = other RNA
ggcgcttctatctgattactctgagcgccatcaccagcgactatgtctagtggtaaagctccctcttcggagggagcatcagagaggaggacggcctggc

SEQ ID NO: 2 - RNA, 10 nt, synthetic construct
  source 1..10: mol_type = other RNA
ggcgcttcta

SEQ ID NO: 3 - RNA, 100 nt, synthetic construct
  source 1..100: mol_type = other RNA
  modified_base 1: mod_base = OTHER; note = 2'-O-methyladenosine
ngcgcttctatctgattactctgagcgccatcaccagcgactatgtctagtggtaaagctccctcttcggagggagcatcagagaggaggacggcctggc

SEQ ID NO: 4 - RNA, 90 nt, synthetic construct
  source 1..90: mol_type = other RNA
  modified_base 1: mod_base = OTHER; note = 2'-O-methyladenosine
ngtgattactctgagcgccatcaccagcgactatgtctagtggtaaagctccctcttcggagggagcatcagagaggaggacggcctggc

SEQ ID NO: 5 - RNA, 90 nt, synthetic construct
  source 1..90: mol_type = other RNA
  modified_base 1: mod_base = OTHER; note = 2'-O-methyladenosine
  modified_base 86: mod_base = cm
  modified_base 90: mod_base = cm
  modified_base 87: mod_base = um
  modified_base 88: mod_base = gm
  modified_base 89: mod_base = gm
  misc_feature 85..90: note = phosphorothioate linkage between the adjacent nucleotides at positions 85 through 90
ngtgattactctgagcgccatcaccagcgactatgtctagtggtaaagctccctcttcggagggagcatcagagaggaggacggcnnnnn

SEQ ID NO: 6 - RNA, 80 nt, synthetic construct
  source 1..80: mol_type = other RNA
  modified_base 1: mod_base = OTHER; note = 2'-O-methyladenosine
nggagcgccatcaccagcgactatgtctagtggtaaagctccctcttcggagggagcatcagagaggaggacggcctggc

SEQ ID NO: 7 - RNA, 80 nt, synthetic construct
  source 1..80: mol_type = other RNA
  modified_base 1: mod_base = OTHER; note = 2'-O-methyladenosine
  modified_base 76: mod_base = cm
  modified_base 77: mod_base = um
  modified_base 78: mod_base = gm
  modified_base 79: mod_base = gm
  modified_base 80: mod_base = cm
  misc_feature 75..80: note = phosphorothioate linkage between the adjacent nucleotides at positions 75 through 80
nggagcgccatcaccagcgactatgtctagtggtaaagctccctcttcggagggagcatcagagaggaggacggcnnnnn

SEQ ID NO: 8 - RNA, 70 nt, synthetic construct
  source 1..70: mol_type = other RNA
  modified_base 1: mod_base = OTHER; note = 2'-O-methyladenosine
ngaccagcgactatgtctagtggtaaagctccctcttcggagggagcatcagagaggaggacggcctggc

SEQ ID NO: 9 - RNA, 70 nt, synthetic construct
  source 1..70: mol_type = other RNA
  modified_base 1: mod_base = OTHER; note = 2'-O-methyladenosine
  modified_base 66: mod_base = cm
  modified_base 67: mod_base = um
  modified_base 68: mod_base = gm
  modified_base 69: mod_base = gm
  modified_base 70: mod_base = cm
  misc_feature 65..70: note = phosphorothioate linkage between the adjacent nucleotides at positions 65 through 70
ngaccagcgactatgtctagtggtaaagctccctcttcggagggagcatcagagaggaggacggcnnnnn